(19) United States
(12) Patent Application Publication
(10) Pub. No.: US 2023/0234992 A1
(43) Pub. Date: Jul. 27, 2023

(54) MODIFIED BETACORONAVIRUS SPIKE PROTEINS

(71) Applicant: GLAXOSMITHKLINE BIOLOGICALS SA, Rixensart (BE)

(72) Inventors: Marco BIANCUCCI, Rockville, MD (US); Joel David KARPIAK, Collegeville, PA (US); Jason Paul LALIBERTE, Rockville, MD (US); Anna Ulrika LOWEGARD, Stevenage, Hertfordshire (GB); Enrico MALITO, Rockville, MD (US); Newton Muchugu WAHOME, Rockville, MD (US)

(73) Assignee: GLAXOSMITHKLINE BIOLOGICALS SA, Rixensart (BE)

(21) Appl. No.: 18/007,931

(22) PCT Filed: Jun. 4, 2021
(86) PCT No.: PCT/IB2021/054903

§ 371 Date: Dec. 2, 2022

Related U.S. Application Data
(60) Provisional application No. 63/035,319, filed on Jun. 5, 2020.

(51) Int. Cl. C07K 14/165; C12N 15/11; A61K 39/215; A61P 31/14

(52) U.S. Cl. CPC C07K 14/165; C12N 15/11; A61K 39/215; A61P 31/14; A61K 2039/575

(57) ABSTRACT

Betacoronavirus Spike proteins, or fragments thereof, including substitution mutations designed to increase stability, decrease the risk of antibody dependent enhancement, or both; and that are useful in, for example, immunogenic compositions.

### CROSS-REFERENCE TO RELATED APPLICATION

This application is related to and claims priority to U.S. Provisional Application No. 63/035,319 filed on Jun. 5, 2020, the entire contents of which is hereby incorporated by reference.

### SEQUENCE LISTING

The instant application contains an electronically submitted Sequence Listing in ASCII text file format (Name: 2021-06-02 2801-0358PWO1_ST25.txt; Size 1.23 MB; created Jun. 2, 2021) which is hereby incorporated by reference in its entirety.

### BACKGROUND

Coronaviruses are spherical and enveloped, positive-sense single-stranded RNA viruses. They have the largest genomes (26-32 kb) among known RNA viruses, and are phylogenetically divided into four genera (alpha, beta, gamma, delta), with betacoronaviruses further subdivided into four lineages (A, B, C, D). Coronaviruses infect a wide range of avian and mammalian species, including humans. Of the seven known coronaviruses to emerge in the human population, four of them (HCoV-OC43 (betacoronavirus), HCoV-229E (alphacoronavirus), HCoV-HKU1 (betacoronavirus) and HCoV-NL63 (alphacoronavirus)) are known to circulate annually in humans and generally cause mild upper respiratory diseases in immunocompetent hosts, although severe infections can be caused in infants, young children, elderly individuals, and the immunocompromised. Both HCoV-OC43 and HCoV-HKU1 cause self-limiting, common cold-like illnesses. Wang et al. 2020 Cell 181: 894-904. In contrast, the Middle East respiratory syndrome coronavirus (MERS-CoV) and the severe acute respiratory syndrome coronavirus 1 (SARS-CoV-1), belonging to betacoronavirus lineages C and B, respectively, are highly pathogenic. Cui et al. 2019 Nat. Rev. Microbiol. 17(3):181-192. Recent work on prefusion coronavirus spike proteins and their use is reported in WO 2018/081318. This publication discusses, in particular, recombinant coronavirus spike (S) proteins, such as Middle East respiratory syndrome (MERS-CoV) and severe acute respiratory coronavirus (SARS-CoV) S proteins, that are stabilized in a prefusion conformation by one or more amino acid substitutions. For example, it is reported in Carnell et al. 2021 doi.org/10.1101/2021.01.14.426695 and Xiong et al. 2020 Nat Struct Mol Biol 27(10):934-941 that two cysteine residues can be introduced that form a disulfide bond that constrains the trimer in a closed state, which results in improvement of trimer stability.

It is unclear whether the latest betacoronavirus to emerge in the human population, severe acute respiratory syndrome coronavirus 2 (SARS-CoV-2), also of lineage B, will circulate annually in humans. What is unfortunately clear, is that SARS-CoV-2, like MERS-CoV and SARS-CoV-1, is highly pathogenic. MERS-CoV, SARS-CoV-1, and SARS-CoV-2 all crossed the species barrier into humans and caused outbreaks of severe, often fatal, respiratory diseases: MERS-CoV in about 2012, SARS-CoV-1 in about 2002/2003, and SARS-CoV-2 in about 2019/2020. See Letko et al. 2020 Nat. Microbio. 5: 562-569.

The high fatality rate and absence of prophylactic or therapeutic measures against betacoronaviruses have created an urgent need for an effective treatment or prevention of betacoronavirus infections and the disease(s) such infections cause. In the context of vaccination, this is a need to provide a betacoronavirus antigen that may be delivered to the body for presentation to the immune system.

### SUMMARY OF THE INVENTION

The present inventors provide modified betacoronavirus antigens, specifically modified Spike (S) proteins or S protein fragments, that include one or more substitution mutations designed to increase stability or decrease the risk of antibody dependent enhancement; features desirable of a candidate betacoronavirus vaccine antigen.

Certain embodiments provide a betacoronavirus Spike (S) protein, or fragment thereof, comprising an amino acid sequence that has amino acid substitutions, wherein said amino acid substitutions are selected from those listed in one of columns #4-13 in Table 1. Certain further embodiments provide a betacoronavirus Spike (S) protein, or fragment thereof, comprising an amino acid sequence that has at least 80% sequence identity to the entire sequence of at least one of SEQ ID NOs: 5-14.

Certain embodiments provide a betacoronavirus Spike (S) protein, or fragment thereof, comprising an amino acid sequence that has amino acid substitutions, wherein said amino acid substitutions are selected from those listed in one of columns #4-18 in Table 2. Certain further embodiments provide a betacoronavirus Spike (S) protein, or fragment thereof, comprising an amino acid sequence that has at least 80% sequence identity to the entire sequence of at least one of SEQ ID NOs: 15-29.

Certain embodiments provide a betacoronavirus Spike (S) protein, or fragment thereof, comprising an amino acid sequence that has amino acid substitutions, wherein said amino acid substitutions are selected from those listed in one of columns #4-8 in Table 3. Certain further embodiments provide a betacoronavirus Spike (S) protein, or fragment thereof, comprising an amino acid sequence that has at least 80% sequence identity to the entire sequence of at least one of SEQ ID NOs: 30-34.

Certain embodiments provide a betacoronavirus Spike (S) protein, or fragment thereof, comprising an amino acid sequence that has disulfide bridge mutations, for example:

Cysteines at the positions that correspond to residues 744 and 989 of the sequence SEQ ID NO: 3,

Cysteines at the positions that correspond to residues 813 and 836 of the sequence SEQ ID NO: 3,

Cysteines at the positions that correspond to residues 544 and 941 of the sequence SEQ ID NO: 3,

Cysteines at the positions that correspond to residues 824 and 560 of the sequence SEQ ID NO: 3,

Cysteines at the positions that correspond to residues 387 and 961 of the sequence SEQ ID NO: 3,

Cysteines at the positions that correspond to residues 357 and 959 of the sequence SEQ ID NO: 3,

Cysteines at the positions that correspond to residues 356 and 957 of the sequence SEQ ID NO: 3,

Cysteines at the positions that correspond to residues 15 and 494 of the sequence SEQ ID NO: 3,

Cysteines at the positions that correspond to residues 496 and 518 of the sequence SEQ ID NO: 3, or

Cysteines at the positions that correspond to residues 495 and 538 of the sequence SEQ ID NO: 3.

Certain further embodiments provide a betacoronavirus Spike (S) protein, or fragment thereof, comprising an amino acid sequence that has at least 80% sequence identity to the entire sequence of at least one of SEQ ID NOs: 35-64. Certain embodiments provide a betacoronavirus Spike (S) protein, or a fragment thereof, comprising an amino acid sequence that has amino acid substitutions, wherein the amino acid substitutions:

do not consist of Cysteines at the positions that correspond to residues 357 and 959 of the sequence SEQ ID NO: 3,

do not consist of Cysteines at the positions that correspond to residues 359 and 385 of the sequence SEQ ID NO: 3,

do not consist of Cysteines at the positions that correspond to residues 387 and 961 of the sequence SEQ ID NO: 3, and/or

do not consist of Cysteines at the positions that correspond to residues 643 and 840 of the sequence SEQ ID NO: 3.

Certain embodiments provide a betacoronavirus Spike (S) protein, or fragment thereof, comprising an amino acid sequence that has one or more receptor binding mutation, for example:

F, L, M, W, or Y at the position that corresponds to residue 391 of the sequence SEQ ID NO: 3;

A at the position that corresponds to residue 423 of the sequence SEQ ID NO: 3;

A at the position that corresponds to residue 427 of the sequence SEQ ID NO: 3;

A, H, M, N, or W at the position that corresponds to residue 429 of the sequence SEQ ID NO: 3;

H, I, W, or Y at the position that corresponds to residue 430 of the sequence SEQ ID NO: 3;

W at the position that corresponds to residue 447 of the sequence SEQ ID NO: 3;

M at the position that corresponds to residue 449 of the sequence SEQ ID NO: 3;

T at the position that corresponds to residue 450 of the sequence SEQ ID NO: 3;

H, I, L, M, N, P, T, W, or Y at the position that corresponds to residue 460 of the sequence SEQ ID NO: 3;

F, L, M, or Q at the position that corresponds to residue 461 of the sequence SEQ ID NO: 3; or

A, Y, F, R, M, C, G, or V at the position that corresponds to residue 467 of the sequence SEQ ID NO: 3.

Certain further embodiments provide a betacoronavirus Spike (S) protein, or fragment thereof, comprising an amino acid sequence that has at least 80% sequence identity to the entire sequence of at least one of SEQ ID NOs: 65-104.

Certain embodiments provide a betacoronavirus Spike (S) protein, or fragment thereof, comprising an amino acid sequence that has one or more glycan mutation, for example:

N at the position that corresponds to residue 391 of the sequence SEQ ID NO: 3 and T at the position that corresponds to residue 393 of the sequence SEQ ID NO: 3;

N at the position that corresponds to residue 423 of the sequence SEQ ID NO: 3 and T at the position that corresponds to residue 425 of the sequence SEQ ID NO: 3;

N at the position that corresponds to residue 427 of the sequence SEQ ID NO: 3 and T at the position that corresponds to residue 429 of the sequence SEQ ID NO: 3;

N at the position that corresponds to residue 429 of the sequence SEQ ID NO: 3 and T at the position that corresponds to residue 431 of the sequence SEQ ID NO: 3;

N at the position that corresponds to residue 430 of the sequence SEQ ID NO: 3 and T at the position that corresponds to residue 432 of the sequence SEQ ID NO: 3;

N at the position that corresponds to residue 447 of the sequence SEQ ID NO: 3 and T at the position that corresponds to residue 449 of the sequence SEQ ID NO: 3;

N at the position that corresponds to residue 449 of the sequence SEQ ID NO: 3 and T at the position that corresponds to residue 451 of the sequence SEQ ID NO: 3;

N at the position that corresponds to residue 450 of the sequence SEQ ID NO: 3;

T at the position that corresponds to residue 463 of the sequence SEQ ID NO: 3; or

N at the position that corresponds to residue 467 of the sequence SEQ ID NO: 3 and T at the position that corresponds to residue 469 of the sequence SEQ ID NO: 3.

Certain further embodiments provide a betacoronavirus Spike (S) protein, or fragment thereof, comprising an amino acid sequence that has at least 80% sequence identity to the entire sequence of at least one of SEQ ID NOs: 105-114.

Certain further embodiments provide a betacoronavirus Spike (S) protein, or fragment thereof, comprising an amino acid sequence that has at least 80% sequence identity to the entire sequence of at least one of SEQ ID NOs: 5-114.

Certain embodiments provide a betacoronavirus Spike (S) protein, or a fragment thereof, comprising an amino acid sequence that has amino acid substitutions, wherein the amino acid substitutions:

do not consist of a Leucine at the position corresponding to residue 544 of the sequence SEQ ID NO: 3, an Isoleucine at the position corresponding to residue 546 of the sequence SEQ ID NO: 3, a Tyrosine at the position corresponding to residue 829 of the sequence SEQ ID NO: 3, and an Isoleucine at the position corresponding to residue 830 of the sequence SEQ ID NO: 3;

do not consist of a Leucine at the position corresponding to residue 372 of the sequence SEQ ID NO: 3, Leucine at the position corresponding to residue 488 of the sequence SEQ ID NO: 3, and Leucine at the position corresponding to residue 490 of the sequence SEQ ID NO: 3; and/or

do not consist of Isoleucine at the position corresponding to residue 480 of the sequence SEQ ID NO: 3 and Leucine at the position corresponding to residue 544 of the sequence SEQ ID NO: 3.

In certain embodiments, the betacoronavirus Spike (S) protein, or fragment thereof, is a lineage B or C betacoronavirus Spike (S) protein, or fragment thereof (such as MERS-CoV, SARS-CoV1, SARS-CoV2). Certain further embodiments provide a lineage B betacoronavirus Spike (S) protein, or fragment thereof (such as SARS-CoV1, SARS-CoV2). Certain other embodiments provide a MERS-CoV, SARS-CoV1, or SARS-CoV2 Spike (S) protein, or fragment thereof. Certain other embodiments provide a SARS-CoV1 or SARS-CoV2 Spike (S) protein, or fragment thereof. Certain other embodiments provide a SARS-CoV2 Spike (S) protein, or fragment thereof.

In certain embodiments, the modified betacoronavirus S protein or S protein fragment comprises a transmembrane domain (such as a Full Length or CT-Deleted betacoronavirus S protein). In certain further embodiments, the S protein fragment is the Receptor Binding Domain. Certain other embodiments provide a non-human host cell or cell culture comprising the modified betacoronavirus S protein or S protein fragment.

In certain embodiments, the betacoronavirus S protein or S protein fragment, or a polynucleotide encoding the betacoronavirus S protein or S protein fragment, is operably linked to a nanoparticle. In certain further embodiments the S protein fragment is the Receptor Binding Domain.

In certain embodiments, is provided a nucleic acid molecule comprising a polynucleotide sequence that encodes the betacoronavirus S protein, or S protein fragment. In certain embodiments, the nucleic acid molecule is a Self-Amplifying RNA Molecule. In certain further embodiments, the Self-Amplifying RNA Molecule comprises, from 5′-3′, a polynucleotide comprising the sequence SEQ ID NO: 119; a polynucleotide sequence that encodes the betacoronavirus S protein, or S protein fragment; and a polynucleotide comprising the sequence SEQ ID NO: 120. In certain embodiments, the polynucleotide encodes a betacoronavirus S protein or S protein fragment that comprises a transmembrane domain (such as a Full Length or CT-Deleted betacoronavirus S protein). In certain further embodiments, the S protein fragment is the Receptor Binding Domain. Certain other embodiments provide a non-human host cell, cell culture, or vector (e.g., recombinant vector) comprising the nucleic acid molecule.

Certain embodiments provide an immunogenic composition comprising (i) the betacoronavirus S protein, or S protein fragment, optionally further comprising an adjuvant; or (ii) a nucleic acid molecule comprising a polynucleotide sequence that encodes the betacoronavirus S protein, or S protein fragment. In certain embodiments, the immunogenic composition comprises a carrier (e.g., a nanoparticle). In certain embodiments, the immunogenic composition is for use in inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases. Certain embodiments provide use of the immunogenic composition for inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases. Certain embodiments provide use of the immunogenic composition for the manufacture of a medicament for inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases.

Certain embodiments provide a method of inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases; comprising: delivering to a subject an immunologically effective amount of the immunogenic composition. In certain embodiments, delivering comprises administering to a human subject an immunologically effective amount of an immunogenic composition that comprises a modified betacoronavirus S protein, or S protein fragment. In certain embodiments, delivering comprises administering to a human subject an immunologically effective amount of an immunogenic composition that comprises a nucleic acid molecule comprising a polynucleotide sequence that encodes a modified betacoronavirus S protein, or S protein fragment.

In certain further embodiments, the immunogenic composition further comprises an adjuvant.

Certain embodiments provide a method of making a modified betacoronavirus Spike (S) protein, or S protein fragment, comprising: culturing, under suitable conditions, a non-human host cell that comprises a nucleic acid molecule that encodes the modified betacoronavirus Spike (S) protein or S protein fragment. In certain further embodiments, the modified betacoronavirus S protein or S protein fragment is purified from the non-human host cells or culture media.

In another embodiment, the present invention is directed to a betacoronavirus Spike (S) protein, or a fragment thereof, according to any of the above or below embodiments of the invention, wherein the betacoronavirus Spike (S) protein, or a fragment thereof has one or more of the following characteristics: the mammalian cellular expression of said protein or fragment is greater than 5 fold of that of SEQ ID NO: 4; the ACE2 Receptor binding of said protein or fragment is less than the ACE2 Receptor binding to that of SEQ ID NO:4; the binding of neutralizing antibodies to said protein or fragment is greater than the binding of neutralizing antibodies to that of SEQ ID NO:4, and/or the thermostability of said protein or fragment is greater than that of SEQ ID NO:4.

In another embodiment, the present invention also relates modified betacoronavirus antigens that are based on the mutant strain B.1.351 strain (20H/501Y.V2, a South African strain, Madhi et al. 2021 N Engl J Med 384: 1885-1898, Cele et al. 2021 medRxiv doi.org/10.1101/2021.01.26.21250224, www.beiresources.org/Catalog/animalviruses/NR-54009.aspx), where the Wuhan wild-type S protein sequence (SEQ ID NO: 2) was mutated with the D215G, K417N, E484K, N501Y, D614G mutations, specifically modified Spike (S) proteins or S protein fragments, that include one or more substitution mutations designed to increase stability or decrease the risk of antibody dependent enhancement; features desirable of a candidate betacoronavirus vaccine antigen. The D215G, K417N, E484K, N501Y, D614G mutation in the mutant strain B.1.351 strain corresponds to the D202G, K404N, E471K, N488Y, D601G mutations, respectively, shown in SEQ ID NOs:125-134 (in bold type and underlined). These modified betacorona virus antigens are identified as SEQ ID NOs:125-134. Thus, as to the antigens that are based on the mutant strain B.1.351 strain (20H/501Y.V2), the features of the invention also apply to these modified betacoronavirus antigens that are based on the mutant strain B.1.351 strain. For example, in the above description, where a sequence identify of at a specific % or at least a specific % to the entire sequence of a specified sequence or sequences is discussed, those same sequence identity requirements would apply to a comparison with the same specified sequence or sequences, alternatively, the corresponding part of the sequence of mutant strain B.1.351. To the extent that other descriptions of modified betacoronavirus antigens (including preparation thereof, formulations thereof, uses thereof and the like) are not inconsistent, all descriptions of this embodiment of invention (the embodiment based on the mutant strain B.1.351 strain and exemplified by SEQ ID NOs:125-134) apply to modified betacoronavirus antigens based on mutant strain B.1.351 strain.

Other embodiments of the invention include the following:

1. A betacoronavirus Spike (S) protein, or fragment thereof, comprising an amino acid sequence that has amino acid substitutions, wherein said amino acid substitutions are selected from:

the substitute amino acids listed throughout rows 3-134 of column #4 in Table 1, wherein each substitute amino acid is located at the position that corresponds to the residue number of SEQ ID NO: 3 that is listed in the same row of column #1 in Table 1;

the substitute amino acids listed throughout rows 3-134 of column #5 in Table 1, wherein each substitute amino acid is located at the position that corresponds to the residue number of SEQ ID NO: 3 that is listed in the same row of column #1 in Table 1;

the substitute amino acids listed throughout rows 3-134 of column #6 in Table 1, wherein each substitute amino acid is located at the position that corresponds to the residue number of SEQ ID NO: 3 that is listed in the same row of column #1 in Table 1;

the substitute amino acids listed throughout rows 3-134 of column #7 in Table 1, wherein each substitute amino acid is located at the position that corresponds to the residue number of SEQ ID NO: 3 that is listed in the same row of column #1 in Table 1;

the substitute amino acids listed throughout rows 3-134 of column #8 in Table 1, wherein each substitute amino acid is located at the position that corresponds to the residue number of SEQ ID NO: 3 that is listed in the same row of column #1 in Table 1;

the substitute amino acids listed throughout rows 3-134 of column #9 in Table 1, wherein each substitute amino acid is located at the position that corresponds to the residue number of SEQ ID NO: 3 that is listed in the same row of column #1 in Table 1;

the substitute amino acids listed throughout rows 3-134 of column #10 in Table 1, wherein each substitute amino acid is located at the position that corresponds to the residue number of SEQ ID NO: 3 that is listed in the same row of column #1 in Table 1;

the substitute amino acids listed throughout rows 3-134 of column #11 in Table 1, wherein each substitute amino acid is located at the position that corresponds to the residue number of SEQ ID NO: 3 that is listed in the same row of column #1 in Table 1;

the substitute amino acids listed throughout rows 3-134 of column #12 in Table 1, wherein each substitute amino acid is located at the position that corresponds to the residue number of SEQ ID NO: 3 that is listed in the same row of column #1 in Table 1; or

the substitute amino acids listed throughout rows 3-134 of column #13 in Table 1, wherein each substitute amino acid is located at the position that corresponds to the residue number of SEQ ID NO: 3 that is listed in the same row of column #1 in Table 1.

2. The betacoronavirus Spike (S) protein, or fragment thereof, of embodiment 1 comprising:

an amino acid sequence that has the substitutions of (a) and has at least 80% sequence identity to the entire sequence SEQ ID NO: 5,

an amino acid sequence that has the substitutions of (b) and has at least 80% sequence identity to the entire sequence SEQ ID NO: 6,

an amino acid sequence that has the substitutions of (c) and has at least 80% sequence identity to the entire sequence SEQ ID NO: 7,

an amino acid sequence that has the substitutions of (d) and has at least 80% sequence identity to the entire sequence SEQ ID NO: 8,

an amino acid sequence that has the substitutions of (e) and has at least 80% sequence identity to the entire sequence SEQ ID NO: 9,

an amino acid sequence that has the substitutions of (f) and has at least 80% sequence identity to the entire sequence SEQ ID NO: 10,

an amino acid sequence that has the substitutions of (g) and has at least 80% sequence identity to the entire sequence SEQ ID NO: 11,

an amino acid sequence that has the substitutions of (h) and has at least 80% sequence identity to the entire sequence SEQ ID NO: 12,

an amino acid sequence that has the substitutions of (i) and has at least 80% sequence identity to the entire sequence SEQ ID NO: 13, or

an amino acid sequence that has the substitutions of (j) and has at least 80% sequence identity to the entire sequence SEQ ID NO: 14.

3. A betacoronavirus Spike (S) protein, or fragment thereof, comprising an amino acid sequence that has amino acid substitutions, wherein said amino acid substitutions are selected from:

the substitute amino acids listed throughout rows 3-145 of column #4 in Table 2, wherein each substitute amino acid is located at the position that corresponds to the residue number of SEQ ID NO: 3 that is listed in the same row of column #1 in Table 2;

the substitute amino acids listed throughout rows 3-145 of column #5 in Table 2, wherein each substitute amino acid is located at the position that corresponds to the residue number of SEQ ID NO: 3 that is listed in the same row of column #1 in Table 2;

the substitute amino acids listed throughout rows 3-145 of column #6 in Table 2, wherein each substitute amino acid is located at the position that corresponds to the residue number of SEQ ID NO: 3 that is listed in the same row of column #1 in Table 2;

the substitute amino acids listed throughout rows 3-145 of column #7 in Table 2, wherein each substitute amino acid is located at the position that corresponds to the residue number of SEQ ID NO: 3 that is listed in the same row of column #1 in Table 2;

the substitute amino acids listed throughout rows 3-145 of column #8 in Table 2, wherein each substitute amino acid is located at the position that corresponds to the residue number of SEQ ID NO: 3 that is listed in the same row of column #1 in Table 2;

the substitute amino acids listed throughout rows 3-145 of column #9 in Table 2, wherein each substitute amino acid is located at the position that corresponds to the residue number of SEQ ID NO: 3 that is listed in the same row of column #1 in Table 2;

the substitute amino acids listed throughout rows 3-145 of column #10 in Table 2, wherein each substitute amino acid is located at the position that corresponds to the residue number of SEQ ID NO: 3 that is listed in the same row of column #1 in Table 2;

the substitute amino acids listed throughout rows 3-145 of column #11 in Table 2, wherein each substitute amino acid is located at the position that corresponds to the residue number of SEQ ID NO: 3 that is listed in the same row of column #1 in Table 2;

the substitute amino acids listed throughout rows 3-145 of column #12 in Table 2, wherein each substitute amino acid is located at the position that corresponds to the residue number of SEQ ID NO: 3 that is listed in the same row of column #1 in Table 2;

the substitute amino acids listed throughout rows 3-145 of column #13 in Table 2, wherein each substitute amino acid is located at the position that corresponds to the residue number of SEQ ID NO: 3 that is listed in the same row of column #1 in Table 2;

the substitute amino acids listed throughout rows 3-145 of column #14 in Table 2, wherein each substitute amino acid is located at the position that corresponds to the residue number of SEQ ID NO: 3 that is listed in the same row of column #1 in Table 2;

the substitute amino acids listed throughout rows 3-145 of column #15 in Table 2, wherein each substitute amino acid is located at the position that corresponds to the residue number of SEQ ID NO: 3 that is listed in the same row of column #1 in Table 2;

the substitute amino acids listed throughout rows 3-145 of column #16 in Table 2, wherein each substitute amino acid is located at the position that corresponds to the residue number of SEQ ID NO: 3 that is listed in the same row of column #1 in Table 2;

the substitute amino acids listed throughout rows 3-145 of column #17 in Table 2, wherein each substitute amino acid is located at the position that corresponds to the residue number of SEQ ID NO: 3 that is listed in the same row of column #1 in Table 2; or

the substitute amino acids listed throughout rows 3-145 of column #18 in Table 2, wherein each substitute amino acid is located at the position that corresponds to the residue number of SEQ ID NO: 3 that is listed in the same row of column #1 in Table 2.

4. The betacoronavirus Spike (S) protein, or fragment thereof, of embodiment 3 comprising:

an amino acid sequence that has the substitutions of (k) and has at least 80% sequence identity to the entire sequence SEQ ID NO: 15,

an amino acid sequence that has the substitutions of (l) and has at least 80% sequence identity to the entire sequence SEQ ID NO: 16,

an amino acid sequence that has the substitutions of (m) and has at least 80% sequence identity to the entire sequence SEQ ID NO: 17,

an amino acid sequence that has the substitutions of (n) and has at least 80% sequence identity to the entire sequence SEQ ID NO: 18,

an amino acid sequence that has the substitutions of (o) and has at least 80% sequence identity to the entire sequence SEQ ID NO: 19,

an amino acid sequence that has the substitutions of (p) and has at least 80% sequence identity to the entire sequence SEQ ID NO: 20,

an amino acid sequence that has the substitutions of (q) and has at least 80% sequence identity to the entire sequence SEQ ID NO: 21,

an amino acid sequence that has the substitutions of (r) and has at least 80% sequence identity to the entire sequence SEQ ID NO: 22,

an amino acid sequence that has the substitutions of (s) and has at least 80% sequence identity to the entire sequence SEQ ID NO: 23,

an amino acid sequence that has the substitutions of (t) and has at least 80% sequence identity to the entire sequence SEQ ID NO: 24,

an amino acid sequence that has the substitutions of (u) and has at least 80% sequence identity to the entire sequence SEQ ID NO: 25,

an amino acid sequence that has the substitutions of (v) and has at least 80% sequence identity to the entire sequence SEQ ID NO: 26,

an amino acid sequence that has the substitutions of (w) and has at least 80% sequence identity to the entire sequence SEQ ID NO: 27,

an amino acid sequence that has the substitutions of (x) and has at least 80% sequence identity to the entire sequence SEQ ID NO: 28, or

an amino acid sequence that has the substitutions of (y) and has at least 80% sequence identity to the entire sequence SEQ ID NO: 29.

5. A betacoronavirus Spike (S) protein, or fragment thereof, comprising an amino acid sequence that has amino acid substitutions, wherein said amino acid substitutions are selected from:

the substitute amino acids listed throughout rows 3-34 of column #4 in Table 3, wherein each substitute amino acid is located at the position that corresponds to the residue number of SEQ ID NO: 3 that is listed in the same row of column #1 in Table 3;

the substitute amino acids listed throughout rows 3-34 of column #5 in Table 3, wherein each substitute amino acid is located at the position that corresponds to the residue number of SEQ ID NO: 3 that is listed in the same row of column #1 in Table 3;

the substitute amino acids listed throughout rows 3-34 of column #6 in Table 3, wherein each substitute amino acid is located at the position that corresponds to the residue number of SEQ ID NO: 3 that is listed in the same row of column #1 in Table 3;

the substitute amino acids listed throughout rows 3-34 of column #7 in Table 3, wherein each substitute amino acid is located at the position that corresponds to the residue number of SEQ ID NO: 3 that is listed in the same row of column #1 in Table 3; or

the substitute amino acids listed throughout rows 3-34 of column #8 in Table 3, wherein each substitute amino acid is located at the position that corresponds to the residue number of SEQ ID NO: 3 that is listed in the same row of column #1 in Table 3.

6. The betacoronavirus Spike (S) protein, or fragment thereof, of embodiment 5 comprising:

an amino acid sequence that has the substitutions of (I) and has at least 80% sequence identity to the entire sequence SEQ ID NO: 30,

an amino acid sequence that has the substitutions of (II) and has at least 80% sequence identity to the entire sequence SEQ ID NO: 31,

an amino acid sequence that has the substitutions of (III) and has at least 80% sequence identity to the entire sequence SEQ ID NO: 32,

an amino acid sequence that has the substitutions of (IV) and has at least 80% sequence identity to the entire sequence SEQ ID NO: 33, or

an amino acid sequence that has the substitutions of (V) and has at least 80% sequence identity to the entire sequence SEQ ID NO: 34.

7. A betacoronavirus Spike (S) protein, or fragment thereof, comprising an amino acid sequence that has amino acid substitutions, wherein said amino acid substitutions are selected from:

(A)

Glycine (G) at the position that corresponds to residue 588 of the sequence SEQ ID NO: 3,

G at the position that corresponds to residue 656 of the sequence SEQ ID NO: 3, Serine (S) at the position that corresponds to residue 657 of the sequence SEQ ID NO: 3,

S at the position that corresponds to residue 659 of the sequence SEQ ID NO: 3, Proline (P) at the position that corresponds to residue 960 of the sequence SEQ ID NO: 3,

P at the position that corresponds to residue 961 of the sequence SEQ ID NO: 3, and one of (i)-(x):

(i) Cysteines at the positions that correspond to residues 744 and 989 of the sequence SEQ ID NO: 3,

(ii) Cysteines at the positions that correspond to residues 813 and 836 of the sequence SEQ ID NO: 3,

(iii) Cysteines at the positions that correspond to residues 544 and 941 of the sequence SEQ ID NO: 3,

(iv) Cysteines at the positions that correspond to residues 824 and 560 of the sequence SEQ ID NO: 3,

(v) Cysteines at the positions that correspond to residues 387 and 961 of the sequence SEQ ID NO: 3,

(vi) Cysteines at the positions that correspond to residues 357 and 959 of the sequence SEQ ID NO: 3,

(vii) Cysteines at the positions that correspond to residues 356 and 957 of the sequence SEQ ID NO: 3,

(viii) Cysteines at the positions that correspond to residues 15 and 494 of the sequence SEQ ID NO: 3,

(ix) Cysteines at the positions that correspond to residues 496 and 518 of the sequence SEQ ID NO: 3,

(x) Cysteines at the positions that correspond to residues 495 and 538 of the sequence SEQ ID NO: 3;

(B) the substitute amino acids listed throughout rows 3-134 of column #4 in Table 1, wherein each substitute amino acid is located at the position that corresponds to the residue number of SEQ ID NO: 3 that is listed in the same row of column #1 in Table 1, and one of (i)-(iv):

(i) Cysteines at the positions that correspond to residues 744 and 989 of the sequence SEQ ID NO: 3,

(ii) Cysteines at the positions that correspond to residues 813 and 836 of the sequence SEQ ID NO: 3,

(iii) Cysteines at the positions that correspond to residues 544 and 941 of the sequence SEQ ID NO: 3,

(iv) Cysteines at the positions that correspond to residues 824 and 560 of the sequence SEQ ID NO: 3;

(C) the substitute amino acids listed throughout rows 3-134 of column #9 in Table 1, wherein each substitute amino acid is located at the position that corresponds to the residue number of SEQ ID NO: 3 that is listed in the same row of column #1 in Table 1, and one of (i)-(iv):

(i) Cysteines at the positions that correspond to residues 744 and 989 of the sequence SEQ ID NO: 3,

(ii) Cysteines at the positions that correspond to residues 813 and 836 of the sequence SEQ ID NO: 3,

(iii) Cysteines at the positions that correspond to residues 544 and 941 of the sequence SEQ ID NO: 3,

(iv) Cysteines at the positions that correspond to residues 824 and 560 of the sequence SEQ ID NO: 3;

(D) the substitute amino acids listed throughout rows 3-145 of column #13 in Table 2, wherein each substitute amino acid is located at the position that corresponds to the residue number of SEQ ID NO: 3 that is listed in the same row of column #1 in Table 2, and one of (i)-(iv):

(i) Cysteines at the positions that correspond to residues 744 and 989 of the sequence SEQ ID NO: 3,

(ii) Cysteines at the positions that correspond to residues 813 and 836 of the sequence SEQ ID NO: 3,

(iii) Cysteines at the positions that correspond to residues 544 and 941 of the sequence SEQ ID NO: 3,

(iv) Cysteines at the positions that correspond to residues 824 and 560 of the sequence SEQ ID NO: 3;

(E) the substitute amino acids listed throughout rows 3-145 of column #18 in Table 2, wherein each substitute amino acid is located at the position that corresponds to the residue number of SEQ ID NO: 3 that is listed in the same row of column #1 in Table 2, and one of (i)-(iv):

(i) Cysteines at the positions that correspond to residues 744 and 989 of the sequence SEQ ID NO: 3,

(ii) Cysteines at the positions that correspond to residues 813 and 836 of the sequence SEQ ID NO: 3,

(iii) Cysteines at the positions that correspond to residues 544 and 941 of the sequence SEQ ID NO: 3,

(iv) Cysteines at the positions that correspond to residues 824 and 560 of the sequence SEQ ID NO: 3;

(F) the substitute amino acids listed throughout rows 3-34 of column #4 in Table 3, wherein each substitute amino acid is located at the position that corresponds to the residue number of SEQ ID NO: 3 that is listed in the same row of column #1 in Table 3, and one of (i)-(iv):

(i) Cysteines at the positions that correspond to residues 744 and 989 of the sequence SEQ ID NO: 3,

(ii) Cysteines at the positions that correspond to residues 813 and 836 of the sequence SEQ ID NO: 3,

(iii) Cysteines at the positions that correspond to residues 544 and 941 of the sequence SEQ ID NO: 3,

(iv) Cysteines at the positions that correspond to residues 824 and 560 of the sequence SEQ ID NO: 3.

8. The betacoronavirus Spike (S) protein, or fragment thereof, of embodiment 7 comprising:

an amino acid sequence that has the substitutions of (A)(i) and has at least 80% sequence identity to the entire sequence SEQ ID NO: 35,

an amino acid sequence that has the substitutions of (A)(ii) and has at least 80% sequence identity to the entire sequence SEQ ID NO: 36,

an amino acid sequence that has the substitutions of (A)(iii) and has at least 80% sequence identity to the entire sequence SEQ ID NO: 37,

an amino acid sequence that has the substitutions of (A)(iv) and has at least 80% sequence identity to the entire sequence SEQ ID NO: 38,

an amino acid sequence that has the substitutions of (A)(v) and has at least 80% sequence identity to the entire sequence SEQ ID NO: 39,

an amino acid sequence that has the substitutions of (A)(vi) and has at least 80% sequence identity to the entire sequence SEQ ID NO: 40,

an amino acid sequence that has the substitutions of (A)(vii) and has at least 80% sequence identity to the entire sequence SEQ ID NO: 41,

an amino acid sequence that has the substitutions of (A)(viii) and has at least 80% sequence identity to the entire sequence SEQ ID NO: 42,

an amino acid sequence that has the substitutions of (A)(ix) and has at least 80% sequence identity to the entire sequence SEQ ID NO: 43,

an amino acid sequence that has the substitutions of (A)(x) and has at least 80% sequence identity to the entire sequence SEQ ID NO: 44,

an amino acid sequence that has the substitutions of (B)(i) and has at least 80% sequence identity to the entire sequence SEQ ID NO: 45,

an amino acid sequence that has the substitutions of (B)(ii) and has at least 80% sequence identity to the entire sequence SEQ ID NO: 50,

an amino acid sequence that has the substitutions of (B)(iii) and has at least 80% sequence identity to the entire sequence SEQ ID NO: 55,

an amino acid sequence that has the substitutions of (B)(iv) and has at least 80% sequence identity to the entire sequence SEQ ID NO: 60,

an amino acid sequence that has the substitutions of (C)(i) and has at least 80% sequence identity to the entire sequence SEQ ID NO: 46,

an amino acid sequence that has the substitutions of (C)(ii) and has at least 80% sequence identity to the entire sequence SEQ ID NO: 51,

an amino acid sequence that has the substitutions of (C)(iii) and has at least 80% sequence identity to the entire sequence SEQ ID NO: 56,

an amino acid sequence that has the substitutions of (C)(iv) and has at least 80% sequence identity to the entire sequence SEQ ID NO: 61,

an amino acid sequence that has the substitutions of (D)(i) and has at least 80% sequence identity to the entire sequence SEQ ID NO: 47,

an amino acid sequence that has the substitutions of (D)(ii) and has at least 80% sequence identity to the entire sequence SEQ ID NO: 52,

an amino acid sequence that has the substitutions of (D)(iii) and has at least 80% sequence identity to the entire sequence SEQ ID NO: 57,

an amino acid sequence that has the substitutions of (D)(iv) and has at least 80% sequence identity to the entire sequence SEQ ID NO: 62,

an amino acid sequence that has the substitutions of (E)(i) and has at least 80% sequence identity to the entire sequence SEQ ID NO: 48,

an amino acid sequence that has the substitutions of (E)(ii) and has at least 80% sequence identity to the entire sequence SEQ ID NO: 53,

an amino acid sequence that has the substitutions of (E)(iii) and has at least 80% sequence identity to the entire sequence SEQ ID NO: 58,

an amino acid sequence that has the substitutions of (E)(iv) and has at least 80% sequence identity to the entire sequence SEQ ID NO: 63,

an amino acid sequence that has the substitutions of (F)(i) and has at least 80% sequence identity to the entire sequence SEQ ID NO: 49,

an amino acid sequence that has the substitutions of (F)(ii) and has at least 80% sequence identity to the entire sequence SEQ ID NO: 54,

an amino acid sequence that has the substitutions of (F)(iii) and has at least 80% sequence identity to the entire sequence SEQ ID NO: 59, or

an amino acid sequence that has the substitutions of (F)(iv) and has at least 80% sequence identity to the entire sequence SEQ ID NO: 64.

9. A betacoronavirus Spike (S) protein, or fragment thereof, comprising an amino acid sequence that has amino acid substitutions, wherein said amino acid substitutions are characterized by (A) and one of (i)-(xi):

(A)

Glycine (G) at the position that corresponds to residue 588 of the sequence SEQ ID NO: 3,

G at the position that corresponds to residue 656 of the sequence SEQ ID NO: 3,

Serine (S) at the position that corresponds to residue 657 of the sequence SEQ ID NO: 3,

S at the position that corresponds to residue 659 of the sequence SEQ ID NO: 3,

Proline (P) at the position that corresponds to residue 960 of the sequence SEQ ID NO: 3,

P at the position that corresponds to residue 961 of the sequence SEQ ID NO: 3,

(i) F, L, M, W, or Y at the position that corresponds to residue 391 of the sequence SEQ ID NO: 3;

(ii) A at the position that corresponds to residue 423 of the sequence SEQ ID NO: 3;

(iii) A at the position that corresponds to residue 427 of the sequence SEQ ID NO: 3;

(iv) A, H, M, N, or W at the position that corresponds to residue 429 of the sequence SEQ ID NO: 3;

(v) H, I, W, or Y at the position that corresponds to residue 430 of the sequence SEQ ID NO: 3;

(vi) W at the position that corresponds to residue 447 of the sequence SEQ ID NO: 3;

(vii) M at the position that corresponds to residue 449 of the sequence SEQ ID NO: 3;

(viii) T at the position that corresponds to residue 450 of the sequence SEQ ID NO: 3;

(ix) H, I, L, M, N, P, T, W, or Y at the position that corresponds to residue 460 of the sequence SEQ ID NO: 3;

(x) F, L, M, or Q at the position that corresponds to residue 461 of the sequence SEQ ID NO: 3; or

(xi) A, Y, F, R, M, C, G, or V at the position that corresponds to residue 467 of the sequence SEQ ID NO: 3.

10. The betacoronavirus Spike (S) protein, or fragment thereof, of embodiment 9 comprising an amino acid sequence that has at least 80% sequence identity to the entire sequence of one or more of SEQ ID NOs: 65-104. A betacoronavirus Spike (S) protein, or fragment thereof, comprising an amino acid sequence that has amino acid substitutions, wherein said amino acid substitutions are characterized by (A) and one of (i)-(x):(A)

Glycine (G) at the position that corresponds to residue 588 of the sequence SEQ ID NO: 3,

G at the position that corresponds to residue 656 of the sequence SEQ ID NO: 3,

Serine (S) at the position that corresponds to residue 657 of the sequence SEQ ID NO: 3,

S at the position that corresponds to residue 659 of the sequence SEQ ID NO: 3,

Proline (P) at the position that corresponds to residue 960 of the sequence SEQ ID NO: 3,

P at the position that corresponds to residue 961 of the sequence SEQ ID NO: 3,

(i) N at the position that corresponds to residue 391 of the sequence SEQ ID NO: 3 and T at the position that corresponds to residue 393 of the sequence SEQ ID NO: 3;

(ii) N at the position that corresponds to residue 423 of the sequence SEQ ID NO: 3 and T at the position that corresponds to residue 425 of the sequence SEQ ID NO: 3;

(iii) N at the position that corresponds to residue 427 of the sequence SEQ ID NO: 3 and T at the position that corresponds to residue 429 of the sequence SEQ ID NO: 3;

(iv) N at the position that corresponds to residue 429 of the sequence SEQ ID NO: 3 and T at the position that corresponds to residue 431 of the sequence SEQ ID NO: 3;

(v) N at the position that corresponds to residue 430 of the sequence SEQ ID NO: 3 and T at the position that corresponds to residue 432 of the sequence SEQ ID NO: 3;

(vi) N at the position that corresponds to residue 447 of the sequence SEQ ID NO: 3 and T at the position that corresponds to residue 449 of the sequence SEQ ID NO: 3;

(vii) N at the position that corresponds to residue 449 of the sequence SEQ ID NO: 3 and T at the position that corresponds to residue 451 of the sequence SEQ ID NO: 3;

(viii) N at the position that corresponds to residue 450 of the sequence SEQ ID NO: 3;

(ix) T at the position that corresponds to residue 463 of the sequence SEQ ID NO: 3; or

(x) N at the position that corresponds to residue 467 of the sequence SEQ ID NO: 3 and T at the position that corresponds to residue 469 of the sequence SEQ ID NO: 3.

12. The betacoronavirus Spike (S) protein, or fragment thereof, of embodiment 11 comprising:

an amino acid sequence that has the substitutions of (A)(i) and has at least 80% sequence identity to the entire sequence SEQ ID NO: 105,

an amino acid sequence that has the substitutions of (A)(ii) and has at least 80% sequence identity to the entire sequence SEQ ID NO: 106,

an amino acid sequence that has the substitutions of (A)(iii) and has at least 80% sequence identity to the entire sequence SEQ ID NO: 107,

an amino acid sequence that has the substitutions of (A)(iv) and has at least 80% sequence identity to the entire sequence SEQ ID NO: 108,

an amino acid sequence that has the substitutions of (A)(v) and has at least 80% sequence identity to the entire sequence SEQ ID NO: 109,

an amino acid sequence that has the substitutions of (A)(vi) and has at least 80% sequence identity to the entire sequence SEQ ID NO: 110,

an amino acid sequence that has the substitutions of (A)(vii) and has at least 80% sequence identity to the entire sequence SEQ ID NO: 111,

an amino acid sequence that has the substitutions of (A)(viii) and has at least 80% sequence identity to the entire sequence SEQ ID NO: 112,

an amino acid sequence that has the substitutions of (A)(ix) and has at least 80% sequence identity to the entire sequence SEQ ID NO: 113, or

an amino acid sequence that has the substitutions of (A)(x) and has at least 80% sequence identity to the entire sequence SEQ ID NO: 114.

13. The betacoronavirus S protein, or S protein fragment, of any one of embodiments 1-12 comprising an amino acid sequence with at least 80% sequence identity to the entire sequence of one or more of SEQ ID NOs: 5-114.

14. A betacoronavirus Spike (S) protein, or fragment thereof, of embodiment 1, which comprises one of the following SEQ ID NOs: 22-29.

15. A nucleic acid molecule comprising a polynucleotide sequence that encodes the betacoronavirus S protein, or S protein fragment, of any one of embodiments 1-14.

16. The nucleic acid molecule of embodiment 15 that is a Self-Amplifying RNA Molecule comprising, from 5′-3′, a polynucleotide comprising the sequence SEQ ID NO: 119; a polynucleotide sequence that encodes the betacoronavirus S protein, or S protein fragment, of any one of embodiments 1-13; and a polynucleotide comprising the sequence SEQ ID NO: 120.

17. A betacoronavirus Spike (S) protein, or fragment thereof, comprising an amino acid sequence that has amino acid substitutions, wherein said amino acid substitutions are characterized by (A) and one of (i)-(v):

(A)
G at the position that corresponds to residue 202 of any of SEQ ID NOS: 125-134;
Asparagine (N) at the position that corresponds to residue 404 of any of SEQ ID NOS: 125-134;
Lysine (K) at the position that corresponds to residue 471 of any of SEQ ID NOS: 125-134;
Tyrosine (Y) at the position that corresponds to residue 488 of any of SEQ ID NOS: 125-134;
G at the position that corresponds to residue 601 of any of SEQ ID NOS: 125-134; and
Isoleucine (I) at the position that corresponds to residue 692 and Glutamine (Q) that corresponds to residue 727 of any of SEQ ID NOS: 125-134;

(i) P at the positions that correspond to residues 691, 693, 818, and 1101 of any of SEQ ID NOS: 125-134;

(ii) Glutamate (E) at the position that corresponds to residue 756 of any of SEQ ID NOS: 125-134;

(iii) Y at the position that corresponds to residue 801 of any of SEQ ID NOS:125-134;

(iv) Serine (S) at the position that corresponds to residue 879 of any of SEQ ID NOS:125-134; and

(v) K at the position that corresponds to residue 916 of any of SEQ ID NOS: 125-134.

18. The betacoronavirus Spike (S) protein, or fragment thereof, of embodiment 17 comprising:
an amino acid sequence that has the substitutions of (A)(i) and has at least 80% sequence identity to the entire sequence SEQ ID NO: 125;
an amino acid sequence that has the substitutions of (A)(i) and has at least 80% sequence identity to the entire sequence SEQ ID NO: 126;
an amino acid sequence that has the substitutions of (A)(i) and has at least 80% sequence identity to the entire sequence SEQ ID NO: 127;
an amino acid sequence that has the substitutions of (A)(i) and has at least 80% sequence identity to the entire sequence SEQ ID NO: 128; and
an amino acid sequence that has the substitutions of (A)(i) and has at least 80% sequence identity to the entire sequence SEQ ID NO: 129.

19. The betacoronavirus Spike (S) protein, or fragment thereof, of embodiment 18, comprising an amino acid sequence of any one of SEQ ID NOs: 125-129.

20. A betacoronavirus Spike (S) protein, or fragment thereof, comprising an amino acid sequence that has amino acid substitutions, wherein said amino acid substitutions are characterized by (A) and one of (i)-(v):

(A)
G at the position that corresponds to residue 202 of any of SEQ ID NOS: 125-134;
Asparagine (N) at the position that corresponds to residue 404 of any of SEQ ID NOS: 125-134;
Lysine (K) at the position that corresponds to residue 471 of any of SEQ ID NOS: 125-134;
Tyrosine (Y) at the position that corresponds to residue 488 of any of SEQ ID NOS: 125-134;
G at the position that corresponds to residue 601 of any of SEQ ID NOS: 125-134; and
Isoleucine (I) at the position that corresponds to residue 692 and Glutamine (Q) that corresponds to residue 727 of any of SEQ ID NOS: 125-134;

(i) S at the position that corresponds to residue 691 of any of SEQ ID NOS:125-134;

(ii) A at the positions that correspond to residues 693 and 818 of any of SEQ ID NOS: 125-134;

(iii) I at the position that corresponds to residue 1101 of any of SEQ ID NOS: 125-134;

(iv) G at the position that corresponds to residue 756 of any of SEQ ID NOS: 125-134;

(v) K at the position that corresponds to residue 801 of any of SEQ ID NOS:125-134;

(iv) A at the position that corresponds to residue 879 of any of SEQ ID NOS:125-134; and

(v) S at the position that corresponds to residue 916 of any of SEQ ID NOS:125-134.

21. The betacoronavirus Spike (S) protein, or fragment thereof, of embodiment 20 comprising:
an amino acid sequence that has the substitutions of (A)(i) and has at least 80% sequence identity to the entire sequence SEQ ID NO: 130;
an amino acid sequence that has the substitutions of (A)(i) and has at least 80% sequence identity to the entire sequence SEQ ID NO: 131;
an amino acid sequence that has the substitutions of (A)(i) and has at least 80% sequence identity to the entire sequence SEQ ID NO: 132;
an amino acid sequence that has the substitutions of (A)(i) and has at least 80% sequence identity to the entire sequence SEQ ID NO: 133; and
an amino acid sequence that has the substitutions of (A)(i) and has at least 80% sequence identity to the entire sequence SEQ ID NO: 134.

22. The betacoronavirus Spike (S) protein, or fragment thereof, of embodiment 21, comprising an amino acid sequence of any one of SEQ ID NOs: 130-134.

23. A nucleic acid molecule comprising a polynucleotide sequence that encodes the betacoronavirus S protein, or S protein fragment, of embodiment 17 or 20.

24. The nucleic acid molecule of embodiment 23 that is a Self-Amplifying RNA Molecule comprising, from 5′-3′, a polynucleotide comprising the sequence SEQ ID NO: 119; a polynucleotide sequence that encodes the betacoronavirus S protein, or S protein fragment, of embodiment 17 or 20; and a polynucleotide comprising the sequence SEQ ID NO: 120.

25. An immunogenic composition comprising (i) the betacoronavirus S protein, or S protein fragment of any one of embodiments 1-14, 17 or 20, optionally further comprising an adjuvant; or (ii) the nucleic acid molecule of embodiment 15 or 16.

26. A method of inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases; comprising

delivering to a subject an immunologically effective amount of the immunogenic composition of embodiment 25.

27. Use of the immunogenic composition of embodiment 25 for inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases.

28. Use of the immunogenic composition of embodiment 25 for the manufacture of a medicament for inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases.

29. The immunogenic composition of embodiment 25 for use in inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases.

### BRIEF DESCRIPTION OF THE DRAWINGS

FIG. 1A—Schematic of the SARS-CoV-2 Spike (S) protein primary structure by domain (from Wrapp et al. 2020 Science 367(6483):1260-1263). SS, signal sequence; S2′, S2′ protease cleavage site; FP, fusion peptide; HR1, heptad repeat 1; CH, central helix; CD, connector domain; HR2, heptad repeat 2; TM, transmembrane domain; CT, cytoplasmic tail. Arrows denote protease cleavage sites.

FIG. 1B—Schematic diagram of the MERS-CoV Spike (S) glycoprotein organization (from Yuan et al. 2017 Nat. Comm. 8(15092), 9 pgs). NTD, N-terminal domain; L, linker region; RBD, receptor-binding domain; SD, subdomain; UH, upstream helix; FP, fusion peptide; CR, connecting region; HR, heptad repeat; CH, central helix; BH, b-hairpin; TM, transmembrane region/domain; CT, cytoplasmic tail.

FIG. 1C—Schematic diagram of the SARS-CoV-1 Spike (S) glycoprotein organization (from Yuan et al. 2017 Nat. Comm. 8(15092), 9 pgs). The abbreviations of elements are the same as in FIG. 1B.

FIGS. 1D and 1E—Schematic diagram of the SARS-CoV-2 ectodomain of assay control proteins, S-2P (FIG. 1D, with 2 proline substitutions) and HexaPro (FIG. 1E, with 6 proline substitutions).

FIG. 2—Rosetta Energies (kcal/mol) of modified SARS-CoV-2 Spike (S) proteins designed to include stabilizing mutations (relative to PDB Accession Number 6VYB) that target sites on the S2 (circles) or S (squares) domains, on a model of the full S antigen (hexagon, “6VYB” meaning the sequence published as PDB Accession Number 6VYB).

FIG. 3—Rosetta Energies (kcal/mol) of modified SARS-CoV-2 Spike (S) proteins designed to include stabilizing point mutations in the S domain (S, squares), S2 and N-terminal domains (S2_NTD, diamonds) or S2 domain only (S2, circles) compared to a prefusion SARS-CoV-2 S protein having the sequence SEQ ID NO: 4 (“preS”, hexagon) which was produced according to Wrapp et al. 2020 Science 367(6483):1260-1263, with the D614G drift mutation as identified by internal phylogenetic analysis and by Korber et al. 2020 bioRxiv (HyperTextTransferProtocolSecure: //doi.org/10.1101/2020.04.29.069054) and Brufsky 20 Apr. 2020 J Med Virol, 7 pages, doi: 10.1002/jmv.25902.

FIGS. 4A and 4B—Rosetta Energies (kcal/mol) results from a combined Rosetta HBNet-PROSS workflow targeting the S or S2 domains from SARS-CoV-2 S protein, on a model of the full S protein (preS_6VYB). The design protocol performs hydrogen-bond network optimization, plus combinatorial sequence design based on evolutionary sequences obtained from the non-redundant BLAST database. The combined protocol indicates that HBNet-PROSS (S_hbnet_pross, circles) is destabilizing for the HBNet design (S_hbnet, squares) of the full S protein (preS_6VYB, hexagon) (FIG. 4A) and stabilizing for the HBNet design targeted towards the S2 domain (S2 hbnet_pross, circles), which contains the core virus fusion machinery and is mostly helical in nature, versus the HBNet design (S2_hbnet, squares) (FIG. 4B).

FIG. 5—Rosetta Energies (kcal/mol) results from a single point mutation design to knock-out binding at the interface between hACE2 and SARS CoV-2 S protein RBD (using interface residues shown by the x-ray structure of Lan et al. (2020 Nature HyperTextTransferProtocolSecure://doi.org/10.1038/s41586-020-2180-5, 16 pgs), revealing some mutations that reduce binding affinity (greater than 2 kcal/mol) while maintaining folding stability, according to in silico Rosetta energetics.

FIG. 6—Rosetta Energy (kcal/mol) results of introducing NxT glycan motifs through in silico mutation design to mask the binding site at the interface between hACE2 and SARS CoV-2 S protein RBD (using interface residues shown by the x-ray structure of Lan et al. (2020 Nature HyperTextTransferProtocolSecure: //doi.org/10.1038/s41586-020-2180-5, 16 pgs). These results show that the motifs have varying clusters of stabilization energies, indicating that substitutions at A475 and K417 might maintain folding stability equivalent to the wildtype.

FIGS. 7A and 7B—The designed S antigens were produced in a high-throughput expression system, identifying constructs with >5 or 6-fold protein yield, relative to S-2P. HexaPro 1 and HexaPro 2 have the same chemical and physical properties as HexaPro, differing only by the technician who handled the control S protein. S-2P 1 and S-2P 2 have the same chemical and physical properties as S-2P, differing only by the technician who handled the control S protein.

FIG. 8A-8D In a HT binding screen in supernatant (Octet BLI), the ACE2 receptor and 3 antibodies (CR3022: RBD Specific Antibody, VRC 118: NTD Specific Antibody, VRC 112: S2 Specific Antibody) were used to test the conformational and antigenic integrity of the designs. VRC112 and VRC118 were obtained under an agreement with National Institute of Allergy and Infectious Diseases (NIAID).

FIG. 8E—Binding Affinity assay, performed using SPR, shows reduced binding affinity of SEQ ID NO: 25 to CR3022 IgG and ACE2 receptor.

FIGS. 9A-9C—Thermal unfolding of the S antigens was screened (Nano DSF), indicating that some constructs had increased stability depending on mutation site.

FIG. 10—PROSS designs of CoV-2 variant B.1.351 spike glycoprotein, introducing mutations into S2 domain (black) or buried residue with less than 25% exposure in the S2 domain (gray).

### DETAILED DESCRIPTION

### Terms

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

“About” or “approximately”, when used to modify a numeric value, means a number that is not statistically different from the referenced numeric value and, when the numeric value relates to the amount of a composition component, means a number not more than 10% below or above the numeric value (not more than 10% below or above the endpoint values if the numeric value is a range). As an example, a composition comprising “about 25 μg” of component A means the composition comprises “22.5-27.5 μg” of component A (10% of 25 is 2.5, so 10% below 25 is 22.5 and 10% above 25 is 27.5; resulting in the range 22.5-27.5). As an example, a composition comprising “approximately 25 μg” of component A means the composition comprises “22.5-27.5 μg” of component A. As a further example, a composition comprising “about 25-30 μg” of component A means the composition comprises “22.5-33 μg” of component A (10% below 25 is 22.5 and 10% above 30 is 33). As a further example, a composition comprising “approximately 25-30 μg” of component A means the composition comprises “22.5-33 μg” of component A.

“Adjuvant” means an agent that, or composition comprising an agent, that modulates an immune response in a non-specific manner and accelerates, prolongs, and/or enhances the immune response to an antigen. Such an agent may be an “immunostimulant”. An “adjuvant” herein may be a composition that comprises one or more immunostimulants (in particular, an immunostimulating effective amount of one or more immunostimulants (e.g., a saponin)). A “pharmaceutical-grade adjuvant” means an adjuvant suitable for pharmaceutical use (e.g., an adjuvant comprising one or more purified immunostimulant, in particular comprising an immunologically effective amount of a purified immunostimulant). Therefore and for clarity, an adjuvant administered with an antigen produces an accelerated, prolonged, and/or enhanced immune response than the antigen alone does.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone). Similarly, the word “or” is intended to include each of the listed elements individually as well as any combination of the elements (i.e., “or” herein encompasses “and”), unless the context clearly indicates otherwise.

“Antibody” means a protein molecule produced by the immune system to help eliminate an antigen (or recombinant versions thereof) and includes a monoclonal antibody, polyclonal antibody, multispecific antibody (e.g., bispecific antibodies), labelled antibody, or antibody fragment (so long as the fragment exhibits or maintains the desired antigen-binding activity). Unless stated otherwise, by “antibody” herein it is meant a neutralizing antibody. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Antigen” means a molecule, structure, compound, or substance (e.g., a polynucleotides (DNA, RNA), polypeptides, protein complexes) that can stimulate an immune response by producing antigen-specific antibodies and/or an antigen-specific T cell response in a subject (e.g., a human subject). Antigens may be live, inactivated, purified, and/or recombinant. For clarity, an adjuvant is not an antigen at least because an adjuvant cannot (alone) induce antigen-specific immune response. As used herein, an antigen is immunogenic. The term “antigen” includes all related antigenic epitopes. The term “epitope” means that portion of an antigen that determines its immunological specificity and refers to a site on an antigen to which B and/or T cells respond. “Predominant antigenic epitopes” are those epitopes to which a functionally significant host immune response (e.g., an antibody response or a T-cell response) is made. Thus, the predominant antigenic epitopes are those antigenic moieties that, when recognized by the host immune system, result in a protective immune response. The term “T-cell epitope” refers to an epitope that, when bound to an appropriate MHC molecule, is specifically bound by a T cell (via a T cell receptor). A “B-cell epitope” is an epitope that is specifically bound by an antibody (or B cell receptor molecule).

“Antigenicity” means a molecule's, structure's, compound's, or substance's (e.g., an antigen's) ability to combine with an antibody. An “increased antigenicity” or “enhanced antigenicity” means an increased binding affinity of an antibody to the molecule, structure, compound, or substance (e.g., an antigen). An increased binding affinity may be provided as a decreased dissociation constant (K_{d}) value (in nM). See generally, e.g., Ma et al. 2011 PLoS Path. 7(9), e1002200. For clarity, antigenicity does not mean immunogenicity—a molecule may bind an antibody (antigenicity) without eliciting an immune response (immunogenicity).

“Comparably to” or “comparable to” means equivalent, analogous, substitutes, not statistically different than, not materially different in structure and/or function. For example, recombinant molecule or recombinant structure said to be “comparable to wild type” or “comparable to its wild type counterpart” or an “analog” means the recombinant molecule/structure may be substituted for its wild type counterpart without material change to or effect (e.g., in eliciting an immunogenic response). An “analog” herein includes synthetic molecules or structures meant to mimic the function of its counterpart (in that way, an analog's structure may be distinct from its counterpart's but the analog's function or effect is comparable to its counterpart's function or effect).

“Corresponding to” or “corresponds to” (as in, e.g., “at the position location that corresponds to residue # within sequence Y”) is used to reference a nucleic acid or amino acid residue of a second sequence (e.g., a subject sequence) that “aligns to” a referenced residue (structure and/or location) of a first (e.g., query sequence) (e.g., by pairwise, global sequence alignment). This terminology is used to accommodate the well-recognized fact that structural variation that may exist between functionally comparable sequences. Due to sequence variation (e.g., natural sequence variation) between the a first (query) sequence and the second (subject) sequences, the subject residue may have an identical structure as the query residue, but be located at a different location and therefore have a different residue number than the query residue when aligned thereto. Also perhaps due to sequence variation (e.g., natural sequence variation), the subject residue may not have an identical structure as the query residue (e.g., may be a so-called conserved substitute) and nonetheless align to the same location (i.e., have the same residue number) as the query residue within the first (query) sequence. “Aligns to” may be used herein as an alternate to “corresponding to”. Whether or not a nucleic/amino acid residue within a subject sequence “corresponds to” a nucleic/amino acid residue within a query sequence is determined by sequence alignment, preferably by pairwise, global alignment with the Needleman-Wunsch algorithm using default parameters (defined elsewhere herein). As an example, “the nucleic amino acid residue corresponding to residue ## of SEQ ID NO: ###” means the nucleic/amino acid that aligns to the referenced residue (“ . . . residue ## of SEQ ID NO: ###”), such as after pairwise, global alignment with the Needleman-Wunsch algorithm using default parameters. This terminology is useful, for example, when the second/subject sequence comprises one or more gap(s), insertions, or deletions as compared to the first/query sequence (thus changing residue numbering). As a further example, “the nucleic amino acid residue at the position corresponding to ‘X’ of SEQ ID NO: ###” or simply “at the position corresponding to ‘X’ of SEQ ID NO: ###” means the nucleic/amino acid (regardless of its chemical structure) that aligns to the referenced location (where “‘X’ of SEQ ID NO: ###” is located), such as after pairwise, global alignment with the Needleman-Wunsch algorithm using default parameters. This is useful, for example, when describing the location of a sequence feature (e.g., where a domain is) or modification (e.g., where to make a nucleic amino acid substitution) amongst sequences of varying lengths. In certain embodiments and for readability, “numbered with respect to”, “numbered according to”, “with respect to”, or similar phrases may be used to reference a residue or sequence feature. As a demonstration, “amino acid corresponding to F17 of the sequence SEQ ID NO: 3” encompasses the amino acid (regardless of its chemical structure) that aligns to F17 of SEQ ID NO: 3 such as F34 of the SARS-CoV-1 spike (S) protein sequence SEQ ID NO: 116. Also, “a serine (S) at a position corresponding to residue 17 of SEQ ID NO: 3” encompasses both the F17S mutant of the SARS-CoV-2 spike (S) protein sequence SEQ ID NO: 3 as well as the F34S mutant of the SARS-CoV-1 S protein sequence SEQ ID NO: 116 (because F17 of SEQ ID NO: 3 aligns to F34 of SEQ ID NO: 116 as shown below). This language is also useful for describing resultant modifications (e.g., amino acid substitutions) when the original residue may be one of several, for example, “an asparagine (N) at a position corresponding to residue 391 of SEQ ID NO: 3” encompasses both the K391N mutant of SARS-CoV-2 S protein sequence SEQ ID NO: 3 as well as the V391N mutant of SARS-CoV-1 S protein sequence SEQ ID NO: 116 (see alignment below). Below is a pairwise, global alignment using Needleman-Wunsch algorithm with default parameters of SARS-CoV-2 Spike (S) protein sequence SEQ ID NO: 3 to SARS-CoV-1 S protein sequence SEQ ID NO: 116—alignment conducted using EMBOSS Needle (pair output format), the reported aligned region is 1265 amino acids in length with 840 identical matches meaning the percent sequence identity calculation is (840/1265)×100 (=66.4%), if rounded down to the nearest whole number provides 66% identity between SEQ ID NOs: 3 and 116; referenced residues/positions are double underlined. Please note that the length of the aligned region (1265 residues) includes any gaps in the length and is, here, neither the length of SEQ ID NO: 3 (1121) nor SEQ ID NO: 116 (1242).

| | | |
|---|---|---|
| # | Aligned_sequences: | 2 |
| # | 1: | SEQ_ID_NO_3 |
| # | 2: | SEQ_ID_NO_116 |
| # | Matrix: | EBLOSUM62 |
| # | Gap_penalty: | 10.0 |
| # | Extend_penalty: | 0.5 |
| # | | |
| # | Length: | 1265 |
| # | Identity: | 840/1265 (66.4%) |
| # | Similarity: | 973/1265 (76.9%) |
| # | Score: | 4523.5 |

| | | | |
|---|---|---|---|
| SEQ_ID_NO_3 | 1 | ------------------AYTNSFTRGVYYPDKVFRSSVLHSTQDLFLPF | 32 |
| | | .:\|:\|. \|\|\|\|\|\|\|::\|\|\|..\|:.\|\|\|\|\|\|\|\| | |
| SEQ_ID_NO_116 | 1 | SDLDRCTTFDDVQAPNYTQHTSSM-RGVYYPDEIFRSDTLYLTQDLFLPF | 49 |
| SEQ_ID_NO_3 | 33 | FSNVTWFHAIHVSGTNGTKRFDNPVLPFNDGVYFASTEKSNIIRGWIFGT | 82 |
| | | :\|\|\|\|.\|\|.\| \|.\| \|.\|\|\|:\|\|.\|\|:\|\|\|:\|\|\|\|\|::\|\|\|:\|\|: | |
| SEQ_ID_NO_116 | 50 | YSNVTGFHTI-----NHT--FGNPVIPFKDGIYFAATEKSNVVRGWVFGS | 92 |
| SEQ_ID_NO_3 | 83 | TLDSKTQSLLIVNNATNVVIKVCEFQFCNDPFLGVYYHKNNKSWMESEFR | 132 |
| | | \|:::\|:\|\|::\|:\|\|:\|\|\|\|\|:.\|.\|:.\|::\|\|..\| :.....::... | |
| SEQ_ID_NO_116 | 93 | TMNNKSQSVIIINNSTNVVIRACNFELCDNPFFAV----SKPMGTQTHTM | 138 |
| SEQ_ID_NO_3 | 133 | VYSSANNCTFEYVSQPFLMDLEGKQGNFKNLREFVFKNIDGYFKIYSKHT | 182 |
| | | ::.:\|.\|\|\|\|\|\|:\|..\|.:\|:..\|.\|\|\|\|:\|\|\|\|\|\|\|\|.\|\|:..:\|..:. | |
| SEQ_ID_NO_116 | 139 | IFDNAFNCTFEYISDAFSLDVSEKSGNFKHLREFVFKNKDGFLYVYKGYQ | 188 |
| SEQ_ID_NO_3 | 183 | PINLVRDLPQGFSALEPLVDLPIGINITRFQTLLALHRSYLTPGDSSSGW | 232 |
| | | \|\|::\|\|\|\|\|.\|\|:.\|:\|:..\|\|:\|\|\|\|\|.\|:.:\| :..:\|.... \| | |
| SEQ_ID_NO_116 | 189 | PIDVVRDLPSGFNTLKPIFKLPLGINITNFRAIL----TAFSPAQDI--W | 232 |
| SEQ_ID_NO_3 | 23 | TAGAAAYYVGYLQPRTFLLKYNENGTITDAVDCALDPLSETKCTLKSFTV | 282 |
| | | ...\|\|\|\|:\|\|\|\|:\|.\|\|:\|\|\|:\|\|\|\|\|\|\|\|\|\|\|:.:\|\|:\|.\|\|::\|\|\|.: | |
| SEQ_ID_NO_116 | 233 | GTSAAAYFVGYLKPTTFMLKYDENGTITDAVDCSQNPLAELKCSVKSFEI | 282 |
| SEQ_ID_NO_3 | 283 | EKGIYQTSNFRVQPTESIVRFPNITNLCPFGEVFNATRFASVYAWNRKRI | 332 |
| | | :\|\|\|\|\|\|\|\|\|\|\|.\|:..:\|\|\|\|\|\|\|\|\|\|\|\|\|\|\|\|\|\|\|:\|.\|\|\|\|\|.\|\|:\| | |
| SEQ_ID_NO_116 | 283 | DKGIYQTSNFRVVPSGDVVRFPNITNLCPFGEVFNATKFPSVYAWERKKI | 332 |
| SEQ_ID_NO_3 | 333 | SNCVADYSVLYNSASFSTFKCYGVSPTKLNDLCFTNVYADSEVIRGDEVR | 382 |
| | | \|\|\|\|\|\|\|\|\|\|\|\|\|..\|\|\|\|\|\|\|\|\|\|.\|\|\|\|\|\|\|\|:\|\|\|\|\|\|\|\|::\|\|:\|\| | |
| SEQ_ID_NO_116 | 333 | SNCVADYSVLYNSTFFSTFKCYGVSATKLNDLCFSNVYADSFVVKGDDVR | 382 |
| SEQ_ID_NO_3 | 383 | QIAPGQTGKIADYNYKLPDDFTGCVIAWNSNNLDSKVGGNYNYLYRLFRK | 432 |
| | | \|\|\|\|\|\|\|\|.\|\|\|\|\|\|\|\|\|\|\|\|.\|\|\|:\|\|\|:.\|:\|:...\|\|\|\|\|.\|\|..\|. | |
| SEQ_ID_NO_116 | 383 | QIAPGQTGVIADYNYKLPDDFMGCVLAWNTRNIDATSTGNYNYKYRYLRH | 432 |
| SEQ_ID_NO_3 | 433 | SNLKPFERDISTEIYQAGSTPCNGVEGFNCYFPLQSYGFQPTNGVGYQPY | 482 |
| | | ..\|:\|\|\|\|\|\|\|...:.....\|\|. ....\|\|\|:\|\|..\|\|\|..\|.\|:\|\|\|\|\| | |
| SEQ_ID_NO_116 | 433 | GKLRPFERDISNVPFSPDGKPCT-PPALNCYWPLNDYGFYTTTGIGYQPY | 481 |
| SEQ_ID_NO_3 | 483 | RVVVLSFELLHAPATVCGPKKSTNLVKNKCVNFNFNGLTGTGVLTESNKK | 532 |
| | | \|\|\|\|\|\|\|\|\|\|:\|\|\|\|\|\|\|\|\|.\|\|:\|:\|\|:\|\|\|\|\|\|\|\|\|\|\|\|\|\|\|\|.\|:\|: | |
| SEQ_ID_NO_116 | 482 | RVVVLSFELLNAPATVCGPKLSTDLIKNQCVNFNFNGLTGTGVLTPSSKR | 531 |
| SEQ_ID_NO_3 | 533 | FLPFQQFGRDIADTTDAVRDPQTLEILDITPCSFGGVSVITPGTNTSNQV | 582 |
| | | \|.\|\|\|\|\|\|\|\|::\|.\|\|:\|\|\|\|:\|.\|\|\|\|\|:\|\|\|\|\|\|\|\|\|\|\|\|\|\|\|.\|::\| | |
| SEQ_ID_NO_116 | 532 | FQPFQQFGRDVSDFTDSVRDPKTSEILDISPCSFGGVSVITPGTNASSEV | 581 |
| SEQ_ID_NO_3 | 583 | AVLYQDVNCTEVPVAIHADQLTPTWRVYSTGSNVFQTRAGCLIGAEHVNN | 632 |
| | | \|\|\|\|\|\|\|\|\|\|:\|..\|\|\|\|\|\|\|\|\|.\|\|:\|\|\|\|:\|\|\|\|\|:\|\|\|\|\|\|\|\|\|\|:. | |
| SEQ_ID_NO_116 | 582 | AVLYQDVNCTDVSTAIEADQLTPAWRIYSTGNNVFQTQAGCLIGAEHVDT | 631 |
| SEQ_ID_NO_3 | 633 | SYECDIPIGAGICASYQTQTNSPRRARSVASQSIIAYTMSLGAENSVAYS | 682 |
| | | \|\|\|\|\|\|\|\|\|\|\|\|\|\|\|\|.\|.: ..\|\|.:.:\|\|:\|\|\|\|\|\|\|\|::\|:\|\|\| | |
| SEQ_ID_NO_116 | 632 | SYECDIPIGAGICASYHTVS----LLRSTSQKSIVAYTMSLGADSSTAYS | 677 |
| SEQ_ID_NO_3 | 683 | NNSIAIPTNFTISVTTEILPVSMTKTSVDCTMYICGDSTECSNLLLQYGS | 732 |
| | | \|\|:\|\|\|\|\|\|\|:\|\|:\|\|\|::\|\|\|\|.\|\|\|\|\|\|.\|\|\|\|\|\|\|\|\|\|:\|\|\|\|\|\|\|\| | |
| SEQ_ID_NO_116 | 678 | NNTIAIPTNFSISITTEVMPVSMAKTSVDCNMYICGDSTECANLLLQYGS | 727 |
| SEQ_ID_NO_3 | 733 | FCTQLNRALTGIAVEQDKNTQEVFAQVKQIYKTPPIKDFGGFNFSQILED | 782 |
| | | \|\|\|\|\|\|\|\|\|:\|\|\|.\|\|\|:\|\|:\|\|\|\|\|\|\|\|:\|\|\|\|.:\|.\|\|\|\|\|\|\|\|\|\|\|\| | |
| SEQ_ID_NO_116 | 728 | FCTQLNRALSGIAAEQDRNTREVFAQVKQMYKTPTLKYFGGFNFSQILPD | 777 |
| SEQ_ID_NO_3 | 783 | PSKPSKKSFLEDLLENKVTLADAGFIKQYGDCLGDLAAKDLICAQRENGL | 832 |
| | | \|.\|\|:\|\|\|\|\|\|\|\|\|\|\|\|\|\|\|\|\|\|\|\|:\|\|\|\|:\|\|\|\|\|.\|\|\|\|\|\|\|\|\|\|\|\|\| | |
| SEQ_ID_NO_116 | 778 | PLKPTKRSFIEDLLFNKVTLADAGEMKQYGECLGDINARDLICAQKFNGL | 827 |
| SEQ_ID_NO_3 | 833 | TVLPPLLTDEMIAQYTSALLAGTITSGWTFGAGAALQIPFAMQMAYRFNG | 882 |
| | | \|\|\|\|\|\|\|\|\|:\|\|\|.\|\|:\|\|::\|\|.\|:\|\|\|\|\|\|\|\|\|\|\|\|\|\|\|\|\|\|\|\|\|\|\|\| | |
| SEQ_ID_NO_116 | 828 | TVLPPLLTDDMIAAYTAALVSGTATAGWTFGAGAALQIPFAMQMAYRFNG | 877 |
| SEQ_ID_NO_3 | 883 | IGVTQNVLYENQKLIANQFNSAIGKIQDSLSSTASALGKLQDVVNQNAQA | 932 |
| | | \|\|\|\|\|\|\|\|\|\|\|\|\|.\|\|\|\|\|\|.\|\|.:\|\|:\|\|::\|::\|\|\|\|\|\|\|\|\|\|\|\|\|\|\| | |
| SEQ_ID_NO_116 | 878 | IGVTQNVLYENQKQIANQFNKAISQIQESLTTTSTALGKLQDVVNQNAQA | 927 |
| SEQ_ID_NO_3 | 933 | LNTLVKQLSSNFGAISSVLNDILSRLDKVEAEVQIDRLITGRLQSLQTYV | 982 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SEQ_ID_NO_116 | 928 | LNTLVKQLSSNFGAISSVLNDILSRLDKVEAEVQIDRLITGRLQSLQTYV | 977 |
| SEQ_ID_NO_3 | 983 | TQQLIRAAEIRASANLAATKMSECVLGQSKRVDFCGKGYHLMSFPQSAPH | 1032 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|:\|\|\| | |
| SEQ_ID_NO_116 | 978 | TQQLIRAAEIRASANLAATKMSECVLGQSKRVDFCGKGYHLMSFPQAAPH | 1027 |
| SEQ_ID_NO_3 | 1033 | GVVFLHVTYVPAQEKNFTTAPAICHDGKAHFPREGVFVSNGTHWFVTQRN | 1082 |
| | | \|\|\|\|\|\|\|\|\|\|\|:\|\|:\|\|\|\|\|\|\|\|\|\|:\|\|\|:\|\|\|\|\|\|\|\|.\|\|\|.\|\|:\|\|\|\| | |
| SEQ_ID_NO_116 | 1028 | GVVFLHVTYVPSQERNFTTAPAICHEGKAYFPREGVFVFNGTSWFITQRN | 1077 |
| SEQ_ID_NO_3 | 1083 | FYEPQIITTDNTFVSGNCDVVIGIVNNTVYDPLQPELDS----------- | 1121 |
| | | \|:.\|\|\|\|\|\|\|\|\|\|\|\|\|\|\|\|\|\|\|\|\|:\|\|\|\|\|\|\|\|\|\|\|\|\|\| | |
| SEQ_ID_NO_116 | 1078 | FFSPQIITTDNTFVSGNCDVVIGIINNTVYDPLQPELDSFKEELDKYFKN | 1127 |
| SEQ_ID_NO_3 | 1122 | -------------------------------------------------- | 1121 |
| SEQ_ID_NO_116 | 1128 | HTSPDVDLGDISGINASVVNIQKEIDRLNEVAKNLNESLIDLQELGKYEQ | 1177 |
| SEQ_ID_NO_3 | 1122 | -------------------------------------------------- | 1121 |
| SEQ_ID_NO_116 | 1178 | YIKWPWYVWLGFIAGLIAIVMVTILLCCMTSCCSCLKGACSCGSCCKFDE | 1227 |
| SEQ_ID_NO_3 | 1122 | --------------- | 1121 |
| SEQ_ID_NO_116 | 1228 | DDSEPVLKGVKLHYT | 1242 |

“Delivering” herein (e.g., as in methods of “delivering a betacoronavirus S protein or fragment thereof to a subject”) is used to generically refer to the breadth and variety of known delivery methods (e.g., DNA, RNA, subunit, or other) that may be utilized for that purpose (see herein below). In that way, for example, “delivery of a betacoronavirus S protein or S protein fragment” encompasses both the administration of a polynucleotide (DNA or RNA) encoding that betacoronavirus S protein or fragment as well as administration of that betacoronavirus S protein or fragment itself (i.e., subunit approach). If a particular delivery method or formulation is meant, such will be specified.

“Host cell” as used herein does not encompass a (whole) human organism.

“Human dose” means a dose which is in a volume suitable for human use (“human dose volume”) such as 0.25-1.5 ml. For example, a composition formulated in a volume of about 0.5 ml; specifically a volume of 0.45-0.55 ml; or more specifically a volume of 0.5 ml.

An “immune response” is a response of a cell of the immune system (such as a B cell, T cell, or monocyte) to a stimulus (e.g., an antigen). An immune response can be a B cell response (or “humoral immune response”), which results in the production of specific antibodies, such as antigen-specific neutralizing antibodies. A “neutralizing antibody response” may be complement-dependent or complement-independent. A neutralizing antibody response may be cross-neutralizing (a neutralizing antibody generated against an antigen from one virus strain, e.g., is neutralizing against the comparable antigen from another strain of that virus). An immune response can also be a T cell response, such as a CD4+ T cell response or a CD8+ T cell response. In some cases, the response is specific for a particular antigen (that is, an “antigen-specific response”), in particular, a modified betacoronavirus S protein or S protein fragment. If the antigen is derived from a pathogen, the antigen-specific response is a “pathogen-specific response” (e.g., a “MERS-CoV-specific immune response”, “a SARS-CoV-1-specific immune response”, or a “SARS-CoV-2-specific immune response”). A “protective immune response” is an immune response that reduces a detrimental function or activity of a pathogen, reduces infection by a pathogen (including cell entry), reduces cell-to-cell spread of a pathogen, and/or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo. It may be further specified that the humoral immune response, CD4 T cell response, or CD8 T cell response is “at natural immunity”, “comparable to natural immunity”, or “above natural immunity”. It would be understood that what constitutes “natural immunity” is determined by analysis of patient subpopulations' immune responses to natural infection and whether or not a candidate vaccine elicits an immune response that is comparable to or greater than (above) natural immunity is a common consideration by regulatory bodies for a vaccine's market approval. Methods for measuring an immune response are known and may include, for measure of the humoral response, the Geometric Mean Titre (GMT) with 95% Confidence Interval (CI) of neutralizing antibodies and/or, for measure of the cell-mediated/cellular response, the concentration of T cell cytokines. For example, induction of proliferation or effector function of the particular lymphocyte type of interest (e.g., B cells, T cells, T cell lines, and T cell clones) may be assessed; for example, spleen cells from immunized mice can be isolated and the capacity of cytotoxic T lymphocytes to lyse autologous target cells that contain a polynucleotide (e.g., a self-replicating RNA molecule) that encodes the modified betacoronavirus S protein or S protein fragment. In addition, T helper cell differentiation can be analyzed by measuring proliferation or production of TH1 (IL-2, TNF-α, or IFN-γ) cytokines and/or TH2 (IL-4 or IL-5) cytokines by ELISA or directly in CD4+ T cells by cytoplasmic cytokine staining and flow cytometry. Contemporary techniques for such analysis often include Enzyme-Linked Immunospot (ELIspot) and Flow Cytometry (FCM)-based detection. Certain cytokines are associated with certain classes of T cell(s) and, thus, the measure of those cytokines is associated with a cellular (T cell) immune response. Exemplary cytokines and their associated class of T cell(s) are below. Literature on detecting and quantifying an immune response includes: Plebanski et al. 2010 Expert Rev. Vaccines 9(6):596-600; Todryk 2018 Vaccines (Basel) 6(4): 84; Folds and Schmitz 2003 J. Allergy Clinical Immunology 111(2) Supplement 2: S702-S711; and Falchetti et al. 1998 Immunology 95:346-351.

| Cytokines | Class of T cell |
|---|---|
| IFNγ, TNFα, IL-2 | Th1 |
| IL-4 , IL-5, IL-6, IL-9, IL-10, IL-13 | Th2 |
| IL-17 A/F, IL-22, IL-21, IL-25, | Th17 |
| IL-26 | |

“At natural immunity” or an immune response “comparable to natural immunity” means not materially different or not statistically different than natural immune response. An immune response that is “at or above natural immunity” means an immune response comparable to natural immunity or greater than natural immunity by a statistically significant amount. Where a natural immune response would include both a humoral and cellular response, saying a vaccine induced immune response is “at or above natural immunity” means the vaccine-induced response solicited a humoral response that is comparable to or above the natural humoral response, solicited a cellular response that is comparable to or above the natural cellular response, or both (solicited both humoral and cellular responses that are comparable to or above the natural humoral and cellular responses, respectively). An immune response may be quantified by the measure of the humoral response (e.g., Geometric Mean Titre (GMT) with 95% Confidence Interval (CI) of neutralizing antibodies) and/or the cell-mediated/cellular response (e.g., concentration of T cell cytokines) of a test group subject(s) who received the candidate vaccine composition and that of a control group subject(s) who did not receive the candidate vaccine composition, then comparing them. If the test group values are not statistically different from the control group values (may be averaged values), then the test group's immune response is “at natural immunity” or “comparable to natural immunity”. If the test group values are above the control group's values (statistically different), then the test group values are “above natural immunity”.

“Immunogenicity” refers to an antigen's or composition's ability to induce an immune response. See generally, e.g., Ma et al., 2011 PLoS Path. 7(9), e1002200. An “immunogenic composition” is a composition that comprises one or more antigens that, administered to a subject, will induce an immune response. An immunogenic composition may also comprise an adjuvant (e.g., an immunostimulating adjuvant). As used herein, an immunogenic composition (e.g., a prophylactic or therapeutic vaccine composition) means that which is suitable for pharmaceutical use (e.g., comprises purified antigen(s)), including use for administration to a human subject.

An “effective amount” means an amount sufficient to cause the referenced outcome. An “effective amount” can be determined empirically and in a routine manner using known techniques in relation to the stated purpose. An “immunologically effective amount”, with respect to an antigen or immunogenic composition, is a quantity sufficient to elicit a measurable immune response in a subject (e.g., 1-100 μg of antigen). With respect to an adjuvant, an “adjuvanting effective amount” or “immunostimulating effective amount” (in the case of an adjuvant that is an immunostimulant) is a quantity sufficient to modulate an immune response (e.g., 1-100 μg of adjuvant). To obtain a protective immune response against a pathogen, it can require multiple administrations of an immunogenic composition. So in the context of, for example, a protective immune response, an “immunologically effective amount” encompasses a fractional dose that contributes in combination with previous or subsequent administrations to attaining a protective immune response.

“Enhanced thermostability” or “increased thermostability” means the molecule (e.g., modified S protein or S protein fragment) has at least a lower rate of unfolding, under comparable conditions, than a wild type S protein (e.g., comprising SEQ ID NO: 3) or control S protein (e.g., comprising SEQ ID NO: 4) (neither of which comprise a stabilizing mutation). As a specific example, a modified betacoronavirus S protein sequence, or fragment thereof, comprising one or more stabilizing mutations and that has enhanced thermostability means the modified betacoronavirus S protein or fragment unfolds slower or has an increased shelf life, under comparable conditions (e.g., the same conditions), than a wild type or control betacoronavirus S protein or S protein fragment that does not comprise one or more stabilizing mutation. As the context requires, the thermostability of two or more stabilized mutants may be compared and one may be said to be more thermostable than the other. “Conditions” as used herein includes experimental and physiological conditions. It may be specified that a composition comprising a stabilized mutant has an increased shelf life as compared to a composition comprising its wild type counterpart or a control (non-stabilized-mutant) molecule (i.e., the molecule does not comprise one or more stabilizing mutation). See, e.g., U.S. Pub. No. 2011/0229507; Clapp et al., 2011 J. Pharm. Sci. 100(2): 388-401, discussing increased stability via adjuvants and assessing antigen stability in altered pH, hydration, and temperature conditions; and Rossi et al., 2016 Infect. Immun. 84(6): 1735-1742. Stability herein may be provided by the delta stability (dStability or dS) scoring method, which is the computationally-determined difference between the relative thermostability of an in-silico mutant protein and that of the corresponding wild type or control (i.e., non-stabilized-mutant) protein. Methods of determining dStability are known (WO 2020/079586 (PCT/IB2019/058777), MALITO et al.) and may include the use of tools such as Molecular Operating Environment (MOE) software (REF: Molecular Operating Environment (MOE) software; Chemical Computing Group Inc., available at WorldWideWeb(www).chemcomp.com). dS is measured by kcal/mol. Lower dS values indicate higher protein stability, while higher dS values indicate lower protein stability. It may be specified that the mutant polypeptides of the present invention have a higher relative thermostability (in kcal/mol) as compared to a non-mutant polypeptide under the same experimental conditions. It may be further specified that the mutant polypeptides of the present invention have a lower dS value than a non-mutant polypeptide under the same experimental conditions. It will be understood from the present invention that a mutant polypeptide having a lower dS value as compared to a non-mutant polypeptide under the same experimental conditions is more stable than the non-mutant polypeptide. The stability enhancement can be assessed using differential scanning calorimetry (DSC) as discussed in Bruylants et al. 2005 Curr. Med. Chem. 12: 2011-2020 and Calorimetry Sciences Corporation's “Characterizing Protein stability by DSC” (Life Sciences Application Note, Doc. No. 2021102136 February 2006) or by differential scanning fluorimetry (DSF). An increase in (thermo)stability may be characterized as an at least about 2° C. increase in thermal transition midpoint (Tₘ), as assessed by DSC or DSF. See, for example, Thomas et al., 2013 Hum. Vaccin. Immunother. 9(4): 744-752. A “significant” increase in, or enhancement of, thermostability is defined as an increase of at least 5° C. in the calculated Tm of a complex (calculated by, for example, the protocol provided at Example 4.7 of WO 2020/079586 (PCT/IB2019/058777), MALITO et al.).

“Fragment,” refers to a portion (that is, a subsequence) of a polynucleotide/polypeptide and is generated by cleaving one or more residues from either end of the reference polynucleotide/polypeptide sequence (e.g., deletion of the transmembrane domain). In this way, a fragment is an exemplary deletion mutant. A fragment is at least 100, 200, 300, 400, 500, 600, 700, 800, 900, 1000, or 1100 amino acids in length (and any integer value in between). An “immunogenic fragment” is a portion of a polynucleotide/polypeptide that elicits an immune response (in the case of an antigen fragment) or modulates an immune response (in the case of an immunostimulant fragment). An “immunogenic fragment” refers to a molecule containing one or more epitopes (e.g., linear, conformational or both) capable of stimulating a host's immune system to make a humoral and/or cellular antigen-specific immunological response (i.e. an immune response which specifically recognizes a naturally occurring polypeptide, e.g., a viral or bacterial protein). An immunogenic fragment of an antigen retains at least one immunogenic epitope of its reference (“source”) polynucleotide/polypeptide. An “epitope” is that portion of an antigen that determines its immunological specificity. T- and B-cell epitopes can be identified empirically (e.g. using PEPSCAN or similar methods). Herein, when the reference (“source”) polynucleotide/polypeptide is described as having one or more specific amino acid substitutions (e.g., “an S protein comprising an F17S substitution, numbered according to SEQ ID NO: 3”), it is meant that a “fragment thereof” also comprises that one or more specific amino acid substitutions (e.g., the fragment thereof would also comprise the F17S substitution, numbered according to SEQ ID NO: 3). An exemplary immunogenic fragment for use herein consists a SARS-βCoV spike protein Receptor Binding Domain (RBD), such as an immunogenic fragment comprising the amino acids corresponding to residues 330-521 of any one of SEQ ID NOs: 5-114, optionally linked to a pharmaceutically acceptable carrier (e.g. a nanoparticle or IgG1 Fc), or delivered to a subject through an adeno-associated virus (AAV) or a Self-Amplifying RNA Molecule (SAM). Such immunogenic fragments consisting of a spike protein RBD were previously described for candidate MERS-CoV and SARS-CoV-1 vaccines (including Fc chimeric proteins and AAV delivery) (Zheng B J et al. 2008 Hong Kong Med J 14(Suppl 4):S39-43; Du L. et al. 2009 Nat. Rev. Microbio. 7:226-236; Wang et al. 2016 Antiviral Research 133: 165-177). For clarity and with respect to the substitution mutations provided herein, if the fragment is of a protein (e.g., an S protein) and that protein is said to comprise one or more of the presently provided substitution mutations; the “fragment thereof” also comprises those one or more substitution mutations.

“Immunodominance” is the immunological phenomenon in which immune responses are mounted against only a subset of the antigenic peptides produced by a pathogen. Immunodominance has been evidenced for antibody-mediated and cell-mediated immunity. As used herein, an “immunodominant antigen” is an antigen which comprises immunodominant epitopes. In contrast, a “subdominant antigen” is an antigen which does not comprise immunodominant epitopes, or in other terms, only comprises subdominant epitopes. As used herein, an “immunodominant epitope” is an epitope that is dominantly targeted, or targeted to a higher degree, during an immune response to a pathogen. As used herein, a “subdominant epitope” is an epitope that is not targeted, or targeted to a lower degree, during an immune response to a pathogen.

By “linked” it is meant the two or more referenced molecules or structures are connected, attached, fused, bound, or ligated. The two or more molecules and/or structures may be linked naturally (e.g., by the action of an endogenous enzyme and including the covalent or non-covalent bonds that naturally form between two proteins) or recombinantly (e.g., contacting two polynucleotides with a heterologous enzyme to ligate the polynucleotides together or recombinantly inserting one or more linkers between two proteins so that the proteins form a complex); and/or linked reversibly or irreversibly. For clarity, the two or more molecules and/or structures may be linked chemically (e.g., chemical conjugation of a protein and a sugar) or biologically (e.g., enzymatic conjugation of a protein and a sugar). “Linked” does not mean the two or more molecules and/or structures have to be next to each other (“adjacent”) without any other molecule or structure between them (“immediately adjacent to”)—it is well known, for example, that a gene's coding sequence may be linked to a control sequence (e.g., a promoter, enhancer, or IRES) and that the coding sequence may not be immediately adjacent to the control sequence: a coding sequence may be hundreds of base pairs away from its enhancer. Similarly, two genes located on the same chromosome (with hundreds or thousands of base pairs between them) are said to be “linked” in the field.

By “modify” or “modified”, it is meant that molecule (such as a peptide or polypeptide or nucleic acid or polynucleic acid) is changed in structure with reference to a reference molecule by changing the structure thereof. When referring to molecules that are not naturally occurring, the modified molecules do not include naturally occurring molecules and/or naturally occurring mutation.

By “mutation”, it is meant an insertion, deletion, or substitution (e.g., point mutation) of a nucleic acid residue or amino acid residue. A substitution herein excludes an “identical mutation,” which is the substitution of a nucleic/amino acid residue with a natural or synthetically produced residue having the same chemical structure. By way of example, the substitution of alanine at position 27 of the sequence SEQ ID NO: 3 with an alanine analog (A′) as in A27A′ is an “identical mutation” as used herein and is not within the meaning of “substitution” here. A mutation herein may be clarified with the proviso that an identical mutation is excluded. A “receptor binding mutation” means one or more mutations (sequence modifications) at a location that, in the wild type or control sequence, is involved in receptor binding (e.g., receptor recognition or binding per se). A variety of approaches may be implemented, independently or together, through the introduction of receptor binding mutations such as, for example, knock-down (KD) or knock-out (KO) approach whereby residues involved in wild type receptor binding are mutated (“receptor binding knock-down mutations” or “receptor binding knock-out mutations”, respectively); another approach being the introduction of glycosylation sites (e.g., introduction of the N-linked glycosylation N—X-T or N—X—S motif, where X is not proline) so that residues involved in wild type receptor binding are shielded (encumbered) (“receptor binding glycan mutations” or “receptor binding N-glycan mutations”).

The term “nucleic acid” in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus, the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap. Nucleic acid molecules as disclosed herein can take various forms (e.g. single-stranded, double-stranded) but are nonetheless recombinant and may comprise heterologous sequences (e.g., a heterologous signal sequence polynucleotide operably linked to an S protein polynucleotide).

“Operably linked” means two or more molecules (e.g., DNA, RNA, protein, peptides, chemical compounds, or a combination thereof) are linked or attached (e.g., directly or indirectly in a covalent or non-covalent, perhaps reversible, manner) such that the function of the two or more molecules is maintained. In the context of regulatory elements, for example, such as an enhancer and a promoter, it is well understood that non-adjacent DNA sequences are “linked” in that they are within the same polynucleotide sequence and “operably linked” in that each performs its function (as an enhancer and as a promoter, respectively). In the context of a fusion/chimeric protein comprising, for example, a carrier (such as a nanoparticle, antibody, or antibody fragment) operably linked to a protein antigen, it would be understood that a variety of linkage techniques may be used and that “operably linked” would refer to the function of the nanoparticle (or antibody or antibody fragment) as carrier and of the protein as antigen being maintained.

“Purified” means removed from its natural environment and substantially free of impurities from that natural environment (such as other chromosomal and extra-chromosomal DNA and RNA, organelles, and proteins (including other proteins, lipids, or polysaccharides which are also secreted into culture medium or result from lysis of host cells). For clarity and as used herein, an antigen within a pharmaceutical, immunogenic, vaccine, or adjuvant composition is a purified antigen (whether or not the word “purified” is recited). It is understood in the field that for an antigen, agent, adjuvant, additive, vector, molecule, compound, or composition in general to be suitable for pharmaceutical or vaccine use (i.e., “pharmaceutically acceptable”), it must be purified (i.e., not crude). It would be further understood that “purified” is a relative term and that absolute (100%) purity is not required for, e.g., pharmaceutical or vaccine use. A molecule may be at a purity of at least 85%, 86%, 87%, 88%, 89%, 90%, 91%, 92%, 93%, 94% or 95% of a composition's total proteinaceous mass (determined by, e.g., gel electrophoresis). Methods of purification are known and include, e.g., various types of chromatography such as High Performance Liquid Chromatography (HPLC), hydrophobic interaction, ion exchange, affinity, chelating, and size exclusion; electrophoresis; density gradient centrifugation; or solvent extraction. “Isolated” means removed from its natural environment and not linked to a recombinant molecule or structure (e.g., not bound to a recombinant antibody or antibody fragment) including not linked to a laboratory tool (e.g., not linked to a chromatography tool such as not bound to an affinity chromatography column). Hence, an “isolated betacoronavirus antigen”, such as an “isolated modified betacoronavirus Spike protein or Spike protein fragment”, is not on the surface of a betacoronavirus-infected cell or within an infectious betacoronavirus virion or bound to a recombinant antibody or recombinant antibody fragment (which occurs in an ELISA assay, for example). It would be understood that an antigen being bound to an antibody or antibody fragment (through epitope recognition, for example) is different than an antigen being operably linked to an antibody or antibody fragment (operable linkage in that case would use recombinant techniques and produces a molecule that does not occur in nature).

“Recombinant” when used to describe a biological molecule or biological structure (e.g., protein, nucleic acid, organism, cell, vesicle, sacculi, or membrane) means the biological molecule or biological structure is artificially produced (e.g., by laboratory methods), synthetic, and/or has a different structure and or function than the molecule or structure from which it was obtained or than its wild type counterpart. For clarity, a recombinant molecule or recombinant structure that is synthetic may nonetheless function comparably to its wild type counterpart. For clarification, a “recombinant nucleic acid” or “recombinant polynucleotide” means a nucleic acid/polynucleotide that, by virtue of its origin or manipulation (e.g., by laboratory methods), (1) is not associated with all or a portion of the polynucleotide with which it is associated in nature; and/or (2) is linked to a polynucleotide other than that to which it is linked in nature. A “recombinant protein/polypeptide” thereby encompasses a protein/polypeptide produced by expression of a recombinant polynucleotide. For clarification, a “purified protein” (e.g., a protein suitable for pharmaceutical use) is encompassed within the term “recombinant protein” because a purified protein is both artificially produced and has a different function than the crude protein (or extract or culture) from which it was obtained. A biological molecule or biological structure of the present invention may be described as “artificially produced”. “Heterologous” denotes that the two referenced biological molecules or biological structures are not naturally associated with each other (would not contact each other but-for the hand of man) or that the referenced biological molecule/structure is not in its natural environment. For example, when a nucleic acid molecule is operably linked to another polynucleotide that it is not associated with in nature, the nucleic acid molecule may be referred to as “heterologous” (i.e., the nucleic acid molecule is heterologous to at least the polynucleotide). Similarly, when a polypeptide is in contact with or in a complex with another protein that it is not associated with in nature, the polypeptide may be referred to as “heterologous” (i.e., the polypeptide is heterologous to the protein). Further, when a host cell comprises a nucleic acid molecule or polypeptide that it does not naturally comprise, the nucleic acid molecule and polypeptide may be referred to as “heterologous” (i.e., the nucleic acid molecule is heterologous to the host cell and the polypeptide is heterologous to the host cell).

“Reducing” means to lower or eliminate (i.e., “reduce/-ing” includes zero or 100% reduction). “Lowering” as used herein does not include zero (i.e., excludes 100% reduction or elimination). “Prevention” means to inhibit or stop (i.e., “prevent/-ing/-ion” includes zero or 100% blockage). “Inhibition” as used herein does not include zero (i.e., “inhibit/-ing/-ion” excludes 100% blockage or stopping).

Consistent with the official naming conventions in the art, the Severe Acute Respiratory Syndrome (SARS) betacoronavirus human pathogen which caused the international 2019/2020 pandemic may be referred to as “SARS-CoV-2” (the official name, 2020 Nat. Microbiol. 5(4):536:544; see Wang et al. 2020 Cell 181:894-904, with previous names being “WH-Human1” (see Wu et al. 2020 Nature 579:265-269) and “2019-nCoV” (see Wrapp et al. 2020 Science 367(6483):1260-1263). The respiratory disease(s) caused by SARS-CoV2 may be referred to as “COVID-19” (2020 Nat. Microbiol. 5(4):536:544), e.g. viral pneumonia having exemplary symptoms of fever, cough, and/or dyspnea). For clarity, “SARS-CoV-1” is used herein to refer to the SARS betacoronavirus, lineage B human pathogen which caused an epidemic in 2002/2003 (see Li et al. 2005 Science 309:1864-1868). What is “SARS-CoV-1” herein is usually referred to as just “SARS-CoV” in the art. “SARS-βCoV” may be used herein to refer to SARS betacoronaviruses in general (including MERS-CoV, SARS-CoV-1, and SARS-CoV02). “SARS-β, BCoV” may be used to refer to SARS beta, lineage B coronaviruses in general (including SARS-CoV-1 and SARS-CoV-2).

“Sequence identity” as used herein means matches between two nucleic acids or two amino acids. As would be understood within the field, a “match” during sequence alignment is assigned when the two nucleic/amino acids are the same or comparable to the other (such as when one is a synthetic analog of the other). To be clear, as used herein a sequence “match”, and therefore “sequence identity”, does not encompass what are known as “conserved substitutions” or “conservatively substituted residues” by the field. Unless specified otherwise, “sequence identity” as used herein means the nucleic/amino acids are the same (identical) and not merely similar or “conserved substitutions” of each other. “Sequence identity” is determined by sequence alignment, such as by pairwise, global alignment using the Needleman-Wunsch algorithm and default parameters. Pairwise sequence alignment and the various algorithms therefor, is well understood in the art (Mullan 2005 Briefings in Bioinformatics 7(1):113-115); as are multiple sequence alignment methodologies and algorithms (Daugelaite et al. 2013 ISRN Biomathematics 2013 (Article ID 615630): 14 pages). As an example, Clustal Omega is a popular multiple sequence alignment (MSA) tool by EMBL-EBI and COBALT is a popular MSA tool by NCBI (each with its own functionalities). For clarification, N-terminal or C-terminal (or 5′ or 3′) residues such as signal peptides, tags, or leader sequences may be excluded from an alignment. With many alignment tools, an asterisk (*) denotes identity between residues, a colon (:) denotes highly similar residues, a period (.) denotes weakly similar residues, and a space ( ) denotes no similarity; a hyphen (-) denotes a gap. “Percent sequence identity” between two amino acid sequences or between two nucleic acid sequences means the percentage of nucleic/amino acid residue matches between the two sequences over the reported aligned region (including any gaps in the length); such as the percentage of identical residue matches between the two sequences over the reported aligned region following pairwise, global alignment using the Needleman-Wunsch algorithm and default parameters. It is well understood in the field that two sequences may be identical but-for one or more inserted or deleted residues (gaps). Such gaps may be “end gaps” (i.e., insertions or deletions at the N-terminal or C-terminal (for protein) or 5′ or 3′ (for polynucleotide) ends of the sequence) or “internal gaps” (gaps in the length of a sequence, i.e., are not located at the end (first or last residue) of the sequence). Therefore, use of an alignment algorithm that accounts for at least internal gaps is preferred. One such alignment algorithm is the pairwise, global Needleman-Wunsch algorithm. Percent sequence identity herein is preferably determined by pairwise, global alignment with the Needleman-Wunsch algorithm (Needleman and Wunsch, 1970 J. Mol. Biol. 48(3): 443-453), using default parameters (“Needleman-Wunsch algorithm with default parameters” means: Gap opening penalty (GAP OPEN) 10.0 and with Gap extension penalty (GAP EXTEND) 0.5, with no penalty for end Gaps (END GAP PENALTY FALSE), and using the EBLOSUM62 scoring matrix (BLOSUM62 scoring table) for amino acid sequences or EDNAFULL scoring matrix for nucleotide sequences). The Needleman-Wunsch algorithm and these default parameters is implemented in the publicly available Needle tool in the EMBL-EBI EMBOSS package (Rice et al. 2000 Trends Genetics 16: 276-277; see also the World Wide Web at ebi.ac.uk/Tools/psa/emboss_needle). Preferably, the default “pair” output format from EMBOSS Needle is used. It may therefore be specified herein that “X has Y % sequence identity to the sequence SEQ ID NO: W, as determined by the Needleman and Wunsch algorithm with default parameters”. Percent sequence identity” is calculated by dividing the [total number of identical residues] (numerator) by the [total number of aligned residues](denominator) and then multiplying that result by 100; optionally then rounding down to the next nearest whole number. See the example alignment herein above. It is notable that the denominator for a percent sequence identity calculation following alignment with the Needleman and Wunsch algorithm with default parameters may not be equal to the total length of either sequence (see the example alignment herein above at the description of “corresponding to” and “corresponds to”). Provided herein are polypeptides (e.g., Spike proteins) comprising an amino acid sequence with at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from the group consisting of SEQ ID NOs: 5-114 (or also to SEQ ID NOs 125-134). Provided herein are polypeptides (e.g., Spike proteins such as Spike protein fragments) comprising a Receptor Binding Domain consisting of an amino acid sequence with at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the residues corresponding to 330-521 of the sequence selected from the group consisting of SEQ ID NOs: 5-114 (or also to SEQ ID NOs 125-134).

“Stabilizing mutation” means a mutation in a betacoronavirus S protein (or S protein fragment) polynucleotide or amino acid sequence that has the effect of “stabilizing” the mutant S protein (or mutant S protein fragment). A “stabilized” protein or protein fragment has, for example, decreased misfolding, reduced protein domain movements, reduced protein domain rearrangements, increased half-life in-vitro or in-vivo, increased melting temperature (Tm), and/or increased thermostability as compared to a wild type protein (e.g., wild type S protein SEQ ID NO: 3), control protein, or control protein fragment (e.g., control S protein fragment SEQ ID NO: 4). See McCallum et al. 2020 bioRxiv HyperTextTransferProtocolSecure://doi.org/10/1101/2020.06.03.129817; Henderson et al. 2020 bioRxiv HyperTextTransferProtocolSecure://doi.org/10.1101/2020.05.18.102087. Stabilizing mutations include the HBNet mutations, PROSS mutations, HBNet-PROSS mutations, and/or Disulfide Mutations summarized within tables herein. See also SEQ ID NOs: 5-64. A stabilizing mutation is not detrimental to the use of the resultant mutant protein (e.g., S protein or S protein fragment) as an antigen. In particular, the HBNet mutations, PROSS mutations, HBNet-PROSS mutations, and Disulfide Mutations of the tables herein were designed to conserve putative S protein epitopes and tertiary/three-dimensional structure generally so that resultant mutant S proteins remain immunogenic (regarding SARS-CoV-2 epitopes, see Grifoni et al. 2020 Cell 181:1-13 and Supplementary Materials; Kiyotani et al. 2020 J. Hum. Genet. HyperTextTransferProtocolSecure://doi.org/10.1038/s10038-020-0771-5). A molecule comprising one or more stabilizing mutation may be referred to as a “stabilized mutant”. A disulfide bridge forms between two cysteine (C) residues within a polypeptide (or between two cysteine residues that are each within a different polypeptide, as in the context of protein complexes). Therefore, a “disulfide bridge mutation” means the substitution mutations for introducing a disulfide bridge into the molecule (e.g., modified S protein or S protein fragment). If the molecule already comprises a cysteine residue at the target disulfide bridge location (e.g., one cysteine residue innately exists there within the wild type sequence), then one substitution mutation to cysteine (C) may be sufficient to introduce a disulfide bridge (and thereby increase the stability of the resultant mutant molecule). Alternatively, two substitution mutations to cysteine (C) will be needed at the target disulfide bridge location.

A “subject” is a living multi-cellular vertebrate organism and as used herein, a mammal. In the context of this disclosure, the subject can be an experimental subject, such as a non-human mammal, e.g., a mouse, a guinea pig, a cotton rat, or a non-human primate. Alternatively, the subject can be a human subject. In particular, a subject herein may be a human subject at risk of being infected or reinfected with a betacoronavirus (e.g., MERS-CoV, SARS-CoV-1, or SARS-CoV-2), at risk of reactivation, antibody-dependent enhancement of disease, or at risk of respiratory disease (e.g., COVID-19). A subject which has been infected with the virus prior to being treated with an immunogenic composition herein may have shown clinical signs of the infection (symptomatic subject) or may not have shown clinical signs of the viral infection (asymptomatic subject). In one embodiment, the symptomatic subject has shown several episodes with clinical symptoms of infections over time (recurrences) separated by periods without clinical symptoms.

As used herein, the terms “treat” and “treatment” as well as words stemming therefrom, are not meant to imply a “cure” of the condition being treated in all individuals, or 100% effective treatment in any given population. Rather, there are varying degrees of treatment which one of ordinary skill in the art recognizes as having beneficial therapeutic effect(s). In this respect, the methods and uses herein can provide any level of treatment of betacoronavirus infection and, in particular, MERS-CoV, SARS-CoV-1, or SARS-CoV-2 related disease in a subject in need of such treatment, and may comprise reduction in the severity, duration, or number of recurrences over time, of one or more conditions or symptoms of betacoronavirus (e.g., MERS-CoV, SARS-CoV-1, or SARS-CoV-2) infection, and in particular SARS-CoV-2 related disease (e.g., COVID-19).

As used herein, “therapeutic immunization” or “therapeutic vaccination” refers to administration of the immunogenic compositions of the invention to a subject, preferably a human subject, who is known to be infected with a pathogen (e.g., a betacoronavirus such as MERS-CoV, SARS-CoV-1, and/or SARS-CoV-2) at the time of administration, to treat the infection or pathogen-related disease or to prevent reinfection or reactivation. As used herein, “prophylactic immunization” or “prophylactic vaccination” refers to administration of the immunogenic compositions of the invention to a subject, preferably a human subject, within whom pathogen cannot be detected (e.g., who is not infected with pathogen) at the time of administration, to prevent infection or pathogen-related disease.

A “total dose” means the sum of doses (e.g., sum of partial doses co-administered or administered in close temporal sequence). When there is only one dose administration, that dose is the “total dose.”

As used herein, a “variant” is a nucleic acid molecule or peptide that differs in sequence from a reference nucleic acid molecule or peptide, respectively, but retains essential properties of the reference molecule/peptide. Changes in the sequence of variants are limited or conservative, so that its sequence is highly similar overall and, in many regions, identical to the sequence of the reference molecule/peptide. A variant and reference molecule/peptide can differ in sequence by one or more substitutions, additions or deletions in any combination. A variant of a nucleic acid molecule or peptide can be naturally occurring, such as an allelic variant (e.g., several SARS-CoV-2 spike protein variants are known in the art, see Wrapp et al. 2020 Science 367(6483):1260-1263). Non-naturally occurring variants of nucleic acids and peptides may be made by mutagenesis techniques or by direct synthesis.

The singular terms “a,” “an,” and “the” include plural referents unless context clearly indicates otherwise. Similarly, the word “or” is intended to include “and” unless the context clearly indicates otherwise (see also “and/or” herein). The term “plurality” refers to two or more.

The term “comprises” is open-ended and means “includes.” Thus, unless the context requires otherwise, the word “comprises” or “has”, and variations thereof (including “comprise” and “comprising” or “have” and “having”, respectively), will be understood to imply the inclusion of a stated compound(s), molecule(s), composition(s), or steps, but not to the exclusion of any other compound(s), molecule(s), composition(s), or steps. The terms “comprising” and “having” when used as a transition phrase herein are open-ended whereas the term “consisting of” when used as a transition phrase herein is closed (i.e., limited to that which is listed and nothing more). In certain embodiments and for readability, the word “is” may be used as a substitute for “consists of” or “consisting of”. The abbreviation, “e.g.” is derived from the Latin exempli gratia, and is used herein to indicate a non-limiting example. Thus, the abbreviation “e.g.” is synonymous with the term “for example.”

Unless specifically stated otherwise, providing a numeric range (e.g., “25-30”) is inclusive of endpoints (i.e., includes the values 25 and 30). An endpoint of a range may be excluded by reciting “exclusive of lower endpoint” or “exclusive of upper endpoint”. Both endpoints may be excluded by reciting “exclusive of endpoints”.

Unless specifically stated, a process comprising a step of mixing two or more components does not require any specific order of mixing. Thus, components can be mixed in any order. Where there are three components then two components can be combined with each other, and then the combination may be combined with the third component, etc. Similarly, while steps of a method may be numbered (such as (1), (2), (3), etc. or (i), (ii), (iii)), the numbering of the steps does not mean that the steps must be performed in that order (i.e., step 1 then step 2 then step 3, etc.). The word “then” may be used to specify the order of a method's steps.

The following terminology may be used to reference amino acid residues: Alanine (Ala or A), Arginine (Arg or R), Asparagine (Asn or N), Aspartic acid (Asp or D), Cysteine (Cys or C), Glutamic acid (Glu or E), Glutamine (Gln or Q), Glycine (Gly or G), Histidine (His or H), Isoleucine (Ile or I), Leucine (Leu or L), Lysine (Lys or K), Methionine (Met or M), Phenylalanine (Phe or F), Proline (Pro or P), Serine (Ser or S), Threonine (Thr or T), Tryptophan (Trp or W), Tyrosine (Tyr or Y), Valine (Val or V).

### Spike Proteins

Coronaviral infections initiate with binding of virus particles to host surface cellular receptors. Receptor recognition is therefore an important determinant of the cell and tissue tropism of the virus. In addition, the virus must be able to bind to the receptor counterparts in other species for inter-species-transmission to occur. With the exception of HCoV-OC43 and HKU1, both of which engage sugars for cell attachment, human coronaviruses (HCoVs) recognize proteinaceous receptors. HCoV-229E binds to human aminopeptidase N (hAPN); MERS-CoV interacts with human dipeptidyl peptidase 4 (hDPP4 or hCD26); and all three of SARS-CoV-1, hCoV-NL63, and SARS-CoV-2 interact with human angiotensin-converting enzyme 2 (hACE2). See Wang et al. 2020 Cell 181: 894-904.

Structural proteins are encoded by one-third of coronavirus (CoV) genomes (one-third from the 3′ end), such structural proteins including the spike (S) glycoprotein, small envelope protein (E), integral membrane protein (M), and genome-associated nucleocapsid protein (N). See SEQ ID NO: 1. Some CoVs also contain a hemagglutinin esterase (HE). Interspersed between these genes, are several genes coding for accessory proteins, many of which are involved in regulating the host immune system. The proteins E, M, and N are mainly responsible for the assembly of the virions, while the S protein has an essential role in virus entry and determines tissue and cell tropism, as well as host range. Wang et al. 2016 Antiviral Research 133: 165-177.

In CoVs, the process for entry into host cells is mediated by the densely glycosylated, envelope-embedded, surface-located spike (S) glycoprotein (“S protein”). The S protein is a homotrimeric class I fusion protein with two subunits in each spike monomer (or “protomer”), called “S1” and “S2”, which are responsible for receptor recognition and membrane fusion, respectively. Wrapp et al. 2020 Science 367(6483):1260-1263. The S protein is in a metastable prefusion conformation that, when triggered by the S1 subunit binding to a host cell receptor, undergoes a substantial structural rearrangement to fuse the viral membrane with the host cell membrane. Wrapp et al. 2020 Science 367(6483):1260-1263 and Wang et al. 2020 Cell 181: 894-904. Receptor binding destabilizes the prefusion homotrimer, resulting in the shedding of the S1 subunit and transition of the S2 subunit to a stable postfusion conformation (in the case of MERS-CoV and SARS-CoV-2, but not SARS-CoV-1, the S protein is cleaved by host proteases (furin) into the S1 and S2 subunits, enabling S2 to form its stable postfusion conformation). Wrapp et al. 2020 Science 367(6483):1260-1263 and Wang et al. 2020 Cell 181: 894-904; see also Follis et al. 2006 Virology 350:358-369. The S1 subunit can be further divided into an N-terminal domain (NTD) and a Receptor Binding Domain (RBD) (the RBD is also called a C-terminal domain (CTD)). See Wrapp et al. 2020 Science 367(6483):1260-1263 & Suppl. Material as well as Wang et al. 2020 Cell 181: 894-904 for the structures of SARS-CoV-1 and SARS-CoV-2; see also Yuan et al. 2017 Nat. Comm. 8(15092), 9 pgs & Suppl. Materials for the structures of MERS-CoV and SARS-CoV-1. hCoV-NL63, SARS-CoV-1, and SARS-CoV-2 all utilize the RBD to interact with the hACE2 receptor. Wang et al. 2020 Cell 181: 894-904. A “full length betacoronavirus S protein” herein means it comprises (from N-terminus to C-terminus) the NTD through to, and including, the cytoplasmic tail (CT). A “CT-deleted betacoronavirus S protein fragment” herein means it comprises the NTD through to, and including, the transmembrane (TM) domain. A “TM-deleted betacoronavirus S protein fragment” means it comprises the NTD up to, and excluding, the TM domain (but a TM-deleted betacoronavirus S protein fragment may be operably linked at the C-terminus to a cytoplasmic tail or other (optionally heterologous) amino acid(s)).

In the context of vaccination by delivery of a betacoronavirus S protein or S protein fragment, it is desirable to deliver a prefusion conformation betacoronavirus S protein or S protein fragment. To lock a betacoronavirus S protein or S protein fragment in prefusion conformation, one or more proline substitutions may be introduced into its sequence, preferably one or two proline substitutions, and introduced at or near (e.g., within two residues N- or C-terminal to, or within two residues C-terminal to) the boundary between the Heptad Repeat 1 (HR1) and the Central Helix (CH). The HR1/CH boundary within SARS-CoV-2 sequence SEQ ID NO: 3 is between D959 and K960, within SARS-CoV-1 sequence SEQ ID NO: 116 the HR1/CH boundary is between D954 and K955 (see Wrapp et al. 2020 Science 367(6483):1260-1263 at Suppl. Materials FIG. S5); which residues correspond to D1040 and K1041, respectively, of MERS-CoV sequence SEQ ID NO: 118. To lock SARS-CoV-2 S protein in prefusion conformation, it is sufficient to introduce one proline residue. In particular, it is sufficient to substitute K960, numbered according to SEQ ID NO: 3, with proline (P). Therefore, a preferred embodiment provides a modified betacoronavirus S protein or fragment thereof comprising a proline (P) at the residue corresponding to 960 of the sequence SEQ ID NO: 3 (see, e.g., SEQ ID NO: 39). It was previously demonstrated that the introduction of two proline residues at or near the boundary between the SARS-CoV-2 S protein HR1 and CH is sufficient to lock the S protein in prefusion conformation (see WO2018/081318 (PCT/US2017/058370), GRAHAM B. et al. and Wrapp et al. 2020 Science 367(6483):1260-1263). In particular, the substitution of both K960 and V961, numbered according to SEQ ID NO: 3, to proline was shown to lock SARS-CoV-2 S protein in prefusion conformation (WO2018/081318 (PCT/US2017/058370), GRAHAM B. et al. and Wrapp et al. 2020 Science 367(6483):1260-1263). Therefore, another embodiment provides a modified betacoronavirus S protein or fragment thereof comprising the mutation of two immediately adjacent residues at or within two residues of the HR1/CH boundary wherein the mutations are substitutions to proline. A further preferred embodiment provides a modified betacoronavirus S protein or fragment thereof comprising prolines (P) at the residues corresponding to 960 and 961 of the sequence SEQ ID NO: 3.

To provide a prefusion conformation betacoronavirus S protein or S protein fragment or to promote the formation of trimeric complexes, it may be desirable to insert a trimerization domain (e.g., the T4 fibritin trimerization (foldon) motif) into the C-terminus of the S protein or S protein fragment. In particular, a betacoronavirus S protein fragment having an inactive transmembrane domain (e.g., inactive by deletion) or, optionally, lacking the entire C-terminus (e.g., lacking by deletion), comprises the ectodomain sequence operably linked (e.g., through the inclusion of one or more linker residues) to a trimerization domain sequence (e.g., a heterologous trimerization domain) such as the T4 fibritin trimerization (foldon) motif (see an example of this technique with MERS-CoV and SARS-CoV-1 by Yuan et al. 2017 Nat. Comm. 8(15092), 9 pgs & Suppl. Materials).

In the context of vaccination by delivery of a betacoronavirus S protein or S protein fragment, it is desirable to keep the S1 and S2 subunits operably linked, especially if prefusion conformation is desired and/or cell surface protein expression or protein secretion is desired. In the context of MERS-CoV or SARS-CoV-2 S proteins, it is thus desirable to prevent furin cleavage of the S1 and S2 subunits. For betacoronavirus vaccination by delivery of a MERS-CoV or SARS-CoV-2 S protein or S protein fragment, it is therefore desirable to deliver a furin-cleavage abrogated S protein or S protein fragment. Furin-cleavage abrogation may be achieved by introducing substitution mutations into the R—X—X—R furin recognition/cleavage motif (where the arginines (R) are “furin motif arginines” and where X is any amino acid) as was previously shown for the ⁶⁵⁶RRAR⁶⁵⁹ SARS-CoV-2 S1/S2 furin recognition site (see Wrapp et al. 2020 Science 367(6483):1260-1263, numbered according to SEQ ID NO: 3) and for the ⁷³⁰RSVR⁷³³ MERS-CoV S1/S2 furin recognition site (see Millet and Whittaker 2014 PNAS 111(42):15214-15219, numbered according to SEQ ID NO: 118). Yuan et al. (2017 Nat. Comm. 8(15092), 9 pgs & Suppl. Materials) also demonstrate a furin abrogated MERS-CoV S protein by mutation within the furin recognition motif. It is notable that wild type SARS-CoV-1 S protein maintains the residue corresponding to the C-terminal furin motif arginine (R), not the N-terminal furin motif arginine (see Wrapp et al. 2020 Science 367(6483):1260-1263 Supplemental Materials at FIG. S5). In particular, furin-cleavage abrogation may be achieved by introducing one or more substitution mutations into the furin motif, wherein the one or more substitution mutations comprise a substitution of one or both of the furin motif arginines (R). An embodiment therefore provides a betacoronavirus (βCoV) S protein or fragment thereof comprising one or more substitution mutations at the residues corresponding to R656-R659 of the sequence SEQ ID NO: 3, wherein the one or more substitution mutations include the substitution of one or both of the residues corresponding to R656 and R659 of the sequence SEQ ID NO: 3; optionally wherein the wild type or control βCoV S protein is cleaved by furin (e.g., MERS-CoV or SARS-CoV-2 S protein).

Natural sequence variation exists between betacoronavirus S proteins, even between S proteins from the same virus. As an example, 9 naturally occurring amino acid variations have been identified between SARS-CoV-2 S proteins: 3 in the NTD (F321, H49Y, S247R); 3 in the RBD (N354D, D364Y, V367F); 1 in the SD2 (D614G); and 2 in the S2 (V1129L, E1262G) (numbered according to SEQ ID NO: 3, see Wrapp et al. 2020 Science 367(6483):1260-1263 and Supplemental Materials thereof). In certain embodiments is provided a modified betacoronavirus S protein or fragment thereof having a sequence that does not include the substitution F32I, H49Y, S247R, N354D, D364Y, V367F, D614G, V1129L, or E1262G, or combinations thereof, numbered according to SEQ ID NO: 3. A particular embodiment provides a modified betacoronavirus S protein or fragment thereof having a sequence that does not include the substitution F32I, H49Y, S247R, N354D, D364Y, V367F, V 1129L, or E1262G, or combinations thereof, numbered according to SEQ ID NO: 3. It would alternatively be understood that one or more of such naturally occurring sequence variants may be included within a modified betacoronavirus S protein or S protein fragment sequence of this invention. In the context of vaccination, inclusion of one or more natural S protein sequence variants may be desirable if such variant is suspected of having a functional effect. As an example, the SD2 D614G substitution (numbered according to SEQ ID NO: 3) is believed to impact SARS-CoV-2 virulence (Brufsky 20 Apr. 2020 J Med Virol, 7 pages, doi: 10.1002/jmv.25902; Korber et al. 2020 bioRxiv (HyperTextTransferProtocolSecure: //doi.org/10.1101/2020.04.29.069054)). Therefore, an embodiment herein provides a modified betacoronavirus S protein or fragment thereof comprising a glycine (G) at the position corresponding to residue 614 of the sequence SEQ ID NO: 3 (see, e.g., the S protein fragment sequence SEQ ID NO: 4). A particular embodiment provides a modified SARS-CoV-2 S protein or fragment thereof comprising a glycine (G) at the position corresponding to residue 614 of the sequence SEQ ID NO: 3 (see, e.g., the S protein fragment sequence SEQ ID NO: 4).

Generally, there exists an inverse relationship between the flexibility of a protein and the stability of that protein (as was recently shown for the Lipase A enzyme from the mesophilic organism Bacillus subtilis, see Rathi et al., 2015 PLOS ONE 19(7): e0130289; DOI: 10.1371/journal.pone.0130289; 24 pages). One may reduce protein flexibility, and thereby increase stability, by modifying the protein's structure such as by introducing one or more mutations into the protein's amino acid sequence. Increased stability of antigens has been previously linked with improved immunogenicity such as, for example, for the pre-fusion conformation of the Respiratory Syncytial Virus (RSV) fusion protein (McLellan et al. 2013 Science 342(6158): 592-598) and the Neisseria meningitidis factor H binding protein (fHbp) (Rossi et al. 2016 Infect. Immun. 84(6): 1735-1742). Certain stabilizing mutations of a SARS-CoV-2 Spike protein have been suggested (See McCallum et al. 2020 bioRxiv HyperTextTransferProtocolSecure://doi.org/10/1101/2020.06.03.129817; Henderson et al. 2020 bioRxiv HyperTextTransferProtocolSecure://doi.org/10.1101/2020.05.18.102087). It is expected that improved stability of a betacoronavirus S protein or fragment thereof will have a desirable impact on protein preparation and production (e.g., manufacturing processes) and/or on immunogenicity. It is therefore desirable that in certain embodiments, the betacoronavirus S protein sequence, or fragment thereof, comprises one or more stabilizing mutations (such as one or more of the HBNet, PROSS, HBNet-PROSS, or Disulfide Bridge mutations provided in the Examples). In certain embodiments is provided a modified betacoronavirus S protein or fragment thereof comprising one or more of the mutations listed in Tables 1-5. See also SEQ ID NOs: 5-64. In certain embodiments is provided a modified betacoronavirus S protein, or fragment thereof, comprising an amino acid sequence that comprises one or more of the mutations listed in Tables 1-5 and wherein the modified S protein, or fragment thereof, has an increased stability as compared to a wild type (e.g., the S protein comprising the sequence SEQ ID NO: 3) or control (e.g., the S protein comprising the sequence SEQ ID NO: 4) betacoronavirus S protein.

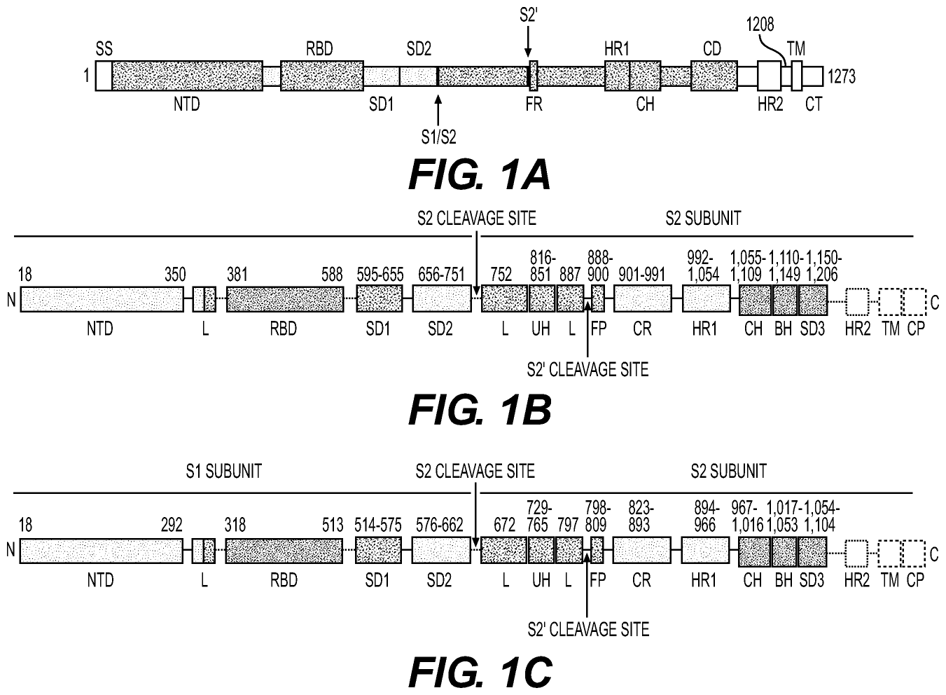

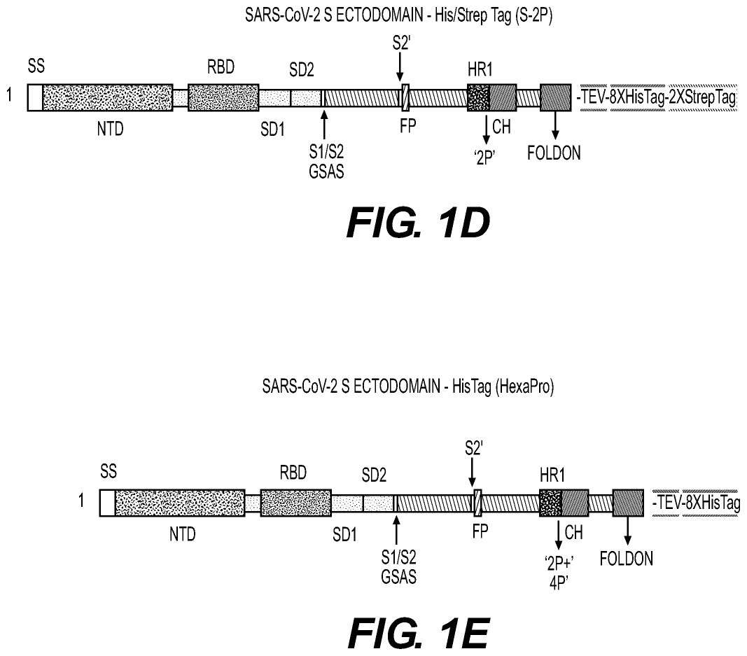

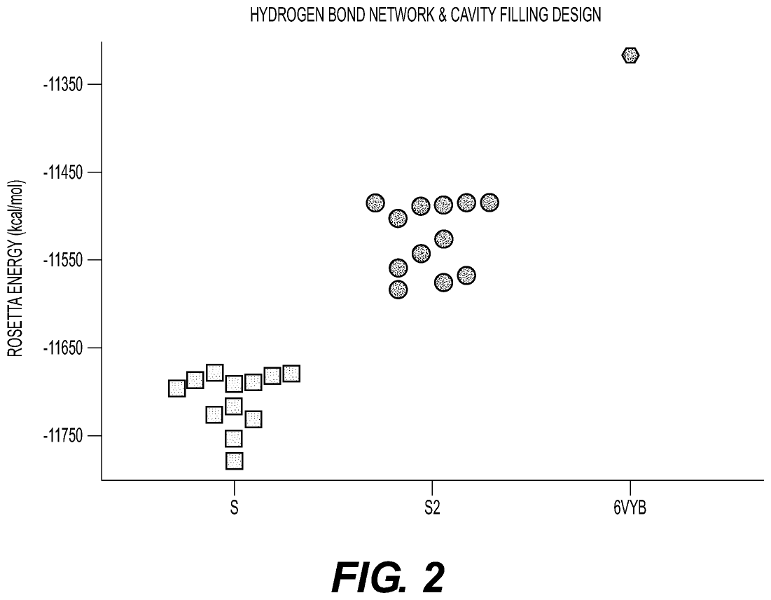

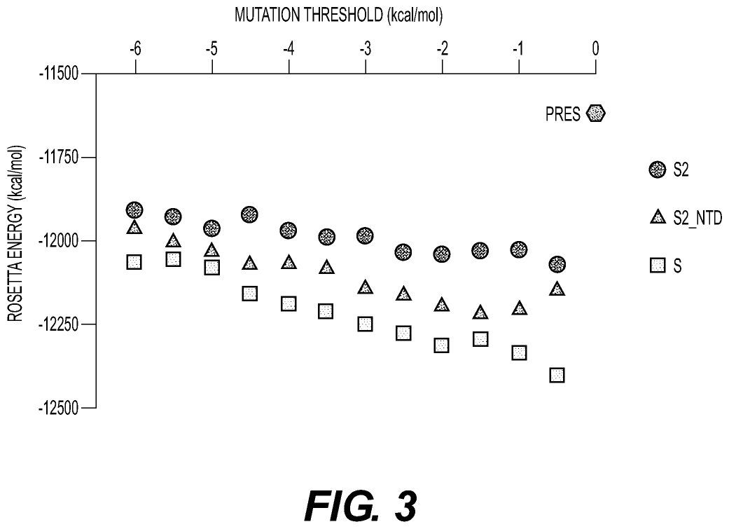

In the context of vaccine design, antibody-dependent enhancement (ADE) of viral infection or disease is a concern (see Tirado and Yoon 2003 Viral Immunol. 16(1):69-86). ADE has been observed for coronaviruses (Wan et al. 2020 94(5):e02015-19, 15 pages; Walls et al. 2019 Cell 176:1026-1039). One approach to reduce the risk of ADE in the context of vaccination by delivering an antigen to a subject, is to introduce receptor binding mutations (as defined herein above) into the antigen sequence. Where the antigen is a modified betacoronavirus S protein or fragment thereof, wherein its wild type counterpart binds hACE2 as receptor (e.g., hCoV-NL63, SARS-CoV-1, and/or SARS-CoV-2), it may therefore be desirable for the antigen sequence to comprise one or more receptor binding mutations (e.g., receptor binding knock-down mutations, receptor binding knock-out mutations, or receptor binding glycan mutations) to avoid eliciting antibodies that are comparable to hACE2 and thereby avoid, for example, enhancing the possibility of triggering conformational changes from pre- to post-fusion S protein during the course of natural SARS-β, BCoV infection. The RBDs of at least SARS-CoV-1 and SARS-CoV-2 have already been characterized and compared, providing identification of corresponding residues (Tai et al. 2020 Cell. & Mol. Imm. at FIG. 1, available before print HyperTextTransferProtocolSecure: //doi.org/10.1038/s41423-020-0400-4). Certain substitution mutations of the SARS-CoV-2 S protein RBD are provided herein (see the knock-out mutations at Example 2, Table 6 and glycan mutations at Example 2, Table 7), so certain embodiments provide a modified betacoronavirus S protein or fragment thereof (e.g., hCoV-NL63, SARS-CoV-1, and/or SARS-CoV-2 S protein or fragment thereof) with an amino acid sequence comprising an “RBD mutation” residue listed in column #2 of Table 6 at a position corresponding to the residue number in column #1 (“Target Residue in SEQ ID NO: 3”) of that same row in Table 6. Optionally one such modified betacoronavirus S protein or fragment has an amino acid sequence comprising one of SEQ ID NOs: 65-104, optionally wherein the S protein or fragment comprises a transmembrane domain or both a transmembrane domain and a cytoplasmic tail (such as a full length, modified betacoronavirus S protein).

Optionally, to facilitate expression and recovery, the modified spike protein or fragment sequence may include a signal peptide at the N-terminus. A signal peptide can be selected from among numerous signal peptides known in the art, and is typically chosen to facilitate production and processing in a system selected for recombinant expression. In one embodiment, the signal peptide is the one naturally present in the native viral spike protein (see, e.g., the summary of SEQ ID NO: 1 herein below). In another embodiment, the signal peptide is a Gaussian Luciferase signal sequence, a human CD5 signal sequence, a human CD33 signal sequence, a human IL2 signal sequence, a human IgE signal sequence, a human Light Chain Kappa signal sequence, a JEV short signal sequence, a JEV long signal sequence, a Mouse Light Chain Kappa signal sequence, a SSP signal sequence, or a Gaussian Luciferase (AKP). As used herein, a “mature” sequence means it lacks the N-terminal signal sequence (signal peptide).

A modified betacoronavirus S protein or S protein fragment amino acid sequence may comprise heterologous amino acid residues, such as one or more tags to facilitate detection (e.g. an epitope tag for detection by monoclonal antibodies) and/or purification (e.g. a polyhistidine-tag to allow purification on a nickel-chelating resin) of the protein or fragment. In a certain embodiment, the protein or fragment sequence further comprises a cleavable linker. A cleavable linker allows for the tag to be separated from the S protein or S protein fragment, for example, by the addition of an agent capable of cleaving the linker. A number of different cleavable linkers are known to those of skill in the art. In certain embodiments it may thus be necessary to truncate the ectodomain, so certain embodiments provide a modified betacoronavirus S protein fragment having a truncated, function ectodomain that lacks 1, 2, 3, 4, 5, 6, 7, 8, 9, 10, 11, 12, 13, 14, 15, 16, 17, 18, 19 or 20 amino acid residues of the natural ectodomain.

A polypeptide with an inactive transmembrane domain (e.g., inactive by having a truncated TM domain (“TM-truncated”, such as a deleted TM domain “TM-deleted”) cannot reside within a lipid bilayer and may, therefore, be more easily purified and at higher yield. Especially in the context of a subunit vaccination approach, it may be desirable to increase the solubility of a betacoronavirus S protein or S protein fragment by, for example, providing a TM-inactive (e.g., TM-truncated or TM-deleted) betacoronavirus S protein fragment. In certain embodiments is provided a TM-truncated betacoronavirus S protein fragment that is operably linked at its C-terminus to a heterologous amino acid sequence (such as a cytoplasmic tail (CT)). In certain embodiments is provided a betacoronavirus S protein fragment consisting of 0, 1, 2, 3, 4, 5, 6, 7, 8, 9 or 10 amino acids of the natural TM domain. For a DNA- or RNA-based vaccine approach to delivering proteins whose wild type counterparts are cell-membrane bound, it would be undesirable to inactivate the protein's transmembrane domain.

In certain embodiments is provided a betacoronavirus S protein fragment with a truncated cytoplasmic domain. In certain embodiments is provided a betacoronavirus S protein fragment consisting of 0, 1, 2, 3, 4, 5, 6, 7, 8, 9 or 10 amino acids of the natural cytoplasmic domain.

In certain embodiments is provided a purified or isolated, modified betacoronavirus S protein or fragment thereof. In certain embodiments is provided a purified or isolated, modified MERS-CoV, SARS-CoV-1, or SARS-CoV2 S protein or fragment thereof. In certain other embodiments is provided a purified or isolated, modified SARS-β, BCoV S protein or fragment thereof (such as a purified or isolated, modified SARS-CoV-1 SARS-CoV-2 S protein or fragment thereof).

It would be well understood that amino acid sequences for use in, for example, transient expression (such as those for use in preclinical studies) may be modified to make them suitable for stable expression (in advance of clinical studies, for example). Techniques for making an amino acid sequence more suitable for stable expression includes, for example, the removal of purification tags, amino acid substitution or deletion (e.g., in the ectodomain) to reduce C-terminal heterogeneity, as well as the deletion of hydrophobic residues (e.g., in the ectodomain) to increase solubility. Application of these techniques to the presently provided betacoronavirus S protein or S protein fragment sequences is envisaged.

In certain embodiments is provided a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 5-114 (or also to SEQ ID NOs 125-134). In certain embodiments is provided a polynucleotide encoding a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 5-114 (or also to SEQ ID NOs 125-134).

In certain embodiments is provided a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 5-64 (or also to SEQ ID NOs 125-134). In certain embodiments is provided a polynucleotide encoding a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 5-64 (or also to SEQ ID NOs 125-134).

In certain embodiments is provided a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 65-114 (or also to SEQ ID NOs 125-134). In certain embodiments is provided a polynucleotide encoding a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 65-114 (or also to SEQ ID NOs 125-134).

In certain embodiments is provided a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 65-104 (or also to SEQ ID NOs 125-134). In certain embodiments is provided a polynucleotide encoding a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 65-104 (or also to SEQ ID NOs 125-134).

In certain embodiments is provided a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 105-114 (or also to SEQ ID NOs 125-134). In certain embodiments is provided a polynucleotide encoding a modified betacoronavirus S protein, or fragment thereof, that has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from the group consisting of SEQ ID NOs: 105-114 (or also to SEQ ID NOs 125-134).

If desired, the modified betacoronavirus S protein or fragment thereof (or polynucleotide sequence encoding it such as the self-replicating RNA molecule) can be screened or analyzed to confirm their therapeutic and prophylactic properties using various in vitro or in vivo testing methods that are known to those of skill in the art. For example, they can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a polynucleotide (e.g., a self-replicating RNA molecule) that encodes the modified betacoronavirus S protein or S protein fragment. In addition, T helper cell differentiation can be analyzed by measuring proliferation or production of TH1 (IL-2, TNF-α, or IFN-γ) cytokines and/or TH2 (IL-4 or IL-5) cytokines by ELISA or directly in CD4+ T cells by cytoplasmic cytokine staining and flow cytometry.

Self-replicating RNA molecules that encode a modified betacoronavirus S protein or S protein fragment can also be tested for ability to induce humoral immune responses, as evidenced, for example, by induction of B cell production of antibodies specific for a modified betacoronavirus S protein or S protein fragment of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded modified betacoronavirus S protein or S protein fragment by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

An immunogenic composition for use herein delivers 1 to 100 μg of betacoronavirus S protein or S protein fragment per dose (e.g., per human dose)—1 to 100 μg being the total amount of all betacoronavirus S proteins or S protein fragments delivered to the subject (e.g., if the composition comprises a mix of S protein sequences having/encoding variable structures such as one or more being the modified betacoronavirus S proteins or S protein fragments provided herein). For example, an immunogenic composition may deliver about 25 μg (such as 22.5-27.5 μg) or about 50 μg (such as 45-55 μg) of betacoronavirus S protein or S protein fragment. For administration of an immunogenic composition, two or more doses of the immunogenic composition may be administered so that the total dose of betacoronavirus S protein or S protein fragment delivered is 1 to 100 μg per dose (e.g., human dose) (such as about 25 μg (such as 22.5-27.5 μg) or about 50 μg (such as 45-55 μg) of betacoronavirus S protein or S protein fragment). Especially in a subunit approach, a suitable amount of betacoronavirus S protein or S protein fragment protein is, for example, 1 to 100 μg (w/v) per dose (e.g., human dose) of the immunogenic composition; such as about 25 μg or about 50 μg of betacoronavirus S protein or S protein fragment protein (w/v) per human dose of the immunogenic composition (for example, 22.5-27.5 μg or 45-55 μg of betacoronavirus S protein or S protein fragment (w/v) per human dose of the immunogenic composition).

### Adjuvant

Adjuvants are included in vaccines to improve humoral and cellular immune responses, particularly in the case of poorly immunogenic subunit vaccines. Similar to natural infections by pathogens, adjuvants rely on the activation of the innate immune system to promote long-lasting adaptive immunity and in particular to (1) increase the immunogenicity of weak antigens; (2) enhance the speed and duration of the immune response; (3) modulate antibody avidity, specificity, isotype or subclass distribution; (4) stimulate cell mediated immunity; (5) promote the induction of mucosal immunity; (6) enhance immune responses in immunologically immature or senescent individuals; (7) decrease the dose of antigen in the vaccine and/or (8) help to overcome antigen competition in combination vaccines (Rajuput et al. Adjuvant effects of saponins on animal immune responses 2007 J Zhejiang Univ Sci. B. 8(3):153-161). Adjuvants can deeply influence the quality of an immune response, and therefore, their selection may be fundamental in a vaccine formulation.

Adjuvants are classified according to the source of their constituents, their physiochemical properties, or their mechanism of action and are generally grouped into two subheadings: molecular adjuvants (including genetic adjuvants) that act directly on the immune system to enhance immune response against antigen(s) (e.g., TLR ligands, cytokines, plasmids expressing cytokines, chemokines, saponins, and bacterial exotoxins) and carrier systems that promote antigen(s) in the most appropriate way to the immune system while also exhibiting controlled release and depot effects, thereby increasing the immune response (e.g., mineral salts, emulsions, liposomes, virosomes, biodegradable polymer micro/nano particles and immune stimulating complexes-ISCOMS). Gulce-Iz and Saglam-Metiner April 2019 “Current State of the Art in DNA Vaccine Delivery and Molecular Adjuvants: Bcl-xL Anti-Apoptotic Protein as a Molecular Adjuvant” in IMMUNE RESPONSE ACTIVATION AND IMMUNOMODULATION DOI:10.5772/intechopen.82203. In certain embodiments, the presently provided immunogenic composition comprises an adjuvant. Examples of suitable adjuvants include but are not limited to inorganic adjuvants (e.g. inorganic metal salts such as aluminium phosphate or aluminium hydroxide), organic adjuvants (e.g. saponins, such as QS21, or squalene), oil-based adjuvants (e.g. Freund's complete adjuvant and Freund's incomplete adjuvant), oil-in-water emulsions, cytokines (e.g. IL-1β, IL-2, IL-7, IL-12, IL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A, such as 3-de-O-acylated monophosphoryl lipid A (3D-MPL), or muramyl peptides), synthetic adjuvants (e.g. non-ionic block copolymers, muramyl peptide analogues, or synthetic lipid A), synthetic polynucleotides adjuvants (e.g polyarginine or polylysine), Toll-like receptor (TLR) agonists (including TLR-1, TLR-2, TLR-3, TLR-4, TLR-5, TLR-6, TLR-7, TLR-8 and TLR-9 agonists) and immunostimulatory oligonucleotides containing unmethylated CpG dinucleotides (“CpG”).

In a preferred embodiment, the adjuvant comprises a TLR agonist and/or an immunologically active saponin. Preferably still, the adjuvant may comprise or consist of a TLR agonist and a saponin in a liposomal formulation. The ratio of TLR agonist to saponin may be 5:1, 4:1, 3:1, 2:1 or 1:1.

The use of TLR agonists in adjuvants is well-known in art and has been reviewed e.g. by Lahiri et al. (2008) Vaccine 26:6777. TLRs that can be stimulated to achieve an adjuvant effect include TLR2, TLR4, TLR5, TLR7, TLR8 and TLR9. TLR2, TLR4, TLR7 and TLR8 agonists, particularly TLR4 agonists, are preferred.

Suitable TLR4 agonists include lipopolysaccharides, such as monophosphoryl lipid A (MPL) and 3-O-deacylated monophosphoryl lipid A (3D-MPL). U.S. Pat. No. 4,436,727 discloses MPL and its manufacture. U.S. Pat. No. 4,912,094 and reexamination certificate B1 4,912,094 discloses 3D-MPL and a method for its manufacture. Another TLR4 agonist is glucopyranosyl lipid adjuvant (GLA), a synthetic lipid A-like molecule (see, e.g. Fox et al. (2012) Clin. Vaccine Immunol 19:1633). In a further embodiment, the TLR4 agonist may be a synthetic TLR4 agonist such as a synthetic disaccharide molecule, similar in structure to MPL and 3D-MPL or may be synthetic monosaccharide molecules, such as the aminoalkyl glucosaminide phosphate (AGP) compounds disclosed in, for example, WO9850399, WO0134617, WO0212258, WO3065806, WO04062599, WO06016997, WO0612425, WO03066065, and WO0190129. Such molecules have also been described in the scientific and patent literature as lipid A mimetics. Lipid A mimetics suitably share some functional and/or structural activity with lipid A, and in one aspect are recognised by TLR4 receptors. AGPs as described herein are sometimes referred to as lipid A mimetics in the art. In a preferred embodiment, the TLR4 agonist is 3D-MPL.TLR4 agonists, such as 3-O-deacylated monophosphoryl lipid A (3D-MPL), and their use as adjuvants in vaccines has e.g. been described in WO 96/33739 and WO2007/068907 and reviewed in Alving et al. (2012) Curr Opin in Immunol 24:310.

Suitably, the adjuvant comprises an immunologically active saponin, such as an immunologically active saponin fraction, such as QS21.

Adjuvants comprising saponins have been described in the art. Saponins are described in: Lacaille-Dubois and Wagner (1996) A review of the biological and pharmacological activities of saponins, Phytomedicine vol 2:363. Saponins are known as adjuvants in vaccines. For example, Quil A (derived from the bark of the South American tree Quillaja Saponaria Molina), was described by Dalsgaard et al. in 1974 (“Saponin adjuvants”, Archiv. fur die gesamte Virusforschung, Vol. 44, Springer Verlag, Berlin, 243) to have adjuvant activity. Purified fractions of Quil A have been isolated by HPLC which retain adjuvant activity without the toxicity associated with Quil A (Kensil et al. (1991) J. Immunol. 146: 431). Quil A fractions are also described in U.S. Pat. No. 5,057,540 and “Saponins as vaccine adjuvants”, Kensil, C. R., Crit Rev Ther Drug Carrier Syst, 1996, 12 (1-2):1-55.

Two Quil A such fractions, suitable for use in the present invention, are QS7 and QS21 (also known as QA-7 and QA-21). QS21 is a preferred immunologically active saponin fraction for use in the present invention. QS21 has been reviewed in Kensil (2000) In O'Hagan: Vaccine Adjuvants: preparation methods and research protocols, Homana Press, Totowa, N.J., Chapter 15. Particulate adjuvant systems comprising fractions of Quil A, such as QS21 and QS7, are e.g. described in WO 96/33739, WO 96/11711 and WO2007/068907.

In addition to the other components, the adjuvant preferably comprises a sterol. The presence of a sterol may further reduce reactogenicity of compositions comprising saponins, see e.g. EP0822831. Suitable sterols include beta-sitosterol, stigmasterol, ergosterol, ergocalciferol and cholesterol. Cholesterol is particularly suitable. Suitably, the immunologically active saponin fraction is QS21 and the ratio of QS21:sterol is from 1:100 to 1:1 (w/w), suitably between 1:10 to 1:1 (w/w), and preferably 1:5 to 1:1 (w/w). Suitably excess sterol is present, the ratio of QS21:sterol being at least 1:2 (w/w). In one embodiment, the ratio of QS21:sterol is 1:5 (w/w). The sterol is suitably cholesterol.

In a preferred embodiment, the adjuvant comprises a TLR4 agonist and an immunologically active saponin. In a more preferred embodiment, the TLR4 agonist is 3D-MPL and the immunologically active saponin is QS21.

In some embodiments, the adjuvant is presented in the form of an oil-in-water emulsion, e.g. comprising squalene, alpha-tocopherol and a surfactant (see e.g. WO95/17210) or in the form of a liposome. A liposomal presentation is preferred.

The term “liposome” when used herein refers to uni- or multilamellar (particularly 2, 3, 4, 5, 6, 7, 8, 9, or 10 lamellar depending on the number of lipid membranes formed) lipid structures enclosing an aqueous interior. Liposomes and liposome formulations are well known in the art. Liposomal presentations are e.g. described in WO 96/33739 and WO2007/068907. Lipids which are capable of forming liposomes include all substances having fatty or fat-like properties. Lipids which can make up the lipids in the liposomes may be selected from the group comprising glycerides, glycerophospholipids, glycerophospholipids, glycerophospholipids, sulfolipids, sphingolipids, phospholipids, isoprenolides, steroids, stearines, sterols, archeolipids, synthetic cationic lipids and carbohydrate containing lipids. In a particular embodiment of the invention the liposomes comprise a phospholipid. Suitable phospholipids include (but are not limited to): phosphocholine (PC) which is an intermediate in the synthesis of phosphatidylcholine; natural phospholipid derivates: egg phosphocholine, egg phosphocholine, soy phosphocholine, hydrogenated soy phosphocholine, sphingomyelin as natural phospholipids; and synthetic phospholipid derivates: phosphocholine (didecanoyl-L-a-phosphatidylcholine [DDPC], dilauroylphosphatidylcholine [DLPC], dimyristoylphosphatidylcholine [DMPC], dipalmitoyl phosphatidylcholine [DPPC], Distearoyl phosphatidylcholine [DSPC], Dioleoyl phosphatidylcholine, [DOPC], 1-palmitoyl, 2-oleoylphosphatidylcholine [POPC], Dielaidoyl phosphatidylcholine [DEPC]), phosphoglycerol (1,2-Dimyristoyl-sn-glycero-3-phosphoglycerol [DMPG], 1,2-dipalmitoyl-sn-glycero-3-phosphoglycerol [DPPG], 1,2-distearoyl-sn-glycero-3-phosphoglycerol [DSPG], 1-palmitoyl-2-oleoyl-sn-glycero-3-phosphoglycerol [POPG]), phosphatidic acid (1,2-dimyristoyl-sn-glycero-3-phosphatidic acid [DMPA], dipalmitoyl phosphatidic acid [DPPA], distearoyl-phosphatidic acid [DSPA]), phosphoethanolamine (1,2-dimyristoyl-sn-glycero-3-phosphoethanolamine [DMPE], 1,2-Dipalmitoyl-sn-glycero-3-phosphoethanolamine [DPPE], 1,2-distearoyl-sn-glycero-3-phosphoethanolamine [DSPE], 1,2-Dioleoyl-sn-Glycero-3-Phosphoethanolamine [DOPE]), phosphoserine, polyethylene glycol [PEG] phospholipid.

Liposome size may vary from 30 nm to several μm depending on the phospholipid composition and the method used for their preparation. In particular embodiments of the invention, the liposome size will be in the range of 50 nm to 500 nm and in further embodiments 50 nm to 200 nm. Dynamic laser light scattering is a method used to measure the size of liposomes well known to those skilled in the art.

In a particularly suitable embodiment, liposomes used in the invention comprise DOPC and a sterol, in particular cholesterol. Thus, in a particular embodiment, compositions of the invention comprise QS21 in any amount described herein in the form of a liposome, wherein said liposome comprises DOPC and a sterol, in particular cholesterol.

In a more preferred embodiment, the adjuvant comprises a 3D-MPL and QS21 in a liposomal formulation.

In one embodiment, the adjuvant comprises between 25 and 75, such as between 35 and 65 micrograms (for example about or exactly 50 micrograms) of 3D-MPL and between 25 and 75, such as between 35 and 65 (for example about or exactly 50 micrograms) of QS21 in a liposomal formulation.

In another embodiment, the adjuvant comprises between 12.5 and 37.5, such as between 20 and 30 micrograms (for example about or exactly 25 micrograms) of 3D-MPL and between 12.5 and 37.5, such as between 20 and 30 micrograms (for example about or exactly 25 micrograms) of QS21 in a liposomal formulation.

In another embodiment of the present invention, the adjuvant comprises or consists of an oil-in-water emulsion. Suitably, an oil-in-water emulsion comprises a metabolisable oil and an emulsifying agent. A particularly suitable metabolisable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast. In one embodiment, the metabolisable oil is present in the immunogenic composition in an amount of 0.5% to 10% (v/v) of the total volume of the composition. A particularly suitable emulsifying agent is polyoxyethylene sorbitan monooleate (POLYSORBATE 80 or TWEEN 80). In one embodiment, the emulsifying agent is present in the immunogenic composition in an amount of 0.125 to 4% (v/v) of the total volume of the composition. The oil-in-water emulsion may optionally comprise a tocol. Tocols are well known in the art and are described in EP0382271 B1. Suitably, the tocol may be alpha-tocopherol or a derivative thereof such as alpha-tocopherol succinate (also known as vitamin E succinate). In one embodiment, the tocol is present in the adjuvant composition in an amount of 0.25% to 10% (v/v) of the total volume of the immunogenic composition. The oil-in-water emulsion may also optionally comprise sorbitan trioleate (SPAN 85).

In an oil-in-water emulsion, the oil and emulsifier should be in an aqueous carrier. The aqueous carrier may be, for example, phosphate buffered saline or citrate.

In the context of betacoronavirus vaccine candidates, certain adjuvants may be preferred including an adjuvant that comprises MF59, AS03 (e.g., AS03(A)), AS04, aluminum hydroxide, potassium aluminum phosphate (alum), a TLR agonist (e.g., a TLR3 agonist such as polyriboinosinic acid (poly I:C) (including alum and poly IC) or polyadenylic-polyuridylic acid (poly(A:U)); a TLR4 agonist such as lipopolysaccharide (LPS); or a TLR7 agonist such as polyuridylic acid (polyU)), cysteine-phosphate-guanine (CpG) oligodeoxynucleotides (ODN) (including alum and CpG ODN), delta inulin microparticle-based, a biphosphonate, melatonin (N-acetyl-5-methoxytryptamine), Monophosphoryl Lipid A, a water-in-oil emulsion such as MONTANIDE ISA 51 (or “ISA 51”) or a saponin adjuvant (e.g., an adjuvant comprising Quillaja saponins such as MATRIX-M or AS01 (e.g., AS01(B)).

In particular, the oil-in-water emulsion systems used in the present invention have a small oil droplet size in the sub-micron range. Suitably the droplet sizes will be in the range 120 to 750 nm, more particularly sizes from 120 to 600 nm in diameter. Even more particularly, the oil-in water emulsion contains oil droplets of which at least 70% by intensity are less than 500 nm in diameter, more particular at least 80% by intensity are less than 300 nm in diameter, more particular at least 90% by intensity are in the range of 120 to 200 nm in diameter.

It will be understood that the modified betacoronavirus S protein, immunogenic fragment thereof, or its encoding polynucleotide may be stored separately from the adjuvant and admixed with the adjuvant prior to administration (ex tempo) to a subject. The modified betacoronavirus S protein, immunogenic fragment thereof, or its encoding polynucleotide and the adjuvant may also be administered separately, but concomitantly, to a subject.

In one aspect, there is provided a kit comprising or consisting of a modified betacoronavirus S protein, or immunogenic fragment thereof, as described herein and an adjuvant.

Where the adjuvant is in a liquid form to be combined with a liquid form of an antigen composition, the adjuvant composition will be in a human-dose-suitable volume which is approximately half of the intended final volume of the human dose, for example a 360 μl volume for an intended human dose of 0.7 ml, or a 250 μl volume for an intended human dose of 0.5 ml. The adjuvant composition is diluted when combined with the antigen composition to provide the final human dose of vaccine. The final volume of such dose will of course vary dependent on the initial volume of the adjuvant composition and the volume of antigen composition added to the adjuvant composition. Alternatively, liquid adjuvant is used to reconstitute a lyophilised antigen composition. In such cases, the human dose suitable volume of the adjuvant composition is approximately equal to the final volume of the human dose. The liquid adjuvant composition is added to the vial containing the lyophilised antigen composition.

The final human dose can vary between, for example, 0.25 to 1.5 ml.

### Expression Methods

The polypeptides may be produced by any suitable means, including by recombinant expression production or by chemical synthesis. Polypeptides may be recombinantly expressed and purified using any suitable method as is known in the art, and the product characterized using methods as known in the art, e.g., by Nano-Differential Scanning Fluorimetry (Nano-DSF), Surface Plasmon Resonance (SPR), and Electron Microscopy, to confirm the polypeptides of the present invention form correct conformation.

The method comprises the steps of (a) culturing a recombinant host cell under conditions conducive to the expression of the polypeptide. The method may further comprise recovering, isolating, or purifying the expressed polypeptide. In one embodiment, multiple copies of a subunit polypeptide are expressed in a host cell, where every three of the subunit polypeptides forms homogeneous trimer of polypeptides within the host cell. The formed trimer of polypeptides can then be recovered, isolated or purified from the cell or the culture medium in which the cell is grown.

The expressed polypeptide may include a linker peptide and a purification tag. Various expression systems are known, including those using human (e.g., HeLa) host cells, mammalian (e.g., Chinese Hamster Ovary (CHO)) host cells, prokaryotic host cells (e.g., E. coli), or insect host cells. The host cell is typically transformed with the recombinant nucleic acid sequence encoding the desired polypeptide product, cultured under conditions suitable for expression of the product. The expressed product may be purified from the cell or culture medium. Cell culture conditions are particular to the cell type and expression vector.

When a recombinant host cell of the present invention is cultured under suitable conditions, the recombinant nucleic acid expresses a subunit polypeptide as described herein. The polypeptide can form polypeptide trimer within the cell. Suitable host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster)), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof.

Host cells can be cultured in conventional nutrient media modified as appropriate and as will be apparent to those skilled in the art (e.g., for activating promoters). Culture conditions, such as temperature, pH and the like, may be determined using knowledge in the art, see e.g., Freshney (1994) Culture of Animal Cells, a Manual of Basic Technique, third edition, Wiley-Liss, New York and the references cited therein. In bacterial host cell systems, a number of expression vectors are available including, but not limited to, multifunctional E. coli cloning and expression vectors such as BLUESCRIPT (Stratagene) or pET vectors (Novagen, Madison Wis.). In mammalian host cell systems, a number of expression systems, including both plasmids and viral-based systems, are available commercially.

Eukaryotic or microbial host cells expressing polypeptides of the invention can be disrupted by any convenient method (including freeze-thaw cycling, sonication, mechanical disruption), and polypeptides can be recovered and purified from recombinant cell culture by any suitable method known in the art (including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxyapatite chromatography, and lectin chromatography). Size Exclusion Chromatography (SEC) can be employed in the final purification steps.

In general, expression of a recombinantly encoded polypeptide of the present invention involves preparation of an expression vector comprising a recombinant polynucleotide under the control of one or more promoters, such that the promoter stimulates transcription of the polynucleotide and promotes expression of the encoded polypeptide. “Recombinant Expression” as used herein refers to such a method.

In a further aspect, the present invention provides recombinant expression vectors comprising a recombinant nucleic acid sequence of any embodiment of the invention operatively linked to a suitable control sequence. “Recombinant expression vector” includes vectors that operatively link a nucleic acid coding region or gene to any control sequences capable of effecting expression of the gene product. “Control sequences” are nucleic acid sequences capable of effecting the expression of the nucleic acid molecules and need not be contiguous with the nucleic acid sequences, so long as they function to direct the expression thereof. Recombinant expression ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxyapatite chromatography, and lectin chromatography). Size Exclusion Chromatography (SEC) can be employed in the final purification steps.

In general, expression of a recombinantly encoded polypeptide of the present invention involves preparation of an expression vector comprising a recombinant polynucleotide under the control of one or more promoters, such that the promoter stimulates transcription of the polynucleotide and promotes expression of the encoded polypeptide. “Recombinant Expression” as used herein refers to such a method.

In a further aspect, the present invention provides recombinant expression vectors comprising a recombinant nucleic acid sequence of any embodiment of the invention operatively linked to a suitable control sequence. “Recombinant expression vector” includes vectors that operatively link a nucleic acid coding region or gene to any control sequences capable of effecting expression of the gene product. “Control sequences” are nucleic acid sequences capable of effecting the expression of the nucleic acid molecules and need not be contiguous with the nucleic acid sequences, so long as they function to direct the expression thereof. Recombinant expression vectors can be of any type known in the art, including but not limited to plasmid and viral-based expression vectors. The control sequence used to drive expression of the disclosed nucleic acid sequences in a mammalian system may be constitutive or inducible. The construction of expression vectors for use in transfecting prokaryotic cells is also well known. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the selected host organism either as an episome or by integration into host chromosomal DNA. In non-limiting embodiments, the expression vector is a plasmid vector or a viral vector. Expression vectors suitable for use in a given host-expression system and containing the encoding nucleic acid sequence and transcriptional/translational control sequences, may be made by any suitable technique as is known in the art. Typical expression vectors contain suitable promoters, enhancers, and terminators that are useful for regulation of the expression of the coding sequence(s) in the expression construct. The vectors may also comprise selection markers to provide a phenotypic trait for selection of transformed host cells (such as conferring resistance to antibiotics such as ampicillin or neomycin). Nucleic acid or vector modification may be undertaken in a manner known by the art, see e.g., WO 2012/049317 (corresponding to US 2013/0216613) and WO 2016/092460 (corresponding to US 2018/0265551). For example, the nucleic acid sequence encoding an NP subunit polypeptide as described herein is cloned into a vector suitable for introduction into the selected cell system, e.g., bacterial or mammalian cells (e.g., CHO cells). Transformed cells are expanded, e.g., by culturing.

Suitable host cells can be either prokaryotic or eukaryotic, such as mammalian cells. The cells can be transiently or stably transfected. Such transfection of expression vectors into prokaryotic and eukaryotic cells can be accomplished via any technique known in the art, including but not limited to standard bacterial transformations, calcium phosphateco-precipitation, electroporation, or liposome mediated-, DEAE dextran mediated-, polycationic mediated-, or viral mediated transfection or transduction. (See, for example, Molecular Cloning: A Laboratory Manual (Sambrook, et al., 1989, Cold Spring Harbor Laboratory Press; Culture of Animal Cells: A Manual of Basic Technique, 2.sup.nd Ed. (R. I. Freshney.1987. Liss, Inc. New York, N.Y.).

The expressed subunit polypeptides forms trimer or other types of oligomer, and could be further recovered (e.g., purified, isolated, or enriched).

### Purification

The term “purified” as used herein refers to the separation or isolation of a defined product (e.g., a recombinantly expressed polypeptide) from a composition containing other components (e.g., a host cell or host cell medium). A polypeptide composition that has been fractionated to remove undesired components, and which composition retains its biological activity, is considered ‘purified’. ‘Purified’ is a relative term and does not require that the desired product be separated from all traces of other components. Stated another way, “purification” or “purifying” refers to the process of removing undesired components from a composition or host cell or culture. Various methods for use in purifying polypeptides of the present invention are known in the art, e.g., centrifugation, dialysis, affinity or size based chromatography, gel electrophoresis, filtration, precipitation and combinations thereof. The polypeptides of the present invention may be expressed with a tag operable for affinity purification, such as a 6×Histidine tag as is known in the art. A His-tagged polypeptide may be purified using, for example, Ni-NTA column chromatography or using anti-6×His antibody fused to a solid support.

Thus, the term “purified” does not require absolute purity; rather, it is intended as a relative term. A “substantially pure” preparation of polypeptides or nucleic acid molecules is one in which the desired component represents at least 50% of the total polypeptide (or nucleic acid) content of the preparation. In certain embodiments, a substantially pure preparation will contain at least 60%, at least 70%, at least 80%, at least 85%, at least 90%, at least 95%, at least 97%, at least 98%, or at least 99% or more of the total polypeptide (or nucleic acid) content of the preparation. Methods for quantifying the degree of purification of expressed polypeptides are known in the art and include, for example, assessing the number of polypeptides within a fraction by SDS/PAGE analysis, or assessing the ratio of desired polypeptides to undesired components in final purified product by Size Exclusion Chromatography (SEC).

Thus, in the sense of the present invention, a “purified” or an “isolated” biological component (such as a polypeptide, or a nucleic acid molecule) has been substantially separated or purified away from other biological components in which the component naturally occurs or was recombinantly produced. The term embraces polypeptides, and nucleic acid molecules prepared by chemical synthesis as well as by recombinant expression in a host cell.

### Biophysical Characterization

The biophysical property of purified polypeptides may be tested by various means. Herein the biophysical property includes but not limited to thermal stability and antigenicity. Thermal stability refers to the quality of a substance (e.g. the polypeptides of the invention), to resist irreversible change in its chemical or physical structure at a high relative temperature. It could be measured by NanoDSF technique, which detects the changes of intrinsic tryptophan fluorescence caused by unfolding of polypeptide structure. Antigenicity refers to the capacity of polypeptides to bind to specific antibody molecules. A strong binding capacity of polypeptides to a specific antibody usually indicates the structural integrity of the binding site (epitopes) on polypeptide. The antigenicity of a polypeptide can be measured by Surface Plasmon Resonance technology, which is a standard tool for measuring the rate of molecule-molecule association and dissociation. The ratio of dissociation rate to association rate defined as ‘binding affinity’ with unites of picomolar.

### Compositions

### Immunogenic Compositions

Immunogenic compositions (e.g., vaccine compositions) may be prophylactic (i.e. to prevent disease) or therapeutic (i.e. to lower, reduce, or eliminate the symptoms of a disease). Nonetheless, immunogenic compositions herein elicit an immune response. In certain embodiments is provided an immunogenic composition that elicits a humoral (e.g., a neutralizing antibody response) and/or cellular immune response in a subject and wherein the immune response is comparable to or greater than that of natural immunity.

Immunogenic compositions herein may be used to, e.g., induce an immune response, but also to, e.g., prevent betacoronavirus infection or reinfection of a subject, reduce betacoronavirus cell entry (e.g., as compared to that of natural infection) or reduce betacoronavirus cell-to-cell spread (e.g., as compared to that of natural infection). Furthermore, immunogenic compositions herein may be used to prevent, or reduce the severity of, betacoronavirus-associated disease (e.g., SARS-CoV-2-associated disease such as COVID-19), such as following delivery of an immunogenic composition to a subject selected for having already been infected (which may be determined by testing the subject's blood for virus-specific antibodies).

Certain embodiments provide an immunogenic composition comprising a modified betacoronavirus S protein or fragment thereof and one or more adjuvants (e.g., wherein the one or more adjuvants comprises MF59, AS03 [e.g., AS03(A)], AS04, aluminum hydroxide, potassium aluminum phosphate (alum), a TLR agonist [e.g., a TLR3 agonist such as polyriboinosinic acid (poly I:C) (including alum and poly IC) or polyadenylic-polyuridylic acid (poly(A:U)); a TLR4 agonist such as lipopolysaccharide (LPS); or a TLR7 agonist such as polyuridylic acid (polyU)], cysteine-phosphate-guanine (CpG) oligodeoxynucleotides (ODN) (including alum and CpG ODN), delta inulin microparticle-based, a biphosphonate, melatonin (N-acetyl-5-methoxytryptamine), Monophosphoryl Lipid A, a water-in-oil emulsion such as MONTANIDE ISA 51 (or “ISA 51”) or a saponin adjuvant [e.g., an adjuvant comprising Quillaja saponins such as MATRIX-M or AS01 (e.g., AS01(B)]. Immunogenic compositions comprising a nucleic acid that encodes a modified betacoronavirus S protein or fragment thereof can also include an adjuvant.

The immunogenic compositions herein are not limited to consisting of a modified betacoronavirus S protein or fragment thereof, or a polynucleotide encoding a modified betacoronavirus S protein or fragment thereof; but rather may also comprise other betacoronavirus antigens (optionally a mix of antigens and optionally from a mix of betacoronaviruses such as at least two betacoronavirus antigens optionally wherein the at least two antigens do not originate from the same betacoronavirus but rather originate from at least two of MERS-CoV, SARS-CoV-1, and SARS-CoV-2). In the context of SARS-CoV-2, for example, other antigens may be one or more of N, M, nsp3, nsp4, ORF3s, ORF7a, nsp12, or ORF8. See Grifoni et al. 2020 Cell 181:1-13 and Supplemental Materials. A certain embodiment therefore provides an immunogenic composition comprising a modified betacoronavirus S protein, or fragment thereof, and an N, an M, or both an N and an M protein, or fragment thereof.

Immunogenic compositions herein may comprise one or more nucleic acid molecules that encode a modified spike protein or fragment thereof (specifically, encode a modified MERS-CoV, SARS-CoV-1, or SARS-CoV-2 spike protein or fragment thereof) such that, following administration to a subject, recombinant modified spike protein or fragment thereof are delivered to a cell of the subject. Exemplary effective amounts of a nucleic acid component can be between 1 ng and 100 μg, such as between 1 ng and 1 μg (e.g., 100 ng-1 μg), or between 1 μg and 100 μg, such as 10 ng, 50 ng, 100 ng, 150 ng, 200 ng, 250 ng, 500 ng, 750 ng, or 1 μg. Effective amounts of a nucleic acid can also include from 1 μg to 500 μg, such as between 1 μg and 200 μg, such as between 10 and 100 μg, for example 1 μg, 2 μg, 5 μg, 10 μg, 20 μg, 50 μg, 75 μg, 100 μg, 150 μg, or 200 μg. Alternatively, an exemplary effective amount of a nucleic acid can be between 100 μg and 1 mg, such as from 100 μg to 500 μg, for example, 100 μg, 150 μg, 200 μg, 250 μg, 300 μg, 400 μg, 500 μg, 600 μg, 700 μg, 800 μg, 900 μg or 1 mg. The nucleic acid molecule encoding a modified betacoronavirus spike protein or fragment thereof (e.g., betacoronavirus, lineage B spike protein or fragment thereof such as MERS-CoV, SARS-CoV-1, or SARS-CoV-2 spike protein or fragment thereof) may be codon optimized. By “codon optimized” is intended modification with respect to codon usage that may increase translation efficacy and/or half-life of the nucleic acid. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methyltransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-0] N), which may further increase translation efficacy. The nucleic acids may comprise one or more nucleotide analogs or modified nucleotides. A “nucleotide analog” herein includes a nucleotide that contains one or more chemical modifications (e.g., substitutions) in or on the nitrogenous base of the nucleoside (e.g. cytosine (C), thymine (T) or uracil (U)), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate. The preparation of nucleotides and modified nucleotides and nucleosides are well-known in the art and many modified nucleosides and modified nucleotides are commercially available. Modified nucleobases which can be incorporated into modified nucleosides and nucleotides and be present in an RNA molecule include: m5C (5-methylcytidine), m5U (5-methyluridine), m6A (N6-methyladenosine), s2U (2-thiouridine), Um (2-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2-0-ribosyladenosine (phosphate)); I (inosine); mil (1-methylinosine); m′1m (1,2′-0-dimethylinosine); m3C (3-methylcytidine); Cm (2T-0-methylcytidine); s2C (2-thiocytidine); ac4C (N4-acetylcytidine); £5C (5-fonnylcytidine); m5Cm (5,2-O-dimethylcytidine); ac4Cm (N4acetyl2TOmethylcytidine); k2C (lysidine); mlG (1-methylguanosine); m2G (N2-methylguanosine); m7G (7-methylguanosine); Gm (2′-0-methylguanosine); m22G (N2,N2-dimethylguanosine); m2Gm (N2,2′-0-dimethylguanosine); m22Gm (N2,N2,2′-0-trimethylguanosine); Gr(p) (2′-0-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-0-dimethyluridine); s4U (4-thiouridine); m5s2U (5-methyl-2-thiouridine); s2Um (2-thio-2′-0-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-Omethyl uridine); cmnm5s2U (5-carboxymethylaminomethyl-2-thiouridine); m62A (N6,N6-dimethyladenosine); Tm (2′-0-methylinosine); m4C (N4-methylcytidine); m4Cm (N4,2-0-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-0-dimethyluridine); m5D (5-methyldihydrouridine); £5Cm (5-formyl-2′-0-methylcytidine); mlGm (1,2′-0-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-Ce)-alkyluracil, 5-methyluracil, 5-(C2-C6)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(Ci-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-0-methyl-U.

### Formulations

The pH of a composition for use herein is usually between 6 and 8, and more preferably between 6.5 and 7.5 (e.g. about 7). Stable pH may be maintained by the use of a buffer (e.g. an acetate, citrate, histidine, maleate, phosphate, succinate, tartrate, or Tris buffer, a citrate buffer, phosphate buffer, or a histidine buffer). Thus, a composition will generally include a buffer. A composition may be sterile and/or pyrogen-free. Compositions may be isotonic with respect to humans.

It is well known that for parenteral administration solutions should have a pharmaceutically acceptable osmolality to avoid cell distortion or lysis. A pharmaceutically acceptable osmolality will generally mean that solutions will have an osmolality which is approximately isotonic or mildly hypertonic. Suitably the compositions of the present invention when reconstituted will have an osmolality in the range of 250 to 750 mOsm/kg, for example, the osmolality may be in the range of 250 to 550 mOsm/kg, such as in the range of 280 to 500 mOsm/kg. In a particularly preferred embodiment, the osmolality may be in the range of 280 to 310 mOsm/kg.

Osmolality may be measured according to techniques known in the art, such as by the use of a commercially available osmometer, for example the Advanced™ Model 2020 available from Advanced Instruments Inc. (USA).

An “isotonicity agent” is a compound that is physiologically tolerated and imparts a suitable tonicity to a formulation to prevent the net flow of water across cell membranes that are in contact with the formulation. In some embodiments, the isotonicity agent used for the composition is a salt (or mixtures of salts), conveniently the salt is sodium chloride, suitably at a concentration of approximately 150 nM. In other embodiments, however, the composition comprises a non-ionic isotonicity agent and the concentration of sodium chloride in the composition is less than 100 mM, such as less than 80 mM, e.g. less than 50 mM, such as less 40 mM, less than 30 mM and especially less than 20 mM. The ionic strength in the composition may be less than 100 mM, such as less than 80 mM, e.g. less than 50 mM, such as less 40 mM or less than 30 mM.

In a particular embodiment, the non-ionic isotonicity agent is a polyol, such as sucrose and/or sorbitol. The concentration of sorbitol may e.g. between about 3% and about 15% (w/v), such as between about 4% and about 10% (w/v). Adjuvants comprising an immunologically active saponin fraction and a TLR4 agonist wherein the isotonicity agent is salt or a polyol have been described in WO2012/080369.

A human dose volume for use herein is between 0.25-1.5 ml (such as between 0.5 and 1.0 ml, e.g. a volume of about 0.5 ml; specifically a volume of 0.45-0.55 ml; or more specifically a volume of 0.5 ml). The volumes of the compositions used may depend on the delivery route and location, with smaller doses being given by the intradermal route. A unit dose container may contain an overage to allow for proper manipulation of materials during administration of the unit dose.

An adjuvant may be administered separately from an antigen or co-administered (i.e., combined, either during manufacturing or extemporaneously, with an antigen into an immunogenic composition for combined administration).

Immunogenic compositions for use herein may further comprise one or more pharmaceutically acceptable additives such as buffers, carriers, excipients, tonicity agents, wetting or emulsifying agents, detergents, antimicrobials, and diluents. Pharmaceutically acceptable additives are known in the field (e.g., in Remington's Pharmaceutical Sciences, by E. W. Martin, Mack Publishing Co., Easton, Pa., 15th Edition (1975)).

A pharmaceutically acceptable additive for use herein may be sodium salts (e.g. sodium chloride) to give tonicity. A concentration of 1.0±2 mg/ml NaCl is typical.

Suitable carriers are typically large, slowly metabolized macromolecules such as proteins (e.g., nanoparticles), polysaccharides, polylactic acids, polyglycolic acids, polymeric amino acids, amino acid copolymers, sucrose, trehalose, lactose, lipid aggregates (such as oil droplets or liposomes), and inactive virus particles. Sterile pyrogen-free, phosphate-buffered physiologic saline is a typical carrier. Such carriers are well known in the art. A pharmaceutically acceptable additive for use herein may comprise a sugar alcohol (e.g. mannitol) or a disaccharide (e.g., sucrose or trehalose), e.g., at around 15-30 mg/ml (e.g. 25 mg/ml).

The additive may comprise a pharmaceutically acceptable diluent (e.g., sterile water), saline, glycerol, etc. Additionally, a pharmaceutically acceptable additive may comprise auxiliary substances, such as wetting or emulsifying agents, or pH buffering substances.

The additive may comprise a pharmaceutically acceptable excipient. Such excipients include, without limitation: glycerol, polyethylene glycol (PEG), glass forming polyols (such as, sorbitol, trehalose) N-lauroylsarcosine (e.g., sodium salt), L-proline, non-detergent sulfobetaine, guanidine hydrochloride, urea, trimethylamine oxide, KCl, Ca2+, Mg2+, Mn2+, Zn2+(and other divalent cation related salts), dithiothreitol (DTT), dithioerythrol, ß-mercaptoethanol, Detergents (including, e.g., Tween80, Tween20, Triton X-100, NP-40, Empigen BB, Octylglucoside, Lauroyl maltoside, Zwittergent 3-08, Zwittergent 3-10, Zwittergent 3-12, Zwittergent 3-14, Zwittergent 3-16, CHAPS, sodium deoxycholate, sodium dodecyl sulphate, and cetyltrimethylammonium bromide.

A pharmaceutically acceptable additive for use herein may be an antimicrobial, particularly when packaged in multiple dose format. Antimicrobials such as thiomersal and 2 phenoxyethanol are commonly found in vaccines, but it is preferred to use either a mercury-free preservative or no preservative at all. In certain embodiments, the antigen(s) may be conjugated to a bacterial toxoid, such as a toxoid from diphtheria, tetanus, cholera, H. pylori, or another pathogen.

A pharmaceutically acceptable additive for use herein may be a detergent, e.g., a TWEEN (polysorbate), such as TWEEN80. Detergents are generally present at low levels e.g. <0.01%.

In general, the nature of the pharmaceutically acceptable additive will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. In certain formulations (for example, solid compositions, such as powder forms), a liquid diluent is not employed. In such formulations, non-toxic solid carriers can be used, including for example, pharmaceutical grades of trehalose, mannitol, lactose, starch or magnesium stearate.

In certain embodiments, the pharmaceutically acceptable additive comprises a carrier, wherein the carrier is a pharmaceutically acceptable Fc domain of a human IgG1 antibody. In certain embodiments, an antigen (e.g., a SARS-βCoV spike protein or fragment thereof) is operably linked (directly or indirectly) to a pharmaceutically acceptable IgG1 antibody or Fc thereof (i.e., a chimeric protein). Such an approach was investigated as a candidate SARS-CoV-1 vaccine whereby the Receptor Binding Domain (RBD) of the SARS-CoV-1 spike protein was fused with an IgG1 Fc (RBD-Fc) and shown to elicit an immune response (Zheng B J et al. 2008 Hong Kong Med J 14(Suppl 4):S39-43; Du L. et al. 2009 Nat. Rev. Microbio. 7:226-236).

In certain embodiments, the pharmaceutically acceptable additive comprises a carrier, wherein the carrier is a pharmaceutically acceptable nanoparticle. In certain embodiments, an antigen (e.g., a SARS-βCoV spike protein or fragment thereof) is operably linked (directly or indirectly) to a pharmaceutically acceptable nanoparticle (e.g., lumazine synthase nanoparticle, ferritin nanoparticle, or an aldolase-based nanoparticle). See, e.g., WO2015/156870 (PCT/US2015/011534, DENG Z.), describing nanoparticle-polypeptide conjugates linked through an isopeptide bond (see also Bruun et al. 2018 ACS Nano 12(9):8855-8866 describing operable linkage to aldolase nanoparticles through isopeptide bond (“SpyTag-SpyCatcher”)). Pharmaceutically acceptable nanoparticles as carriers, as well as methods of using them to present an antigen, are known and include lumazine synthase, ferritin, or aldolase-based nanoparticles (or nanocages) or nanoparticles derived therefrom (see WO 2005/121330; WO 2013/044203; WO 2016/037154; and Bruun et al. 2018 ACS Nano 12(9):8855-8866). Such nanoparticles may be “self-assembling” (see WO 2015/048149). In the context of nanoparticles (or nanocages) as carriers, operable linkage of antigens onto a nanoparticle can be achieved through a variety of techniques including spontaneous isopeptide bond formation, chemical conjugation, genetic fusion, or bio-orthogonal chemistry with unnatural amino acids (see Bruun et al. 2018 ACS Nano 12(9):8855-8866 at 8855 and references therein). Linkers may be Universal T cell epitopes or Glycine/Serine/Alanine linkers (8 to 14 amino acid residues containing repeats of Glycine, Serine, or Alanine such as that shown in SEQ ID NO: 121) or Universal T cell epitopes (such as PADRE (SEQ ID NO: 122), D (SEQ ID NO: 123), TpD (SEQ ID NO: 124). In the context of betacoronavirus vaccination, T cell epitopes from a betacoronavirus antigen may be used (such as a T cell epitope from SARS CoV-2 M, N, or Spike (S) proteins). Bacterial lumazine synthase (LS) has been investigated for use as a pharmaceutically acceptable carrier. LS acts in the biosynthesis of riboflavin and is present in organisms including bacteria, plants, and eubacteria. Jardine et al. reported LS from the bacterium Aquifex aeolicus fused to an HIV gp120 antigen self-assembled into a 60-mer nanoparticle. Jardine et al., Science 340:711-716 (2013). Expression of wild-type A. aeolicus LS has been reported in E. coli; Jardine et al. described use of mammalian cells to produce LS nanoparticles comprising the HIV gp120 antigen. H. pylori bacterial ferritin (see PDB Accession Number 3BVE) has been investigated for use as a pharmaceutically acceptable carrier. H. pylori bacterial ferritin consists of 24 identical polypeptide subunits that self-assemble into a spherical nanoparticle. Li et al. reported preparation of a nucleotide sequence encoding a fusion of bacterial (H. pylori) ferritin subunit polypeptide, a rotavirus VP6 antigen, and a histidine tag to aid in purification, with expression in a prokaryotic (E. coli) system and removal of the His-tag. The expressed fusion polypeptides are described as self-assembling into spherical NPs displaying the rotavirus capsid protein VP6, and capable of inducing an immune response in mice. (Li et al., J Nanobiotechnol 17:13 (2019)). Wang et al. designed chimeric polypeptides comprising H. pylori ferritin and antigenic peptides from N. gonorrhoeae; the chimeric polypeptide is described as assembling into a 24-mer nanoparticle displaying the antigenic peptides on the NP exterior surface. (Wang et al., FEBS Open Bio 7(8):1196 (2017)). Kanekiyo et al. described a self-assembling recombinant bacterial (H. pylori) ferritin nanoparticle (24-mer), comprising fusions of the ferritin subunit polypeptide and influenza HA antigenic peptides, which displayed influenza HA trimers on its surface (Kanekiyo et al., Nature 499(7456):102 (2013)). Helicobacter pylori Neutrophil Activating Protein (HP-NAP) is a self-assembling nanoparticle known for its adjuvanting properties (WO 2007/039451 (PCT/EP2006/066507, DEL PRETE et al.)) that may be used as a carrier in certain embodiments. Nanoparticles based on insect ferritin have been investigated for use as a pharmaceutically acceptable carrier, in particular comprising both heavy and light chain subunit polypeptides for use in displaying, on the NP surface, trimeric antigens (WO2018/005558 (PCT/US2017/039595), Kwong et al.). Also, Li et al. described a nanoparticle made of recombinant fusion polypeptides comprising a human ferritin light-chain subunit and a short HIV-1 antigenic peptide attached to the amino terminus of the ferritin light-chain sequence, with self-assembly of these fusion polypeptides resulting in placement of the HIV-1 antigenic peptide at the exterior surface of the NP. Li et al., Ind. Biotechnol. 2:143-47 (2006)). Nanoparticles (nanocages) based on the Thermotoga maritima 2-keto-3-deoxy-phosphogluconate (KDPG) aldolase (PDB Accession Number 1WA3) for use as carriers and antigen display are also known and may be used (e.g., what is referred to as “i301” or “I3-01” in the field (Hsia et al. 2016 Nature 535(7610):136-139; PDB Accession Number 5KP9)—modified i301 nanocages are also known, e.g. what is referred to as “mi3” in the field (Bruun et al. 2018 ACS Nano 12(9):8855-8866)).

### Production and Delivery

Compositions of the invention will generally be administered directly to a subject (e.g., a human subject). Direct delivery may be accomplished by parenteral injection (e.g. subcutaneously, intraperitoneally, transdermally, intravenously, intramuscularly, intranasal, or to the interstitial space of a tissue), or by any other suitable route. Intramuscular administration is preferred e.g. to the thigh or the upper arm. Injection may be via a needle (e.g. a hypodermic needle), but needle-free injection may alternatively be used. In certain embodiments, a presently provided immunogenic composition is administered to a subject intranasally or intramuscularly. Intranasal and intramuscular vaccination was previously examined, with success, for candidate SARS-CoV-1 vaccines (Zheng B J et al. 2008 Hong Kong Med J 14(Suppl 4): S39-43). In some embodiments, the presently provided modified spike proteins or fragments thereof are delivered to a subject by administration of an immunologically effective amount of one or more recombinant nucleic acid molecules that together encode the modified spike proteins or fragments thereof, thereby producing an immune response to the modified spike proteins or fragments thereof. In some embodiments, nucleic acids encoding the modified spike proteins or fragments thereof are prepared by in vitro transcription (IVT), as discussed elsewhere herein. Such nucleic acid molecules useful for delivery to a subject and/or useful for nucleic acid production are thus embodiments of the invention.

The nucleic acid molecule of the invention may, for example, be RNA or DNA, such as a plasmid DNA. In one aspect, the invention provides a nucleic acid sequence comprising a construct encoding the modified spike proteins or fragments thereof, and further comprising additional sequence elements. For instance, the nucleic acid may comprise sequence elements useful for the functioning of a mRNA, a self-replicating RNA, a plasmid, or the like.

In some embodiments, the recombinant nucleic acid molecule is a DNA molecule. In one embodiment, the invention relates to a recombinant DNA molecule that encodes a mRNA molecule as described herein. In one embodiment, the invention relates to a recombinant DNA molecule that encodes a self replicating RNA molecule as described herein. In some embodiments, the recombinant DNA molecule is a plasmid and may serve as a template for synthesis of RNA in vitro. In such embodiments, the plasmid may comprise a bacteriophage (T7 or SP6) promoter upstream of the mRNA- or self-replicating-RNA encoding region to facilitate the synthesis of RNA in vitro. The plasmid may further comprise a restriction site at the end of the poly-A tail-encoding region, or a hepatitis delta virus (HDV) ribozyme immediately downstream of the poly(A)-tail generates the correct 3′-end through its self-cleaving activity. In some embodiments, the recombinant DNA molecule includes a mammalian promoter that drives transcription of the encoded self replicating RNA molecule as described herein. A recombinant DNA molecule that encodes a self replicating RNA molecule as described herein that is useful in accordance with the invention, can be prepared by the techniques described in WO 2012/051211 A2.

In some embodiments, the recombinant DNA molecule is an adenoviral vector, such as a simian adenoviral vector, encoding the modified spike proteins or fragments thereof. In embodiments of the adenoviral vectors of the invention, the adenoviral DNA is capable of entering a mammalian target cell, i.e. it is infectious. An infectious recombinant adenovirus of the invention can be used as a prophylactic or therapeutic vaccine and for gene therapy. Thus, in an embodiment, the recombinant adenovirus comprises an endogenous molecule for delivery into a target cell, such as a human cell. Such adenoviral vectors are known, see, e.g., WO 2018/104919. The endogenous molecule for delivery into a target cell can be an expression cassette. In an embodiment of the invention, the vector is a functional or an immunogenic derivative of an adenoviral vector. By “derivative of an adenoviral vector” is meant a modified version of the vector, e.g., one or more nucleotides of the vector are deleted, inserted, modified or substituted.

In a preferred embodiment, the nucleic acid molecule is an RNA molecule. In such embodiments, the RNA molecule comprises a construct encoding the modified spike proteins or fragments thereof disclosed herein. In a further preferred embodiment, the RNA molecule comprises mRNA sequence elements such as a cap, 5′-UTR, 3′-UTR, and poly-A tail. In a more preferred embodiment, the RNA molecule is a self-amplifying RNA molecule (“SAM”).

Self-amplifying (or self-replicating) RNA molecules are well known in the art and can be produced by using replication elements derived from, e.g., alphaviruses, and substituting the structural viral proteins with a nucleotide sequence encoding a protein of interest. A self-amplifying RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells. One suitable system for achieving self-replication in this manner is to use an alphavirus-based replicon. These replicons are +-stranded RNAs which lead to translation of a replicase (or replicase-transcriptase) after delivery to a cell. The replicase is translated as a polyprotein which auto-cleaves to provide a replication complex which creates genomic-strand copies of the +-strand delivered RNA. These −-strand transcripts can themselves be transcribed to give further copies of the +-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see WO2005/113782.

In one embodiment, the self-amplifying RNA molecule described herein encodes (i) an RNA-dependent RNA polymerase which can transcribe RNA from the self-amplifying RNA molecule and (ii) a presently provided modified spike protein or fragments thereof. The polymerase can be an alphavirus replicase e.g. comprising one or more of alphavirus proteins nsP1, nsP2, nsP3 and nsP4.

In certain embodiments, the self-amplifying RNA molecule is an alphavirus-derived RNA replicon as discussed herein.

Whereas natural alphavirus genomes encode structural virion proteins in addition to the non-structural replicase polyprotein, in certain embodiments, the self-amplifying RNA molecules do not encode alphavirus structural proteins. Thus, the self-amplifying RNA can lead to the production of genomic RNA copies of itself in a cell, but not to the production of RNA-containing virions. The inability to produce these virions means that, unlike a wild-type alphavirus, the self-amplifying RNA molecule cannot perpetuate itself in infectious form. The alphavirus structural proteins which are necessary for perpetuation in wild-type viruses are absent from self-amplifying RNAs of the present disclosure and their place is taken by gene(s) encoding the immunogen of interest, such that the subgenomic transcript encodes the immunogen rather than the structural alphavirus virion proteins. Thus, a self-amplifying RNA molecule useful with the invention may have two open reading frames. The first (5′) open reading frame encodes a replicase; the second (3′) open reading frame encodes an antigen. In some embodiments the RNA may have additional (e.g. downstream) open reading frames e.g. to encode further antigens or to encode accessory polypeptides.

Suitably, the self-amplifying RNA molecule disclosed herein has a 5′ cap (e.g. a 7-methylguanosine) which can enhance in vivo translation of the RNA. A self-amplifying RNA molecule may have a 3′ poly-A tail. It may also include a poly-A polymerase recognition sequence (e.g. AAUAAA) near its 3′ end. Self-amplifying RNA molecules can have various lengths but they are typically 5000-25000 nucleotides long. Self-amplifying RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

The self-amplifying RNA can conveniently be prepared by in vitro transcription (IVT). IVT can use a (cDNA) template created and propagated in plasmid form in bacteria or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-amplifying RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-amplifying RNA can include (in addition to any 5′ cap structure) one or more nucleotides having a modified nucleobase. An RNA used with the invention ideally includes only phosphodiester linkages between nucleosides, but in some embodiments, it can contain phosphoramidate, phosphorothioate, and/or methylphosphonate linkages.

The self-replicating RNA molecule may encode a single heterologous polypeptide antigen (i.e., be “monocistronic” encoding, e.g., a betacoronavirus S protein or fragment thereof) or, optionally, two or more heterologous polypeptide antigens (i.e., be “polycistronic”). Further details concerning use of polycistronic vectors to provide nucleic acid sequences that encode two or more proteins in desired relative amounts are provided in WO 2012/051211 A2, which is incorporated by reference for its teachings relating to expression of proteins for antigen delivery for vaccines. These teachings can be applied to expression of two or more betacoronavirus spike proteins in accordance with the present invention. Two or more heterologous polypeptides generated from a self-replicating RNA molecule may be expressed as a fusion polypeptide (fusion protein) or as separate polypeptides. The self-replicating RNA molecules described herein may be engineered to express multiple nucleotide sequences, from two or more open reading frames, thereby allowing co-expression of proteins, such as one or more betacoronavirus proteins (e.g., including one or more S protein or S protein fragment open reading frames), together with cytokines or other immunomodulators, which can enhance the generation of an immune response. Such a self-replicating RNA molecule might be particularly useful, for example, in the production of various gene products (e.g., proteins) at the same time, for example, as a bivalent or multivalent vaccine.

In some embodiments a self-replicating RNA molecule is provided comprising, from 5′ to 3′, polynucleotide sequences selected from the following: (A) a polynucleotide sequence having SEQ ID NO: 119; a polynucleotide sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 119; or a polynucleotide sequence that is a fragment of SEQ ID NO: 119; (B) a polynucleotide sequence encoding a betacoronavirus S protein or S protein fragment as described elsewhere herein; and (C) a polynucleotide sequence having SEQ ID NO: 120; a polynucleotide sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 120; or a polynucleotide sequence that is a fragment of SEQ ID NO: 120; wherein a fragment of SEQ ID NO: 119 or SEQ ID NO: 120 comprises a contiguous stretch of the nucleic acid sequence of the full-length sequence up to 1, 2, 3, 4, 5, 6, 7, 8, 9, 10, 15, 20, 25, 26, 27, 28, 29, or 30 nucleic acids shorter than full-length sequence.

In some embodiments is provided a self-replicating RNA molecule comprising, from 5′ to 3′, polynucleotide sequences selected from the following:

a polynucleotide sequence having SEQ ID NO: 119; a polynucleotide sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 119; or a polynucleotide sequence that is a fragment of SEQ ID NO: 119;

a polynucleotide sequence encoding a polypeptide having a sequence selected from the group consisting of SEQ ID NOS: 5-114; a polynucleotide sequence encoding a polypeptide having a sequence at least 70%, at least 75%, at least 80%, at least 85%, at least 90%, at least 91%, at least 92%, at least 93%, at least 94%, at least 95%, at least 96%, at least 97%, at least 98%, or at least 99% identical to a polypeptide sequence selected from the group consisting of SEQ ID NOS: 5-114; or a polynucleotide sequence encoding a fragment of a polypeptide having a sequence selected from the group consisting of SEQ ID NOS: 5-114; and

a polynucleotide sequence having SEQ ID NO: 120; a polynucleotide sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 120; or a polynucleotide sequence that is a fragment of SEQ ID NO: 120;

wherein a fragment of SEQ ID NO: 119 or SEQ ID NO: 120 comprises a contiguous stretch of the nucleic acid sequence of the full-length sequence up to 1, 2, 3, 4, 5, 6, 7, 8, 9, 10, 15, 20, 25, 26, 27, 28, 29, or 30 nucleic acids shorter than full-length sequence.

In some embodiments is provided a self-replicating RNA molecule comprising, from 5′ to 3′, a polynucleotide sequence having SEQ ID NO: 119, a polynucleotide sequence encoding a polynucleotide sequence encoding a betacoronavirus S protein or S protein fragment as described elsewhere herein, and a polynucleotide sequence having SEQ ID NO: 120. In some embodiments is provided a self-replicating RNA molecule comprising, from 5′ to 3′, a polynucleotide sequence having SEQ ID NO: 119, a polynucleotide sequence encoding a polypeptide having a sequence selected from the group consisting of SEQ ID NOs: 5-114, and a polynucleotide sequence having SEQ ID NO: 120. In some embodiments, the self-replicating RNA molecules comprise from 5′ to 3′ a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 119, a polynucleotide sequence encoding a polypeptide having a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence selected from the group consisting of SEQ ID NOS: 5-114, and a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 120. In some embodiments, the self-replicating RNA molecule comprises from 5′ to 3′ a sequence that is a fragment of SEQ ID NO: 119, a fragment of a full-length polynucleotide sequence encoding a polypeptide sequence selected from the group consisting of SEQ ID NOS: 5-114, and a sequence that is a fragment of SEQ ID NO: 120, wherein a fragment comprises a contiguous stretch of the nucleic acid sequence of the full-length sequence up to 1, 2, 3, 4, 5, 6, 7, 8, 9, 10, 15, 20, 25, 26, 27, 28, 29, or 30 nucleic acids shorter than full-length sequence.

The nucleic acid molecule of the invention may be associated with a viral or a non-viral delivery system. The delivery system (also referred to herein as a delivery vehicle) may have an adjuvant effects which enhance the immunogenicity of the encoded betacoronavirus Spike (S) protein or fragment thereof. For example, the nucleic acid molecule may be encapsulated in liposomes, non-toxic biodegradable polymeric microparticles or viral replicon particles (VRPs), or complexed with particles of a cationic oil-in-water emulsion. In some embodiments, the nucleic acid molecule is associated with a non-viral delivery material such as to form a cationic nano-emulsion (CNE) delivery system or a lipid nanoparticle (LNP) delivery system. In some embodiments, the nucleic acid molecule is associated with a non-viral delivery system, i.e., the nucleic acid molecule is substantially free of viral capsid. Alternatively, the nucleic acid molecule may be associated with viral replicon particles. In other embodiments, the nucleic acid molecule may comprise a naked nucleic acid, such as naked RNA (e.g. mRNA).

In a preferred embodiment, the RNA molecule or self-amplifying RNA molecule is associated with a non-viral delivery material, such as to form a cationic nanoemulsion (CNE) or a lipid nanoparticle (LNP).

CNE delivery systems and methods for their preparation are described in WO2012/006380. In a CNE delivery system, the nucleic acid molecule (e.g. RNA) which encodes the antigen is complexed with a particle of a cationic oil-in-water emulsion. Cationic oil-in-water emulsions can be used to deliver negatively charged molecules, such as an RNA molecule to cells. The emulsion particles comprise an oil core and a cationic lipid. The cationic lipid can interact with the negatively charged molecule thereby anchoring the molecule to the emulsion particles. Further details of useful CNEs can be found in WO2012/006380; WO2013/006834; and WO2013/006837 (the contents of each of which are incorporated herein in their entirety).

Thus, in one embodiment, an RNA molecule, such as a self-amplifying RNA molecule, encoding the modified spike proteins or fragments thereof may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. In some embodiments, the CNE comprises squalene and a cationic lipid, such as 1,2-dioleoyloxy-3-(trimethylammonio)propane (DOTAP). In some preferred embodiments, the delivery system is a non-viral delivery system, such as CNE, and the nucleic acid molecule comprises a self-amplifying RNA (mRNA). This may be particularly effective in eliciting humoral and cellular immune responses.

LNP delivery systems and non-toxic biodegradable polymeric microparticles, and methods for their preparation are described in WO2012/006376 (LNP and microparticle delivery systems); Geall et al. (2012) PNAS USA. September 4; 109(36): 14604-9 (LNP delivery system); and WO2012/006359 (microparticle delivery systems). LNPs are non-virion liposome particles in which a nucleic acid molecule (e.g. RNA) can be encapsulated. The particles can include some external RNA (e.g. on the surface of the particles), but at least half of the RNA (and ideally all of it) is encapsulated. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9. An LNP delivery approach is utilized for a candidate SARS-CoV-2 vaccine comprising LNP-encapsulated mRNA encoding spike (S) protein (see Le et al. 2020 Nat Rev Drug Disc 19:305-306).

In a further aspect, the invention provides a vector comprising a nucleic acid according to the invention.

A vector for use according to the invention may be any suitable nucleic acid molecule including naked DNA or RNA, a plasmid, a virus, a cosmid, phage vector such as lambda vector, an artificial chromosome such as a BAC (bacterial artificial chromosome), or an episome. For example, electroporation delivery of a DNA plasmid encoding spike (S) protein is being investigated as a candidate SARS-CoV-2 vaccine (see Le et al. 2020 Nat Rev Drug Disc 19:305-306). Alternatively, a vector may be a transcription and/or expression unit for cell-free in vitro transcription or expression, such as a T7-compatible system. The vectors may be used alone or in combination with other vectors such as adenovirus sequences or fragments, or in combination with elements from non-adenovirus sequences. Suitably, the vector has been substantially altered (e.g., having a gene or functional region deleted and/or inactivated) relative to a wild type sequence, and replicates and expresses the inserted polynucleotide sequence, when introduced into a host cell. For example, an Adenovirus type 5 (Ad5) vector that expresses spike (S) protein is being investigated as a candidate SARS-CoV-2 vaccine (see Le et al. 2020 Nat Rev Drug Disc 19:305-306). An adeno-associated virus (AAV) approach was also investigated as a candidate SARS-CoV-1 vaccine (intramuscular or mucosal delivery of an AAV-based vaccine containing the spike protein Receptor Binding Domain fragment, see Zheng B J et al. 2008 Hong Kong Med J 14(Suppl 4):S39-43 and Du L. et al. 2009 Nat. Rev. Microbio. 7:226-236).

In a further aspect, the invention provides a cell comprising a modified spike protein or fragment thereof, a nucleic acid encoding a presently provided modified spike protein or fragment thereof, or a vector according to the invention.

In one embodiment, the heterodimer according to the invention is expressed from a multicistronic vector. Suitably, the heterodimer is expressed from a single vector in which the nucleic sequences encoding the modified spike protein or fragment thereof are separated by an internal ribosomal entry site (IRES) sequence (Mokrejš, Martin, et al. “IRESite: the database of experimentally verified IRES structures (World Wide Web. iresite.org).” Nucleic acids research 34.suppl_1 (2006): D125-D130). Alternatively, the two nucleic sequences can be separated by a viral 2A or ‘2A-like’ sequence, which results in production of two separate polypeptides. 2A sequences are known from various viruses, including foot-and-mouth disease virus, equine rhinitis A virus, Thosea asigna virus, and porcine theschovirus-1. See e.g., Szymczak et al., Nature Biotechnology 22:589-594 (2004), Donnelly et al., J Gen Virol.; 82(Pt 5): 1013-25 (2001).

When a host cell herein is cultured under suitable conditions, the nucleic acid can express the modified spike protein or fragment thereof the modified spike protein or fragment thereof may then be purified from the host cell. Suitable host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster)), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof. Suitably, the host cell should be one that has enzymes that mediate glycosylation.

Suitable mammalian cells include, for example, Chinese hamster ovary (CHO) cells, human embryonic kidney cells (HEK-293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like.

In certain embodiments, the modified spike protein or fragment polynucleotide sequence is codon optimized for expression in a selected prokaryotic or eukaryotic host cell.

The modified spike protein or fragment can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxyapatite chromatography, and lectin chromatography. Protein refolding steps can be used, as desired, in completing configuration of the mature protein. Finally, high performance liquid chromatography (HPLC) can be employed in the final purification steps. In addition to the references noted above, a variety of purification methods are well known in the art, including, e.g., those set forth in Sandana (1997) Bioseparation of Proteins, Academic Press, Inc.; and Bollag et al. (1996) Protein Methods, 2nd Edition Wiley-Liss, NY; Walker (1996) The Protein Protocols Handbook Humana Press, N.J., Harris and Angal (1990) Protein Purification Applications: A Practical Approach IRL Press at Oxford, Oxford, U.K.; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

The term “purification” or “purifying” here refers to the process of removing components from a composition or host cell or culture, the presence of which is not desired. Purification is a relative term, and does not require that all traces of the undesirable component be removed from the composition. In the context of vaccine production, purification includes such processes as centrifugation, dialyzation, ion-exchange chromatography, and size-exclusion chromatography, affinity-purification or precipitation. Immunogenic molecules or antigens or antibodies which have not been subjected to any purification steps (i.e., the molecule as it is found in nature) are not suitable for pharmaceutical (e.g., vaccine) use.

### Use of Immunogenic Compositions

The immunogenic compositions herein may be administered on a single dose or multidose schedule. Certain embodiments provide delivery (e.g., administration) to a non-human mammal (e.g., mice) on a three dose schedule with dose delivery every about three weeks (such as on days 1, 22, and 43) or about three weeks post-last-dose. Certain embodiments provide delivery to a human subject on a three dose schedule with dose delivery once every about 1-6 months (e.g., dose delivery between about one and six months post-last-dose) such as

second delivery about one month post-first-dose and third delivery about six months post-first-dose or, said another way, third delivery about five months post-second-dose (i.e., 0-1-6 schedule);

second delivery about two months post-first-dose and third delivery about six months post-first-dose or, said another way, third delivery about four months post-second-dose (i.e., 0-2-6 schedule) or

second delivery about one month post-first-dose and third delivery about three months post-first dose or, said another way, third delivery about two months post-first-dose (i.e., 0-1-3 schedule).

Certain embodiments provide delivery of an immunogenic composition to a human subject intramuscularly as a 3-dose vaccination course on a 0, 1, and 6 months schedule. A particular embodiment provides delivery of the immunogenic composition to a human subject intramuscularly as a 3-dose vaccination course on a 0, 1, and 6 months schedule. A particular embodiment provides delivery of the immunogenic composition to a human subject intramuscularly as a 3-dose vaccination course on a 0, 2, and 6 months schedule. A particular embodiment provides delivery of the immunogenic composition to a human subject intramuscularly as a 3-dose vaccination course on a 0, 1, and 3 months schedule. Another embodiment provides delivery to a human subject on a two dose schedule with a second dose delivery about one month, about two months, or about six months post-first-dose (i.e., delivery of an immunogenic composition to a human subject as a 2-dose vaccination course on a 0, 1; 0, 2; or 0, 6 months schedule). In a particular example, the immunogenic composition is administered to a human subject intramuscularly as a 2-dose vaccination course on a 0 and 1 months schedule. In a particular example, the immunogenic composition is administered to a human subject intramuscularly as a 2-dose vaccination course on a 0 and 6 months schedule.

A prime-boost regimen may be used. Prime-boost refers to eliciting two separate immune responses in the same individual: (i) an initial priming of the immune system followed by (ii) a secondary or boosting of the immune system weeks or months after the primary immune response has been established. Preferably, a boosting composition is administered about two to about 12 weeks after administering the priming composition to the subject, for example about 2, 3, 4, 5 or 6 weeks after administering the priming composition. In one embodiment, a boosting composition is administered one or two months after the priming composition. In one embodiment, a first boosting composition is administered one or two months after the priming composition and a second boosting composition is administered one or two months after the first boosting composition. A prime-boost regimen was previously examined, with success, for a candidate SARS-CoV-1 vaccine (Zheng B J et al. 2008 Hong Kong Med J 14(Suppl 4): S39-43); in particular priming with administration of an adeno-associated virus (AAV) containing SARS-CoV-1 spike protein RBD and boosting with RBD-specific peptides (Du L. et al. 2009 Nat. Rev. Microbio. 7:226-236).

### EXAMPLES

### Example 1: Stabilizing Mutants

### Symmetric Interface Design Using Rosetta HBNet Workflow, Targeting Cross-Protomer Residues:

HBNet is a computational design method/algorithm that runs within the Rosetta Commons (rosettacommons.org) scripts framework. HBNet detects and designs Hydrogen Bond Networks (hence, “HBNet”) within the user-defined design space and that meet user-defined criteria.

This study was to design stabilizing mutations of the Spike (S) protein from the SARS CoV-2 antigen using (1) hydrogen bonding networks and (2) cavity-filling substitutions to enhance the structural and conformational integrity of the pre-fusion trimer.

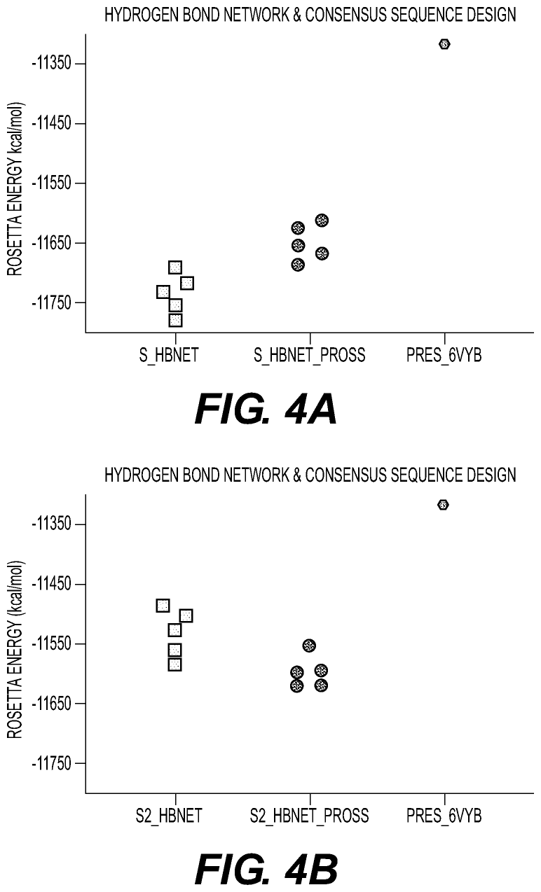

Rosetta comparative modeling (RosettaCM) (Song et al. 2013 Structure 21: 1735-1742) with symmetry restraints (DiMaio et al. 2011 PLoS ONE 6(6): e20450, doi:10.1371/journal.pone.0020450) was used to build a model of the SARS CoV-2 S antigen with the receptor binding domain (RBD) in the open conformation (PDB Accession Numbers: 6VSB, 6VYB), using combinations of x-ray and cryo-EM structures (PDB Accession Numbers: 6VYB, 6VW1, 6NB7 (SARS-CoV-1). As of Jun. 5, 2020, there were two “wild type” SARS-CoV-2 Spike Proteins described in the art. One was PDB 6VYB (from Vessler) and the other was PDB 6VSB (by Mcllelum). Unless otherwise noted, in the present application, the Vessler structure was used. Symmetric interface design was performed on the lowest energy RosettaCM structure, using the Monte-Carlo based HBNet algorithm to introduce polar networks between S protein protomers. Sequence design was done on the full S protein targeting the S1 & S2 domains or the S2 domain only (FIG. 2).

Fixed backbone design was performed after the generation of hydrogen bond networks, using RosettaHoles (Sheffler and Baker 2009 Protein Science 18:229-239) to detect cavities, and doing sequence design to find the most stabilizing mutant combinations.

The top sequences were selected based on overall Rosetta Energy, relative to the initial structure, indicating a correlation between the number of mutations (S1+S2-specific (i.e., S-specific) or S2-specific) and the difference in in silico stability (FIG. 2).

As these results demonstrate, a mutation(s) in one S protein monomer (protomer) sequence causes each protomer of the resultant S protein homotrimer to also incorporate that mutation(s). In this way, modification of an “S protein” or “S protein fragment” sequence would be understood without further specification of a particular protomer sequence being modified (such specification would instead be irrelevant, even confusing, to an artisan).

### Results:

In Table 1 are provided (from left column to right): certain target residues of wild type SARS-CoV-2 amino acid sequence SEQ ID NO: 3; certain target residues of control SARS-CoV-2 amino acid sequence SEQ ID NO: 4 (which, as compared to SEQ ID NO: 3, is modified to comprise the furin cleavage abrogation mutations and prefusion double proline mutations of Wrapp et al. (2020 Science 367(6483):1260-1263) as well as the D588G consensus mutation of Brufsky (20 Apr. 2020 J Med Virol, 7 pages, doi:10.1002/jmv.25902, therein D614G; see also Korber et al. 2020 bioRxiv (HyperTextTransferProtocolSecure: /doi.org/10.1101/2020.04.29.069054)); the presently provided point mutations of those target residues which were designed with HBNet (“HBNet mutations”) to increase the (thermo)stability of the wild type (SEQ ID NO: 3) or control (SEQ ID NO: 4) S proteins; and then a summary of what amino acids are present at those target residue positions within the designed, modified S protein fragment sequences SEQ ID NOs: 5-14. The sequence SEQ ID NO: 4 was used as the “parent” sequence for modified S proteins comprising HBNet mutations, so all of sequences SEQ ID NO: 5-14 comprise the furin cleavage abrogation mutations and prefusion double proline mutations that SEQ ID NO: 4 comprises. Further, SEQ ID NOs: 10-14 also comprise the D588G consensus mutation that is within SEQ ID NO: 4.

TABLE 1

| | Column | Column | Column | Column | Column | Column | Column | Column | Column | Column | Column | Column | Column |
|---|---|---|---|---|---|---|---|---|---|---|---|---|---|
| | #1 | #2 | #3 | #4 | #5 | #6 | #7 | #8 | #9 | #10 | #11 | #12 | #13 |
| | SEQ ID | SEQ ID | HBNet | SEQ ID | SEQ ID | SEQ ID | SEQ ID | SEQ ID | SEQ ID | SEQ ID | SEQ ID | SEQ ID | SEQ ID |
| Row # | NO: 3 | NO: 4 | mutations | NO: 5 | NO: 6 | NO: 7 | NO: 8 | NO: 9 | NO: 10 | NO: 11 | NO: 12 | NO: 13 | NO: 14 |
| 3 | F17 | | S | S | S | S | S | S | F | F | F | F | F |
| 4 | R18 | | M | M | M | M | M | M | R | R | R | R | R |
| 5 | E198 | | V | E | V | E | E | E | E | E | E | E | E |
| 6 | P199 | | L | L | L | L | L | L | P | P | P | P | P |
| 7 | T258 | | V | V | V | V | V | V | T | T | T | T | T |
| 8 | Q288 | | I or | I | I | D | D | I | Q | Q | Q | Q | Q |
| | | | D | | | | | | | | | | |
| 9 | N291 | | L or | L | L | T | T | L | N | N | N | N | N |
| | | | T | | | | | | | | | | |
| 10 | R293 | | E or | E | K | E | K | K | R | R | R | R | R |
| | | | K | | | | | | | | | | |
| 11 | L492 | | N | N | N | N | N | N | L | L | L | L | L |
| 12 | K531 | | L | L | L | L | L | L | K | K | K | K | K |
| 13 | L534 | | V | V | L | V | V | V | L | L | L | L | L |
| 14 | P535 | | S or | S | E | S | S | S | P | P | P | P | P |
| | | | E | | | | | | | | | | |
| 15 | F536 | | T | T | F | T | T | T | F | F | F | F | F |
| 16 | Q538 | | L | L | L | L | L | L | Q | Q | Q | Q | Q |
| 17 | G540 | | R or | R | H | R | R | M | G | G | G | G | G |
| | | | H or | | | | | | | | | | |
| | | | M | | | | | | | | | | |
| 18 | R541 | | V | V | V | V | V | V | R | R | R | R | R |
| 19 | D542 | | H | H | H | H | H | H | D | D | D | D | D |
| 20 | I543 | | S | S | S | S | S | S | I | I | I | I | I |
| 21 | D545 | | N | N | N | N | N | N | D | D | D | D | D |
| 22 | D548 | | L | L | L | L | L | L | D | D | D | D | D |
| 23 | A549 | | G | A | A | A | A | G | A | A | A | A | A |
| 24 | T562 | | V | V | V | V | V | V | T | T | T | T | T |
| 25 | P563 | | S | S | S | S | S | S | P | P | P | P | P |
| 26 | F566 | | S | S | S | S | S | S | F | F | F | F | F |
| 27 | G568 | | A or | A | A | R | R | A | G | G | G | G | G |
| | | | R | | | | | | | | | | |
| 28 | Q587 | | Y or | Y | Y | R | R | Y | Q | Q | Q | Q | Q |
| | | | R | | | | | | | | | | |
| 29 | D588 | G | N | N | N | N | N | N | G | G | G | G | G |
| 30 | N590 | | W | W | W | W | W | W | N | N | N | N | N |
| 31 | R620 | | K | K | R | K | R | R | R | R | R | R | R |
| 32 | P639 | | A or | A | A | Y | A | Y | P | P | P | P | P |
| | | | Y | | | | | | | | | | |
| 33 | A642 | | G | G | A | G | A | A | A | A | A | A | A |
| 34 | R656 | G | | G | G | G | G | G | G | G | G | G | G |
| 35 | R657 | S | | S | S | S | S | S | S | S | S | S | S |
| 36 | R659 | S | | S | S | S | S | S | S | S | S | S | S |
| 37 | T670 | | W or | W | Q | W | Q | Q | Q | Q | Q | Q | Q |
| | | | Q | | | | | | | | | | |
| 38 | M671 | | I | I | I | I | I | I | I | I | I | I | I |
| 39 | L673 | | T | T | T | T | T | T | T | T | T | T | T |
| 40 | A675 | | S | S | S | S | S | S | S | S | S | S | S |
| 41 | E676 | | W | W | W | W | W | W | W | W | W | W | W |
| 42 | A680 | | D or | D | D | E | D | D | E | D | D | D | D |
| | | | E | | | | | | | | | | |
| 43 | Y681 | | N | N | N | N | N | N | N | N | N | N | N |
| 44 | N684 | | D | D | D | D | D | D | D | D | D | D | D |
| 45 | S685 | | A | A | A | A | A | A | A | A | A | A | A |
| 46 | I688 | | V | I | I | I | V | I | I | V | I | I | I |
| 47 | P689 | | A | A | A | A | A | A | A | A | A | A | A |
| 48 | S709 | | W or | W | W | H | H | W | W | W | W | W | W |
| | | | H | | | | | | | | | | |
| 49 | D711 | | I | I | I | D | D | I | D | D | D | D | D |
| 50 | M714 | | L | L | L | L | L | L | L | L | L | L | L |
| 51 | D719 | | G | G | G | G | G | G | G | G | G | G | G |
| 52 | L728 | | A | A | A | A | A | A | A | A | A | A | A |
| 53 | Y730 | | H | Y | H | Y | H | H | Y | H | Y | Y | Y |
| 54 | Q736 | | E | E | E | E | E | E | E | E | E | E | E |
| 55 | A740 | | M | A | A | M | M | A | M | A | M | M | M |
| 56 | Q753 | | W | W | W | W | W | W | W | W | W | W | W |
| 57 | Q758 | | T | Q | T | T | T | T | T | T | T | T | T |
| 58 | K760 | | R | R | R | R | R | R | R | R | R | R | R |
| 59 | Q761 | | T | T | T | T | T | T | T | T | T | T | T |
| 60 | Y763 | | F | F | F | F | F | F | F | F | F | F | F |
| 61 | K764 | | H | H | H | H | H | H | H | H | H | H | H |
| 62 | P767 | | S | S | S | S | S | S | S | S | S | S | S |
| 63 | L823 | | S | L | L | L | L | L | S | S | S | S | S |
| 64 | I824 | | S | S | S | S | S | S | S | S | S | S | S |
| 65 | A826 | | H | H | H | H | H | H | A | A | A | A | A |
| 66 | K828 | | D | D | D | D | D | D | K | K | K | K | K |
| 67 | F829 | | S or | S | S | S | S | S | A | A | A | A | A |
| | | | A | | | | | | | | | | |
| 68 | N830 | | R or | R | H | R | H | H | N | N | N | N | N |
| | | | H | | | | | | | | | | |
| 69 | T833 | | N | N | N | N | N | N | N | N | N | N | N |
| 70 | V834 | | I | I | I | I | I | I | I | I | I | I | I |
| 71 | P836 | | S | S | S | S | S | S | S | S | S | S | S |
| 72 | P837 | | S or | S | S | S | S | S | S | H | S | H | S |
| | | | H | | | | | | | | | | |
| 73 | M843 | | L | L | L | L | L | L | L | L | L | L | L |
| 74 | Q846 | | E | E | E | E | E | E | E | E | E | E | E |
| 75 | Y847 | | F | F | F | F | F | F | F | F | F | F | F |
| 76 | S858 | | A | A | A | A | A | A | A | A | A | A | A |
| 77 | W860 | | H or | W | H | W | T | W | W | T | S | W | W |
| | | | T or | | | | | | | | | | |
| | | | S | | | | | | | | | | |
| 78 | T861 | | S | S | T | S | T | S | S | T | T | S | T |
| 79 | G863 | | T or | T | T | T | L | L | T | L | I | L | L |
| | | | L or | | | | | | | | | | |
| | | | I | | | | | | | | | | |
| 80 | A866 | | H | H | H | A | H | A | A | H | H | H | A |
| 81 | L868 | | S or | L | S | L | C | L | L | C | L | L | L |
| | | | C | | | | | | | | | | |
| 82 | Q869 | | N | N | N | N | N | N | N | N | N | N | N |
| 83 | F872 | | W | W | W | W | W | W | W | W | W | W | W |
| 84 | A873 | | W | A | W | W | W | W | W | W | W | A | A |
| 85 | M874 | | Vor | V | A | A | A | A | A | A | A | E | V |
| | | | A or | | | | | | | | | | |
| | | | E | | | | | | | | | | |
| 86 | Y878 | | W or | W | W | W | W | W | W | Q | W | W | W |
| | | | Q | | | | | | | | | | |
| 87 | N881 | | A or | A | A | A | A | K | A | A | A | A | A |
| | | | K | | | | | | | | | | |
| 88 | Q887 | | E | E | E | E | E | E | E | E | E | E | E |
| 89 | N888 | | W | N | N | N | N | W | N | N | N | N | N |
| 90 | Y891 | | A | A | A | A | A | A | A | A | A | A | A |
| 91 | E892 | | K or | K | K | K | K | I | K | K | K | K | K |
| | | | I | | | | | | | | | | |
| 92 | N934 | | D or | D | D | D | A | D | A | A | A | A | A |
| | | | A | | | | | | | | | | |
| 93 | T935 | | E or | E | E | E | E | E | E | E | E | E | Q |
| | | | Q | | | | | | | | | | |
| 94 | V937 | | E | E | E | E | E | E | E | E | E | E | E |
| 95 | K938 | | R | R | R | R | R | R | K | K | K | K | K |
| 96 | Q939 | | E or | E | E | E | E | E | E | E | E | E | T |
| | | | T | | | | | | | | | | |
| 97 | R957 | | N or | N | N | N | N | N | N | H | N | N | N |
| | | | H | | | | | | | | | | |
| 98 | K960 | P | | P | P | P | P | P | P | P | P | P | P |
| 99 | V961 | P | | P | P | P | P | P | P | P | P | P | P |
| 100 | T972 | | L | L | L | L | L | L | L | L | L | L | L |
| 101 | Q976 | | M or | M | L | M | L | L | M | L | M | M | M |
| | | | L | | | | | | | | | | |
| 102 | S977 | | A | A | A | A | A | A | A | A | A | A | A |
| 103 | Q979 | | A | A | A | A | A | Q | A | Q | A | A | A |
| 104 | T980 | | A | A | A | A | A | A | A | A | A | A | A |
| 105 | Y981 | | F | F | F | F | F | F | F | F | F | F | F |
| 106 | Q984 | | A | A | A | A | A | A | A | A | A | A | A |
| 107 | L986 | | A | L | L | A | A | L | A | L | A | A | A |
| 108 | T1001 | | L | T | T | L | T | T | T | T | T | T | T |
| 109 | S1004 | | A or | A | R | R | R | R | R | R | R | R | R |
| | | | R | | | | | | | | | | |
| 110 | E1005 | | I | E | E | I | E | E | E | E | E | E | E |
| ill | L1008 | | A or | A | A | A | A | A | A | A | N | A | A |
| | | | N | | | | | | | | | | |
| 112 | R1013 | | L | L | L | L | L | L | L | L | L | L | L |
| 113 | V1014 | | W or | V | V | V | W | W | V | W | H | W | W |
| | | | H | | | | | | | | | | |
| 114 | D1015 | | G | G | G | G | G | G | G | G | G | G | G |
| 115 | K1019 | | E | E | E | E | E | E | E | E | E | E | E |
| 116 | Y1021 | | W or | W | W | W | W | W | W | F | W | W | F |
| | | | F | | | | | | | | | | |
| 117 | Y1041 | | L | L | L | L | L | L | L | Y | L | L | Y |
| 118 | P1043 | | A | A | A | A | A | A | A | A | A | A | A |
| 119 | A1044 | | G | G | G | G | G | G | G | G | G | G | G |
| 120 | E1046 | | T or | T | T | Y | T | L | Y | T | S | S | Y |
| | | | Y or | | | | | | | | | | |
| | | | L or | | | | | | | | | | |
| | | | S | | | | | | | | | | |
| 121 | P1053 | | L | L | P | P | P | P | P | P | P | L | L |
| 122 | F1063 | | I or | I | I | I | I | V | I | I | I | I | I |
| | | | V | | | | | | | | | | |
| 123 | R1065 | | S or | R | R | S | R | R | R | R | R | R | R |
| | | | R | | | | | | | | | | |
| 124 | E1066 | | N or | N | T | T | N | I | N | N | N | N | N |
| | | | T or | | | | | | | | | | |
| | | | I | | | | | | | | | | |
| 125 | V1068 | | T | V | V | V | V | V | V | T | T | V | V |
| 126 | R1081 | | E or | E | E | E | E | E | E | D | W | E | E |
| | | | D or | | | | | | | | | | |
| | | | W | | | | | | | | | | |
| 127 | N1082 | | Q or | Q | N | Q | E | Q | Q | N | E | Q | N |
| | | | E | | | | | | | | | | |
| 128 | E1085 | | F | E | E | E | E | F | E | E | E | E | E |
| 129 | Q1087 | | L | L | Q | Q | L | L | L | L | L | L | L |
| 130 | N1093 | | L | L | N | L | L | L | L | L | L | L | L |
| 131 | T1094 | | V | V | V | V | V | V | V | V | V | V | V |
| 132 | F1095 | | L or | L | F | I | L | L | L | L | L | L | L |
| | | | I | | | | | | | | | | |
| 133 | V1102 | | D | D | D | D | D | D | D | D | D | D | D |
| 134 | L1115 | | K | K | L | L | L | L | K | L | L | L | L |

Design with Evolutionary Constraints in the Rosetta PROSS Design Workflow:

The Protein Repair One-Stop Shop (or “PROSS”) provides an algorithm for computational design of sequences that should result in a protein having a desirable function such as, for example, improved expression levels, improved expression in E. coli or other heterologous systems, improved solubility, less misfolding (i.e., when the protein is innately soluble and folded, but in an inactive conformation), less aggregation, longer half-life in-vitro or in-vivo, or higher melting temperature (Tm) (HyperTextTransferProtocol Secure://pross.weizmann.ac.il/about/).

This study was to design mutations of the S protein from SARS CoV-2 using evolutionary constraints for the introduction of stabilizing residues.

Homologous sequences were obtained from the non-redundant BLAST database and narrowed to 500 glycoprotein sequences. These aligned sequences were calculated into a position-specific scoring matrix (PSSM) with the PSI-BLAST algorithm. The matrix represents the likelihood of the 20 amino acids being present at each residue position, within the aligned sequences.

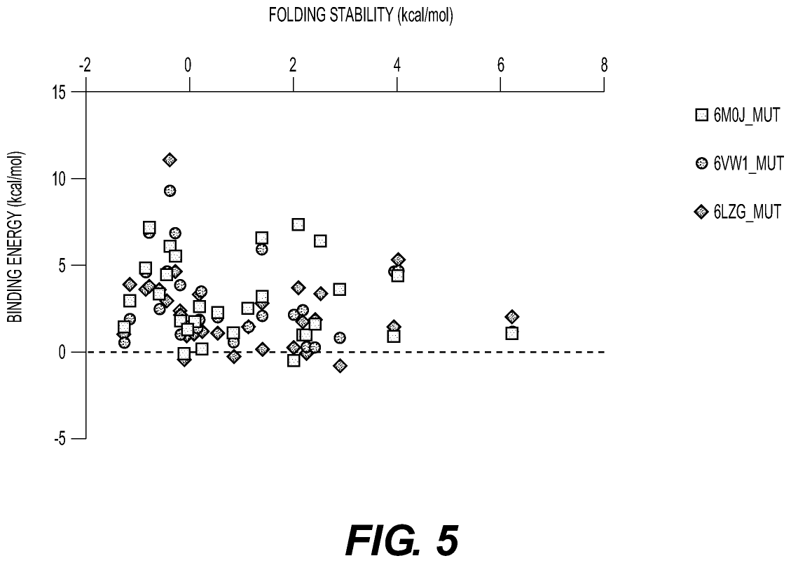

The starting structure for the S antigen in the open conformation was built in RosettaCM and designed using an updated version of the PROSS algorithm (with symmetry restraints and the beta energy scoring function). Goldenzweig et al. 2016 Molecular Cell 63(2):337-346. The Rosetta FilterScan mover was used to perform single point mutagenesis of all the residues to the preferred PSSM mutations, targeting the S domain, N-terminal domain (NTD) plus S2 domain, or the S2 domain only. The mutation scan was binned within twelve different energy thresholds (−0.5, −1, −1.5, −2, −2.5, −3, −3.5, −4, −4.5, −5, −5.5, −6 kcal/mol) to increase mutation sequence diversity (FIG. 3). For example, a combination of −6 kcal/mol single point mutations would result in fewer mutations due to a higher energetic barrier for introducing new mutations.

A RosettaScripts algorithm that energetically combined the proposed single mutations was used to reduce the search space, yielding twelve total stabilizing designs for each round of mutations, and representing each energy threshold (FIG. 3).

In summary, the design protocol performs an alignment to non-redundant glycoprotein sequences in the BLAST database, followed by single point mutagenesis (at different energy thresholds: −0.5, −1, −1.5, −2, −2.5, −3, −3.5, −4, −4.5, −5, −5.5, −6 kcal/mol) and combinatorial design to yield the most stabilizing residues (highlighted in cyan).

### Results:

In Table 2 are provided (from left column to right): certain target residues of wild type SARS-CoV-2 amino acid sequence SEQ ID NO: 3; certain target residues of control SARS-CoV-2 amino acid sequence SEQ ID NO: 4; the presently provided point mutations of those target residues which were designed with PROSS (“PROSS mutations”) to increase the (thermo)stability of the wild type (SEQ ID NO: 3) or control (SEQ ID NO: 4) S proteins; and then a summary of what amino acids are present at those target residue positions within the designed, modified S protein fragment sequences SEQ ID NOs: 15-29. The sequence SEQ ID NO: 4 was used as the “parent” sequence for modified S proteins comprising PROSS mutations, so all of sequences SEQ ID NO: 15-29 comprise the furin cleavage abrogation mutations and prefusion double proline mutations that SEQ ID NO: 4 comprises. Further, SEQ ID NOs: 17, 19, and 22-29 also comprise the D588G consensus mutation that is within SEQ ID NO: 4.

TABLE 2

| | | | | | | | | | | Column | Column | Column | Column | Column | Column | Column | Column | Column |
|---|---|---|---|---|---|---|---|---|---|---|---|---|---|---|---|---|---|---|
| | Column #1 | Column #2 | Column #3 | Column #4 | Column #5 | Column #6 | Column #7 | Column #8 | Column #9 | #10 | #11 | #12 | #13 | #14 | #15 | #16 | #17 | #18 |
| | SEQ ID | SEQ ID | PROSS | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| Row # | NO: 3 | NO: 4 | Mutations | NO: 15 | NO: 16 | NO: 17 | NO: 18 | NO: 19 | NO: 20 | NO: 21 | NO: 22 | NO: 23 | NO: 24 | NO: 25 | NO: 26 | NO: 27 | NO: 28 | NO: 29 |
| 3 | T7 | | R | R | T | T | T | T | R | T | T | T | T | T | T | T | T | T |
| 4 | V16 | | I | V | V | V | V | V | I | V | V | V | V | V | V | V | V | V |
| 5 | S20 | | N | N | N | N | N | N | N | N | N | N | N | S | S | S | S | S |
| 6 | S24 | | L | L | L | L | L | L | L | L | L | L | L | S | S | S | S | S |
| 7 | H43 | | N | N | N | H | H | H | N | N | H | H | H | H | H | H | H | H |
| 8 | S68 | | A | A | S | S | S | S | A | S | S | S | S | S | S | S | S | S |
| 9 | S72 | | N | S | S | S | S | S | N | S | S | S | S | S | S | S | S | S |
| 10 | T82 | | S | S | T | T | T | T | S | T | T | T | T | T | T | T | T | T |
| 11 | S90 | | T | T | S | S | S | S | T | S | S | S | S | S | S | S | S | S |
| 12 | A97 | | G | G | G | G | A | A | G | G | G | A | A | A | A | A | A | A |
| 13 | V100 | | I | V | V | V | V | V | I | V | V | V | V | V | V | V | V | V |
| 14 | K103 | | R | R | R | R | K | K | R | R | R | K | K | K | K | K | K | K |
| 15 | Q108 | | N | N | N | Q | Q | Q | N | N | N | Q | Q | Q | Q | Q | Q | Q |
| 16 | N111 | | E | E | E | E | E | E | E | E | E | E | E | N | N | N | N | N |
| 17 | D112 | | N | D | N | D | D | D | N | N | D | D | D | D | D | D | D | D |
| 18 | M127 | | L or S | L | M | M | M | M | S | M | M | M | M | M | M | M | M | M |
| 19 | E130 | | G | G | G | G | E | E | G | G | G | E | E | E | E | E | E | E |
| 20 | R132 | | H | H | H | H | H | H | H | H | H | H | H | R | R | R | R | R |
| 21 | S135 | | D or T | D | T | S | S | S | D | T | S | S | S | S | S | S | S | S |
| 22 | Q147 | | H | H | H | Q | Q | Q | H | H | Q | Q | Q | Q | Q | Q | Q | Q |
| 23 | L150 | | I | I | I | L | L | L | I | I | L | L | L | L | L | L | L | L |
| 24 | K156 | | D | D | D | D | K | K | D | D | D | K | K | K | K | K | K | K |
| 25 | Q157 | | S | S | S | S | Q | Q | S | S | S | Q | Q | Q | Q | Q | Q | Q |
| 26 | N162 | | H | H | H | N | N | N | H | H | N | N | N | N | N | N | N | N |
| 27 | V167 | | I | I | I | I | V | V | I | I | I | V | V | V | V | V | V | V |
| 28 | Y174 | | W | W | W | W | Y | Y | W | W | W | Y | Y | Y | Y | Y | Y | Y |
| 29 | K176 | | H or L | H | H | H | K | K | L | K | H | H | K | K | K | K | K | K |
| 30 | K180 | | S | S | K | K | K | K | S | K | K | K | K | K | K | K | K | K |
| 31 | R188 | | T | T | T | R | R | R | T | T | R | R | R | R | R | R | R | R |
| 32 | Q192 | | A or E | A | A | E | E | Q | A | A | E | E | Q | Q | Q | Q | Q | Q |
| 33 | P199 | | L | L | L | P | P | p | L | L | P | P | P | P | P | P | P | P |
| 34 | T214 | | I | I | I | I | I | I | I | I | I | I | I | T | T | T | T | T |
| 35 | S229 | | R | R | R | R | R | S | R | R | R | R | S | S | S | S | S | S |
| 36 | A234 | | R | R | R | R | A | A | R | R | R | A | A | A | A | A | A | A |
| 37 | A238 | | V | V | V | A | A | A | V | V | V | A | A | A | A | A | A | A |
| 38 | N254 | | D | N | N | N | N | N | D | N | N | N | N | N | N | N | N | N |
| 39 | S271 | | A | A | A | S | S | S | A | A | A | S | S | S | S | S | S | S |
| 40 | Q295 | | R | R | Q | Q | Q | Q | R | Q | Q | Q | Q | Q | Q | Q | Q | Q |
| 41 | P311 | | D | D | D | D | D | D | P | P | P | P | P | P | P | P | P | P |
| 42 | G313 | | S or D | S | S | D | D | S | G | G | G | G | G | G | G | G | G | G |
| 43 | V341 | | S | S | S | V | V | V | V | V | V | V | V | V | V | V | V | V |
| 44 | A346 | | T | T | T | T | T | T | A | A | A | A | A | A | A | A | A | A |
| 45 | K352 | | H or W | H | K | W | K | K | K | K | K | K | K | K | K | K | K | K |
| 46 | S357 | | D | D | D | S | S | S | S | S | S | S | S | S | S | S | S | S |
| 47 | T359 | | K | K | T | T | T | T | T | T | T | T | T | T | T | T | T | T |
| 48 | I384 | | L | L | L | L | L | L | I | I | I | I | I | I | I | I | I | I |
| 49 | K391 | | E | E | E | E | E | K | K | K | K | K | K | K | K | K | K | K |
| 50 | S417 | | A | A | A | A | A | S | S | S | S | S | S | S | S | S | S | S |
| 51 | K418 | | R | R | R | R | R | R | K | K | K | K | K | K | K | K | K | K |
| 52 | V419 | | K | K | K | V | V | V | V | V | V | V | V | V | V | V | V | V |
| 53 | G420 | | S | S | S | S | S | G | G | G | G | G | G | G | G | G | G | G |
| 54 | K432 | | N or H | N | H | K | K | K | K | K | K | K | K | K | K | K | K | K |
| 55 | S433 | | G | G | G | G | G | G | S | S | S | S | S | S | S | S | S | S |
| 56 | K436 | | R | R | R | R | K | K | K | K | K | K | K | K | K | K | K | K |
| 57 | A449 | | L | L | A | A | A | A | A | A | A | A | A | A | A | A | A | A |
| 58 | S451 | | D | D | D | S | S | S | S | S | S | S | S | S | S | S | S | S |
| 59 | G470 | | D or N | D | D | D | N | G | G | G | G | G | G | G | G | G | G | G |
| 60 | V477 | | S | S | S | S | S | V | V | V | V | V | V | V | V | V | V | V |
| 61 | G478 | | E or S | E | S | H | G | G | G | G | G | G | G | G | G | G | G | G |
| 62 | A494 | | G | G | G | G | G | A | A | A | A | A | A | A | A | A | A | A |
| 63 | S504 | | N | N | N | N | N | N | S | S | S | S | S | S | S | S | S | S |
| 64 | N506 | | S | S | S | N | N | N | N | N | N | N | N | N | N | N | N | N |
| 65 | N518 | | Y | Y | Y | Y | N | N | N | N | N | N | N | N | N | N | N | N |
| 66 | L520 | | Y | Y | Y | Y | Y | L | L | L | L | L | L | L | L | L | L | L |
| 67 | P535 | | S | S | S | S | S | S | P | P | P | P | P | P | P | P | P | P |
| 68 | Q538 | | L | L | L | Q | Q | Q | Q | Q | Q | Q | Q | Q | Q | Q | Q | Q |
| 69 | I543 | | S | S | S | S | S | S | I | I | I | I | I | I | I | I | I | I |
| 70 | A544 | | S | S | A | A | A | A | A | A | A | A | A | A | A | A | A | A |
| 71 | L556 | | N | N | N | N | N | N | L | L | L | L | L | L | L | L | L | L |
| 72 | L559 | | Y | Y | Y | Y | L | L | L | L | L | L | L | L | L | L | L | L |
| 73 | N577 | | D | D | N | N | N | N | D | N | N | N | N | N | N | N | N | N |
| 74 | Q581 | | E | E | Q | Q | Q | Q | E | Q | Q | Q | Q | Q | Q | Q | Q | Q |
| 75 | D588 | G | N | N | N | G | N | G | N | N | G | G | G | G | G | G | G | G |
| 76 | T592 | | S | S | T | T | T | T | S | T | T | T | T | T | T | T | T | T |
| 77 | V596 | | T | V | V | V | V | V | T | V | V | V | V | V | V | V | V | V |
| 78 | D601 | | N | N | N | D | D | D | N | N | D | D | D | D | D | D | D | D |
| 79 | V609 | | R | R | R | R | R | V | R | R | R | R | V | V | V | V | V | V |
| 80 | V616 | | I | I | I | V | V | V | I | I | I | V | V | V | V | V | V | V |
| 81 | H629 | | F or Y | F | Y | Y | H | H | F | H | Y | Y | H | H | H | H | H | H |
| 82 | Q649 | | D | D | D | D | D | Q | D | D | D | D | Q | Q | Q | Q | Q | Q |
| 83 | P655 | | R | P | P | P | P | p | R | P | P | P | p | p | p | p | p | p |
| 84 | R656 | G | | G | G | G | G | G | G | G | G | G | G | G | G | G | G | G |
| 85 | R657 | S | | S | S | S | S | S | S | S | S | S | S | S | S | S | S | S |
| 86 | R659 | S | | S | S | S | S | S | S | S | S | S | S | S | S | S | S | S |
| 87 | A675 | | S or E | S | S | E | A | A | S | S | E | A | A | S | S | E | E | A |
| 88 | A680 | | S | S | S | S | A | A | S | S | S | A | A | S | S | S | A | A |
| 89 | S682 | | D | S | S | S | S | S | S | S | D | S | S | S | S | D | S | S |
| 90 | N684 | | D or T | D | D | N | N | N | T | D | N | N | N | D | D | N | N | N |
| 91 | L701 | | I | I | I | I | I | I | I | I | I | I | I | I | I | I | I | I |
| 92 | T706 | | P or Q | P | Q | Q | Q | P | P | Q | Q | Q | P | P | Q | Q | Q | P |
| 93 | T708 | | V | V | V | V | T | T | V | V | V | T | T | V | V | V | V | T |
| 94 | T713 | | K | K | K | T | T | T | K | K | T | T | T | K | K | T | T | T |
| 95 | S720 | | H | H | H | H | S | S | H | H | H | S | S | H | H | H | S | S |
| 96 | T721 | | S or E | S | S | E | T | T | S | S | S | T | T | S | S | S | E | T |
| 97 | S724 | | K | S | S | S | S | S | K | S | S | S | S | S | S | S | S | S |
| 98 | T742 | | H | H | H | H | H | T | H | H | H | H | T | H | H | H | H | T |
| 99 | G743 | | E | E | E | E | E | E | E | E | E | E | E | E | E | E | E | E |
| 100 | V746 | | E | E | E | V | V | V | E | E | V | V | V | E | E | V | V | V |
| 101 | T752 | | M or L | M | T | T | T | T | L | T | T | T | T | M | T | T | T | T |
| 102 | Q753 | | L or R | L | L | Q | Q | Q | R | L | L | Q | Q | R | L | L | Q | Q |
| 103 | K760 | | R | R | K | K | K | K | R | K | K | K | K | R | K | K | K | K |
| 104 | Q778 | | L | L | L | Q | Q | Q | L | L | Q | Q | Q | L | L | Q | Q | Q |
| 105 | P786 | | S | S | S | S | S | S | P | P | P | P | P | P | P | P | P | P |
| 106 | F791 | | A | A | A | A | F | F | A | A | A | F | F | A | A | A | A | F |
| 107 | T801 | | K | K | K | T | T | T | K | K | T | T | T | K | K | T | T | T |
| 108 | K809 | | E | E | E | K | K | K | E | E | K | K | K | E | E | K | K | K |
| 109 | Q810 | | G | G | G | G | G | G | G | G | G | G | G | G | G | G | G | G |
| 110 | Q846 | | A | A | A | A | A | A | A | A | A | A | A | A | A | A | A | A |
| 111 | S849 | | A | A | A | S | S | S | A | A | S | S | S | A | A | S | S | S |
| 112 | S858 | | A | A | A | A | A | A | A | A | A | A | A | A | A | A | A | A |
| 113 | A866 | | S | S | S | A | A | A | S | S | S | A | A | S | S | S | A | A |
| 114 | Q869 | | V | Q | Q | Q | Q | Q | Q | V | Q | Q | Q | Q | Q | Q | Q | Q |
| 115 | S903 | | K | K | A | A | A | A | A | A | A | A | A | A | A | A | A | A |
| 116 | K907 | | A | A | A | A | K | K | A | A | A | K | K | A | A | A | K | K |
| 117 | D910 | | E | E | E | D | D | D | E | E | D | D | D | E | E | D | D | D |
| 118 | S911 | | G | G | G | G | G | G | G | G | G | G | G | G | G | G | G | G |
| 119 | S913 | | D | D | D | D | S | S | D | D | D | S | S | D | D | D | S | S |
| 120 | S914 | | E or A | E | A | S | S | S | E | A | S | S | S | E | A | S | S | S |
| 121 | S917 | | E | E | E | S | S | S | E | E | S | S | S | E | E | S | S | S |
| 122 | Q931 | | E | Q | Q | Q | Q | Q | E | Q | Q | Q | Q | Q | Q | Q | Q | Q |
| 123 | V950 | | S | S | V | V | V | V | S | V | V | V | V | S | V | V | V | V |
| 124 | K960 | P | | P | P | P | P | P | P | P | P | P | P | P | P | P | P | P |
| 125 | V961 | P | | P | P | P | P | P | P | P | P | P | P | P | P | P | P | P |
| 126 | T972 | | N | N | N | N | N | N | N | N | N | N | N | N | N | N | N | N |
| 127 | S977 | | A | A | A | A | A | S | A | A | A | A | S | A | A | A | A | S |
| 128 | Q979 | | N | N | N | N | N | Q | N | N | N | N | Q | N | N | N | N | Q |
| 129 | Y981 | | F | F | F | Y | Y | Y | F | F | Y | Y | Y | F | F | Y | Y | Y |
| 130 | Q985 | | L | L | L | Q | Q | Q | L | L | L | Q | Q | L | L | L | Q | Q |
| 131 | N997 | | E | E | E | N | N | N | E | E | N | N | N | E | E | N | N | N |
| 132 | T1001 | | E | E | E | E | E | E | E | E | E | E | E | E | E | E | E | E |
| 133 | S1004 | | N | N | S | S | S | S | N | S | S | S | S | N | S | S | S | S |
| 134 | D1015 | | N | N | D | D | D | D | N | D | D | D | D | N | D | D | D | D |
| 135 | K1019 | | N | N | N | K | K | K | N | N | K | K | K | N | N | K | K | K |
| 136 | S1029 | | A | A | A | A | S | S | A | A | A | S | S | A | A | A | S | S |
| 137 | A1044 | | T | T | T | T | T | T | T | T | T | T | T | T | T | T | T | T |
| 138 | Q1045 | | S or D or E | S | D | Q | Q | Q | D | D | Q | Q | Q | E | D | Q | Q | Q |
| 139 | E1046 | | H or Y or F | H | Y | H | F | Y | H | Y | Y | Y | H | Y | Y | Y | Y | H |
| 140 | K1047 | | R | R | R | K | K | K | R | R | K | K | K | R | R | K | K | K |
| 141 | D1058 | | N | N | N | N | D | D | N | N | N | D | D | N | N | N | N | D |
| 142 | E1066 | | D | D | E | E | E | E | D | E | E | E | E | D | E | E | E | E |
| 143 | I1088 | | P | P | I | P | P | I | P | P | I | P | I | I | I | P | I | I |
| 144 | N1099 | | D | D | D | D | N | N | D | D | D | N | N | D | D | D | D | N |
| 145 | Q1116 | | K | Q | Q | Q | Q | Q | K | Q | Q | Q | Q | Q | Q | Q | Q | Q |

Design of Symmetric Interfaces with Evolutionary Constraints:

This study was to design mutations of the S antigen from SARS CoV-2 using optimized hydrogen bond networks and evolutionary constraints for the introduction of stabilizing residues.

The lowest energy structures from the previous HBNet design round, derived from structures of the S protein displaying the RBD in the open conformation (PDB Accession Numbers: 6VSB and 6VYB) and targeting mutations on the S or S2 domains, were used for evolutionary design in PROSS against sequences from the non-redundant BLAST database. PSSM matrices were generated for each of the HBNet structures and used for defining the design space during the PROSS protocol.

The starting structures from the HBNet models were designed with the Rosetta FilterScan mover, targeting single point mutations conserved in the evolutionary pool of sequences. The point mutation scan was binned within twelve different energy thresholds (−0.5, −1, −1.5, −2, −2.5, −3, −3.5, −4, −4.5, −5, −5.5, −6 kcal/mol), with each reduction in permitted energy leading to an increase mutation sequence diversity. Combinatorial design was performed on models in these binned energy thresholds, yielding twelve structures for each of the runs.

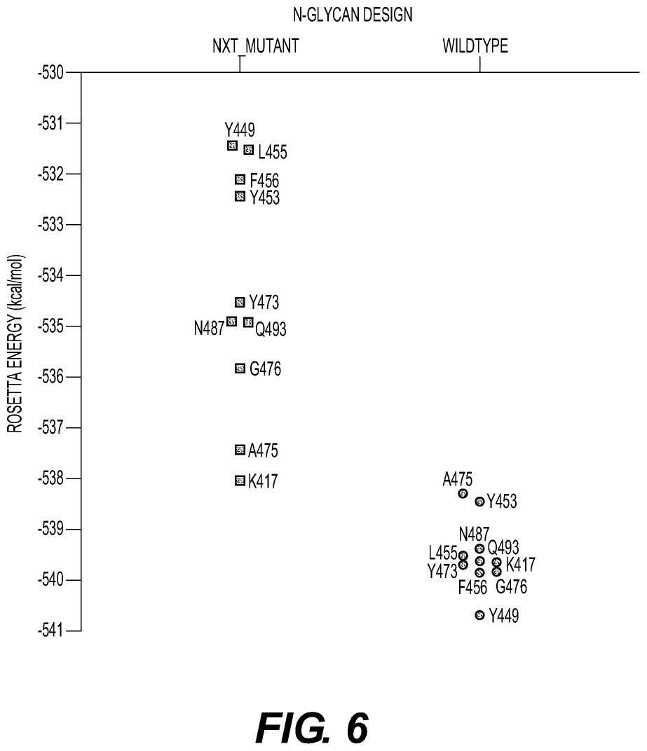

The top five structures (from energy thresholds −5.5 kcal/mol or −6 kcal/mol) were chosen from this combined HBNet-PROSS protocol, either targeting the full S protein or the S2 domain only. The full S HBNet-PROSS design did not yield better energetics than HBNet on its own, indicating the challenge of re-designing an already optimized interface (Cannon et al. 2020 Protein Science 29(4):919-929). The S2 domain targeted HBNet-PROSS mutagenesis yielded models that were more stable, per in silico energetics, than the HBNet designs alone (FIGS. 4A and 4B).

### Results:

Based on the modeled stability using HBNet or PROSS of modified S proteins comprising the mutations in Table 1 or 2, certain mutations were combined and are summarized in Table 3 (“HBNet-PROSS mutations”). Table 3 provides (from left column to right): certain target residues of wild type SARS-CoV-2 amino acid sequence SEQ ID NO: 3; certain target residues of control SARS-CoV-2 amino acid sequence SEQ ID NO: 4; the presently provided point mutations of those target residues which were designed with HBNet and PROSS to increase the (thermo)stability of the wild type (SEQ ID NO: 3) or control (SEQ ID NO: 4) S proteins; and then a summary of what amino acids are present at those target residue positions within the designed, modified S protein fragment sequences SEQ ID NOs: 30-34. The sequence SEQ ID NO: 4 was used as the “parent” sequence for modified S proteins comprising HBNet-PROSS mutations, so all of sequences SEQ ID NO: 30-34 comprise the furin cleavage abrogation mutations, prefusion double proline mutations, and D588G consensus mutation that SEQ ID NO: 4 comprises.

TABLE 3

| | | | Column | | | | | |
|---|---|---|---|---|---|---|---|---|
| | Column | Column | #3 | Column | Column | Column | Column | Column |
| | #1 | #2 | HBNet- | #4 | #5 | #6 | #7 | #8 |
| | SEQ ID | SEQ ID | PROSS | SEQ ID | SEQ ID | SEQ ID | SEQ ID | SEQ ID |
| Row # | NO: 3 | NO: 4 | mutations | NO: 30 | NO: 31 | NO: 32 | NO: 33 | NO: 34 |
| 3 | Q581 | | E | Q | Q | Q | Q | E |
| 4 | D588 | G | | G | G | G | G | G |
| 5 | R656 | G | | G | G | G | G | G |
| 6 | R657 | S | | S | S | S | S | S |
| 7 | R659 | S | | S | S | S | S | S |
| 8 | P689 | | A | A | A | A | A | A |
| 9 | T706 | | S | T | T | T | T | S |
| 10 | D719 | | G | G | G | G | G | G |
| 11 | G743 | | E | E | E | E | E | E |
| 12 | Q778 | | L | Q | L | L | L | Q |
| 13 | F791 | | A | A | A | A | A | A |
| 14 | T801 | | K | K | K | K | K | K |
| 15 | Q810 | | G | G | G | G | G | G |
| 16 | L823 | | S | S | S | S | S | S |
| 17 | V834 | | I | I | I | I | I | I |
| 18 | P836 | | S | S | S | S | S | S |
| 19 | P837 | | S or H | S | H | S | H | S |
| 20 | Q846 | | A | A | A | A | A | A |
| 21 | Y847 | | F | F | F | F | F | F |
| 22 | S858 | | A | A | A | A | A | A |
| 23 | N881 | | A | A | A | A | A | A |
| 24 | S903 | | N or K | N | N | N | N | K |
| 25 | S911 | | G | G | G | G | G | G |
| 26 | R957 | | N or H | N | H | N | N | N |
| 27 | K960 | P | | P | P | P | P | P |
| 28 | V961 | P | | P | P | P | P | P |
| 29 | L986 | | A | A | L | A | A | A |
| 30 | R1013 | | L | L | L | L | L | L |
| 31 | P1043 | | A | A | A | A | A | A |
| 32 | A1044 | | T | T | T | T | T | T |
| 33 | E1046 | | Y | Y | Y | Y | Y | Y |
| 34 | N1093 | | L | L | L | L | L | L |

Designed Disulfide Bonds to Stabilize “closed conformation” SARS-CoV-2 Spike (S) Protein: The cryo-EM structures of SARS-CoV-2 S protein revealed the presence of multiple conformational states corresponding to different organizations of the Receptor Binding Domains (RBDs) (Wrapp et al. 2020 Science 367(6483): 1260-1263 and Walls et al. 2020 Cell 181(2): 281-292.e6). Approximately half of the particles collected presented the trimeric S with a single RBD opened (or in “Up” position), whereas the remaining half was either in closed conformation (all RBD in “down” position) or with two RBD opened (“Up-Up-Down”). This conformational variability of RBDs was also found with SARS-CoV-1 S and MERS-CoV S trimers (Gui et al. 2017 Cell Research 27:119-129; Kirchdoerfer et al., 2018 Sci Rep 8:17823, 11 pgs.; Pallesen et al., 2017 PNAS E7348-E7357 available at WorldWideWeb.pnas.org/cgi/doi/10.1073/pnas.1707304114; Song et al., 2018 PLoS Path 14(8):e1007236, 19 pgs.; Walls et al., 2019 Cell 176:1026-1039; Yuan et al. 2017 Nat. Comm. 8(15092), 9 pgs & Suppl. Materials). SARS-CoV-1 S-RBD and MERS-CoV S-RBD were found to be a major target for neutralizing antibodies (NAbs), with the most potent competing with receptor binding, ACE2 and DPP4, respectively. The majority of SARS-Cov-2 neutralizing antibodies, identified from the sera of convalescent patients, target RBD directly competing with ACE-2 receptor (HypertTextTransferProtocol://opig.stats.ox.ac.uk/webapps/coronavirus/index.html). In particular, two antibodies, CR3022 and S309 isolated from SARS-CoV-1 patients, were able to bind both SARS-CoV-1 S-RBD and SARS-CoV-2 S-RBD (Yuan et al., 2020 Science 368(6491): 630-633; and Pinto et al., 2020 Nature HyperTextTransferProtocolSecure://doi.org/10.1038/s41586-020-2349-y). While CR3022 had poor neutralizing activity for SARS-CoV-2, S309 showed potent neutralization. Yuan et al., 2020 Science 368(6491): 630-633. Structural studies revealed that CR3022 binds to a “cryptic” RBD epitope that is not accessible in the closed conformation, while S309 epitope is always accessible and does not overlap with receptor binding site. Yuan et al., 2020 Science 368(6491): 630-633; Tian et al. 2020 Emerg. Microbes Infect. 9:382-385. Although these are still limited evidences, they suggest that open conformation might present more non-neutralizing epitopes than the closed conformation (or the open conformation may occur less frequently for these antibodies to neutralize as efficiently), something that has been reported also for HIV-1 envelope spike (Cai et al., 2017 PNAS 114(17):4477-4482). In rare cases, pathogen-specific antibodies can promote pathology, resulting in the phenomenon known as Antibody-Dependent-Enhancement (ADE) (discussed herein above), which has been reported for several viruses including dengue virus and also for SARS-CoV-1. For SARS-CoV-1, ADE in animal models is mediated by pre-existing SARS-CoV-1-specific antibodies that may promote viral entry into Fc receptor (FcRs) expressing cells such as monocytes, macrophages and B cells. This mechanism is entirely independent of ACE2 expression. Although infection of macrophages does not seem to result in productive viral replication, internalization of virus-antibody immune complexes can promote inflammation and tissue injury (Yasui et al., 2008 Cytokine 41(3):302-306; Juame et al., 2011 J. Virol. 85:10582-10597; Wang et al., 2014 Circ Res. 114(3):421-433). Recently, two NAbs, S230 and Mersmab1 targeting, respectively, SARS-CoV-1 S-RBD and MERS-CoV S-RBD have been shown to inhibit receptor binding (Wan et al., 2020 J. of Virol 94(7):e00127-20, 9 pgs.; Walls et al., 2019 Cell 176:1026-1039) Interestingly, S230 binding triggered the SARS-CoV S transition to the postfusion conformation, functionally mimicking ACE2 activity, while Mersmab1 mediated MERS-CoV pseudovirus entry into Fc receptor-expressing human cells. These data indicate that ADE of coronaviruses might be promoted by NAbs targeting specific epitopes on RBD involved in receptor binding. Thus, future trials with SARS-CoV-2 S antigen would need to evaluate ADE phenomenon to assess vaccine safety, eventually reconsidering the design of the antigen may be required. RBD can bind to the receptor only in the “Up” position, as well as to NAbs competing with receptor binding, suggesting that SARS-CoV-2 S antigen in closed conformation would not raise such kind of NAbs. In addition, a closed conformation would hide potential non-neutralizing epitopes as discussed above. Overall, SARS-CoV-2 S in closed conformation should have unique immunogenic profile, which has not been characterized yet. However, closed and open conformations are in dynamic equilibrium and forcing either one of these states requires engineering the S protein antigen. The inventors provide that disulfide bonds may be introduced at certain RBD interfaces to stabilize the SARS-CoV-2 S protein or S protein fragments.

Structure of closed SARS-CoV-2 S protein (PDB Accession Number 6VXX; Walls et al. 2020 Cell 181(2): 281-292.e6) was analyzed by PISA (HyperTextTransferProtocolSecure://www.ebi.ac.uk/pdbe/pisa/) to search for RBD residues involved in interfaces interaction. Residues selected by PISA were manually analyzed with PyMol and divided into surface patches. Surface patches were run through MOE (Molecule Operating Environment, WorldWideWeb.chemcomp.com) to find proximal inter- and intra-chain residues that could be substituted by cysteines in order to form stabilizing disulfide bonds. Among the disulfide bonds (DS) created by MOE, six were selected after visual inspection, four inter-chain and two intra-chain respectively.

### Results:

The S protein comprising the control sequence SEQ ID NO: 4 or certain of the above stabilized mutant sequences (SEQ ID NOs: 5, 10, 24, 29, and 30) was selected for further stabilization by adding Disulfide Bridge Mutations to it. See Table 5. Table 4 summarizes which so-called “parent” sequences (SEQ ID NOs: 4, 5, 10, 24, 29, or 30) were used to generate the designed S protein sequences comprising disulfide bridge mutations (i.e., SEQ ID NOs: 35-64). Some of the positions at which a disulfide bridge mutation may be inserted corresponds to the position at which an HBNet or PROSS mutation may be inserted (see above Tables 1-2 and S357D [SEQ ID NOs: 15-16]; Q538L [SEQ ID NOs: 5-9, 15-16]; I824S [SEQ ID NOs: 5-14]; and P836S [SEQ ID NOs: 5-14, 30-34]). Sequences described above that include an HBNet or PROSS mutation at S357, Q538, 1824, or P836 (numbered according to SEQ ID NO: 3) were not used here as a parent sequence for designing S protein sequences comprising a disulfide bridge mutation. The parent sequences used here all comprised the wild type amino acid residue at the cysteine substitution location (i.e., for all of SEQ ID NOs: 35-64, the wild type residue, which is the residue at the corresponding position within SEQ ID NO: 3, was mutated to cysteine (C)).

TABLE 4

| Parent Sequence | | SEQ ID NOs: Generated |
|---|---|---|
| SEQ ID NO: | Nomenclature | with That Parent Sequence |
| 4 | CoV2_S | 35-44 |
| 5 | CoV2_S_1_hbnet | 45, 50, 55, 60 |
| 10 | CoV2_S2_1_hbnet | 46, 51, 56, 61 |
| 24 | CoV2_S2_NTD_6_pross | 47, 52, 57, 62 |
| 29 | CoV2_S2_6_pross | 48, 53, 58, 63 |
| 30 | CoV2_S2_1_hbnet_pross | 49, 54, 59, 64 |

Table 5 provides (from left column to right): certain pairs of disulfide bridge mutations (i.e., (numbered according to wild type SARS-CoV-2 amino acid sequence SEQ ID NO: 3) which were designed to increase the stability of the wild type (SEQ ID NO: 3) or control (SEQ ID NO: 4) S proteins; the nomenclature affiliated with those disulfide bridge mutations (i.e., pairs of cysteine substitution mutations); and then a list of presently provided S protein amino acid sequences that comprise those disulfide bridge mutations.

TABLE 5

| Substitution Mutation Pairs | | SEQ ID NO: Comprising That |
|---|---|---|
| of SEQID NO: 3 | Nomenclature | Mutation Pair |
| 1744 C and A989C | openDS1 | 35, 45-49 |
| D813C and P836C | openDS2 | 36, 50-54 |
| A544C and S941C | openDS3 | 37, 55-59 |
| I824C and D560C | openDS4 | 38, 60-64 |
| G387C and V961C | closedDS1 | 39 |
| S357C and D959C | closedDS2 | 40 |
| V356C and R957C | closedDS3 | 41 |
| K15C and A494C | closedDS4 | 42 |
| A496C and N518C | closedDS5 | 43 |
| P495C and Q538C | closedDS6 | 44 |

Note that the S proteins in closed conformation surprisingly induced higher neutralizing antibodies than did the “2P” S protein in open conformation.

### Example 2: Receptor Binding Mutations

Modified S Proteins Fragments with RBD Knock-Out Mutation

This study was to design knockout mutations that inhibit the binding of the angiotensin-converting enzyme 2 (ACE2) receptor to the SARS CoV-2 S protein Receptor Binding Domain (RBD) using computational biophysics tools.

Starting from RBD structures bound by the ACE2 receptor (PDB Accession Numbers: 6M0J, 6VW1, and 6LZG), a combination of Rosetta, OSPREY, and free energy perturbation (FEP) algorithms were used to design single-point mutations that reduce ACE2 binding (Hallen et al. 2018 Computational Chemistry 39(30):2492-2507 regarding OSPREY; Clark et al. 2019 J M B 431(7):1481-1493 and Steinbrecher et al. 2017 J M B 429(7):948-964 for FEP algorithms). Antigens with reduced receptor binding might reduce the risk of eliciting antibodies that are ACE2-like (i.e. comparable to hACE), which have been shown to trigger conformational changes from pre to post-fusion in other coronaviruses, and might be part of a mechanism related to antibody-dependent enhanced (ADE) disease during the course of natural infection after vaccination.

The point mutations proposed by the interface design round, plus a few manually selected alanine mutations, were introduced into crystal structures of the SARS-2 RBD bound to ACE2 (PDB Accession Numbers: 6M0J, 6VW1, 6LZG) with a RosettaScripts algorithm, point_mutant_scan (Froning et al. 2020 Nat. Comm. 11(2330), HyperTextTransferProtocolSecure://doi.org/10.1038/s41467-020-16231-7, 14 pgs). The script calculates the energetics and dynamics of point mutagenesis, based on repacking and minimizing neighboring residues within a 10 Å sphere centered on the target mutation. The algorithm was updated to include interface energy analysis and the beta scoring function.

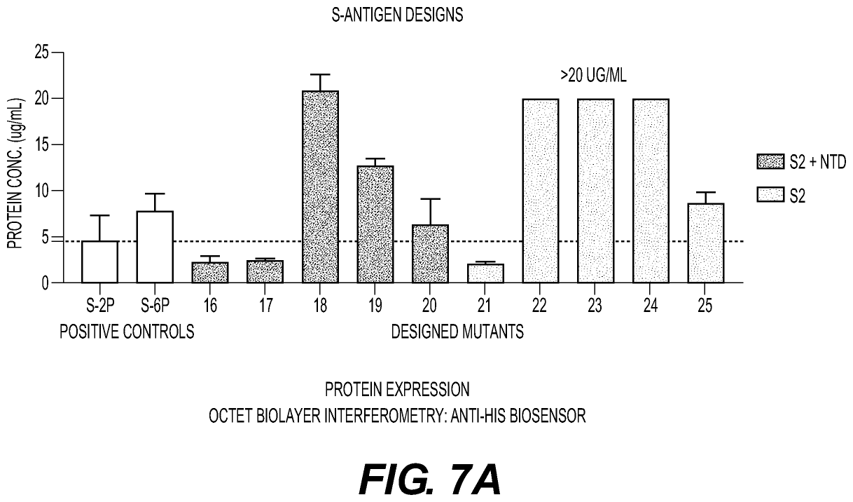

Based on the Rosetta energetics, some of the proposed interface mutations indicate reduced binding energy (more than 2 kcal/mol), relative to ACE2, while maintaining equivalent folding stability to the wildtype structure (in the apo/unbound form, FIG. 5).

### Results:

Certain residues of the wild type SARS-CoV-2 S protein Receptor Binding Domain (RBD) (P330-P531) were targeted for the insertion of substitution mutations designed to knock-out (prevent) binding to the S protein by an antibody comparable to ACE2. In Table 6 are provided (from left column to right): certain target residues of wild type SARS-CoV-2 amino acid sequence SEQ ID NO: 3; the inventor-designed substitution mutations of those target residues (called “RBD Knock-Out Mutations”) to knock-out (prevent) binding to the S protein by an antibody comparable to hACE2; and then a summary of the SEQ ID NO: for an exemplary betacoronavirus S protein amino acid sequence comprising that RBD knock-out mutation. The sequence SEQ ID NO: 4 was used as the “parent” sequence for the modified S protein sequences SEQ ID NOs: 65-104 (i.e., they also comprise the double proline prefusion mutations, furin abrogation mutations, and D588G consensus mutation present within the sequence SEQ ID NO: 4).

TABLE 6

| Column #1 | Column #1 | Column #1 |
|---|---|---|
| Target Residue in | RBD Knock- | SEQ ID NO: |
| SEQ ID NO: 3 | Out Mutations | Comprising Mutation |
| K391 | F | 65 |
| K391 | L | 66 |
| K391 | M | 67 |
| K391 | W | 68 |
| K391 | Y | 69 |
| Y423 | A | 70 |
| Y427 | A | 71 |
| L429 | A | 72 |
| L429 | H | 73 |
| L429 | M | 74 |
| L429 | N | 75 |
| L429 | W | 76 |
| F430 | H | 77 |
| F430 | I | 78 |
| F430 | W | 79 |
| F430 | Y | 80 |
| Y447 | W | 81 |
| A449 | M | 82 |
| G450 | T | 83 |
| F460 | H | 84 |
| F460 | I | 85 |
| F460 | L | 86 |
| F460 | M | 87 |
| F460 | N | 88 |
| F460 | P | 89 |
| F460 | T | 90 |
| F460 | W | 91 |
| F460 | Y | 92 |
| N461 | F | 93 |
| N461 | L | 94 |
| N461 | M | 95 |
| N461 | Q | 96 |
| Q467 | A | 97 |
| Q467 | Y | 98 |
| Q467 | F | 99 |
| Q467 | R | 100 |
| Q467 | M | 101 |
| Q467 | C | 102 |
| Q467 | G | 103 |
| Q467 | V | 104 |

### Introduction of Glycan Motifs to Mask ACE2/SARS CoV-2 S Protein RBD Binding Site:

This study was to design glycan based NxT mutations that mask the binding site of the human angiotensin-converting enzyme 2 (ACE2) receptor on the SARS CoV-2 receptor binding domain (RBD) using computational biophysics tools.

Interface residues between ACE2 and RBD were identified from Lan et al. (2020 Nature HyperTextTransferProtocolSecure://doi.org/10.1038/s41586-020-2180-5, 16 pgs). Rosetta comparative modeling was performed on x-ray structures of the RBD (PDB Accession Numbers: 6M0J, 6VW1, 6LZG), without the ACE2 receptor, to get a starting model to test folding stability. The lowest energy model from PDB Accession Number 6VW1 was chosen based on overall Rosetta statistics. The point_mutant_scan RosettaScripts algorithm was used to introduce mutations that would place an NxT motif at the following 10 interface sites (K417, Y449, Y453, L455, F456, Y473, A475, G476, N487, and Q493, numbered according to SEQ ID NO: 2—for clarity, these residues are where the NxT motif starts and are not necessarily the mutation locations).

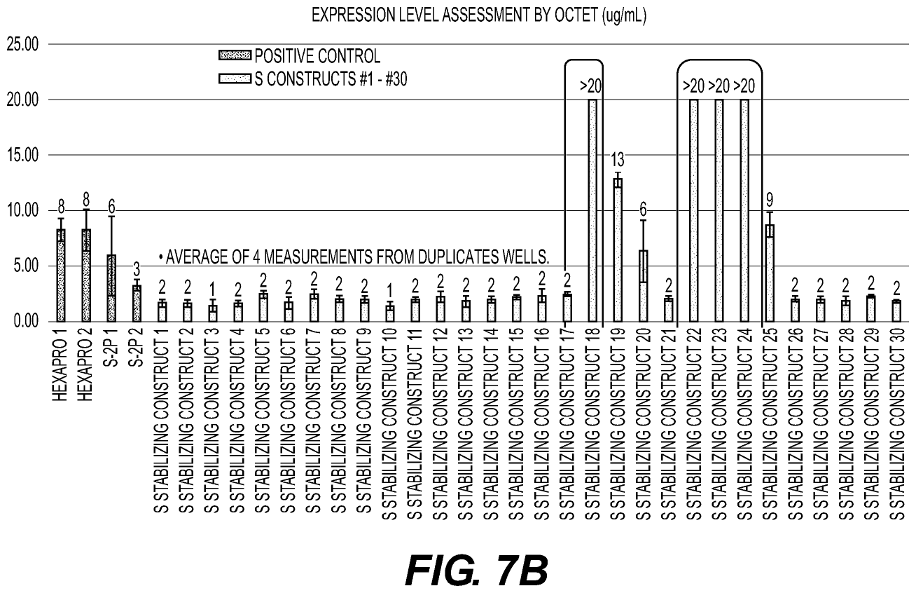

Based on Rosetta folding energetics, the introduction of the 10 NxT motifs yielded different energy clusters relative to the wildtype: equivalent stability (K417, A475), slightly destabilizing (Y473, G476, N487, Q493), and more destabilizing (Y449, Y453, L455, F456) (FIG. 6).

### Results:

Certain residues were targeted in pairs but, in certain instances, it was only necessary to substitute one residue for introduction of the N—X-T motif (see SEQ ID NOs: 112 and 113). Table 7 provides (from left column to right): a first target residue “(A)” of wild type SARS-CoV-2 amino acid sequence SEQ ID NO: 3; the designed substitution mutation of that target residue (called “RBD Glycan Mutations”); as needed, a second target residue “(B)” of wild type SARS-CoV-2 amino acid sequence SEQ ID NO: 3; the inventor-designed RBD glycan mutation of that target residue; and then a summary of the SEQ ID NO: for a presently provided exemplary betacoronavirus S protein amino acid sequence that comprises that pair of RBD Glycan Mutations. The sequence SEQ ID NO: 4 was used as the “parent” sequence for the modified S protein sequences SEQ ID NOs: 105-114 (i.e., SEQ ID NOs: 105-114 also comprise the double proline prefusion mutations, furin abrogation mutations, and D588G consensus mutation present within the sequence SEQ ID NO: 4).

TABLE 7

| | | | | SEQ ID NO: |
|---|---|---|---|---|
| Target Residue | | Target Residue | | Comprising Those |
| (A) in SEQ ID | RBD Glycan | (B) in SEQ ID | RBD Glycan | Mutations of (A) |
| NO: 3 | Mutation of (A) | NO: 3 | Mutation of (B) | or (A) and (B) |
| K391 | N | A393 | T | 105 |
| Y423 | N | Y425 | T | 106 |
| Y427 | N | L429 | T | 107 |
| L429 | N | R431 | T | 108 |
| F430 | N | K432 | T | 109 |
| Y447 | N | A449 | T | 110 |
| A449 | N | S451 | T | 111 |
| G450 | N | | | 112 |
| Y463 | T | | | 113 |
| Q467 | N | Y469 | T | 114 |

The mutations of Examples 1 and 2 were thoughtfully designed to conserve putative S protein epitopes and tertiary/three-dimensional structure generally so that resultant mutant S proteins remain immunogenic (regarding SARS-CoV-2 epitopes, see Grifoni et al. 2020 Cell 181:1-13 and Supplementary Materials; Kiyotani et al. 2020 J. Hum. Genet. HyperTextTransferProtocolSecure://doi.org/10.1038/s10038-020-0771-5).

Without wishing to be bound by theory, it is believed that the SARS-CoV-2 Spike (S) protein modifications described here at Examples 1 and 2, when applied to corresponding positions within other betacoronavirus S proteins (such as a MERS-CoV or SARS-CoV-1 S protein), will have a comparable effect.

### Example 3: Assays to Confirm Antibody Binding and Enhanced Stability

The above-summarized, designed S proteins or S protein fragments can be cloned by recombinant DNA methods (in different combinations), then expressed, purified, and characterized for (i) antibody binding using surface plasmon resonance (SPR) and bio-layer interferometry (BLI) and (ii) thermostability, using differential scanning calorimetry (DSC) or differential scanning fluorimetry (DSF) assays.

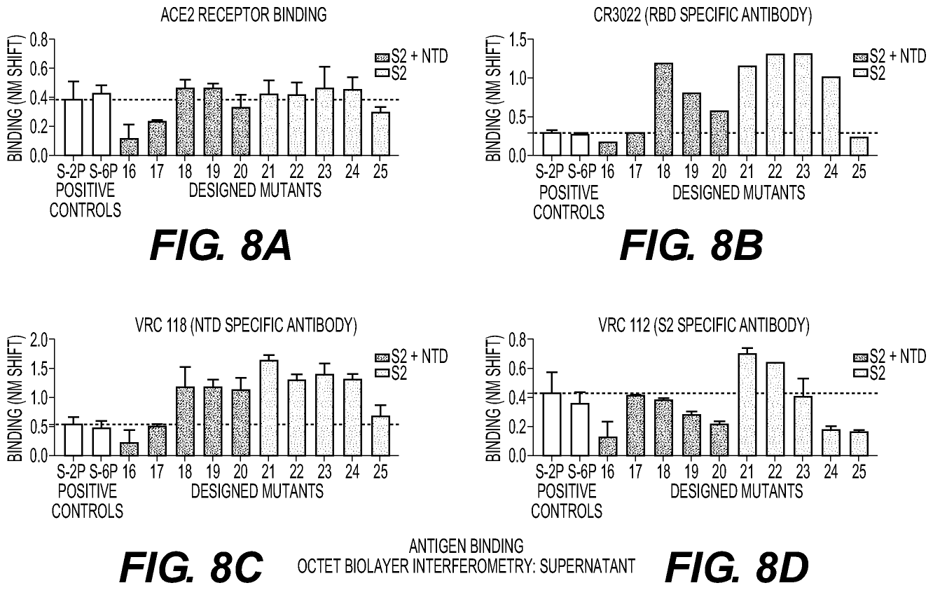

Table 8 lists 30 designed S protein or protein fragments (S Stabilizing Constructs) that were used in in vitro assays to determine levels of cellular expression, antigenicity, and thermostability (FIGS. 7A-9C). On Table 8, each S Stabilizing Construct is listed along with its In silico identifier and SEQ ID NO. The computational designs were based on a SARS-1 structure (PDB: 6NB7), where all RBDs were in the open conformation. Experimental binding to ACE2 shows that there is at least 1 RBD that is in the open conformation. Cyro-EM structure to confirm this is currently not available.

TABLE 8

| S Stabilizing | | |
|---|---|---|
| Construct # | In silico identifier | SEQ ID NO: |
| 1 | COV2_S_1_hbnet | SEQ ID NO: 5-(CoV2_S_1_hbnet) mutant Spike |
| | | (S) protein amino acid sequence |
| 2 | COV2_S_2_hbnet | SEQ ID NO: 6-(CoV2_S_2_hbnet) mutant Spike |
| | | (S) protein amino acid sequence |
| 3 | COV2_S_3_hbnet | SEQ ID NO: 7-(CoV2_S_3_hbnet) mutant Spike |
| | | (S) protein amino acid sequence |
| 4 | COV2_S_4_hbnet | SEQ ID NO: 8-(CoV2_S_4_hbnet) mutant Spike |
| | | (S) protein amino acid sequence |
| 5 | COV2_S_5_hbnet | SEQ ID NO: 9-(CoV2_S_5_hbnet) mutant Spike |
| | | (S) protein amino acid sequence |
| 6 | COV2_S2_1_hbnet | SEQ ID NO: 10-(CoV2_S2_1_hbnet) mutant |
| | | Spike (S) protein amino acid sequence |
| 7 | COV2_S2_2_hbnet | SEQ ID NO: 11-(CoV2_S2_2_hbnet) mutant |
| | | Spike (S) protein amino acid sequence |
| 8 | COV2_S2_3_hbnet | SEQ ID NO: 12-(CoV2_S2_3_hbnet) mutant |
| | | Spike (S) protein amino acid sequence |
| 9 | COV2_S2_4_hbnet | SEQ ID NO: 13-(CoV2_S2_4_hbnet) mutant |
| | | Spike (S) protein amino acid sequence |
| 10 | COV2_S2_5_hbnet | SEQ ID NO: 14-(CoV2_S2_5_hbnet) mutant |
| | | Spike (S) protein amino acid sequence |
| 11 | COV2_S_1_pross | SEQ ID NO: 15-(CoV2_S_1_pross) mutant Spike |
| | | (S) protein amino acid sequence |
| 12 | COV2_S_2_pross | SEQ ID NO: 16-(CoV2_S_2_pross) mutant Spike |
| | | (S) protein amino acid sequence |
| 13 | COV2_S_3_5_pross | SEQ ID NO: 17-(CoV2_S_3_5_pross) mutant |
| | | Spike (S) protein amino acid sequence |
| 14 | COV2_S_5_pross | SEQ ID NO: 18-(CoV2_S_5_pross) mutant Spike |
| | | (S) protein amino acid sequence |
| 15 | COV2_S_6_pross | SEQ ID NO: 19-(CoV2_S_6_pross) mutant Spike |
| | | (S) protein amino acid sequence |
| 16 | COV2 _S2 _NTD_0_5_pross | SEQ ID NO: 20-(CoV2_S2_NTD_0_5_pross) |
| | | mutant Spike (S) protein amino acid sequence |
| 17 | COV2 _S2 _NTD_2_pross | SEQ ID NO: 21-(CoV2_S2_NTD_2_pross) |
| | | mutant Spike (S) protein amino acid sequence |
| 18 | COV2 _S2 _NTD_3_pross | SEQ ID NO: 22-(CoV2_S2_NTD_3_pross) |
| | | mutant Spike (S) protein amino acid sequence |
| 19 | COV2 _S2 _NTD_5_pross | SEQ ID NO: 23-(CoV2_S2_NTD_5_pross) |
| | | mutant Spike (S) protein amino acid sequence |
| 20 | COV2 _S2 _NTD_6_pross | SEQ ID NO: 24-(CoV2_S2_NTD_6_pross) |
| | | mutant Spike (S) protein amino acid sequence |
| 21 | COV2_S2_1_pross | SEQ ID NO: 25-(CoV2_S2_1_pross) mutant |
| | | Spike (S) protein amino acid sequence |
| 22 | COV2_S2_2_pross | SEQ ID NO: 26-(CoV2_S2_2_pross) mutant |
| | | Spike (S) protein amino acid sequence |
| 23 | COV2_S2_3_pross | SEQ ID NO: 27-(CoV2_S2_3_pross) mutant |
| | | Spike (S) protein amino acid sequence |
| 24 | COV2_S2_4_pross | SEQ ID NO: 28-(CoV2_S2_4_pross) mutant |
| | | Spike (S) protein amino acid sequence |
| 25 | COV2_S2_6_pross | SEQ ID NO: 29-(CoV2_S2_6_pross) mutant |
| | | Spike (S) protein amino acid sequence |
| 26 | COV2_S2_1_hbnet_pross | SEQ ID NO: 30-(CoV2_S2_1_hbnet_pross) |
| | | mutant Spike (S) protein amino acid sequence |
| 27 | COV2_S2_2_hbnet_pross | SEQ ID NO: 31-(CoV2_S2_2_hbnet_pross) |
| | | mutant Spike (S) protein amino acid sequence |
| 28 | COV2_S2_3_hbnet_pross | SEQ ID NO: 32-(CoV2_S2_3_hbnet_pross) |
| | | mutant Spike (S) protein amino acid sequence |
| 29 | COV2_S2_4_hbnet_pross | SEQ ID NO: 33-(CoV2_S2_4_hbnet_pross) |
| | | mutant Spike (S) protein amino acid sequence |
| 30 | COV2_S2_5_hbnet_pross | SEQ ID NO: 34-(CoV2_S2_5_hbnet_pross) |
| | | mutant Spike (S) protein amino acid sequence |

### Results

### Expression and Purification of Designed S Protein or S Protein Fragments:

The designed S protein fragments were produced in a high-throughput (HT) expression system (FIGS. 7A and 7B). For quantification of protein expression level, anti-His tag biosensors were dipped into harvest media in each transfection well. The initial binding slope of the mutant constructs to biosensor surface through his tag were measured and converted into concentration by using a standard curve.

The mutant constructs were assayed along with controls S-2P and/or HexaPro. The control S-2P corresponds to amino acid residues 1-1121 of SEQ ID NO:4, but with a D588 (Wrapp et al. 2020 Science 367(6483):1260-1263). The control polypeptide HexaPro (S-6P) corresponds to amino acid residues 1-1121 of SEQ ID NO:4, but with a D588 and proline substitutions (F817P, A892P, A899P, A942P) in addition to the two prolines as in S-2P construct (Hsieh et al. 2020 Science 369(6510): 1501-1505). S-2P (FIG. 1D) consists of two proline substitutions which stabilize the prefusion conformation. HexaPro (S-6P) contains four beneficial proline substitutions (F817P, A892P, A899P, A942P) in addition to the two proline existed in S-2P construct (Hsieh et al. 2020 Science 369(6510): 1501-1505; FIG. 1E). The proline substitutions stabilize the prefusion conformation and further shows higher levels of expression in comparison to S-2P (Hseih et al., 2020 Science 369 (6510: 1501-1505). HexaPro can also withstand heating and freezing (Hseih et al., 2020 Science 369 (6510: 1501-1505).

The Octet quantification assays (FIGS. 7A and 7B) were performed on Octet 96 Red system. Eight anti-HIS biosensors were presoaked in blank spent media for 10 minutes prior to the measurements. 200 μL standard samples were prepared in a black 96-well plate with S-2P or HexaPro standards diluted in media from 20 μg/mL to 0.3125 μg/mL. Standards and mutants binding curve on anti-HIS biosensor were measured. Initial binding rate of standards were plotted against the standards' known concentration to generate a standard calibration curve. This calibration curve is used to calculate the concentration of each mutant in media by fitting its measured initial binding rate to the calibration curve. The expression levels were measured in duplicate wells of each mutant's media and the average readout was reported.

### Results:

Among 30 of the designed mutants tested, #18 (SEQ ID NO: 22), 19 (SEQ ID NO: 23), 20 (SEQ ID NO: 24), 22 (SEQ ID NO: 26), 23 (SEQ ID NO: 27), 24 (SEQ ID NO: 28), and 25 (SEQ ID NO: 29) showed expression levels that were greater than the S-2P control polypeptide (FIG. 7A). Designed mutant #18 (SEQ ID NO: 22), 22 (SEQ ID NO: 26), 23 (SEQ ID NO: 27), and 24 (SEQ ID NO: 28) showed expression levels that were higher than 20 ug/ml, which was a seven-fold higher expression level when compared to S-2P (FIGS. 7A and 7B) and an over three-fold higher expression level when compared to HexaPro (FIG. 7B). Considering their high expression levels, these constructs were ideal constructs for further screening (antigenicity and thermostability) and scaling-up production. #19 (SEQ ID NO: 23), #25 (SEQ ID NO: 29) also show higher or equivalent expression level compared with hexaPro (FIG. 7B).

### Antibody Binding to Designed S Protein or S Protein Fragments:

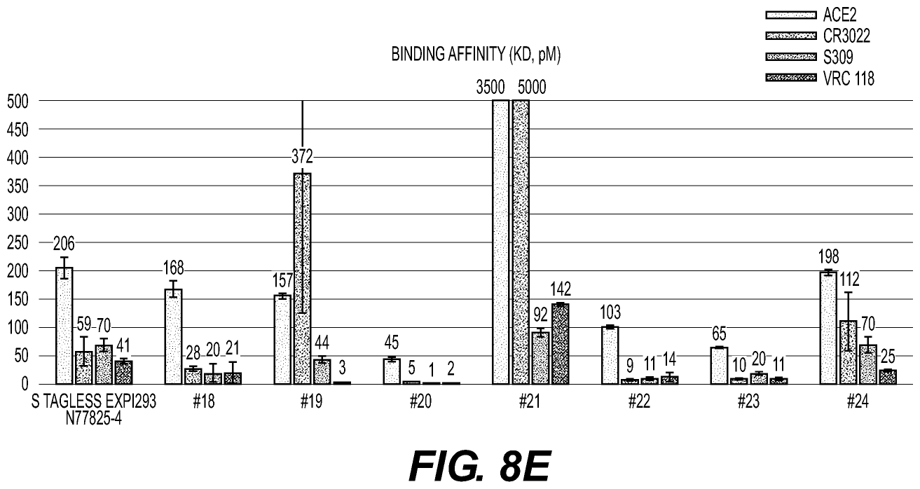

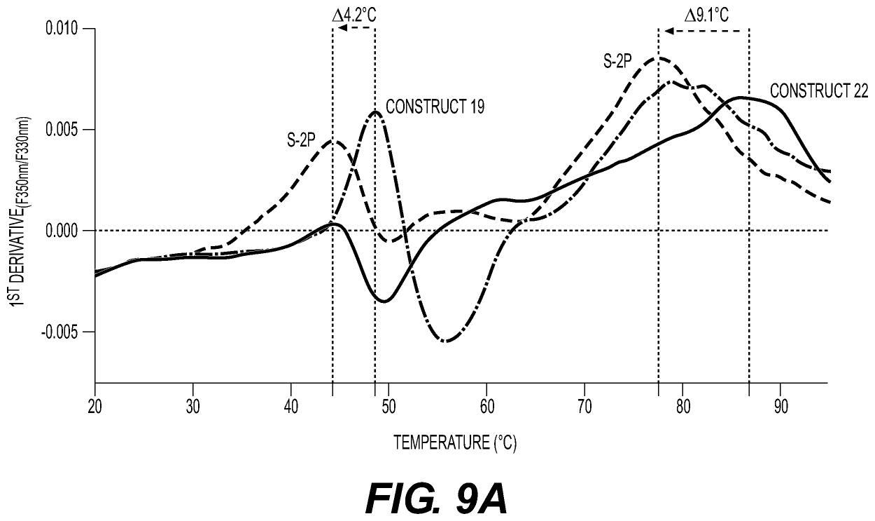

The antigenicity of the designed S protein fragments were tested using a high-throughput binding screen in supernatant (Octet Bio-Layer Interferometry, BLI). The ACE 2 Receptor, CR3022 antibody (RBD Specific Antibody) was originally obtained from a person who, nearly two decades ago, survived a bout of severe acute respiratory syndrome (SARS). The SARS virus is closely related to the novel coronavirus that causes COVID-19. VRC 118 (NTD Specific Antibody), VRC 112 (S2 Specific Antibody), and S309 (Neutralizing Antibody that recognizes a proteoglycan epitope on the receptor-binding domain of SARS-Cov-2; the antibody is composed of 6 complementarity-determining regions (CDR) loops which come in contact with amino acids 337-344, 356-361, and 440-444 in the spike protein.) were used to test the conformational and antigenic integrity of the designs (FIGS. 8A-8E). VRC 112 and VRC 118 were obtained under an agreement with the National Institute of Allergy and Infectious Diseases (NIAID).

The Epitope Integrity Screening assays (FIGS. 8A-8D) were performed on Octet 384 system. SARS-CoV2 mAbs (CR3022, VRC-112 and VRC-118) and ACE2 receptor were loaded on 16 anti-human Fc biosensor at 10 μg/mL. mAb or ACE2-receptor coated biosensors were dipped into each mutant's raw harvest media, and the binding level against each mAb/ACE2 receptor were measured. A non-relevant RSV antigen spike-in media was used as negative control. A blank Expi293 media was used as blank subtraction. Binding levels were measured in duplicate well for each of the mutants' media and the average readout was reported.

The SPR experiment (FIG. 8E) was performed in a running buffer composed of 0.01 M HEPES pH 7.4, 0.15 M NaCl, 3 mM EDTA, 0.005% v/v Surfactant P20 at 25° C. using Biacore 8K (GE Healthcare) Series S protein A sensor chip (GE Healthcare) was used. Briefly, the SARS-COVID S specific antibodies or ACE2 receptor were immobilized to protein A sensor chip (GE Healthcare) at the ligand capture level, around 100RU. Serial dilutions of purified SARS-COVID S protein mutants were injected ranging in concentration from 10 nM to 1.25 nM. The resulting data were fit to a 1:1 binding model using Biacore Evaluation Software (GE Healthcare).

### Results:

The epitopes of constructs #18 (SEQ ID NO: 22), 19 (SEQ ID NO: 23), 20 (SEQ ID NO: 24), 22 (SEQ ID NO: 26), 23 (SEQ ID NO: 27), and 24 (SEQ ID NO: 28) were recognized by CR3022, S309, VRC-118, and their binding sites to ACE2 are not affected (FIG. 8E). #21 (SEQ ID NO: 25) shows a 17-fold affinity decrease to CR3022 and a 100-fold decrease to ACE2 receptor (FIG. 8E). The epitope recognized by VRC-112 was disrupted for all selected candidates (not shown) when measured on a supernatant sample by using the Biacore 8K as described above. When measured by SPR on purified proteins (and also using instrumentation/protocol that is more sensitive), better binding was achieved (data not shown)).

### Thermostability:

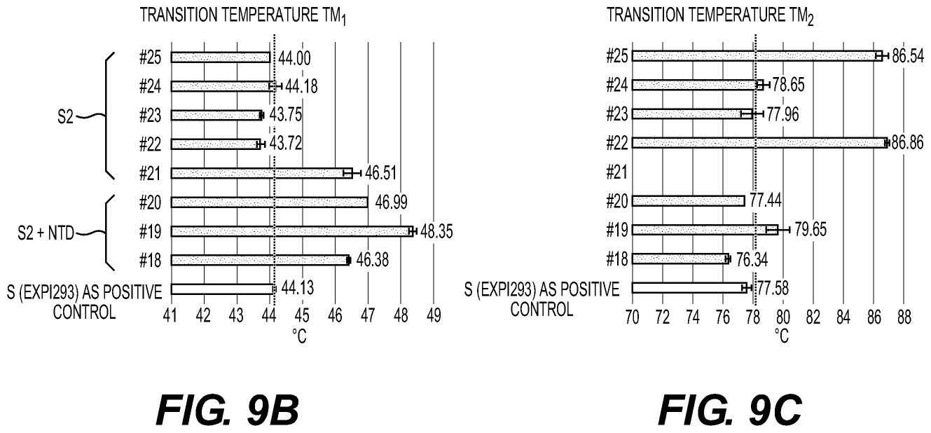

Nano Differential Scanning Fluorimetry (NanoDSF; FIGS. 9A-9C) was used to assess the thermal stability of purified SARS-COVID S protein mutants. Samples were diluted to 0.2 mg/mL by PBS and 20 μL of each sample was loaded into capillary tubes. Temperature ramp was set to 1° C./minute increase from 20° C. to 95° C. The reported values are the mean of 2^{nd} derivative of Ratio 350/330 from 3 independent measurements.

### Results:

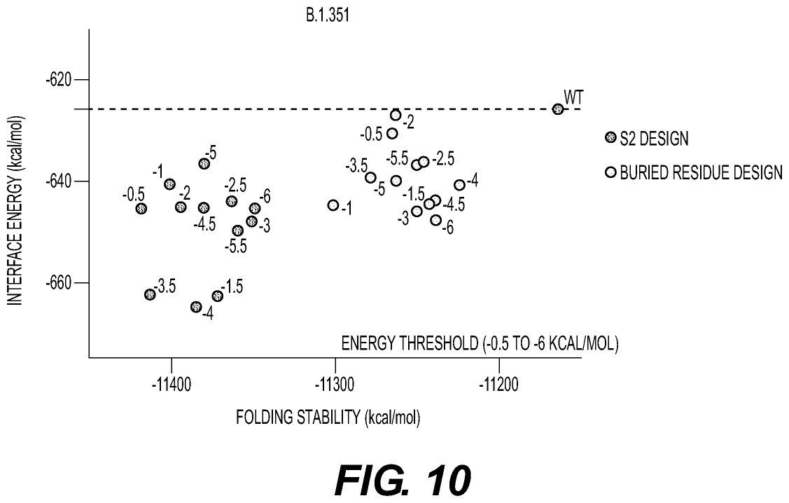

Of the constructs selected for screening, #19 show highest increase in transition temperature 1 (Tₘ1), of 4.2° C., #22 show highest increase in transition temperature 2 (Tₘ2), of 9.1° C. (FIG. 10A-10C). S Stabilizing Construct #18 (SEQ ID NO: 22), 19 (SEQ ID NO: 23), 20 (SEQ ID NO: 24), and 21 (SEQ ID NO: 25) had Tₘ1's greater than the S control (FIG. 10B). S Stabilizing Construct #19 (SEQ ID NO: 23), 22 (SEQ ID NO: 26), 24 (SEQ ID NO: 28), and 25 (SEQ ID NO: 29) had Tₘ2's greater than the S control (FIG. 10C).

### Quaternary Structure of the Designed S Protein or S Protein Fragments:

High-performance liquid chromatography Size Exclusion Chromatography (HPLC SEC) was used to estimate the molecule size of purified SARS-COVID S mutants. 10 μL of purified SARS-COVID S mutants samples were injected into a Superdex 200 INCREASE 3.2/300 column and evaluated using an Alliance HPLC system at a flow rate of 0.1 ml/min. UV214 readings were obtained with a Photodiode Array Detector.

Dynamic Light Scattering (DLS) measurements were performed at 25° C. using a DynaPro Plate Reader II (Wyatt Technology). The samples were diluted in PBS, adjusted to 0.1 mg/ml, and filtered by 0.2 um membrane prior to analysis. The assay was performed in triplicate. DYNAMICS version 7 software from Wyatt Technology was used to analyze the data. The reported values are the mean value of 3 independent measurements.

### Results:

HPLC-SEC: #21 (SEQ ID NO: 25) peak shifts to a longer retention time compared with wild type S-2P positive control sample, indicating a lower molecular weight, which could be a S protein monomer. Other constructs, including #18 (SEQ ID NO: 22), 19 (SEQ ID NO: 23), 22 (SEQ ID NO: 26), 23 (SEQ ID NO: 27), and 24 (SEQ ID NO: 28) could be either S trimer, or mixture of trimer and higher degree oligomers.

DLS: #19 (SEQ ID NO: 23) and 23 (SEQ ID NO: 27) could be dimer of S trimer, while #21 (SEQ ID NO: 25) could be S monomer. #18 (SEQ ID NO: 22), 22 (SEQ ID NO: 26), and 24 (SEQ ID NO: 28) could be S trimer.

### Example 4—Additional Sequences

RNA sequences that encode polypeptides having the sequences reported in SEQ ID Nos: 125-134 were prepared with the goal of making sequences that have high expression and also retain antigenicity.

### Design of CoV-2 B.1.351 Lineage Spike Proteins:

The goal of this study is to perform stabilizing antigen design of spike proteins from coronavirus CoV-2 variant B.1.351 using evolutionary constraints and structural biophysics (PROSS). Symmetric minimization was performed on the closed conformation of the 2.7 Å CoV-2 spike glycoprotein (PDB: 7DF3), using cryo-EM density constraints and Rosetta Comparative Modeling (RosettaCM). The CoV-2 (Wuhan) sequence was mutated to the B.1.351 strain (20H/501Y.V2, a South African strain, Madhi et al. 2021 N Engl J Med 384: 1885-1898) with the D215G, K417N, E484K, N501Y D614G mutations. Mutagenesis with PROSS was focused on the S2 domain design with exposed or buried residues (less than 25% surface exposure) (FIG. 10),

### Results:

Ten constructs (SEQ ID NOs: 125-134) were generated from the PROSS protocol, focusing on full length B.1.351 spike glycoproteins, yielding five S2 designs (energy threshold: −0.5 kcal/mol, −1.5 kcal/mol, −3.5 kcal/mol, −4 kcal/mol, and −5.5 kcal/mol) and five buried S2 domain constructs (energy threshold: −1 kcal/mol, −1.5 kcal/mol, −3 kcal/mol, −5 kcal/mol, and −6 kcal/mol). These designs will be used as a further proof of principle for the S2 domain targeted PROSS method.

Determination of the Preclinical Immunogenicity of Six SARS-CoV2 Stabilized S Protein Designs Adjuvanted with AS03 in BALB/c Mice

### Mouse Immunizations

This in vivo study was performed to assess the preclinical immunogenicity of six new SARS-CoV2 stabilized S protein designs (designated as 18, 19, 21, 22, 23, and 24 in this study). Female BALB/c mice, 7-8 weeks of age at the start of the study, were immunized (N=10 mice/group) with AS03 adjuvanted-stabilized S proteins at two dosage levels of 3 μg and 0.3 μg. Control groups were also included in the study and consisted of saline placebo and AS03 adjuvanted-SARS-CoV2 S_2P protein administered at the same two dosage levels. Mice were injected intramuscularly twice in a 3 week period and bled 3 weeks after the initial immunization (post-I) and 2 weeks after the second immunization (post-II). The serum CoV2-specific antibody response was assessed using a pseudovirus neutralization assay to measure functional antibodies and an ELISA (pre-fusion S_2P protein absorbed to the solid phase) to measure IgG binding antibodies.

### Antibody Responses

All six stabilized S protein designs were immunogenic and induced robust serum neutralizing antibody and IgG binding antibody responses in mice (Tables 9-12). All SARS-CoV2 S immunized animals showed a dose response trend in neutralizing antibody titers following the second immunization (Tables 9 and 10). Interestingly, Design 19 elicited neutralizing antibody responses (GMT=153) post-I at the 3 μg dosage, as did Design 24 albeit to a lesser extent (GMT=37). For both Design 19 and Design 24, there was a dramatic boosting effect following the second immunization and the neutralizing antibody responses increased about 55-fold and 300-fold, respectively. The four other designs did not elicit detectable neutralizing antibody responses post-I at the 3 μg dosage which is consistent with the S_2P protein. None of the six stabilized S protein designs or the S_2P protein elicited neutralizing antibody responses post-I at the 0.3 μg dosage (Tables 9 and 10). All SARS-CoV2 immunized animals elicited strong IgG binding antibody responses after the initial immunization at both the 3 μg and 0.3 μg dosages, and this data also shows a dose response trend in IgG binding antibodies, although more subtle than the dose response trend seen with neutralizing antibodies (Tables 11 and 12). In addition, a strong boosting effect was seen in IgG binding antibodies following the second immunization.

TABLE 9
SARS-CoV2 PNA Titers 3 μg Dosage

| | | Geo- | | | Geo- | | |
|---|---|---|---|---|---|---|---|
| | | metric | | | metric | | |
| SEQ | | Mean | | | Mean | | |
| ID | | Titers | Lower | Upper | Titers | Lower | Upper |
| NO: | Design | Post-I | 95% Cl | 95% Cl | Post-II | 95% Cl | 95% Cl |
| | Saline | 13 | 13 | 13 | 13 | 13 | 13 |
| | CoV2 S 2P | 17 | 12 | 26 | 11000 | 6922 | 17481 |
| 22 | Design 18 | 28 | 16 | 48 | 6421 | 3602 | 11447 |
| 23 | Design 19 | 153 | 76 | 310 | 8488 | 5284 | 13635 |
| 25 | Design 21 | 18 | 13 | 26 | 3240 | 1555 | 6753 |
| 26 | Design 22 | 14 | 11 | 16 | 2212 | 1316 | 3718 |
| 27 | Design 23 | 27 | 18 | 41 | 4872 | 2632 | 9018 |
| 28 | Design 24 | 37 | 18 | 76 | 10802 | 6484 | 17995 |

TABLE 10
SARS-CoV2 PNA Titers 0.3 μg Dosage

| | | Geo- | | | Geo- | | |
|---|---|---|---|---|---|---|---|
| | | metric | | | metric | | |
| SEQ | | Mean | | | Mean | | |
| ID | | Titers | Lower | Upper | Titers | Lower | Upper |
| NO: | Design | Post-I | 95% Cl | 95% Cl | Post-II | 95% Cl | 95% Cl |
| | Saline | 13 | 13 | 13 | 13 | 13 | 13 |
| | CoV2 S 2P | 13 | 13 | 13 | 1105 | 602 | 2028 |
| 22 | Design 18 | 14 | 11 | 17 | 1865 | 1052 | 3307 |
| 23 | Design 19 | 18 | 11 | 28 | 4958 | 2537 | 9689 |
| 25 | Design 21 | 14 | 11 | 16 | 395 | 72 | 2173 |
| 26 | Design 22 | 13 | 13 | 13 | 425 | 218 | 830 |
| 27 | Design 23 | 19 | 11 | 33 | 1733 | 1047 | 2867 |
| 28 | Design 24 | 19 | 11 | 34 | 10057 | 5734 | 17637 |

TABLE 11
SARS-CoV2 S IgG Titers 3 μg Dosage

| | | Geo- | | | Geo- | | |
|---|---|---|---|---|---|---|---|
| | | metric | | | metric | | |
| SEQ | | Mean | | | Mean | | |
| ID | | Titers | Lower | Upper | Titers | Lower | Upper |
| NO: | Design | Post-I | 95% Cl | 95% Cl | Post-II | 95% Cl | 95% Cl |
| | Saline | 31 | 31 | 31 | 31 | 31 | 31 |
| | CoV2 S 2P | 9430 | 6816 | 13045 | 678441 | 530373 | 867846 |
| 22 | Design 18 | 12850 | 10991 | 15023 | 628363 | 536401 | 736092 |
| 23 | Design 19 | 22115 | 17367 | 28161 | 665249 | 557544 | 793759 |
| 25 | Design 21 | 3453 | 2589 | 4605 | 438477 | 339476 | 566348 |
| 26 | Design 22 | 9091 | 6511 | 12692 | 470081 | 357568 | 617997 |
| 27 | Design 23 | 17045 | 13467 | 21575 | 725806 | 503802 | 1045637 |
| 28 | Design 24 | 11763 | 8077 | 17132 | 889688 | 698385 | 1133393 |

TABLE 12
SARS-CoV2 S IgG Titers 0.3 μg Dosage

| | | Geo- | | | Geo- | | |
|---|---|---|---|---|---|---|---|
| | | metric | | | metric | | |
| SEQ | | Mean | | | Mean | | |
| ID | | Titers | Lower | Upper | Titers | Lower | Upper |
| NO: | Design | Post-I | 95% Cl | 95% Cl | Post-II | 95% Cl | 95% Cl |
| | Saline | 31 | 31 | 31 | 31 | 31 | 31 |
| | CoV2 S 2P | 1783 | 1377 | 2309 | 517622 | 420205 | 637624 |
| 22 | Design 18 | 3665 | 2892 | 4646 | 445005 | 368479 | 537425 |
| 23 | Design 19 | 5823 | 4256 | 7968 | 518079 | 459324 | 584350 |
| 25 | Design 21 | 325 | 147 | 720 | 113139 | 68734 | 186232 |
| 26 | Design 22 | 1464 | 1047 | 2047 | 295452 | 231453 | 377148 |
| 27 | Design 23 | 2887 | 1869 | 4460 | 460106 | 369594 | 572784 |
| 28 | Design 24 | 2466 | 1434 | 4242 | 650686 | 513751 | 824120 |

### Example 5: RBD Knockout Screening

In vitro work was carried out test whether the ACE2 binding domain met the criteria for RBD knock out for the following RBD mutant constructs shown in Table 13.

TABLE 13

| SEQ | Plasmid | |
|---|---|---|
| ID NO: | ID | Plasmid Name |
| 68 | 225 | pRS5a-S-RBD-mpSS ACE2 binding mutation K417W |
| 67 | 226* | pRS5a-S-RBD-mpSS ACE2 binding mutation K417M |
| 66 | 229* | pRS5a-S-RBD-mpSS ACE2 binding mutation K417L |
| 90 | 230* | pRS5a-S-RBD-mpSS ACE2 binding mutation F486T |
| 84 | 231* | pRS5a-S-RBD-mpSS ACE2 binding mutation F486H |
| 88 | 232* | pRS5a-S-RBD-mpSS ACE2 binding mutation F486N |
| 87 | 233* | pRS5a-S-RBD-mpSS ACE2 binding mutation F486M |
| 85 | 234 | pRS5a-S-RBD-mpSS ACE2 binding mutation F486I |
| 89 | 235 | pRS5a-S-RBD-mpSS ACE2 binding mutation F486P |
| 91 | 237 | pRS5a-S-RBD-mpSS ACE2 binding mutation F486W |
| 72 | 239 | pRS5a-S-RBD-mpSS ACE2 binding mutation L455A |
| 76 | 241 | pRS5a-S-RBD-mpSS ACE2 binding mutation L455W |
| 75 | 242* | pRS5a-S-RBD-mpSS ACE2 binding mutation L455N |
| 74 | 243 | pRS5a-S-RBD-mpSS ACE2 binding mutation L455M |
| 78 | 244* | pRS5a-S-RBD-mpSS ACE2 binding mutation F456I |
| 80 | 245 | pRS5a-S-RBD-mpSS ACE2 binding mutation F456Y |
| 79 | 246* | pRS5a-S-RBD-mpSS ACE2 binding mutation F456W |
| 77 | 247* | pRS5a-S-RBD-mpSS ACE2 binding mutation F456H |
| 95 | 249 | pRS5a-S-RBD-mpSS ACE2 binding mutation N487M |
| 93 | 250 | pRS5a-S-RBD-mpSS ACE2 binding mutation N487F |
| 96 | 251* | pRS5a-S-RBD-mpSS ACE2 binding mutation N487Q |
| 83 | 252 | pRS5a-S-RBD-mpSS ACE2 binding mutation G476T |
| 81 | 253 | pRS5a-S-RBD-mpSS ACE2 binding mutation Y473W |
| 97 | 255 | pRS5a-S-RBD-mpSS ACE2 binding mutation Q493A |
| 98 | 256 | pRS5a-S-RBD-mpSS ACE2 binding mutation Q493Y |
| 99 | 257 | pRS5a-S-RBD-mpSS ACE2 binding mutation Q493F |
| 100 | 258 | pRS5a-S-RBD-mpSS ACE2 binding mutation Q493R |
| 101 | 259 | pRS5a-S-RBD-mpSS ACE2 binding mutation Q493M |
| 102 | 260 | pRS5a-S-RBD-mpSS ACE2 binding mutation Q493C |
| 103 | 261 | pRS5a-S-RBD-mpSS ACE2 binding mutation Q493G |
| 104 | 262 | pRS5a-S-RBD-mpSS ACE2 binding mutation Q493V |
| 71 | 264 | pRS5a-S-RBD-mpSS ACE2 binding mutation Y453A |
| 105 | 265 | pRS5a-S-RBD-mpSS ACE2 binding mutation glycan K417N A419T |
| — | 266 | pRS5a-S-RBD-mpSS ACE2 binding mutation glycan Y449A Y45 IT |
| — | 268 | pRS5a-S-RBD-mpSS ACE2 binding mutation glycan L455A R457T |
| 111 | 271 | pRS5a-S-RBD-mpSS ACE2 binding mutation glycan A475N S477T |
| 112 | 272 | pRS5a-S-RBD-mpSS ACE2 binding mutation glycan G476N |
| 113 | 273 | pRS5a-S-RBD-mpSS ACE2 binding mutation glycan Y489T |
| 114 | 274 | pRS5a-S-RBD-mpSS ACE2 binding mutation glycan Q493N Y495T |

The RBD knockout mutants were expressed according to the protocols described above and tested for ACE2 binding using BLI using the methodology as described above. RBD ACE2_Kocked out mutants constructs 226, 229, 230, 231, 232, 233, 242, 244, 246, 247 and 251 (* in Table 13) show relatively high expression levels, but have reduced binding against ACE2, indicating the importance of these residues to interactions with the ACE2 binding domain.

SUMMARY OF SEQUENCES
SEQ ID NO: 1-complete genome sequence of Severe Acute Respiratory Syndrome Coronavirus 2 (SARS-
CoV2) (Wu et al. 2020 Nature 579:265-269; GenBank Accession MN908947.3 entitled “Severe Acute
Respiratory Syndrome Coronavirus 2 isolate Wuhan-Hu-1″) having the features 5’-3’ as follows:
5’ UTR nucleotides 1-265
“orf1ab” gene nucleotides 266-21555 with CDS nucleotides (join) 266-13468, 13468-21555 producing
″orf1ab polyprotein” (replicase, protein_id and GenBank Accession QHD43415.1)
“S” gene nucleotides 21563-25384 with CDS nucleotides 21563-25384 (underlined) producing “surface
glycoprotein” (spike (S) protein, protein_id and GenBank Accession QHD43416.1)
“ORF3a” gene nucleotides 25393-26220 with CDS nucleotides 25393-26220 producing “ORF3a protein”
(protein_id and GenBank Accession QHD43417.1)
“E” gene nucleotides 26245-26472 with CDS nucleotides 26245-26472 producing “envelope protein”
(envelope (E) protein, protein id and GenBank Accession QHD43418.1)
“M” gene nucleotides 26523-27191 with CDS nucleotides 26523-27191 producing “membrane
glycoprotein” (membrane (M) protein, protein_id and GenBank Accession QHD43419.1)
“ORF6” gene nucleotides 27202-27387 with CDS nucleotides 27202-27387 producing “ORF6 protein”
(protein_id and GenBank Accession QHD43420.1)
“ORF7a” gene nucleotides 27394-27759 with CDS nucleotides 27394-27759 producing “ORF7a protein”
(protein_id and GenBank Accession QHD43421.1)
“ORF8” gene nucleotides 27894-28259 with CDS nucleotides 27894-28259 producing “ORF8 protein”
(protein id and GenBank Accession QHD43422.1)
“N” gene nucleotides 28274-29533 with CDS nucleotides 28274-29533 producing “nucleocapsid
phosphoprotein ” (nucleocapsid (N) protein, protein_id and GenBank Accession QHD43423.2)
“ORF10” gene nucleotides 29558-29674 with CDS nucleotides 29558-29674 producing “ORF10 protein”
(protein_id and GenBank Accession QHI42199.1)
3’ UTR nucleotides 29675-29903

| ATTAAAGGTT TATACCTTCC CAGGTAACAA ACCAACCAAC TTTCGATCTC TTGTAGATCT | | 60 |
|---|---|---|
| GTTCTCTAAA CGAACTTTAA AATCTGTGTG GCTGTCACTC GGCTGCATGC TTAGTGCACT | | 120 |
| CACGCAGTAT AATTAATAAC TAATTACTGT CGTTGACAGG ACACGAGTAA CTCGTCTATC | | 180 |
| TTCTGCAGGC TGCTTACGGT TTCGTCCGTG TTGCAGCCGA TCATCAGCAC ATCTAGGTTT | | 240 |
| CGTCCGGGTG TGACCGAAAG GTAAGATGGA GAGCCTTGTC CCTGGTTTCA ACGAGAAAAC | | 300 |
| ACACGTCCAA CTCAGTTTGC CTGTTTTACA GGTTCGCGAC GTGCTCGTAC GTGGCTTTGG | | 360 |
| AGACTCCGTG GAGGAGGTCT TATCAGAGGC ACGTCAACAT CTTAAAGATG GCACTTGTGG | | 420 |
| CTTAGTAGAA GTTGAAAAAG GCGTTTTGCC TCAACTTGAA CAGCCCTATG TGTTCATCAA | | 480 |
| ACGTTCGGAT GCTCGAACTG CACCTCATGG TCATGTTATG GTTGAGCTGG TAGCAGAACT | | 540 |
| CGAAGGCATT CAGTACGGTC GTAGTGGTGA GACACTTGGT GTCCTTGTCC CTCATGTGGG | | 600 |
| CGAAATACCA GTGGCTTACC GCAAGGTTCT TCTTCGTAAG AACGGTAATA AAGGAGCTGG | | 660 |
| TGGCCATAGT TACGGCGCCG ATCTAAAGTC ATTTGACTTA GGCGACGAGC TTGGCACTGA | | 720 |
| TCCTTATGAA GATTTTCAAG AAAACTGGAA CACTAAACAT AGCAGTGGTG TTACCCGTGA | | 780 |
| ACTCATGCGT GAGCTTAACG GAGGGGCATA CACTCGCTAT GTCGATAACA ACTTCTGTGG | | 840 |
| CCCTGATGGC TACCCTCTTG AGTGCATTAA AGACCTTCTA GCACGTGCTG GTAAAGCTTC | | 900 |
| ATGCACTTTG TCCGAACAAC TGGACTTTAT TGACACTAAG AGGGGTGTAT ACTGCTGCCG | | 960 |
| TGAACATGAG CATGAAATTG CTTGGTACAC GGAACGTTCT GAAAAGAGCT ATGAATTGCA | | 1020 |
| GACACCTTTT GAAATTAAAT TGGCAAAGAA ATTTGACACC TTCAATGGGG AATGTCCAAA | | 1080 |
| TTTTGTATTT CCCTTAAATT CCATAATCAA GACTATTCAA CCAAGGGTTG AAAAGAAAAA | | 1140 |
| GCTTGATGGC TTTATGGGTA GAATTCGATC TGTCTATCCA GTTGCGTCAC CAAATGAATG | | 1200 |
| CAACCAAATG TGCCTTTCAA CTCTCATGAA GTGTGATCAT TGTGGTGAAA CTTCATGGCA | | 1260 |
| GACGGGCGAT TTTGTTAAAG CCACTTGCGA ATTTTGTGGC ACTGAGAATT TGACTAAAGA | | 1320 |
| AGGTGCCACT ACTTGTGGTT ACTTACCCCA AAATGCTGTT GTTAAAATTT ATTGTCCAGC | | 1380 |
| ATGTCACAAT TCAGAAGTAG GACCTGAGCA TAGTCTTGCC GAATACCATA ATGAATCTGG | | 1440 |
| CTTGAAAACC ATTCTTCGTA AGGGTGGTCG CACTATTGCC TTTGGAGGCT GTGTGTTCTC | | 1500 |
| TTATGTTGGT TGCCATAACA AGTGTGCCTA TTGGGTTCCA CGTGCTAGCG CTAACATAGG | | 1560 |
| TTGTAACCAT ACAGGTGTTG TTGGAGAAGG TTCCGAAGGT CTTAATGACA ACCTTCTTGA | | 1620 |
| AATACTCCAA AAAGAGAAAG TCAACATCAA TATTGTTGGT GACTTTAAAC TTAATGAAGA | | 1680 |
| GATCGCCATT ATTTTGGCAT CTTTTTCTGC TTCCACAAGT GCTTTTGTGG AAACTGTGAA | | 1740 |
| AGGTTTGGAT TATAAAGCAT TCAAACAAAT TGTTGAATCC TGTGGTAATT TTAAAGTTAC | | 1800 |
| AAAAGGAAAA GCTAAAAAAG GTGCCTGGAA TATTGGTGAA CAGAAATCAA TACTGAGTCC | | 1860 |
| TCTTTATGCA TTTGCATCAG AGGCTGCTCG TGTTGTACGA TCAATTTTCT CCCGCACTCT | | 1920 |
| TGAAACTGCT CAAAATTCTG TGCGTGTTTT ACAGAAGGCC GCTATAACAA TACTAGATGG | | 1980 |
| AATTTCACAG TATTCACTGA GACTCATTGA TGCTATGATG TTCACATCTG ATTTGGCTAC | | 2040 |
| TAACAATCTA GTTGTAATGG CCTACATTAC AGGTGGTGTT GTTCAGTTGA CTTCGCAGTG | | 2100 |
| GCTAACTAAC ATCTTTGGCA CTGTTTATGA AAAACTCAAA CCCGTCCTTG ATTGGCTTGA | | 2160 |
| AGAGAAGTTT AAGGAAGGTG TAGAGTTTCT TAGAGACGGT TGGGAAATTG TTAAATTTAT | | 2220 |
| CTCAACCTGT GCTTGTGAAA TTGTCGGTGG ACAAATTGTC ACCTGTGCAA AGGAAATTAA | | 2280 |
| GGAGAGTGTT CAGACATTCT TTAAGCTTGT AAATAAATTT TTGGCTTTGT GTGCTGACTC | | 2340 |
| TATCATTATT GGTGGAGCTA AACTTAAAGC CTTGAATTTA GGTGAAACAT TTGTCACGCA | | 2400 |
| CTCAAAGGGA TTGTACAGAA AGTGTGTTAA ATCCAGAGAA GAAACTGGCC TACTCATGCC | | 2460 |
| TCTAAAAGCC CCAAAAGAAA TTATCTTCTT AGAGGGAGAA ACACTTCCCA CAGAAGTGTT | | 2520 |
| AACAGAGGAA GTTGTCTTGA AAACTGGTGA TTTACAACCA TTAGAACAAC CTACTAGTGA | | 2580 |
| AGCTGTTGAA GCTCCATTGG TTGGTACACC AGTTTGTATT AACGGGCTTA TGTTGCTCGA | | 2640 |
| AATCAAAGAC ACAGAAAAGT ACTGTGCCCT TGCACCTAAT ATGATGGTAA CAAACAATAC | | 2700 |
| CTTCACACTC AAAGGCGGTG CACCAACAAA GGTTACTTTT GGTGATGACA CTGTGATAGA | | 2760 |
| AGTGCAAGGT TACAAGAGTG TGAATATCAC TTTTGAACTT GATGAAAGGA TTGATAAAGT | | 2820 |
| ACTTAATGAG AAGTGCTCTG CCTATACAGT TGAACTCGGT ACAGAAGTAA ATGAGTTCGC | | 2880 |
| CTGTGTTGTG GCAGATGCTG TCATAAAAAC TTTGCAACCA GTATCTGAAT TACTTACACC | | 2940 |
| ACTGGGCATT GATTTAGATG AGTGGAGTAT GGCTACATAC TACTTATTTG ATGAGTCTGG | | 3000 |
| TGAGTTTAAA TTGGCTTCAC ATATGTATTG TTCTTTCTAC CCTCCAGATG AGGATGAAGA | | 3060 |
| AGAAGGTGAT TGTGAAGAAG AAGAGTTTGA GCCATCAACT CAATATGAGT ATGGTACTGA | | 3120 |
| AGATGATTAC CAAGGTAAAC CTTTGGAATT TGGTGCCACT TCTGCTGCTC TTCAACCTGA | | 3180 |
| AGAAGAGCAA GAAGAAGATT GGTTAGATGA TGATAGTCAA CAAACTGTTG GTCAACAAGA | | 3240 |
| CGGCAGTGAG GACAATCAGA CAACTACTAT TCAAACAATT GTTGAGGTTC AACCTCAATT | | 3300 |
| AGAGATGGAA CTTACACCAG TTGTTCAGAC TATTGAAGTG AATAGTTTTA GTGGTTATTT | | 3360 |
| AAAACTTACT GACAATGTAT ACATTAAAAA TGCAGACATT GTGGAAGAAG CTAAAAAGGT | | 3420 |
| AAAACCAACA GTGGTTGTTA ATGCAGCCAA TGTTTACCTT AAACATGGAG GAGGTGTTGC | | 3480 |
| AGGAGCCTTA AATAAGGCTA CTAACAATGC CATGCAAGTT GAATCTGATG ATTACATAGC | | 3540 |
| TACTAATGGA CCACTTAAAG TGGGTGGTAG TTGTGTTTTA AGCGGACACA ATCTTGCTAA | | 3600 |
| ACACTGTCTT CATGTTGTCG GCCCAAATGT TAACAAAGGT GAAGACATTC AACTTCTTAA | | 3660 |
| GAGTGCTTAT GAAAATTTTA ATCAGCACGA AGTTCTACTT GCACCATTAT TATCAGCTGG | | 3720 |
| TATTTTTGGT GCTGACCCTA TACATTCTTT AAGAGTTTGT GTAGATACTG TTCGCACAAA | | 3780 |
| TGTCTACTTA GCTGTCTTTG ATAAAAATCT CTATGACAAA CTTGTTTCAA GCTTTTTGGA | | 3840 |
| AATGAAGAGT GAAAAGCAAG TTGAACAAAA GATCGCTGAG ATTCCTAAAG AGGAAGTTAA | | 3900 |
| GCCATTTATA ACTGAAAGTA AACCTTCAGT TGAACAGAGA AAACAAGATG ATAAGAAAAT | | 3960 |
| CAAAGCTTGT GTTGAAGAAG TTACAACAAC TCTGGAAGAA ACTAAGTTCC TCACAGAAAA | | 4020 |
| CTTGTTACTT TATATTGACA TTAATGGCAA TCTTCATCCA GATTCTGCCA CTCTTGTTAG | | 4080 |
| TGACATTGAC ATCACTTTCT TAAAGAAAGA TGCTCCATAT ATAGTGGGTG ATGTTGTTCA | | 4140 |
| AGAGGGTGTT TTAACTGCTG TGGTTATACC TACTAAAAAG GCTGGTGGCA CTACTGAAAT | | 4200 |
| GCTAGCGAAA GCTTTGAGAA AAGTGCCAAC AGACAATTAT ATAACCACTT ACCCGGGTCA | | 4260 |
| GGGTTTAAAT GGTTACACTG TAGAGGAGGC AAAGACAGTG CTTAAAAAGT GTAAAAGTGC | | 4320 |
| CTTTTACATT CTACCATCTA TTATCTCTAA TGAGAAGCAA GAAATTCTTG GAACTGTTTC | | 4380 |
| TTGGAATTTG CGAGAAATGC TTGCACATGC AGAAGAAACA CGCAAATTAA TGCCTGTCTG | | 4440 |
| TGTGGAAACT AAAGCCATAG TTTCAACTAT ACAGCGTAAA TATAAGGGTA TTAAAATACA | | 4500 |
| AGAGGGTGTG GTTGATTATG GTGCTAGATT TTACTTTTAG ACCAGTAAAA CAACTGTAGC | | 4560 |
| GTCACTTATC AACACACTTA ACGATCTAAA TGAAACTCTT GTTACAATGC CACTTGGCTA | | 4620 |
| TGTAACACAT GGCTTAAATT TGGAAGAAGC TGCTCGGTAT ATGAGATCTC TCAAAGTGCC | | 4680 |
| AGCTACAGTT TCTGTTTCTT CACCTGATGC TGTTACAGCG TATAATGGTT ATCTTACTTC | | 4740 |
| TTCTTCTAAA ACACCTGAAG AACATTTTAT TGAAACCATC TCACTTGCTG GTTCCTATAA | | 4800 |
| AGATTGGTCC TATTCTGGAC AATCTACACA ACTAGGTATA GAATTTCTTA AGAGAGGTGA | | 4860 |
| TAAAAGTGTA TATTACACTA GTAATCCTAC CACATTCCAC CTAGATGGTG AAGTTATCAC | | 4920 |
| CTTTGACAAT CTTAAGACAC TTCTTTCTTT GAGAGAAGTG AGGACTATTA AGGTGTTTAC | | 4980 |
| AACAGTAGAC AACATTAACC TCCACACGCA AGTTGTGGAC ATGTCAATGA CATATGGACA | | 5040 |
| ACAGTTTGGT CCAACTTATT TGGATGGAGC TGATGTTACT AAAATAAAAC CTCATAATTC | | 5100 |
| ACATGAAGGT AAAACATTTT ATGTTTTACC TAATGATGAC ACTCTACGTG TTGAGGCTTT | | 5160 |
| TGAGTACTAC CACACAACTG ATCCTAGTTT TCTGGGTAGG TACATGTCAG CATTAAATCA | | 5220 |
| CACTAAAAAG TGGAAATACC CACAAGTTAA TGGTTTAACT TCTATTAAAT GGGCAGATAA | | 5280 |
| CAACTGTTAT CTTGCCACTG CATTGTTAAC ACTCCAACAA ATAGAGTTGA AGTTTAATCC | | 5340 |
| ACCTGCTCTA CAAGATGCTT ATTACAGAGC AAGGGCTGGT GAAGCTGCTA ACTTTTGTGC | | 5400 |
| ACTTATCTTA GCCTACTGTA ATAAGACAGT AGGTGAGTTA GGTGATGTTA GAGAAACAAT | | 5460 |
| GAGTTACTTG TTTCAACATG CCAATTTAGA TTCTTGCAAA AGAGTCTTGA ACGTGGTGTG | | 5520 |
| TAAAACTTGT | GGACAACAGC AGACAACCCT TAAGGGTGTA GAAGCTGTTA TGTACATGGG | 5580 |

| | |
|---|---|
| CACACTTTCT TATGAACAAT TTAAGAAAGG TGTTCAGATA CCTTGTACGT GTGGTAAACA | 5640 |
| AGCTACAAAA TATCTAGTAC AACAGGAGTC ACCTTTTGTT ATGATGTCAG CACCACCTGC | 5700 |
| TCAGTATGAA CTTAAGCATG GTACATTTAC TTGTGCTAGT GAGTACACTG GTAATTACCA | 5760 |
| GTGTGGTCAC TATAAACATA TAACTTCTAA AGAAACTTTG TATTGCATAG ACGGTGCTTT | 5820 |
| ACTTACAAAG TCCTCAGAAT ACAAAGGTCC TATTACGGAT GTTTTCTACA AAGAAAACAG | 5880 |
| TTACACAACA ACCATAAAAC CAGTTACTTA TAAATTGGAT GGTGTTGTTT GTACAGAAAT | 5940 |
| TGACCCTAAG TTGGACAATT ATTATAAGAA AGACAATTCT TATTTCACAG AGCAACCAAT | 6000 |
| TGATCTTGTA CCAAACCAAC CATATCCAAA CGCAAGCTTC GATAATTTTA AGTTTGTATG | 6060 |
| TGATAATATC AAATTTGCTG ATGATTTAAA CCAGTTAACT GGTTATAAGA AACCTGCTTC | 6120 |
| AAGAGAGCTT AAAGTTACAT TTTTCCCTGA CTTAAATGGT GATGTGGTGG CTATTGATTA | 6180 |
| TAAACACTAC ACACCCTCTT TTAAGAAAGG AGCTAAATTG TTACATAAAC CTATTGTTTG | 6240 |
| GCATGTTAAC AATGCAACTA ATAAAGCCAC GTATAAACCA AATACCTGGT GTATACGTTG | 6300 |
| TCTTTGGAGC ACAAAACCAG TTGAAACATC AAATTCGTTT GATGTACTGA AGTCAGAGGA | 6360 |
| CGCGCAGGGA ATGGATAATC TTGCCTGCGA AGATCTAAAA CCAGTCTCTG AAGAAGTAGT | 6420 |
| GGAAAATCCT ACCATACAGA AAGACGTTCT TGAGTGTAAT GTGAAAACTA CCGAAGTTGT | 6480 |
| AGGAGACATT ATACTTAAAC CAGCAAATAA TAGTTTAAAA ATTACAGAAG AGGTTGGCCA | 6540 |
| CACAGATCTA ATGGCTGCTT ATGTAGACAA TTCTAGTCTT ACTATTAAGA AACCTAATGA | 6600 |
| ATTATCTAGA GTATTAGGTT TGAAAACCCT TGCTACTCAT GGTTTAGCTG CTGTTAATAG | 6660 |
| TGTCCCTTGG GATACTATAG CTAATTATGC TAAGCCTTTT CTTAACAAAG TTGTTAGTAC | 6720 |
| AACTACTAAC ATAGTTACAC GGTGTTTAAA CCGTGTTTGT ACTAATTATA TGCCTTATTT | 6780 |
| CTTTACTTTA TTGCTACAAT TGTGTACTTT TACTAGAAGT ACAAATTCTA GAATTAAAGC | 6840 |
| ATCTATGCCG ACTACTATAG CAAAGAATAC TGTTAAGAGT GTCGGTAAAT TTTGTCTAGA | 6900 |
| GGCTTCATTT AATTATTTGA AGTCACCTAA TTTTTCTAAA CTGATAAATA TTATAATTTG | 6960 |
| GTTTTTACTA TTAAGTGTTT GCCTAGGTTC TTTAATCTAC TCAACCGCTG CTTTAGGTGT | 7020 |
| TTTAATGTCT AATTTAGGCA TGCCTTCTTA CTGTACTGGT TACAGAGAAG GCTATTTGAA | 7080 |
| CTCTACTAAT GTCACTATTG CAACCTACTG TACTGGTTCT ATACCTTGTA GTGTTTGTCT | 7140 |
| TAGTGGTTTA GATTCTTTAG ACACCTATCC TTCTTTAGAA ACTATACAAA TTACCATTTC | 7200 |
| ATCTTTTAAA TGGGATTTAA CTGCTTTTGG CTTAGTTGCA GAGTGGTTTT TGGCATATAT | 7260 |
| TCTTTTCACT AGGTTTTTCT ATGTACTTGG ATTGGCTGCA ATCATGCAAT TGTTTTTCAG | 7320 |
| ctAttttgcA GTACATTTTA TTAGTAATTC TTGGCTTATG TGGTTAATAA TTAATCTTGT | 7380 |
| ACAAATGGCC CCGATTTCAG CTATGGTTAG AATGTACATC TTCTTTGCAT CATTTTATTA | 7440 |
| TGTATGGAAA AGTTATGTGC ATGTTGTAGA CGGTTGTAAT TCATCAACTT GTATGATGTG | 7500 |
| TTACAAACGT AATAGAGCAA CAAGAGTCGA ATGTACAACT ATTGTTAATG GTGTTAGAAG | 7560 |
| GTCCTTTTAT GTCTATGCTA ATGGAGGTAA AGGCTTTTGC AAACTACACA ATTGGAATTG | 7620 |
| TGTTAATTGT GATACATTCT GTGCTGGTAG TACATTTATT AGTGATGAAG TTGCGAGAGA | 7680 |
| CTTGTCACTA CAGTTTAAAA GACCAATAAA TCCTACTGAC CAGTCTTCTT ACATCGTTGA | 7740 |
| TAGTGTTACA GTGAAGAATG GTTCCATCCA TCTTTACTTT GATAAAGCTG GTCAAAAGAC | 7800 |
| TTATGAAAGA CATTCTCTCT CTCATTTTGT TAACTTAGAC AACCTGAGAG CTAATAACAC | 7860 |
| TAAAGGTTCA TTGCCTATTA ATGTTATAGT TTTTGATGGT AAATCAAAAT GTGAAGAATC | 7920 |
| ATCTGCAAAA TCAGCGTCTG TTTACTACAG TCAGCTTATG TGTCAACCTA TACTGTTACT | 7980 |
| AGATCAGGCA TTAGTGTCTG ATGTTGGTGA TAGTGCGGAA GTTGCAGTTA AAATGTTTGA | 8040 |
| TGCTTACGTT AATACGTTTT CATCAACTTT TAACGTACCA ATGGAAAAAC TCAAAACACT | 8100 |
| AGTTGCAACT GCAGAAGCTG AACTTGCAAA GAATGTGTCC TTAGACAATG TCTTATCTAC | 8160 |
| TTTTATTTCA GCAGCTCGGC AAGGGTTTGT TGATTCAGAT GTAGAAACTA AAGATGTTGT | 8220 |
| TGAATGTCTT AAATTGTCAC ATCAATCTGA CATAGAAGTT ACTGGCGATA GTTGTAATAA | 8280 |
| CTATATGCTC ACCTATAACA AAGTTGAAAA CATGACACCC CGTGACCTTG GTGCTTGTAT | 8340 |
| TGACTGTAGT GCGCGTCATA TTAATGCGCA GGTAGCAAAA AGTCACAACA TTGCTTTGAT | 8400 |
| ATGGAACGTT AAAGATTTCA TGTCATTGTC TGAACAACTA CGAAAACAAA TACGTAGTGC | 8460 |
| TGCTAAAAAG AATAACTTAC CTTTTAAGTT GACATGTGCA ACTACTAGAC AAGTTGTTAA | 8520 |
| TGTTGTAACA ACAAAGATAG CACTTAAGGG TGGTAAAATT GTTAATAATT GGTTGAAGCA | 8580 |
| GTTAATTAAA GTTACACTTG TGTTCCTTTT TGTTGCTGCT ATTTTCTATT TAATAACACC | 8640 |
| TGTTCATGTC ATGTCTAAAC ATACTGACTT TTCAAGTGAA ATCATAGGAT ACAAGGCTAT | 8700 |
| TGATGGTGGT GTCACTCGTG ACATAGCATC TACAGATACT TGTTTTGCTA ACAAACATGC | 8760 |
| TGATTTTGAC ACATGGTTTA GCCAGCGTGG TGGTAGTTAT ACTAATGACA AAGCTTGCCC | 8820 |
| ATTGATTGCT GCAGTCATAA CAAGAGAAGT GGGTTTTGTC GTGCCTGGTT TGCCTGGCAC | 8880 |
| GATATTACGC ACAACTAATG GTGACTTTTT GCATTTCTTA CCTAGAGTTT TTAGTGCAGT | 8940 |
| TGGTAACATC TGTTACACAC CATCAAAACT TATAGAGTAC ACTGACTTTG CAACATCAGC | 9000 |
| TTGTGTTTTG GCTGCTGAAT GTACAATTTT TAAAGATGCT TCTGGTAAGC CAGTACCATA | 9060 |
| TTGTTATGAT ACCAATGTAC TAGAAGGTTC TGTTGCTTAT GAAAGTTTAC GCCCTGACAC | 9120 |
| ACGTTATGTG CTCATGGATG GCTCTATTAT TCAATTTCCT AACACCTACC TTGAAGGTTC | 9180 |
| TGTTAGAGTG GTAACAACTT TTGATTCTGA GTACTGTAGG CACGGCACTT GTGAAAGATC | 9240 |
| AGAAGCTGGT GTTTGTGTAT CTACTAGTGG TAGATGGGTA CTTAACAATG ATTATTACAG | 9300 |
| ATCTTTACCA GGAGTTTTCT GTGGTGTAGA TGCTGTAAAT TTACTTACTA ATATGTTTAC | 9360 |
| ACCACTAATT CAACCTATTG GTGCTTTGGA CATATCAGCA TCTATAGTAG CTGGTGGTAT | 9420 |
| TGTAGCTATC GTAGTAACAT GCCTTGCCTA CTATTTTATG AGGTTTAGAA GAGCTTTTGG | 9480 |
| TGAATACAGT CATGTAGTTG CCTTTAATAC TTTACTATTC CTTATGTCAT TCACTGTACT | 9540 |
| CTGTTTAACA CCAGTTTACT CATTCTTACC TGGTGTTTAT TCTGTTATTT ACTTGTACTT | 9600 |
| GACATTTTAT CTTACTAATG ATGTTTCTTT TTTAGCACAT ATTCAGTGGA TGGTTATGTT | 9660 |
| CACACCTTTA GTACCTTTCT GGATAACAAT TGCTTATATC ATTTGTATTT CCACAAAGCA | 9720 |
| TTTCTATTGG TTCTTTAGTA ATTACCTAAA GAGACGTGTA GTCTTTAATG GTGTTTCCTT | 9780 |
| TAGTACTTTT GAAGAAGCTG CGCTGTGCAC CTTTTTGTTA AATAAAGAAA TGTATCTAAA | 9840 |
| GTTGCGTAGT GATGTGCTAT TACCTCTTAC GCAATATAAT AGATACTTAG CTCTTTATAA | 9900 |
| TAAGTACAAG TATTTTAGTG GAGCAATGGA TACAACTAGC TACAGAGAAG CTGCTTGTTG | 9960 |
| TCATCTCGCA AAGGCTCTCA ATGACTTCAG TAACTCAGGT TCTGATGTTC TTTACCAACC | 10020 |
| ACCACAAACC TCTATCACCT CAGCTGTTTT GCAGAGTGGT TTTAGAAAAA TGGCATTCCC | 10080 |
| ATCTGGTAAA GTTGAGGGTT GTATGGTACA AGTAACTTGT GGTACAACTA CACTTAACGG | 10140 |
| TCTTTGGCTT GATGACGTAG TTTACTGTCC AAGACATGTG ATCTGCACCT CTGAAGACAT | 10200 |
| GCTTAACCCT AATTATGAAG ATTTACTCAT TCGTAAGTCT AATCATAATT TCTTGGTACA | 10260 |
| GGCTGGTAAT GTTCAACTCA GGGTTATTGG ACATTCTATG CAAAATTGTG TACTTAAGCT | 10320 |
| TAAGGTTGAT ACAGCCAATC CTAAGACACC TAAGTATAAG TTTGTTCGCA TTCAACCAGG | 10380 |
| ACAGACTTTT TCAGTGTTAG CTTGTTACAA TGGTTCACCA TCTGGTGTTT ACCAATGTGC | 10440 |
| TATGAGGCCC AATTTCACTA TTAAGGGTTC ATTCCTTAAT GGTTCATGTG GTAGTGTTGG | 10500 |
| TTTTAACATA GATTATGACT GTGTCTCTTT TTGTTACATG CACCATATGG AATTACCAAC | 10560 |
| TGGAGTTCAT GCTGGCACAG ACTTAGAAGG TAACTTTTAT GGACCTTTTG TTGACAGGCA | 10620 |
| AACAGCACAA GCAGCTGGTA CGGACACAAC TATTACAGTT AATGTTTTAG CTTGGTTGTA | 10680 |
| CGCTGCTGTT ATAAATGGAG ACAGGTGGTT TCTCAATCGA TTTACCACAA CTCTTAATGA | 10740 |
| CTTTAACCTT GTGGCTATGA AGTACAATTA TGAACCTCTA ACACAAGACC ATGTTGACAT | 10800 |
| ACTAGGACCT CTTTCTGCTC AAACTGGAAT TGCCGTTTTA GATATGTGTG CTTCATTAAA | 10860 |
| AGAATTACTG CAAAATGGTA TGAATGGACG TAGCATATTG GGTAGTGCTT TATTAGAAGA | 10920 |
| TGAATTTACA CCTTTTGATG TTGTTAGACA ATGCTCAGGT GTTACTTTCC AAAGTGCAGT | 10980 |
| GAAAAGAACA ATCAAGGGTA CACACCACTG GTTGTTACTC ACAATTTTGA CTTCACTTTT | 11040 |
| AGTTTTAGTC CAGAGTACTC AATGGTCTTT GTTCTTTTTT TTGTATGAAA ATGCCTTTTT | 11100 |
| ACCTTTTGCT ATGGGTATTA TTGCTATGTC TGCTTTTGCA ATGATGTTTG TCAAACATAA | 11160 |
| GCATGCATTT CTCTGTTTGT TTTTGTTACC TTCTCTTGCC ACTGTAGCTT ATTTTAATAT | 11220 |
| GGTCTATATG CCTGCTAGTT GGGTGATGCG TATTATGACA TGGTTGGATA TGGTTGATAC | 11280 |
| TAGTTTGTCT GGTTTTAAGC TAAAAGACTG TGTTATGTAT GCATCAGCTG TAGTGTTACT | 11340 |
| AATCCTTATG ACAGCAAGAA CTGTGTATGA TGATGGTGCT AGGAGAGTGT GGACACTTAT | 11400 |
| GAATGTCTTG ACACTCGTTT ATAAAGTTTA TTATGGTAAT GCTTTAGATC AAGCCATTTC | 11460 |
| CATGTGGGCT CTTATAATCT CTGTTACTTC TAACTACTCA GGTGTAGTTA CAACTGTCAT | 11520 |
| GTTTTTGGGG AGAGGTATTG TTTTTATGTG TGTTGAGTAT TGCCCTATTT TCTTCATAAC | 11580 |
| TGGTAATACA CTTCAGTGTA TAATGCTAGT TTATTGTTTC TTAGGCTATT TTTGTACTTG | 11640 |
| TTACTTTGGC CTCTTTTGTT TACTCAACCG CTACTTTAGA CTGACTCTTG GTGTTTATGA | 11700 |
| TTACTTAGTT TCTACACAGG AGTTTAGATA TATGAATTCA CAGGGACTAC TCCCACCCAA | 11760 |
| GAATAGCATA GATGCCTTCA AACTCAACAT TAAATTGTTG GGTGTTGGTG GCAAACCTTG | 11820 |
| TATCAAAGTA GCCACTGTAC AGTCTAAAAT GTCAGATGTA AAGTGCACAT CAGTAGTCTT | 11880 |
| ACTCTCAGTT TTGCAACAAC TCAGAGTAGA ATCATCATCT AAATTGTGGG CTCAATGTGT | 11940 |
| CCAGTTACAC AATGACATTC TCTTAGCTAA AGATACTACT GAAGCCTTTG AAAAAATGGT | 12000 |
| TTCACTACTT TCTGTTTTGC TTTCCATGCA GGGTGCTGTA GACATAAACA AGCTTTGTGA | 12060 |
| AGAAATGCTG GACAACAGGG CAACCTTACA AGCTATAGCC TCAGAGTTTA GTTCCCTTCC | 12120 |
| ATCATATGCA GCTTTTGCTA CTGCTCAAGA AGCTTATGAG CAGGCTGTTG CTAATGGTGA | 12180 |
| TTCTGAAGTT GTTCTTAAAA AGTTGAAGAA GTCTTTGAAT GTGGCTAAAT CTGAATTTGA | 12240 |
| CCGTGATGCA GCCATGCAAC GTAAGTTGGA AAAGATGGCT GATCAAGCTA TGACCCAAAT | 12300 |
| GTATAAACAG GCTAGATCTG AGGACAAGAG GGCAAAAGTT ACTAGTGCTA TGCAGACAAT | 12360 |
| GCTTTTCACT ATGCTTAGAA AGTTGGATAA TGATGCACTC AACAACATTA TCAACAATGC | 12420 |
| AAGAGATGGT TGTGTTCCCT TGAACATAAT ACCTCTTACA ACAGCAGCCA AACTAATGGT | 12480 |
| TGTCATACCA GACTATAACA CATATAAAAA TACGTGTGAT GGTACAACAT TTACTTATGC | 12540 |
| ATCAGCATTG TGGGAAATCC AACAGGTTGT AGATGCAGAT AGTAAAATTG TTCAACTTAG | 12600 |
| TGAAATTAGT ATGGACAATT CACCTAATTT AGCATGGCCT CTTATTGTAA CAGCTTTAAG | 12660 |
| GGCCAATTCT GCTGTCAAAT TACAGAATAA TGAGCTTAGT CCTGTTGCAC TACGACAGAT | 12720 |
| GTCTTGTGCT GCCGGTACTA CACAAACTGC TTGCACTGAT GACAATGCGT TAGCTTACTA | 12780 |
| CAACACAACA AAGGGAGGTA GGTTTGTACT TGCACTGTTA TCCGATTTAC AGGATTTGAA | 12840 |
| ATGGGCTAGA TTCCCTAAGA GTGATGGAAC TGGTACTATC TATACAGAAC TGGAACCACC | 12900 |
| TTGTAGGTTT GTTACAGACA CACCTAAAGG TCCTAAAGTG AAGTATTTAT ACTTTATTAA | 12960 |
| AGGATTAAAC AACCTAAATA GAGGTATGGT ACTTGGTAGT TTAGCTGCCA CAGTACGTCT | 13020 |
| ACAAGCTGGT AATGCAACAG AAGTGCCTGC CAATTCAACT GTATTATCTT TCTGTGCTTT | 13080 |
| TGCTGTAGAT GCTGCTAAAG CTTACAAAGA TTATCTAGCT AGTGGGGGAC AACCAATCAC | 13140 |
| TAATTGTGTT AAGATGTTGT GTACACACAC TGGTACTGGT CAGGCAATAA CAGTTACACC | 13200 |
| GGAAGCCAAT ATGGATCAAG AATCCTTTGG TGGTGCATCG TGTTGTCTGT ACTGCCGTTG | 13260 |
| CCACATAGAT CATCCAAATC CTAAAGGATT TTGTGACTTA AAAGGTAAGT ATGTACAAAT | 13320 |
| ACCTACAACT TGTGCTAATG ACCCTGTGGG TTTTACACTT AAAAACACAG TCTGTACCGT | 13380 |
| CTGCGGTATG TGGAAAGGTT ATGGCTGTAG TTGTGATCAA CTCCGCGAAC CCATGCTTCA | 13440 |
| GTCAGCTGAT GCACAATCGT TTTTAAACGG GTTTGCGGTG TAAGTGCAGC CCGTCTTACA | 13500 |
| CCGTGCGGCA CAGGCACTAG TACTGATGTC GTATACAGGG CTTTTGACAT CTACAATGAT | 13560 |
| AAAGTAGCTG GTTTTGCTAA ATTCCTAAAA ACTAATTGTT GTCGCTTCCA AGAAAAGGAC | 13620 |
| GAAGATGACA ATTTAATTGA TTCTTACTTT GTAGTTAAGA GACACACTTT CTCTAACTAC | 13680 |
| CAACATGAAG AAACAATTTA TAATTTACTT AAGGATTGTC CAGCTGTTGC TAAACATGAC | 13740 |
| TTCTTTAAGT TTAGAATAGA CGGTGACATG GTACCACATA TATCACGTCA ACGTCTTACT | 13800 |
| AAATACACAA TGGCAGACCT CGTCTATGCT TTAAGGCATT TTGATGAAGG TAATTGTGAC | 13860 |
| ACATTAAAAG AAATACTTGT CACATACAAT TGTTGTGATG ATGATTATTT CAATAAAAAG | 13920 |
| GACTGGTATG ATTTTGTAGA AAACCCAGAT ATATTACGCG TATACGCCAA CTTAGGTGAA | 13980 |
| CGTGTACGCC AAGCTTTGTT AAAAACAGTA CAATTCTGTG ATGCCATGCG AAATGCTGGT | 14040 |
| ATTGTTGGTG TACTGACATT AGATAATCAA GATCTCAATG GTAACTGGTA TGATTTCGGT | 14100 |
| GATTTCATAC AAACCACGCC AGGTAGTGGA GTTCCTGTTG TAGATTCTTA TTATTCATTG | 14160 |
| TTAATGCCTA TATTAACCTT GACCAGGGCT TTAACTGCAG AGTCACATGT TGACACTGAC | 14220 |
| TTAACAAAGC CTTACATTAA GTGGGATTTG TTAAAATATG ACTTCACGGA AGAGAGGTTA | 14280 |
| AAACTCTTTG ACCGTTATTT TAAATATTGG GATCAGACAT ACCACCCAAA TTGTGTTAAC | 14340 |
| TGTTTGGATG ACAGATGCAT TCTGCATTGT GCAAACTTTA ATGTTTTATT CTCTACAGTG | 14400 |
| TTCCCACCTA CAAGTTTTGG ACCACTAGTG AGAAAAATAT TTGTTGATGG TGTTCCATTT | 14460 |
| GTAGTTTCAA CTGGATACCA CTTCAGAGAG CTAGGTGTTG TACATAATCA GGATGTAAAC | 14520 |
| TTACATAGCT CTAGACTTAG TTTTAAGGAA TTACTTGTGT ATGCTGCTGA CCCTGCTATG | 14580 |
| CACGCTGCTT CTGGTAATCT ATTACTAGAT AAACGCACTA CGTGCTTTTC AGTAGCTGCA | 14640 |
| CTTACTAACA ATGTTGCTTT TCAAACTGTC AAACCCGGTA ATTTTAACAA AGACTTCTAT | 14700 |
| GACTTTGCTG TGTCTAAGGG TTTCTTTAAG GAAGGAAGTT CTGTTGAATT AAAACACTTC | 14760 |
| TTCTTTGCTC AGGATGGTAA TGCTGCTATC AGCGATTATG ACTACTATCG TTATAATCTA | 14820 |
| CCAACAATGT GTGATATGAG ACAACTACTA TTTGTAGTTG AAGTTGTTGA TAAGTACTTT | 14880 |
| GATTGTTACG ATGGTGGCTG TATTAATGCT AACCAAGTCA TCGTCAACAA CCTAGACAAA | 14940 |
| TCAGCTGGTT TTCCATTTAA TAAATGGGGT AAGGCTAGAC TTTATTATGA TTCAATGAGT | 15000 |
| TATGAGGATC AAGATGCACT TTTCGCATAT ACAAAACGTA ATGTCATCCC TACTATAACT | 15060 |
| CAAATGAATC TTAAGTATGC CATTAGTGCA AAGAATAGAG CTCGCACCGT AGCTGGTGTC | 15120 |
| TCTATCTGTA GTACTATGAC CAATAGACAG TTTCATCAAA AATTATTGAA ATCAATAGCC | 15180 |
| GCCACTAGAG GAGCTACTGT AGTAATTGGA ACAAGCAAAT TCTATGGTGG TTGGCACAAC | 15240 |
| ATGTTAAAAA CTGTTTATAG TGATGTAGAA AACCCTCACC TTATGGGTTG GGATTATCCT | 15300 |
| AAATGTGATA GAGCCATGCC TAACATGCTT AGAATTATGG CCTCACTTGT TCTTGCTCGC | 15360 |
| AAACATACAA CGTGTTGTAG CTTGTCACAC CGTTTCTATA GATTAGCTAA TGAGTGTGCT | 15420 |
| CAAGTATTGA GTGAAATGGT CATGTGTGGC GGTTCACTAT ATGTTAAACC AGGTGGAACC | 15480 |
| TCATCAGGAG ATGCCACAAC TGCTTATGCT AATAGTGTTT TTAACATTTG TCAAGCTGTC | 15540 |
| ACGGCCAATG TTAATGCACT TTTATCTACT GATGGTAACA AAATTGCCGA TAAGTATGTC | 15600 |
| CGCAATTTAC AACACAGACT TTATGAGTGT CTCTATAGAA ATAGAGATGT TGACACAGAC | 15660 |
| TTTGTGAATG AGTTTTACGC ATATTTGCGT AAACATTTCT CAATGATGAT ACTCTCTGAC | 15720 |
| GATGCTGTTG TGTGTTTCAA TAGCACTTAT GCATCTCAAG GTCTAGTGGC TAGCATAAAG | 15780 |
| AACTTTAAGT CAGTTCTTTA TTATCAAAAC AATGTTTTTA TGTCTGAAGC AAAATGTTGG | 15840 |
| ACTGAGACTG ACCTTACTAA AGGACCTCAT GAATTTTGCT CTCAACATAC AATGCTAGTT | 15900 |
| AAACAGGGTG ATGATTATGT GTACCTTCCT TACCCAGATC CATCAAGAAT CCTAGGGGCC | 15960 |
| GGCTGTTTTG TAGATGATAT CGTAAAAACA GATGGTACAC TTATGATTGA ACGGTTCGTG | 16020 |
| TCTTTAGCTA TAGATGCTTA CCCACTTACT AAACATCCTA ATCAGGAGTA TGCTGATGTC | 16080 |
| TTTCATTTGT ACTTACAATA CATAAGAAAG CTACATGATG AGTTAACAGG ACACATGTTA | 16140 |
| GACATGTATT CTGTTATGCT TACTAATGAT AACACTTCAA GGTATTGGGA ACCTGAGTTT | 16200 |
| TATGAGGCTA TGTACACACC GCATACAGTC TTACAGGCTG TTGGGGCTTG TGTTCTTTGC | 16260 |
| AATTCACAGA CTTCATTAAG ATGTGGTGCT TGCATACGTA GACCATTCTT ATGTTGTAAA | 16320 |
| TGCTGTTACG ACCATGTCAT ATCAACATCA CATAAATTAG TGTTGTCTGT TAATCCGTAT | 16380 |
| GTTTGCAATG CTCCAGGTTG TGATGTCACA GATGTGACTC AACTTTACTT AGGAGGTATG | 16440 |
| AGCTATTATT GTAAATCACA TAAACCACCC ATTAGTTTTC CATTGTGTGC TAATGGACAA | 16500 |
| GTTTTTGGTT TATATAAAAA TACATGTGTT GGTAGCGATA ATGTTACTGA CTTTAATGCA | 16560 |
| ATTGCAACAT GTGACTGGAC AAATGCTGGT GATTACATTT TAGCTAACAC CTGTACTGAA | 16620 |
| AGACTCAAGC TTTTTGCAGC AGAAACGCTC AAAGCTACTG AGGAGACATT TAAACTGTCT | 16680 |
| TATGGTATTG CTACTGTACG TGAAGTGCTG TCTGACAGAG AATTACATCT TTCATGGGAA | 16740 |
| GTTGGTAAAC CTAGACCACC ACTTAACCGA AATTATGTCT TTACTGGTTA TCGTGTAACT | 16800 |
| AAAAACAGTA AAGTACAAAT AGGAGAGTAC ACCTTTGAAA AAGGTGACTA TGGTGATGCT | 16860 |
| GTTGTTTACC GAGGTACAAC AACTTACAAA TTAAATGTTG GTGATTATTT TGTGCTGACA | 16920 |
| TCACATACAG TAATGCCATT AAGTGCACCT ACACTAGTGC CACAAGAGCA CTATGTTAGA | 16980 |
| ATTACTGGCT TATACCCAAC ACTCAATATC TCAGATGAGT TTTCTAGCAA TGTTGCAAAT | 17040 |
| TATCAAAAGG TTGGTATGCA AAAGTATTCT ACACTCCAGG GACCACCTGG TACTGGTAAG | 17100 |
| AGTCATTTTG CTATTGGCCT AGCTCTCTAC TACCCTTCTG CTCGCATAGT GTATACAGCT | 17160 |
| TGCTCTCATG CCGCTGTTGA TGCACTATGT GAGAAGGCAT TAAAATATTT GCCTATAGAT | 17220 |
| AAATGTAGTA GAATTATACC TGCACGTGCT CGTGTAGAGT GTTTTGATAA ATTCAAAGTG | 17280 |
| AATTCAACAT TAGAACAGTA TGTCTTTTGT ACTGTAAATG CATTGCCTGA GACGACAGCA | 17340 |
| GATATAGTTG TCTTTGATGA AATTTCAATG GCCACAAATT ATGATTTGAG TGTTGTCAAT | 17400 |
| GCCAGATTAC GTGCTAAGCA CTATGTGTAC ATTGGCGACC CTGCTCAATT ACCTGCACCA | 17460 |
| CGCACATTGC TAACTAAGGG CACACTAGAA CCAGAATATT TCAATTCAGT GTGTAGACTT | 17520 |
| ATGAAAACTA TAGGTCCAGA CATGTTCCTC GGAACTTGTC GGCGTTGTCC TGCTGAAATT | 17580 |
| GTTGACACTG TGAGTGCTTT GGTTTATGAT AATAAGCTTA AAGCACATAA AGACAAATCA | 17640 |
| GCTCAATGCT TTAAAATGTT TTATAAGGGT GTTATCACGC ATGATGTTTC ATCTGCAATT | 17700 |
| AACAGGCCAC AAATAGGCGT GGTAAGAGAA TTCCTTACAC GTAACCCTGC TTGGAGAAAA | 17760 |
| GCTGTCTTTA TTTCACCTTA TAATTCACAG AATGCTGTAG CCTCAAAGAT TTTGGGACTA | 17820 |
| CCAACTCAAA CTGTTGATTC ATCACAGGGC TCAGAATATG ACTATGTCAT ATTCACTCAA | 17880 |
| ACCACTGAAA CAGCTCACTC TTGTAATGTA AACAGATTTA ATGTTGCTAT TACCAGAGCA | 17940 |
| AAAGTAGGCA TACTTTGCAT AATGTCTGAT AGAGACCTTT ATGACAAGTT GCAATTTACA | 18000 |
| AGTCTTGAAA TTCCACGTAG GAATGTGGCA ACTTTACAAG CTGAAAATGT AACAGGACTC | 18060 |
| TTTAAAGATT GTAGTAAGGT AATCACTGGG TTACATCCTA CACAGGCACC TACACACCTC | 18120 |
| AGTGTTGACA CTAAATTCAA AACTGAAGGT TTATGTGTTG ACATACCTGG CATACCTAAG | 18180 |
| GACATGACCT ATAGAAGACT CATCTCTATG ATGGGTTTTA AAATGAATTA TCAAGTTAAT | 18240 |
| GGTTACCCTA ACATGTTTAT CACCCGCGAA GAAGCTATAA GACATGTACG TGCATGGATT | 18300 |
| GGCTTCGATG TCGAGGGGTG TCATGCTACT AGAGAAGCTG TTGGTACCAA TTTACCTTTA | 18360 |
| CAGCTAGGTT TTTCTACAGG TGTTAACCTA GTTGCTGTAC CTACAGGTTA TGTTGATACA | 18420 |
| CCTAATAATA CAGATTTTTC CAGAGTTAGT GCTAAACCAC CGCCTGGAGA TCAATTTAAA | 18480 |
| CACCTCATAC CACTTATGTA CAAAGGACTT CCTTGGAATG TAGTGCGTAT AAAGATTGTA | 18540 |
| CAAATGTTAA GTGACACACT TAAAAATCTC TCTGACAGAG TCGTATTTGT CTTATGGGCA | 18600 |
| CATGGCTTTG AGTTGACATC TATGAAGTAT TTTGTGAAAA TAGGACCTGA GCGCACCTGT | 18660 |
| TGTCTATGTG ATAGACGTGC CACATGCTTT TCCACTGCTT CAGACACTTA TGCCTGTTGG | 18720 |
| CATCATTCTA TTGGATTTGA TTACGTCTAT AATCCGTTTA TGATTGATGT TCAACAATGG | 18780 |
| GGTTTTACAG GTAACCTACA AAGCAACCAT GATCTGTATT GTCAAGTCCA TGGTAATGCA | 18840 |
| CATGTAGCTA GTTGTGATGC AATCATGACT AGGTGTCTAG CTGTCCACGA GTGCTTTGTT | 18900 |
| AAGCGTGTTG ACTGGACTAT TGAATATCCT ATAATTGGTG ATGAACTGAA GATTAATGCG | 18960 |
| GCTTGTAGAA AGGTTCAACA CATGGTTGTT AAAGCTGCAT TATTAGCAGA CAAATTCCCA | 19020 |
| GTTCTTCACG ACATTGGTAA CCCTAAAGCT ATTAAGTGTG TACCTCAAGC TGATGTAGAA | 19080 |
| TGGAAGTTCT ATGATGCACA GCCTTGTAGT GACAAAGCTT ATAAAATAGA AGAATTATTC | 19140 |
| TATTCTTATG CCACACATTC TGACAAATTC ACAGATGGTG TATGCCTATT TTGGAATTGC | 19200 |
| AATGTCGATA GATATCCTGC TAATTCCATT GTTTGTAGAT TTGACACTAG AGTGCTATCT | 19260 |
| AACCTTAACT TGCCTGGTTG TGATGGTGGC AGTTTGTATG TAAATAAACA TGCATTCCAC | 19320 |
| ACACCAGCTT TTGATAAAAG TGCTTTTGTT AATTTAAAAC AATTACCATT TTTCTATTAC | 19380 |
| TCTGACAGTC CATGTGAGTC TCATGGAAAA CAAGTAGTGT CAGATATAGA TTATGTACCA | 19440 |
| CTAAAGTCTG CTACGTGTAT AACACGTTGC AATTTAGGTG GTGCTGTCTG TAGACATCAT | 19500 |
| GCTAATGAGT ACAGATTGTA TCTCGATGCT TATAACATGA TGATCTCAGC TGGCTTTAGC | 19560 |
| TTGTGGGTTT ACAAACAATT TGATACTTAT AACCTCTGGA ACACTTTTAC AAGACTTCAG | 19620 |
| AGTTTAGAAA ATGTGGCTTT TAATGTTGTA AATAAGGGAC ACTTTGATGG ACAACAGGGT | 19680 |
| GAAGTACCAG TTTCTATCAT TAATAACACT GTTTACACAA AAGTTGATGG TGTTGATGTA | 19740 |
| GAATTGTTTG AAAATAAAAC AACATTACCT GTTAATGTAG CATTTGAGCT TTGGGCTAAG | 19800 |
| CGCAACATTA AACCAGTACC AGAGGTGAAA ATACTCAATA ATTTGGGTGT GGACATTGCT | 19860 |
| GCTAATACTG TGATCTGGGA CTACAAAAGA GATGCTCCAG CACATATATC TACTATTGGT | 19920 |
| GTTTGTTCTA TGACTGACAT AGCCAAGAAA CCAACTGAAA CGATTTGTGC ACCACTCACT | 19980 |
| GTCTTTTTTG ATGGTAGAGT TGATGGTCAA GTAGACTTAT TTAGAAATGC CCGTAATGGT | 20040 |
| GTTCTTATTA CAGAAGGTAG TGTTAAAGGT TTACAACCAT CTGTAGGTCC CAAACAAGCT | 20100 |
| AGTCTTAATG GAGTCACATT AATTGGAGAA GCCGTAAAAA CACAGTTCAA TTATTATAAG | 20160 |
| AAAGTTGATG GTGTTGTCCA ACAATTACCT GAAACTTACT TTACTCAGAG TAGAAATTTA | 20220 |
| CAAGAATTTA AACCCAGGAG TCAAATGGAA ATTGATTTCT TAGAATTAGC TATGGATGAA | 20280 |
| TTCATTGAAC GGTATAAATT AGAAGGCTAT GCCTTCGAAC ATATCGTTTA TGGAGATTTT | 20340 |
| AGTCATAGTC AGTTAGGTGG TTTACATCTA CTGATTGGAC TAGCTAAACG TTTTAAGGAA | 20400 |
| TCACCTTTTG AATTAGAAGA TTTTATTCCT ATGGACAGTA CAGTTAAAAA CTATTTCATA | 20460 |
| ACAGATGCGC AAACAGGTTC ATCTAAGTGT GTGTGTTCTG TTATTGATTT ATTACTTGAT | 20520 |
| GATTTTGTTG AAATAATAAA ATCCCAAGAT TTATCTGTAG TTTCTAAGGT TGTCAAAGTG | 20580 |
| ACTATTGACT ATACAGAAAT TTCATTTATG CTTTGGTGTA AAGATGGCCA TGTAGAAACA | 20640 |
| TTTTACCCAA AATTACAATC TAGTCAAGCG TGGCAACCGG GTGTTGCTAT GCCTAATCTT | 20700 |
| TACAAAATGC AAAGAATGCT ATTAGAAAAG TGTGACCTTC AAAATTATGG TGATAGTGCA | 20760 |
| ACATTACCTA AAGGCATAAT GATGAATGTC GCAAAATATA CTCAACTGTG TCAATATTTA | 20820 |
| AACACATTAA CATTAGCTGT ACCCTATAAT ATGAGAGTTA TACATTTTGG TGCTGGTTCT | 20880 |
| GATAAAGGAG TTGCACCAGG TACAGCTGTT TTAAGACAGT GGTTGCCTAC GGGTACGCTG | 20940 |
| CTTGTCGATT CAGATCTTAA TGACTTTGTC TCTGATGCAG ATTCAACTTT GATTGGTGAT | 21000 |
| TGTGCAACTG TACATACAGC TAATAAATGG GATCTCATTA TTAGTGATAT GTACGACCCT | 21060 |
| AAGACTAAAA ATGTTACAAA AGAAAATGAC TCTAAAGAGG GTTTTTTCAC TTACATTTGT | 21120 |
| GGGTTTATAC AACAAAAGCT AGCTCTTGGA GGTTCCGTGG CTATAAAGAT AACAGAACAT | 21180 |
| TCTTGGAATG CTGATCTTTA TAAGCTCATG GGACACTTCG CATGGTGGAC AGCCTTTGTT | 21240 |
| ACTAATGTGA ATGCGTCATC ATCTGAAGCA TTTTTAATTG GATGTAATTA TCTTGGCAAA | 21300 |
| CCACGCGAAC AAATAGATGG TTATGTCATG CATGCAAATT ACATATTTTG GAGGAATACA | 21360 |
| AATCCAATTC AGTTGTCTTC CTATTCTTTA TTTGACATGA GTAAATTTCC CCTTAAATTA | 21420 |
| AGGGGTACTG CTGTTATGTC TTTAAAAGAA GGTCAAATCA ATGATATGAT TTTATCTCTT | 21480 |
| CTTAGTAAAG GTAGACTTAT AATTAGAGAA AACAACAGAG TTGTTATTTC TAGTGATGTT | 21540 |
| CTTGTTAACA ACTAAACGAA CAATGTTTGT TTTTCTTGTT TTATTGCCAC TAGTCTCTAG | 21600 |
| TCAGTGTGTT AATCTTACAA CCAGAACTCA ATTACCCCCT GCATACACTA ATTCTTTCAC | 21660 |
| ACGTGGTGTT TATTACCCTG ACAAAGTTTT CAGATCCTCA GTTTTACATT CAACTCAGGA | 21720 |
| CTTGTTCTTA CCTTTCTTTT CCAATGTTAC TTGGTTCCAT GCTATACATG TCTCTGGGAC | 21780 |
| CAATGGTACT AAGAGGTTTG ATAACCCTGT CCTACCATTT AATGATGGTG TTTATTTTGC | 21840 |
| TTCCACTGAG AAGTCTAACA TAATAAGAGG CTGGATTTTT GGTACTACTT TAGATTCGAA | 21900 |
| GACCCAGTCC CTACTTATTG TTAATAACGC TACTAATGTT GTTATTAAAG TCTGTGAATT | 21960 |
| TCAATTTTGT AATGATCCAT TTTTGGGTGT TTATTACCAC AAAAACAACA AAAGTTGGAT | 22020 |
| GGAAAGTGAG TTCAGAGTTT ATTCTAGTGC GAATAATTGC ACTTTTGAAT ATGTCTCTCA | 22080 |
| GCCTTTTCTT ATGGACCTTG AAGGAAAACA GGGTAATTTC AAAAATCTTA GGGAATTTGT | 22140 |
| GTTTAAGAAT ATTGATGGTT ATTTTAAAAT ATATTCTAAG CACACGCCTA TTAATTTAGT | 22200 |
| GCGTGATCTC CCTCAGGGTT TTTCGGCTTT AGAACCATTG GTAGATTTGC CAATAGGTAT | 22260 |
| TAACATCACT AGGTTTCAAA CTTTACTTGC TTTACATAGA AGTTATTTGA CTCCTGGTGA | 22320 |
| TTCTTCTTCA GGTTGGACAG CTGGTGCTGC AGCTTATTAT GTGGGTTATC TTCAACCTAG | 22380 |
| GACTTTTCTA TTAAAATATA ATGAAAATGG AACCATTACA GATGCTGTAG ACTGTGCACT | 22440 |
| TGACCCTCTC TCAGAAACAA AGTGTACGTT GAAATCCTTC ACTGTAGAAA AAGGAATCTA | 22500 |
| TCAAACTTCT AACTTTAGAG TCCAACCAAC AGAATCTATT GTTAGATTTC CTAATATTAC | 22560 |
| AAACTTGTGC CCTTTTGGTG AAGTTTTTAA CGCCACCAGA TTTGCATCTG TTTATGCTTG | 22620 |
| GAACAGGAAG AGAATCAGCA ACTGTGTTGC TGATTATTCT GTCCTATATA ATTCCGCATC | 22680 |
| ATTTTCCACT TTTAAGTGTT ATGGAGTGTC TCCTACTAAA TTAAATGATC TCTGCTTTAC | 22740 |
| TAATGTCTAT GCAGATTCAT TTGTAATTAG AGGTGATGAA GTCAGACAAA TCGCTCCAGG | 22800 |
| GCAAACTGGA AAGATTGCTG ATTATAATTA TAAATTACCA GATGATTTTA CAGGCTGCGT | 22860 |
| TATAGCTTGG AATTCTAACA ATCTTGATTC TAAGGTTGGT GGTAATTATA ATTACCTGTA | 22920 |
| TAGATTGTTT AGGAAGTCTA ATCTCAAACC TTTTGAGAGA GATATTTCAA CTGAAATCTA | 22980 |
| TCAGGCCGGT AGCACACCTT GTAATGGTGT TGAAGGTTTT AATTGTTACT TTCCTTTACA | 23040 |
| ATCATATGGT TTCCAACCCA CTAATGGTGT TGGTTACCAA CCATACAGAG TAGTAGTACT | 23100 |
| TTCTTTTGAA CTTCTACATG CACCAGCAAC TGTTTGTGGA CCTAAAAAGT CTACTAATTT | 23160 |
| GGTTAAAAAC AAATGTGTCA ATTTCAACTT CAATGGTTTA ACAGGCACAG GTGTTCTTAC | 23220 |
| TGAGTCTAAC AAAAAGTTTC TGCCTTTCCA ACAATTTGGC AGAGACATTG CTGACACTAC | 23280 |
| TGATGCTGTC CGTGATCCAC AGACACTTGA GATTCTTGAC ATTACACCAT GTTCTTTTGG | 23340 |
| TGGTGTCAGT GTTATAACAC CAGGAACAAA TACTTCTAAC CAGGTTGCTG TTCTTTATCA | 23400 |
| GGATGTTAAC TGCACAGAAG TCCCTGTTGC TATTCATGCA GATCAACTTA CTCCTACTTG | 23460 |
| GCGTGTTTAT TCTACAGGTT CTAATGTTTT TCAAACACGT GCAGGCTGTT TAATAGGGGC | 23520 |
| TGAACATGTC AACAACTCAT ATGAGTGTGA CATACCCATT GGTGCAGGTA TATGCGCTAG | 23580 |
| TTATCAGACT CAGACTAATT CTCCTCGGCG GGCACGTAGT GTAGCTAGTC AATCCATCAT | 23640 |
| TGCCTACACT ATGTCACTTG GTGCAGAAAA TTCAGTTGCT TACTCTAATA ACTCTATTGC | 23700 |
| CATACCCACA AATTTTACTA TTAGTGTTAC CACAGAAATT CTACCAGTGT CTATGACCAA | 23760 |
| GACATCAGTA GATTGTACAA TGTACATTTG TGGTGATTCA ACTGAATGCA GCAATCTTTT | 23820 |
| GTTGCAATAT GGCAGTTTTT GTACACAATT AAACCGTGCT TTAACTGGAA TAGCTGTTGA | 23880 |
| ACAAGACAAA AACACCCAAG AAGTTTTTGC ACAAGTCAAA CAAATTTACA AAACACCACC | 23940 |
| AATTAAAGAT TTTGGTGGTT TTAATTTTTC ACAAATATTA CCAGATCCAT CAAAACCAAG | 24000 |
| CAAGAGGTCA TTTATTGAAG ATCTACTTTT CAACAAAGTG ACACTTGCAG ATGCTGGCTT | 24060 |
| CATCAAACAA TATGGTGATT GCCTTGGTGA TATTGCTGCT AGAGACCTCA TTTGTGCACA | 24120 |
| AAAGTTTAAC GGCCTTACTG TTTTGCCACC TTTGCTCACA GATGAAATGA TTGCTCAATA | 24180 |
| CACTTCTGCA CTGTTAGCGG GTACAATCAC TTCTGGTTGG ACCTTTGGTG CAGGTGCTGC | 24240 |
| ATTACAAATA CCATTTGCTA TGCAAATGGC TTATAGGTTT AATGGTATTG GAGTTACACA | 24300 |
| GAATGTTCTC TATGAGAACC AAAAATTGAT TGCCAACCAA TTTAATAGTG CTATTGGCAA | 24360 |
| AATTCAAGAC TCACTTTCTT CCACAGCAAG TGCACTTGGA AAACTTCAAG ATGTGGTCAA | 24420 |
| CCAAAATGCA CAAGCTTTAA ACACGCTTGT TAAACAACTT AGCTCCAATT TTGGTGCAAT | 24480 |
| TTCAAGTGTT TTAAATGATA TCCTTTCACG TCTTGACAAA GTTGAGGCTG AAGTGCAAAT | 24540 |
| TGATAGGTTG ATCACAGGCA GACTTCAAAG TTTGCAGACA TATGTGACTC AACAATTAAT | 24600 |
| TAGAGCTGCA GAAATCAGAG CTTCTGCTAA TCTTGCTGCT ACTAAAATGT CAGAGTGTGT | 24660 |
| ACTTGGACAA TCAAAAAGAG TTGATTTTTG TGGAAAGGGC TATCATCTTA TGTCCTTCCC | 24720 |
| TCAGTCAGCA CCTCATGGTG TAGTCTTCTT GCATGTGACT TATGTCCCTG CACAAGAAAA | 24780 |
| GAACTTCACA ACTGCTCCTG CCATTTGTCA TGATGGAAAA GCACACTTTC CTCGTGAAGG | 24840 |
| TGTCTTTGTT TCAAATGGCA CACACTGGTT TGTAACACAA AGGAATTTTT ATGAACCACA | 24900 |
| AATCATTACT ACAGACAACA CATTTGTGTC TGGTAACTGT GATGTTGTAA TAGGAATTGT | 24960 |
| CAACAACACA GTTTATGATC CTTTGCAACC TGAATTAGAC TCATTCAAGG AGGAGTTAGA | 25020 |
| TAAATATTTT AAGAATCATA CATCACCAGA TGTTGATTTA GGTGACATCT CTGGCATTAA | 25080 |
| TGCTTCAGTT GTAAACATTC AAAAAGAAAT TGACCGCCTC AATGAGGTTG CCAAGAATTT | 25140 |
| AAATGAATCT CTCATCGATC TCCAAGAACT TGGAAAGTAT GAGCAGTATA TAAAATGGCC | 25200 |
| ATGGTACATT TGGCTAGGTT TTATAGCTGG CTTGATTGCC ATAGTAATGG TGACAATTAT | 25260 |
| GCTTTGCTGT ATGACCAGTT GCTGTAGTTG TCTCAAGGGC TGTTGTTCTT GTGGATCCTG | 25320 |
| CTGCAAATTT GATGAAGACG ACTCTGAGCC AGTGCTCAAA GGAGTCAAAT TACATTACAC | 25380 |
| ATAAACGAAC TTATGGATTT GTTTATGAGA ATCTTCACAA TTGGAACTGT AACTTTGAAG | 25440 |
| CAAGGTGAAA TCAAGGATGC TACTCCTTCA GATTTTGTTC GCGCTACTGC AACGATACCG | 25500 |
| ATACAAGCCT CACTCCCTTT CGGATGGCTT ATTGTTGGCG TTGCACTTCT TGCTGTTTTT | 25560 |
| CAGAGCGCTT CCAAAATCAT AACCCTCAAA AAGAGATGGC AACTAGCACT CTCCAAGGGT | 25620 |
| GTTCACTTTG TTTGCAACTT GCTGTTGTTG TTTGTAACAG TTTACTCACA CCTTTTGCTC | 25680 |
| GTTGCTGCTG GCCTTGAAGC CCCTTTTCTC TATCTTTATG CTTTAGTCTA CTTCTTGCAG | 25740 |
| AGTATAAACT TTGTAAGAAT AATAATGAGG CTTTGGCTTT GCTGGAAATG CCGTTCCAAA | 25800 |
| AACCCATTAC TTTATGATGC CAACTATTTT CTTTGCTGGC ATACTAATTG TTACGACTAT | 25860 |
| TGTATACCTT ACAATAGTGT AACTTCTTCA ATTGTCATTA CTTCAGGTGA TGGCACAACA | 25920 |
| AGTCCTATTT CTGAACATGA CTACCAGATT GGTGGTTATA CTGAAAAATG GGAATCTGGA | 25980 |
| GTAAAAGACT GTGTTGTATT ACACAGTTAC TTCACTTCAG ACTATTACCA GCTGTACTCA | 26040 |
| ACTCAATTGA GTACAGACAC TGGTGTTGAA CATGTTACCT TCTTCATCTA CAATAAAATT | 26100 |
| GTTGATGAGC CTGAAGAACA TGTCCAAATT CACACAATCG ACGGTTCATC CGGAGTTGTT | 26160 |
| AATCCAGTAA TGGAACCAAT TTATGATGAA CCGACGACGA CTACTAGCGT GCCTTTGTAA | 26220 |
| GCACAAGCTG ATGAGTACGA ACTTATGTAC TCATTCGTTT CGGAAGAGAC AGGTACGTTA | 26280 |
| ATAGTTAATA GCGTACTTCT TTTTCTTGCT TTCGTGGTAT TCTTGCTAGT TACACTAGCC | 26340 |
| ATCCTTACTG CGCTTCGATT GTGTGCGTAC TGCTGCAATA TTGTTAACGT GAGTCTTGTA | 26400 |
| AAACCTTCTT TTTACGTTTA CTCTCGTGTT AAAAATCTGA ATTCTTCTAG AGTTCCTGAT | 26460 |
| CTTCTGGTCT AAACGAACTA AATATTATAT TAGTTTTTCT GTTTGGAACT TTAATTTTAG | 26520 |
| CCATGGCAGA TTCCAACGGT ACTATTACCG TTGAAGAGCT TAAAAAGCTC CTTGAACAAT | 26580 |
| GGAACCTAGT AATAGGTTTC CTATTCCTTA CATGGATTTG TCTTCTACAA TTTGCCTATG | 26640 |
| CCAACAGGAA TAGGTTTTTG TATATAATTA AGTTAATTTT CCTCTGGCTG TTATGGCCAG | 26700 |
| TAACTTTAGC TTGTTTTGTG GTTGCTGCTG TTTACAGAAT AAATTGGATC ACCGGTGGAA | 26760 |
| TTGCTATCGC AATGGCTTGT CTTGTAGGCT TGATGTGGCT CAGCTACTTC ATTGCTTCTT | 26820 |
| TCAGACTGTT TGCGCGTACG CGTTCCATGT GGTCATTCAA TCCAGAAACT AACATTCTTC | 26880 |
| TCAACGTGCC ACTCCATGGC ACTATTCTGA CCAGACCGCT TCTAGAAAGT GAACTCGTAA | 26940 |
| TCGGAGCTGT GATCCTTCGT GGACATCTTC GTATTGCTGG ACACCATCTA GGACGCTGTG | 27000 |
| ACATCAAGGA CCTGCCTAAA GAAATCACTG TTGCTACATC ACGAACGCTT TCTTATTACA | 27060 |
| AATTGGGAGC TTCGCAGCGT GTAGCAGGTG ACTCAGGTTT TGCTGCATAC AGTCGCTACA | 27120 |
| GGATTGGCAA CTATAAATTA AACACAGACC ATTCCAGTAG CAGTGACAAT ATTGCTTTGC | 27180 |
| TTGTACAGTA AGTGACAACA GATGTTTCAT CTCGTTGACT TTCAGGTTAC TATAGCAGAG | 27240 |
| ATATTACTAA TTATTATGAG GACTTTTAAA GTTTCCATTT GGAATCTTGA TTACATCATA | 27300 |
| AACCTCATAA TTAAAAATTT ATCTAAGTCA CTAACTGAGA ATAAATATTC TCAATTAGAT | 27360 |
| GAAGAGCAAC CAATGGAGAT TGATTAAACG AACATGAAAA TTATTCTTTT CTTGGCACTG | 27420 |
| ATAACACTCG CTACTTGTGA GCTTTATCAC TACCAAGAGT GTGTTAGAGG TACAACAGTA | 27480 |
| CTTTTAAAAG AACCTTGCTC TTCTGGAACA TACGAGGGCA ATTCACCATT TCATCCTCTA | 27540 |
| GCTGATAACA AATTTGCACT GACTTGCTTT AGCACTCAAT TTGCTTTTGC TTGTCCTGAC | 27600 |
| GGCGTAAAAC ACGTCTATCA GTTACGTGCC AGATCAGTTT CACCTAAACT GTTCATCAGA | 27660 |
| CAAGAGGAAG TTCAAGAACT TTACTCTCCA ATTTTTCTTA TTGTTGCGGC AATAGTGTTT | 27720 |
| ATAACACTTT GCTTCACACT CAAAAGAAAG ACAGAATGAT TGAACTTTCA TTAATTGACT | 27780 |
| TCTATTTGTG CTTTTTAGCC TTTCTGCTAT TCCTTGTTTT AATTATGCTT ATTATCTTTT | 27840 |
| GGTTCTCACT TGAACTGCAA GATCATAATG AAACTTGTCA CGCCTAAACG AACATGAAAT | 27900 |
| TTCTTGTTTT CTTAGGAATC ATCACAACTG TAGCTGCATT TCACCAAGAA TGTAGTTTAC | 27960 |
| AGTCATGTAC TCAACATCAA CCATATGTAG TTGATGACCC GTGTCCTATT CACTTCTATT | 28020 |
| CTAAATGGTA TATTAGAGTA GGAGCTAGAA AATCAGCACC TTTAATTGAA TTGTGCGTGG | 28080 |
| ATGAGGCTGG TTCTAAATCA CCCATTCAGT ACATCGATAT CGGTAATTAT ACAGTTTCCT | 28140 |
| GTTTACCTTT TACAATTAAT TGCCAGGAAC CTAAATTGGG TAGTCTTGTA GTGCGTTGTT | 28200 |
| CGTTCTATGA AGACTTTTTA GAGTATCATG ACGTTCGTGT TGTTTTAGAT TTCATCTAAA | 28260 |
| CGAACAAACT AAAATGTCTG ATAATGGACC CCAAAATCAG CGAAATGCAC CCCGCATTAC | 28320 |
| GTTTGGTGGA CCCTCAGATT CAACTGGCAG TAACCAGAAT GGAGAACGCA GTGGGGCGCG | 28380 |
| ATCAAAACAA CGTCGGCCCC AAGGTTTACC CAATAATACT GCGTCTTGGT TCACCGCTCT | 28440 |
| CACTCAACAT GGCAAGGAAG ACCTTAAATT CCCTCGAGGA CAAGGCGTTC CAATTAACAC | 28500 |
| CAATAGCAGT CCAGATGACC AAATTGGCTA CTACCGAAGA GCTACCAGAC GAATTCGTGG | 28560 |
| TGGTGACGGT AAAATGAAAG ATCTCAGTCC AAGATGGTAT TTCTACTACC TAGGAACTGG | 28620 |
| GCCAGAAGCT GGACTTCCCT ATGGTGCTAA CAAAGACGGC ATCATATGGG TTGCAACTGA | 28680 |
| GGGAGCCTTG AATACACCAA AAGATCACAT TGGCACCCGC AATCGTGCTA ACAATGCTGC | 28740 |
| AATCGTGCTA CAACTTCCTC AAGGAACAAC ATTGCCAAAA GGCTTCTACG CAGAAGGGAG | 28800 |
| CAGAGGCGGC AGTCAAGCCT CTTCTCGTTC CTCATCACGT AGTCGCAACA GTTCAAGAAA | 28860 |
| TTCAACTCCA GGCAGCAGTA GGGGAACTTC TCCTGCTAGA ATGGCTGGCA ATGGCGGTGA | 28920 |
| TGCTGCTCTT GCTTTGCTGC TGCTTGACAG ATTGAACCAG CTTGAGAGCA AAATGTCTGG | 28980 |
| TAAAGGCCAA CAACAACAAG GCCAAACTGT CACTAAGAAA TCTGCTGCTG AGGCTTCTAA | 29040 |
| GAAGCCTCGG CAAAAACGTA CTGCCACTAA AGCATACAAT GTAACACAAG CTTTCGGCAG | 29100 |
| ACGTGGTCCA GAACAAACCC AAGGAAATTT TGGGGACCAG GAACTAATCA GACAAGGAAC | 29160 |
| TGATTACAAA CATTGGCCGC AAATTGCACA ATTTGCCCCC AGCGCTTCAG CGTTCTTCGG | 29220 |
| AATGTCGCGC ATTGGCATGG AAGTCACACC TTCGGGAACG TGGTTGACCT ACACAGGTGC | 29280 |
| CATCAAATTG GATGACAAAG ATCCAAATTT CAAAGATCAA GTCATTTTGC TGAATAAGCA | 29340 |
| TATTGACGCA TACAAAACAT TCCCACCAAC AGAGCCTAAA AAGGACAAAA AGAAGAAGGC | 29400 |
| TGATGAAACT CAAGCCTTAC CGCAGAGACA GAAGAAACAG CAAACTGTGA CTCTTCTTCC | 29460 |
| TGCTGCAGAT TTGGATGATT TCTCCAAACA ATTGCAACAA TCCATGAGCA GTGCTGACTC | 29520 |
| AACTCAGGCC TAAACTCATG CAGACCACAC AAGGCAGATG GGCTATATAA ACGTTTTCGC | 29580 |
| TTTTCCGTTT ACGATATATA GTCTACTCTT GTGCAGAATG AATTCTCGTA ACTACATAGC | 29640 |
| ACAAGTAGAT GTAGTTAACT TTAATCTCAC ATAGCAATCT TTAATCAGTG TGTAACATTA | 29700 |
| GGGAGGACTT GAAAGAGCCA CCACATTTTC ACCGAGGCCA CGCGGAGTAC GATCGAGTGT | 29760 |
| ACAGTGAACA ATGCTAGGGA GAGCTGCCTA TATGGAAGAG CCCTAATGTG TAAAATTAAT | 29820 |
| TTTAGTAGTG CTATCCCCAT GTGATTTTAA TAGCTTCTTA GGAGAATGAC AAAAAAAAAA | 29880 |
| AAAAAAAAAA AAAAAAAAAA AAA | 29903 |

```
SEQ ID NO: 2-a wild type amino acid sequence of Spike (3) protein of Severe
Acute Respiratory Syndrome Coronavirus 2 (SARS-CoV-2) (Wu et al. 2020 Nature
579:265-269; GenBank Accession QHD43416.1 entitled ″Surface Glycoprotein
[Severe Acute Respiratory Syndrome Coronavirus 2]″-encoded by nucleotides
21563-25384 of SEQ ID NO: 1) having the features N'-C' as follows (see also
Wrapp et al. 2020 Science 367(6483):1260-1263 and Supplementary Materials as
well as corresponding Protein Data Bank (PDB) accession 6VSB version 1.4
entitled ″Prefusion 2019-nCoV spike glycoprotein with a single receptor-
binding domain up″; UniProtKB Accession PODTC2 version 1 dated 22April2020):
Signal peptide residues 1-15 (underlined)
N-Terminal Domain (NTD) residues V16-S305 (double underlined)
Receptor Binding Domain (RBD) residues P330 to P521 (underlined)
Residue D614 (underlined)
Furin Recognition Site (FRS or 31/32 protease cleavage site) residues R682,
R683, A684, and R685 (underlined)
Fusion Peptide (FP) residues S816 to F833 (underlined)
Heptad Repeat 1 (HR1) residues G908 to D985 (double underlined)
Central Helix (CH) residues K986 to G1035 (underlined)
Connector Domain (CD) residues T1076 to L1141 (underlined)
                  10 20 30 40 50 60
MFVFLVLLPL VSSQCVNLTT RTQLPPAYTN SFTRGVYYPD KVFRSSVLHS TQDLFLPFFS
                  70 80 90 100 110 120
NVTWFHAIHV SGTNGTKRFD NPVLPFNDGV YFASTEKSNI IRGWIFGTTL DSKTQSLLIV
                  130 140 150 160 170 180
NNATNVVIKV CEFQFCNDPF LGVYYHKNNK SWMESEFRVY SSANNCTFEY VSQPFLMDLE
                  190 200 210 220 230 240
GKQGNFKNLR EFVFKNIDGY FKIYSKHTPI NLVRDLPQGF SALEPLVDLP IGINITRFQT
                  250 260 270 280 290 300
LLALHRSYLT PGDSSSGWTA GAAAYYVGYL QPRTFLLKYN ENGTITDAVD CALDPLSETK
                  310 320 330 340 350 360
CTLKSFTVEK GIYQTSNFRV QPTESIVRFP NITNLCPFGE VFNATRFASV YAWNRKRISN
                  370 380 390 400 410 420
CVADYSVLYN SASFSTFKCY GVSPTKLNDL CFTNVYADSF VIRGDEVRQI APGQTGKIAD
                  430 440 450 460 470 480
YNYKLPDDFT GCVIAWNSNN LDSKVGGNYN YLYRLFRKSN LKPFERDIST EIYQAGSTPC
                  490 500 510 520 530 540
NGVEGFNCYF PLQSYGFQPT NGVGYQPYRV VVLSFELLHA PATVCGPKKS TNLVKNKCVN
                  550 560 570 580 590 600
FNFNGLTGTG VLTESNKKFL PFQQFGRDIA DTTDAVRDPQ TLEILDITPC SFGGVSVITP
                  610 620 630 640 650 660
GTNTSNQVAV LYQDVNCTEV PVAIHADQLT PTWRVYSTGS NVFQTRAGCL IGAEHVNNSY
                  670 680 690 700 710 720
ECDIPIGAGI CASYQTQTNS PRRARSVASQ SIIAYTMSLG AENSVAYSNN SIAIPTNFTI
                  730 740 750 760 770 780
SVTTEILPVS MTKTSVDCTM YICGDSTECS NLLLQYGSFC TQLNRALTGI AVEQDKNTQE
                  790 800 810 820 830 840
VFAQVKQIYK TPPIKDFGGF NFSQILPDPS KPSKRSFIED LLFNKVTLAD AGFIKQYGDC
                  850 860 870 880 890 900
LGDIAARDLI CAQKFNGLTV LPPLLTDEMI AQYTSALLAG TITSGWTFGA GAALQIPFAM
                  910 920 930 940 950 960
QMAYRFNGIG VTQNVLYENQ KLIANQFNSA IGKIQDSLSS TASALGKLQD VVNQNAQALN
                  970 980 990 1000 1010 1020
TLVKQLSSNF GAISSVLNDI LSRLDKVEAE VQIDRLITGR LQSLQTYVTQ QLIRAAEIRA
                1030 1040 1050 1060 1070 1080
SANLAATKMS ECVLGQSKRV DFCGKGYHLM SFPQSAPHGV VFLHVTYVPA QEKNFTTAPA
                1090 1100 1110 1120 1130 1140
ICHDGKAHFP REGVFVSNGT HWFVTQRNFY EPQIITTDNT FVSGNCDVVI GIVNNTVYDP
                1150 1160 1170 1180 1190 1200
LQPELDSFKE ELDKYFKNHT SPDVDLGDIS GINASVVNIQ KEIDRLNEVA KNLNESLIDL
                1210 1220 1230 1240 1250 1260
QELGKYEQYI KWPWYIWLGF IAGLIAIVMV TIMLCCMTSC CSCLKGCCSC GSCCKFDEDD
                1270 1273
SEPVLKGVKL HYT
SEQ ID NO: 3-residues 27-1208 of the Spike (S) protein amino acid sequence
SEQ ID NO: 2 having the features N'-C' as follows:
A subsequence of the N-Terminal Domain (NTD) , here as residues A1-S279
(double underlined)
Receptor Binding Domain (RBD) residues P304 to P495 (underlined)
Residue D588 (underlined)
Furin Recognition Site (FRS or S1/S2 protease cleavage site) residues R656,
R657, A658, and R659 (underlined)
Fusion Peptide (FP) residues S790 to F807 (underlined)
Heptad Repeat 1 (HR1) residues G882 to D959 (double underlined)
Central Helix (CH) residues K960 to G1009 (underlined)
Connector Domain (CD) residues T1050 to L1115 (underlined)
                  10 20 30 40 50 60
AYTNSFTRGV YYPDKVFRSS VLHSTQDLFL PFFSNVTWFH AIHVSGTNGT KRFDNPVLPF
                  70 80 90 100 110 120
NDGVYFASTE KSNIIRGWIF GTTLDSKTQS LLIVNNATNV VIKVCEFQFC NDPFLGVYYH
                  130 140 150 160 170 180
KNNKSWMESE FRVYSSANNC TFEYVSQPFL MDLEGKQGNF KNLREFVFKN IDGYFKIYSK
                  190 200 210 220 230 240
HTPINLVRDL PQGFSALEPL VDLPIGINIT RFQTLLALHR SYLTPGDSSS GWTAGAAAYY
                  250 260 270 280 290 300
VGYLQPRTFL LKYNENGTIT DAVDCALDPL SETKCTLKSF TVEKGIYQTS NFRVQPTESI
                  310 320 330 340 350 360
VRFPNITNLC PFGEVFNATR FASVYAWNRK RISNCVADYS VLYNSASFST FKCYGVSPTK
                  370 380 390 400 410 420
LNDLCFTNVY ADSFVIRGDE VRQIAPGQTG KIADYNYKLP DDFTGCVIAW NSNNLDSKVG
                  430 440 450 460 470 480
GNYNYLYRLF RKSNLKPFER DISTEIYQAG STPCNGVEGF NCYFPLQSYG FQPTNGVGYQ
                  490 500 510 520 530 540
PYRVVVLSFE LLHAPATVCG PKKSTNLVKN KCVNFNFNGL TGTGVLTESN KKFLPFQQFG
                  550 560 570 580 590 600
RDIADTTDAV RDPQTLEILD ITPCSFGGVS VITPGTNTSN QVAVLYQDVN CTEVPVAIHA
                  610 620 630 640 650 660
DQLTPTWRVY STGSNVFQTR AGCLIGAEHV NNSYECDIPI GAGICASYQT QTNSPRRARS
                  670 680 690 700 710 720
VASQSIIAYT MSLGAENSVA YSNNSIAIPT NFTISVTTEI LPVSMTKTSV DCTMYICGDS
                  730 740 750 760 770 780
TECSNLLLQY GSFCTQLNRA LTGIAVEQDK NTQEVFAQVK QIYKTPPIKD FGGFNFSQIL
                  790 800 810 820 830 840
PDPSKPSKRS FIEDLLFNKV TLADAGFIKQ YGDCLGDIAA RDLICAQKFN GLTVLPPLLT
                  850 860 870 880 890 900
DEMIAQYTSA LLAGTITSGW TFGAGAALQI PFAMQMAYRF NGIGVTQNVL YENQKLIANQ
                  910 920 930 940 950 960
FNSAIGKIQD SLSSTASALG KLQDVVNQNA QALNTLVKQL SSNFGAISSV LNDILSRLDK
                  970 980 990 1000 1010 1020
VEAEVQIDRL ITGRLQSLQT YVTQQLIRAA EIRASANLAA TKMSECVLGQ SKRVDFCGKG
                  1030 1040 1050 1060 1070 1080
YHLMSFPQSA PHGVVFLHVT YVPAQEKNFT TAPAICHDGK AHFPREGVFV SNGTHWFVTQ
                  1090 1100 1110 1120 1121
RNFYEPQIIT TDNTFVSGNC DVVIGIVNNT VYDPLQPELD S
SEQ ID NO: 4-mutant Spike (S) protein amino acid sequence having the
features N'-C' (as compared to SEQ ID NO: 3) as follows (see Brufsky
20April2020 J Med Virol, 7 pages, doi:10.1002/jmv.25902 and Korber et al.
2020 bioRxiv (HyperTextTransferProtocolsecure:
//doi.org/10.1101/2020.04.29.069054); Wrapp et al. 2020 Science
367 (6483):1260-1263 and Supplementary Materials as well as corresponding
Protein Data Bank (PDB) accession 6VSB version 1.4 entitled ″Prefusion 2019-
nCoV spike glycoprotein with a single receptor-binding domain up″):
D588G substitution (underlined) site
R656G,R657S, and R659S Substitutions at the furin recognition
(underlined)
K960P and V961P substitutions at the Central Helix (CH) (underlined)
                  10 20 30 40 50 60
AYTNSFTRGV YYPDKVFRSS VLHSTQDLFL PFFSNVTWFH AIHVSGTNGT KRFDNPVLPF
                  70 80 90 100 110 120
NDGVYFASTE KSNIIRGWIF GTTLDSKTQS LLIVNNATNV VIKVCEFQFC NDPFLGVYYH
                  130 140 150 160 170 180
KNNKSWMESE FRVYSSANNC TFEYVSQPFL MDLEGKQGNF KNLREFVFKN IDGYFKIYSK
                  190 200 210 220 230 240
HTPINLVRDL PQGFSALEPL VDLPIGINIT RFQTLLALHR SYLTPGDSSS GWTAGAAAYY
                  250 260 270 280 290 300
VGYLQPRTFL LKYNENGTIT DAVDCALDPL SETKCTLKSF TVEKGIYQTS NFRVQPTESI
                  310 320 330 340 350 360
VRFPNITNLC PFGEVFNATR FASVYAWNRK RISNCVADYS VLYNSASFST FKCYGVSPTK
                  370 380 390 400 410 420
LNDLCFTNVY ADSFVIRGDE VRQIAPGQTG KIADYNYKLP DDFTGCVIAW NSNNLDSKVG
                  430 440 450 460 470 480
GNYNYLYRLF RKSNLKPFER DISTEIYQAG STPCNGVEGF NCYFPLQSYG FQPTNGVGYQ
                  490 500 510 520 530 540
PYRVVVLSFE LLHAPATVCG PKKSTNLVKN KCVNFNFNGL TGTGVLTESN KKFLPFQQFG
                  550 560 570 580 590 600
RDIADTTDAV RDPQTLEILD ITPCSFGGVS VITPGTNTSN QVAVLYOGVN CTEVPVAIHA
                  610 620 630 640 650 660
DQLTPTWRVY STGSNVFQTR AGCLIGAEHV NNSYECDIPI GAGICASYQT QTNSPGSASS
                  670 680 690 700 710 720
VASQSIIAYT MSLGAENSVA YSNNSIAIPT NFTISVTTEI LPVSMTKTSV DCTMYICGDS
                  730 740 750 760 770 780
TECSNLLLQY GSFCTQLNRA LTGIAVEQDK NTQEVFAQVK QIYKTPPIKD FGGFNFSQIL
                  790 800 810 820 830 840
PDPSKPSKRS FIEDLLFNKV TLADAGFIKQ YGDCLGDIAA RDLICAQKFN GLTVLPPLLT
                  850 860 870 880 890 900
DEMIAQYTSA LLAGTITSGW TFGAGAALQI PFAMQMAYRF NGIGVTQNVL YENQKLIANQ
                  910 920 930 940 950 960
FNSAIGKIQD SLSSTASALG KLQDVVNQNA QALNTLVKQL SSNFGAISSV LNDILSRLDP
                  970 980 990 1000 1010 1020
PEAEVQIDRL ITGRLQSLQT YVTQQLIRAA EIRASANLAA TKMSECVLGQ SKRVDFCGKG
                  1030 1040 1050 1060 1070 1080
YHLMSFPQSA PHGVVFLHVT YVPAQEKNFT TAPAICHDGK AHFPREGVFV SNGTHWFVTQ
                  1090 1100 1110 1120 1121
RNFYEPQIIT TDNTFVSGNC DVVIGIVNNT VYDPLQPELD S
SEQ ID NO: 5-(CoV2_S_1_hbnet) mutant Spike (S) protein amino acid sequence
AYTNSFTRGVYYPDKVSMSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALELLVDLPIGINITRFQTLLALHRSYLTPGDSSSGWTAGAAAYY
VGYLQPRTFLLKYNENGVITDAVDCALDPLSETKCTLKSFTVEKGIYITSLFEVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELNHAPATVCGPKKSTNLVKNKCVNFNFNGLTGTGVLTESNLKFVSTQLFR
VHSANTTLAVRDPQTLEILDIVSCSSGAVSVITPGTNTSNQVAVLYYNVWCTEVPVAIHA
DQLTPTWRVYSTGSNVFQTKAGCLIGAEHVNNSYECDIAIGGGICASYQTQTNSPGSASS
VASQSIIAYWISTGSWNSVDNSNDAIAIATNFTISVTTEILPVSMTKTWVICTLYICGGS
TECSNLLAQYGSFCTELNRALTGIAVEQDKNTWEVFAQVRTIFHTPSIKDFGGFNFSQIL
PDPSKPSKRSFIEDLLFNKVTLADAGFIKQYGDCLGDIAARDLSCHQDSRGLNILSSLLT
DELIAEFTSALLAGTITAGWSFTAGHALNIPWAVQMAWRFAGIGVTENVLAKNQKLIANQ
FNSAIGKIQDSLSSTASALGKLQDVVNQNAQALDELERELSSNFGAISSVLNDILSNLDP
PEAEVQIDRLILGRLMALAAFVTAQLIRAAEIRASANLAATKMAECVAGQSKLVGFCGEG
WHLMSFPQSAPHGVVFLHVTLVAGQTKNFTTALAICHDGKAHIPRNGVFVSNGTHWFVTQ
EQFYEPLIITTDLVLVSGNCDDVIGIVNNTVYDPKQPELDS
SEQ ID NO: 6-(CoV2_S_2_hbnet) mutant Spike (S) protein amino acid sequence
AYTNSFTRGVYYPDKVSMSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALVLLVDLPIGINITRFQTLLALHRSYLTPGDSSSGWTAGAAAYY
VGYLQPRTFLLKYNENGVITDAVDCALDPLSETKCTLKSFTVEKGIYITSLFKVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELNHAPATVCGPKKSTNLVKNKCVNFNFNGLTGTGVLTESNLKFLEFQLFH
VHSANTTLAVRDPQTLEILDIVSCSSGAVSVITPGTNTSNQVAVLYYNVWCTEVPVAIHA
DQLTPTWRVYSTGSNVFQTRAGCLIGAEHVNNSYECDIAIGAGICASYQTQTNSPGSASS
VASQSIIAYQISTGSWNSVDNSNDAIAIATNETISVTTEILPVSMTKTWVICTLYICGGS
TECSNLLAQHGSFCTELNRALTGIAVEQDKNTWEVFATVRTIFHTPSIKDFGGFNFSQIL
PDPSKPSKRSFIEDLLFNKVTLADAGFIKQYGDCLGDIAARDLSCHQDSHGLNILSSLLT
DELIAEFTSALLAGTITAGHTFTAGHASNIPWWAQMAWRFAGIGVTENVLAKNQKLIANQ
FNSAIGKIQDSLSSTASALGKLQDVVNQNAQALDELERELSSNFGAISSVLNDILSNLDP
PEAEVQIDRLILGRLLALAAFVTAQLIRAAEIRASANLAATKMRECVAGQSKLVGFCGEG
WHLMSFPQSAPHGVVFLHVTLVAGQTKNFTTAPAICHDGKAHIPRTGVFVSNGTHWFVTQ
ENFYEPQIITTDNVFVSGNCDDVIGIVNNTVYDPLQPELDS
SEQ ID NO: 7-(CoV2_S_3_hbnet) mutant Spike (S) protein amino acid sequence
AYTNSFTRGVYYPDKVSMSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALELLVDLPIGINITRFQTLLALHRSYLTPGDSSSGWTAGAAAYY
VGYLQPRTFLLKYNENGVITDAVDCALDPLSETKCTLKSFTVEKGIYDTSTFEVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELNHAPATVCGPKKSTNLVKNKCVNFNFNGLTGTGVLTESNLKFVSTQLFR
VHSANTTLAVRDPQTLEILDIVSCSSGRVSVITPGTNTSNQVAVLYRNVWCTEVPVAIHA
DQLTPTWRVYSTGSNVFQTKAGCLIGAEHVNNSYECDIYIGGGICASYQTQTNSPGSASS
VASQSIIAYWISTGSWNSVENSNDAIAIATNFTISVTTEILPVSMTKTHVDCTLYICGGS
TECSNLLAQYGSFCTELNRMLTGIAVEQDKNTWEVFATVRTIFHTPSIKDFGGFNFSQIL
PDPSKPSKRSFIEDLLFNKVTLADAGFIKQYGDCLGDIAARDLSCHQDSRGLNILSSLLT
DELIAEFTSALLAGTITAGWSFTAGAALNIPWWAQMAWRFAGIGVTENVLAKNQKLIANQ
FNSAIGKIQDSLSSTASALGKLQDVVNQNAQALDELERELSSNFGAISSVLNDILSNLDP
PEAEVQIDRLILGRLMALAAFVTAQAIRAAEIRASANLAALKMRICVAGQSKLVGFCGEG
WHLMSFPQSAPHGVVFLHVTLVAGQYKNFTTAPAICHDGKAHIPSTGVFVSNGTHWFVTQ
EQFYEPQIITTDLVIVSGNCDDVIGIVNNTVYDPLQPELDS
SEQ ID NO: 8-(CoV2_S_4_hbnet) mutant Spike (S) protein amino acid sequence
AYTNSFTRGVYYPDKVSMSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALELLVDLPIGINITRFQTLLALHRSYLTPGDSSSGWTAGAAAYY
VGYLQPRTFLLKYNENGVITDAVDCALDPLSETKCTLKSFTVEKGIYDTSTFKVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELNHAPATVCGPKKSTNLVKNKCVNFNFNGLTGTGVLTESNLKFVSTQLFR
VHSANTTLAVRDPQTLEILDIVSCSSGRVSVITPGTNTSNQVAVLYRNVWCTEVPVAIHA
DQLTPTWRVYSTGSNVFQTRAGCLIGAEHVNNSYECDIAIGAGICASYQTQTNSPGSASS
VASQSIIAYQISTGSWNSVDNSNDAIAVATNFTISVTTEILPVSMTKTHVDCTLYICGGS
TECSNLLAQHGSFCTELNRMLTGIAVEQDKNTWEVFATVRTIFHTPSIKDFGGFNFSQIL
PDPSKPSKRSFIEDLLFNKVTLADAGFIKQYGDCLGDIAARDLSCHQDSHGLNILSSLLT
DELIAEFTSALLAGTITAGTTFLAGHACNIPWWAQMAWRFAGIGVTENVLAKNQKLIANQ
FNSAIGKIQDSLSSTASALGKLQDVVNQNAQALAELERELSSNFGAISSVLNDILSNLDP
PEAEVQIDRLILGRLLALAAFVTAQAIRAAEIRASANLAATKMRECVAGQSKLWGFCGEG
WHLMSFPQSAPHGWFLHVTLVAGQTKNFTTAPAICHDGKAHIPRNGVFVSNGTHWFVTQ
EEFYEPLIITTDLVLVSGNCDDVIGIVNNTVYDPLQPELDS
SEQ ID NO: 9-(CoV2_S_5_hbnet) mutant Spike (S) protein amino acid sequence
AYTNSFTRGVYYPDKVSMSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALELLVDLPIGINITRFQTLLALHRSYLTPGDSSSGWTAGAAAYY
VGYLQPRTFLLKYNENGVITDAVDCALDPLSETKCTLKSFTVEKGIYITSLFKVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELNHAPATVCGPKKSTNLVKNKCVNFNENGLTGTGVLTESNLKEVSTQLEM
VHSANTTLGVRDPQTLEILDIVSCSSGAVSVITPGTNTSNQVAVLYYNVWCTEVPVAIHA
DQLTPTWRVYSTGSNVFQTRAGCLIGAEHVNNSYECDIYIGAGICASYQTQTNSPGSASS
VASQSIIAYQISTGSWNSVDNSNDAIAIATNFTISVTTEILPVSMTKTWVICTLYICGGS
TECSNLLAQHGSFCTELNRALTGIAVEQDKNTWEVFATVRTIFHTPSIKDFGGFNFSQIL
PDPSKPSKRSFIEDLLFNKVTLADAGFIKQYGDCLGDIAARDLSCHQDSHGLNILSSLLT
DELIAEFTSALLAGTITAGWSFLAGAALNIPWWAQMAWRFKGIGVTEWVLAINQKLIANQ
FNSAIGKIQDSLSSTASALGKLQDVVNQNAQALDELERELSSNFGAISSVLNDILSNLDP
PEAEVQIDRLILGRLLALQAFVTAQLIRAAEIRASANLAATKMRECVAGQSKLWGFCGEG
WHLMSFPQSAPHGVVFLHVTLVAGQLKNFTTAPAICHDGKAHVPRIGVFVSNGTHWFVTQ
EQFYFPLIITTDLVLVSGNCDDVIGIVNNTVYDPLQPELDS
SEQ ID NO: 10-(CoV2_S2_1_hbnet) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYQISTGSWNSVENSNDAIAIATNFTISVTTEILPVSMTKTWVDCTLYICGGS
TECSNLLAQYGSFCTELNRMLTGIAVEQDKNTWEVFATVRTIFHTPSIKDFGGFNFSQIL
PDPSKPSKRSFIEDLLFNKVTLADAGFIKQYGDCLGDIAARDSSCAQKANGLNILSSLLT
DELIAEFTSALLAGTITAGWSFTAGAALNIPWWAQMAWRFAGIGVTENVLAKNQKLIANQ
FNSAIGKIQDSLSSTASALGKLQDVVNQNAQALAELEKELSSNFGAISSVLNDILSNLDP
PEAEVQIDRLILGRLMALAAFVTAQAIRAAEIRASANLAATKMRECVAGQSKLVGFCGEG
WHLMSFPQSAPHGVVFLHVTLVAGQYKNFTTAPAICHDGKAHIPRNGVFVSNGTHWFVTQ
EQFYEPLIITTDLVLVSGNCDDVIGIVNNTVYDPKQPELDS
SEQ ID NO: 11-(CoV2_S2_2_hbnet) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYQISTGSWNSVDNSNDAIAVATNFTISVTTEILPVSMTKTWVDCTLYICGGS
TECSNLLAQHGSFCTELNRALTGIAVEQDKNTWEVFATVRTIFHTPSIKDFGGFNFSQIL
PDPSKPSKRSFIEDLLFNKVTLADAGFIKQYGDCLGDIAARDSSCAQKANGLNILSHLLT
DELIAEFTSALLAGTITAGTTFLAGHACNIPWWAQMAQRFAGIGVTENVLAKNQKLIANQ
FNSAIGKIQDSLSSTASALGKLQDVVNQNAQALAELEKELSSNFGAISSVLNDILSHLDP
PEAEVQIDRLILGRLLALQAFVTAQLIRAAEIRASANLAATKMRECVAGQSKLWGFCGEG
FHLMSFPQSAPHGVVFLHVTYVAGQTKNFTTAPAICHDGKAHIPRNGTFVSNGTHWFVTQ
DNFYEPLIITTDLVLVSGNCDDVIGIVNNTVYDPLQPELDS
SEQ ID NO: 12-(CoV2_S2_3_hbnet) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTOSLLIVNNATNVVIKVCEFQFCNDPFLGVYYH
KNNKSWMESEFRVYSSANNCTFEYVSQPFLMDLEGKQGNFKNLREEVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYQISTGSWNSVDNSNDAIAIATNFTISVTTEILPVSMTKTWVDCTLYICGGS
TECSNLLAQYGSFCTELNRMLTGIAVEQDKNTWEVFATVRTIFHTPSIKDFGGFNFSQIL
PDPSKPSKRSFIEDLLFNKVTLADAGFIKQYGDCLGDIAARDSSCAQKANGLNILSSLLT
DELIAEFTSALLAGTITAGSTFIAGHALNIPWWAQMAWRFAGIGVTENVLAKNQKLIANQ
FNSAIGKIQDSLSSTASALGKLQDVVNQNAQALAELEKELSSNFGAISSVLNDILSNLDP
PEAEVQIDRLILGRLMALAAFVTAQAIRAAEIRASANLAATKMRECVNGQSKLHGFCGEG
WHLMSFPQSAPHGVVFLHVTLVAGQSKNFTTAPAICHDGKAHIPRNGTFVSNGTHWFVTQ
WEFYEPLIITTDLVLVSGNCDDVIGIVNNTVYDPLQPELDS
SEQ ID NO: 13-(CoV2_S2_4_hbnet) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYQISTGSWNSVDNSNDAIAIATNFTISVTTEILPVSMTKTWVDCTLYICGGS
TECSNLLAQYGSFCTELNRMLTGIAVEQDKNTWEVFATVRTIFHTPSIKDFGGFNFSQIL
PDPSKPSKRSFIEDLLFNKVTLADAGFIKQYGDCLGDIAARDSSCAQKANGLNILSHLLT
DELIAEFTSALLAGTITAGWSFLAGHALNIPWAEQMAWRFAGIGVTENVLAKNQKLIANQ
FNSAIGKIQDSLSSTASALGKLQDVVNQNAQALAELEKELSSNFGAISSVLNDILSNLDP
PEAEVQIDRLILGRLMALAAFVTAQAIRAAEIRASANLAATKMRECVAGQSKLWGFCGEG
WHLMSFPQSAPHGVVFLHVTLVAGQSKNFTTALAICHDGKAHIPRNGVFVSNGTHWFVTQ
EQFYEPLIITTDLVLVSGNCDDVIGIVNNTVYDPLQPELDS
SEQ ID NO: 14-(CoV2_S2_5_hbnet) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYQISTGSWNSVDNSNDAIAIATNFTISVTTEILPVSMTKTWVDCTLYICGGS
TECSNLLAQYGSFCTELNRMLTGIAVEQDKNTWEVFATVRTIFHTPSIKDFGGFNFSQIL
PDPSKPSKRSFIEDLLFNKVTLADAGFIKQYGDCLGDIAARDSSCAQKANGLNILSSLLT
DELIAEFTSALLAGTITAGWTFLAGAALNIPWAVQMAWRFAGIGVTENVLAKNQKLIANQ
FNSAIGKIQDSLSSTASALGKLQDVVNQNAQALAQLEKTLSSNFGAISSVLNDILSNLDP
PEAEVQIDRLILGRLMALAAFVTAQAIRAAEIRASANLAATKMRECVAGQSKLWGFCGEG
FHLMSFPQSAPHGWFLHVTYVAGQYKNFTTALAICHDGKAHIPRNGVFVSNGTHWFVTQ
ENFYEPLIITTDLVLVSGNCDDVIGIVNNTVYDPLQPELDS
SEQ ID NO: 15-(CoV2_S_1_pross) mutant Spike (S) protein amino acid sequence:
AYTNSFRRGVYYPDKVFRSNVLHLTQDLFLPFFSNVTWFHAINVSGTNGTKRFDNPVLPF
NDGVYFAATEKSNIIRGWIFGSTLDSKTQTLLIVNNGTNVVIRVCEFNFCEDPFLGVYYH
KNNKSWLESGFHVYDSANNCTFEYVSHPFIMDLEGDSGNFKHLREFIFKNIDGWFHIYSS
HTPINLVTDLPAGFSALELLVDLPIGINITRFQILLALHRSYLTPGDSRSGWTRGAAVYY
VGYLQPRTFLLKYNENGTITDAVDCALDPLAETKCTLKSFTVEKGIYQTSNFRVRPTESI
VRFPNITNLCDFSEVFNATRFASVYAWNRKRISNCVADYSSLYNSTSFSTFHCYGVDPKK
LNDLCFTNVYADSFVIRGDEVRQLAPGQTGEIADYNYKLPDDFTGCVIAWNSNNLDARKS
GNYNYLYRLFRNGNLRPFERDISTEIYQLGDTPCNGVEGFNCYFPLQSYDFQPTNGSEYQ
PYRVVVLSFELLHGPATVCGPKKNTSLVKNKCVNFNFYGYTGTGVLTESNKKFLSFQLFG
RDSSDTTDAVRDPQTNEIYDITPCSFGGVSVITPGTDTSNEVAVLYQNVNCSEVPVAIHA
NQLTPTWRRYSTGSNIFQTRAGCLIGAEFVNNSYECDIPIGAGICASYDTQTNSPGSASS
VASQSIIAYTMSLGSENSVSYSNDSIAIPTNFTISVTTEIIPVSMPKVSVDCKMYICGDH
SECSNLLLQYGSFCTQLNRALHEIAEEQDKNMLEVFAQVRQIYKTPPIKDFGGFNFSLIL
PDPSKSSKRSAIEDLLFNKVKLADAGFIEGYGDCLGDIAARDLICAQKFNGLTVLPPLLT
DEMIAAYTAALLAGTITAGWTFGAGSALQIPFAMQMAYRFNGIGVTQNVLYENQKLIANQ
FNKAIGAIQEGLDETAEALGKLQDVVNQNAQALNTLVKQLSSNFGAISSSLNDILSRLDP
PEAEVQIDRLINGRLQALNTFVTQLLIRAAEIRASAELAAEKMNECVLGQSKRVNFCGNG
YHLMSFPQAAPHGVVFLHVTYVPTSHRNFTTAPAICHNGKAHFPRDGVFVSNGTHWFVTQ
RNFYEPQPITTDNTFVSGDCDVVIGIVNNTVYDPLQPELDS
SEQ ID NO: 16-(CoV2_S_2_pross) mutant Spike (S) protein amino acid sequence:
AYTNSFTRGVYYPDKVFRSNVLHLTQDLFLPFFSNVTWFHAINVSGTNGTKRFDNPVLPF
NDGVYFASTEKSNIIRGWIFGTTLDSKTQSLLIVNNGTNVVIRVCEFNFCENPFLGVYYH
KNNKSWMESGFHVYTSANNCTFEYVSHPFIMDLEGDSGNFKHLREFIFKNIDGWFHIYSK
HTPINLVTDLPAGFSALELLVDLPIGINITRFQILLALHRSYLTPGDSRSGWTRGAAVYY
VGYLQPRTFLLKYNENGTITDAVDCALDPLAETKCTLKSFTVEKGIYQTSNFRVQPTESI
VRFPNITNLCDFSEVFNATRFASVYAWNRKRISNCVADYSSLYNSTSFSTFKCYGVDPTK
LNDLCFTNVYADSFVIRGDEVRQLAPGQTGEIADYNYKLPDDFTGCVIAWNSNNLDARKS
GNYNYLYRLFRHGNLRPFERDISTEIYQAGDTPCNGVEGFNCYFPLQSYDFQPTNGSSYQ
PYRVVVLSFELLHGPATVCGPKKNTSLVKNKCVNFNFYGYTGTGVLTESNKKFLSFQLFG
RDSADTTDAVRDPQTNEIYDITPCSFGGVSVITPGTNTSNQVAVLYQNVNCTEVPVAIHA
NQLTPTWRRYSTGSNIFQTRAGCLIGAEYVNNSYECDIPIGAGICASYDTQTNSPGSASS
VASQSIIAYTMSLGSENSVSYSNDSIAIPTNFTISVTTEIIPVSMQKVSVDCKMYICGDH
SECSNLLLQYGSFCTQLNRALHEIAEEQDKNTLEVFAQVKQIYKTPPIKDFGGFNFSLIL
PDPSKSSKRSAIEDLLFNKVKLADAGFIEGYGDCLGDIAARDLICAQKFNGLTVLPPLLT
DEMIAAYTAALLAGTITAGWTFGAGSALQIPFAMQMAYRFNGIGVTQNVLYENQKLIANQ
FNKAIGAIQEGLDATAEALGKLQDVVNQNAQALNTLVKQLSSNFGAISSVLNDILSRLDP
PEAEVQIDRLINGRLQALNTFVTQLLIRAAEIRASAELAAEKMSECVLGQSKRVDFCGNG
YHLMSFPQAAPHGVVFLHVTYVPTDYRNFTTAPAICHNGKAHFPREGVFVSNGTHWFVTQ
RNFYEPQIITTDNTFVSGDCDVVIGIVNNTVYDPLQPELDS
SEQ ID NO: 17-(CoV2_S_3_5_pross) mutant Spike (S) protein amino acid
sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNGTNVVIRVCEFQFCEDPFLGVYYH
KNNKSWMESGFHVYSSANNCTFEYVSQPFLMDLEGDSGNFKNLREFIFKNIDGWFHIYSK
HTPINLVRDLPEGFSALEPLVDLPIGINITRFQILLALHRSYLTPGDSRSGWTRGAAAYY
VGYLQPRTFLLKYNENGTITDAVDCALDPLSETKCTLKSFTVEKGIYQTSNFRVQPTESI
VRFPNITNLCDFDEVFNATRFASVYAWNRKRISNCVADYSVLYNSTSFSTFWCYGVSPTK
LNDLCFTNVYADSFVIRGDEVRQLAPGQTGEIADYNYKLPDDFTGCVIAWNSNNLDARVS
GNYNYLYRLFRKGNLRPFERDISTEIYQAGSTPCNGVEGFNCYFPLQSYDFQPTNGSHYQ
PYRVVVLSFELLHGPATVCGPKKNTNLVKNKCVNFNFYGYTGTGVLTESNKKFLSFQQFG
RDSADTTDAVRDPQTNEIYDITPCSFGGVSVITPGTNTSNQVAVLYQGVNCTEVPVAIHA
DQLTPTWRRYSTGSNVFQTRAGCLIGAEYVNNSYECDIPIGAGICASYDTQTNSPGSASS
VASQSIIAYTMSLGEENSVSYSNNSIAIPTNFTISVTTEIIPVSMQKVSVDCTMYICGDH
EECSNLLLQYGSFCTQLNRALHEIAVEQDKNTQEVFAQVKQIYKTPPIKDFGGFNFSQIL
PDPSKSSKRSAIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGAIQDGLDSTASALGKLQDVVNQNAQALNTLVKQLSSNFGAISSVLNDILSRLDP
PEAEVQIDRLINGRLQALNTYVTQQLIRAAEIRASANLAAEKMSECVLGQSKRVDFCGKG
YHLMSFPQAAPHGVVFLHVTYVPTQHKNFTTAPAICHNGKAHFPREGVFVSNGTHWFVTQ
RNFYEPQPITTDNTFVSGDCDVVIGIVNNTVYDPLQPELDS
SEQ ID NO: 18-(CoV2_S_5_pross) mutant Spike (S) protein amino acid sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNATNVVIKVCEFQFCEDPFLGVYYH
KNNKSWMESEFHVYSSANNCTFEYVSQPFLMDLEGKQGNFKNLREFVFKNIDGYFKIYSK
HTPINLVRDLPEGFSALEPLVDLPIGINITRFQILLALHRSYLTPGDSRSGWTAGAAAYY
VGYLQPRTFLLKYNENGTITDAVDCALDPLSETKCTLKSFTVEKGIYQTSNFRVQPTESI
VRFPNITNLCDFDEVFNATRFASVYAWNRKRISNCVADYSVLYNSTSFSTFKCYGVSPTK
LNDLCFTNVYADSFVIRGDEVRQLAPGQTGEIADYNYKLPDDFTGCVIAWNSNNLDARVS
GNYNYLYRLFRKGNLKPFERDISTEIYQAGSTPCNGVEGFNCYFPLQSYNFQPTNGSGYQ
PYRVVVLSFELLHGPATVCGPKKNTNLVKNKCVNFNFNGYTGTGVLTESNKKFLSFQQFG
RDSADTTDAVRDPQTNEILDITPCSFGGVSVITPGTNTSNQVAVLYQNVNCTEVPVAIHA
DQLTPTWRRYSTGSNVFQTRAGCLIGAEHVNNSYECDIPIGAGICASYDTQTNSPGSASS
VASQSIIAYTMSLGAENSVAYSNNSIAIPTNFTISVTTEIIPVSMQKTSVDCTMYICGDS
TECSNLLLQYGSFCTQLNRALHEIAVEQDKNTQEVFAQVKQIYKTPPIKDFGGFNFSQIL
PDPSKSSKRSFIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQALNTYVTQQLIRAAEIRASANLAAEKMSECVLGQSKRVDFCGKG
YHLMSFPQSAPHGVVFLHVTYVPTQFKNFTTAPAICHDGKAHFPREGVFVSNGTHWFVTQ
RNFYEPQPITTDNTFVSGNCDVVIGIVNNTVYDPLQPELDS
SEQ ID NO: 19-(CoV2_S_6_pross) mutant Spike (S) protein amino acid sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNATNVVIKVCEFQFCEDPFLGVYYH
KNNKSWMESEFHVYSSANNCTFEYVSQPFLMDLEGKQGNFKNLREFVFKNIDGYFKIYSK
HTPINLVRDLPQGFSALEPLVDLPIGINITRFQILLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCDFSEVFNATRFASVYAWNRKRISNCVADYSVLYNSTSFSTFKCYGVSPTK
LNDLCFTNVYADSFVIRGDEVRQLAPGQTGKIADYNYKLPDDFTGCVIAWNSNNLDSRVG
GNYNYLYRLFRKGNLKPFERDISTEIYQAGSTPCNGVEGFNCYFPLQSYGFQPTNGVGYQ
PYRVVVLSFELLHAPATVCGPKKNTNLVKNKCVNFNFNGLTGTGVLTESNKKFLSFQQFG
RDSADTTDAVRDPQTNEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEIIPVSMPKTSVDCTMYICGDS
TECSNLLLQYGSFCTQLNRALTEIAVEQDKNTQEVFAQVKQIYKTPPIKDFGGFNFSQIL
PDPSKSSKRSFIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQSLQTYVTQQLIRAAEIRASANLAAEKMSECVLGQSKRVDFCGKG
YHLMSFPQSAPHGVVFLHVTYVPTQYKNFTTAPAICHDGKAHFPREGVFVSNGTHWFVTQ
RNFYEPQIITTDNTFVSGNCDVVIGIVNNTVYDPLQPELDS
SEQ ID NO: 20-(CoV2_S2_NTD_0_5_pross) mutant Spike (S) protein amino acid
sequence:
AYTNSFRRGVYYPDKIFRSNVLHLTQDLFLPFFSNVTWFHAINVSGTNGTKRFDNPVLPF
NDGVYFAATEKNNIIRGWIFGSTLDSKTQTLLIVNNGTNIVIRVCEFNFCENPFLGVYYH
KNNKSWSESGFHVYDSANNCTFEYVSHPFIMDLEGDSGNFKHLREFIFKNIDGWFLIYSS
HTPINLVTDLPAGFSALELLVDLPIGINITRFQILLALHRSYLTPGDSRSGWTRGAAVYY
VGYLQPRTFLLKYDENGTITDAVDCALDPLAETKCTLKSFTVEKGIYQTSNFRVRPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTDTSNEVAVLYQNVNCSEVPTAIHA
NQLTPTWRRYSTGSNIFQTRAGCLIGAEEVNNSYECDIPIGAGICASYDTQTNSRGSASS
VASQSIIAYTMSLGSENSVSYSNTSIAIPTNFTISVTTEIIPVSMPKVSVDCKMYICGDH
SECKNLLLQYGSFCTQLNRALHEIAEEQDKNLREVFAQVRQIYKTPPIKDFGGFNFSLIL
PDPSKPSKRSAIEDLLFNKVKLADAGFIEGYGDCLGDIAARDLICAQKFNGLTVLPPLLT
DEMIAAYTAALLAGTITAGWTFGAGSALQIPFAMQMAYRFNGIGVTQNVLYENQKLIANQ
FNKAIGAIQEGLDETAEALGKLQDVVNQNAEALNTLVKQLSSNFGAISSSLNDILSRLDP
PEAEVQIDRLINGRLQALNTFVTQLLIRAAEIRASAELAAEKMNECVLGQSKRVNFCGNG
YHLMSFPQAAPHGVVFLHVTYVPTDHRNFTTAPAICHNGKAHFPRDGVFVSNGTHWFVTQ
RNFYEPQPITTDNTFVSGDCDVVIGIVNNTVYDPLKPELDS
SEQ ID NO: 21-(CoV2_S2_NTD_2_pross) mutant Spike (S) protein amino acid
sequence:
AYTNSFTRGVYYPDKVFRSNVLHLTQDLFLPFFSNVTWFHAINVSGTNGTKRFDNPVLPF
NDGVYFASTEKSNIIRGWIFGTTLDSKTQSLLIVNNGTNVVIRVCEFNFCENPFLGVYYH
KNNKSWMESGFHVYTSANNCTFEYVSHPFIMDLEGDSGNFKHLREFIFKNIDGWFKIYSK
HTPINLVTDLPAGFSALELLVDLPIGINITRFQILLALHRSYLTPGDSRSGWTRGAAVYY
VGYLQPRTFLLKYNENGTITDAVDCALDPLAETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQNVNCTEVPVAIHA
NQLTPTWRRYSTGSNIFQTRAGCLIGAEHVNNSYECDIPIGAGICASYDTQTNSPGSASS
VASQSIIAYTMSLGSENSVSYSNDSIAIPTNFTISVTTEIIPVSMQKVSVDCKMYICGDH
SECSNLLLQYGSFCTQLNRALHEIAEEQDKNTLEVFAQVKQIYKTPPIKDFGGFNFSLIL
PDPSKPSKRSAIEDLLFNKVKLADAGFIEGYGDCLGDIAARDLICAQKFNGLTVLPPLLT
DEMIAAYTAALLAGTITAGWTFGAGSALVIPFAMQMAYRFNGIGVTQNVLYENQKLIANQ
FNKAIGAIQEGLDATAEALGKLQDVVNQNAQALNTLVKQLSSNFGAISSVLNDILSRLDP
PEAEVQIDRLINGRLQALNTFVTQLLIRAAEIRASAELAAEKMSECVLGQSKRVDFCGNG
YHLMSFPQAAPHGVVFLHVTYVPTDYRNFTTAPAICHNGKAHFPREGVFVSNGTHWFVTQ
RNFYEPQPITTDNTFVSGDCDVVIGIVNNTVYDPLQPELDS
SEQ ID NO: 22-(CoV2_S2_NTD_3_pross) mutant Spike (S) protein amino acid
sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNGTNVVIRVCEFNFCEDPFLGVYYH
KNNKSWMESGFHVYSSANNCTFEYVSQPFLMDLEGDSGNFKNLREFIFKNIDGWFHIYSK
HTPINLVRDLPEGFSALEPLVDLPIGINITRFQILLALHRSYLTPGDSRSGWTRGAAVYY
VGYLQPRTFLLKYNENGTITDAVDCALDPLAETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRRYSTGSNIFQTRAGCLIGAEYVNNSYECDIPIGAGICASYDTQTNSPGSASS
VASQSIIAYTMSLGEENSVSYDNNSIAIPTNFTISVTTEIIPVSMQKVSVDCTMYICGDH
SECSNLLLQYGSFCTQLNRALHEIAVEQDKNTLEVFAQVKQIYKTPPIKDFGGFNFSQIL
PDPSKPSKRSAIEDLLFNKVTLADAGFIKGYGDCLGDIAARDLICAQKFNGLTVLPPLLT
DEMIAAYTSALLAGTITAGWTFGAGSALQIPFAMQMAYRFNGIGVTQNVLYENQKLIANQ
FNKAIGAIQDGLDSTASALGKLQDVVNQNAQALNTLVKQLSSNFGAISSVLNDILSRLDP
PEAEVQIDRLINGRLQALNTYVTQLLIRAAEIRASANLAAEKMSECVLGQSKRVDFCGKG
YHLMSFPQAAPHGVVFLHVTYVPTQYKNFTTAPAICHNGKAHFPREGVFVSNGTHWFVTQ
RNFYEPQIITTDNTFVSGDCDVVIGIVNNTVYDPLQPELDS
SEQ ID NO: 23-(CoV2_S2_NTD_5_pross) mutant Spike (S) protein amino acid
sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNATNVVIKVCEFQFCEDPFLGVYYH
KNNKSWMESEFHVYSSANNCTFEYVSQPFLMDLEGKQGNFKNLREFVFKNIDGYFHIYSK
HTPINLVRDLPEGFSALEPLVDLPIGINITRFQILLALHRSYLTPGDSRSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRRYSTGSNVFQTRAGCLIGAEYVNNSYECDIPIGAGICASYDTQTNSPGSASS
VASQSIIAYTMSLGAENSVAYSNNSIAIPTNFTISVTTEIIPVSMQKTSVDCTMYICGDS
TECSNLLLQYGSFCTQLNRALHEIAVEQDKNTQEVFAQVKQIYKTPPIKDFGGFNFSQIL
PDPSKPSKRSFIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQALNTYVTQQLIRAAEIRASANLAAEKMSECVLGQSKRVDFCGKG
YHLMSFPQSAPHGVVFLHVTYVPTQYKNFTTAPAICHDGKAHFPREGVFVSNGTHWFVTQ
RNFYEPQPITTDNTFVSGNCDVVIGIVNNTVYDPLQPELDS
SEQ ID NO: 24-(CoV2_S2_NTD_6_pross) mutant Spike (S) protein amino acid
sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNATNWIKVCEFQFCEDPFLGVYYH
KNNKSWMESEFHVYSSANNCTFEYVSQPFLMDLEGKQGNFKNLREFVFKNIDGYFKIYSK
HTPINLVRDLPQGFSALEPLVDLPIGINITRFQILLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEIIPVSMPKTSVDCTMYICGDS
TECSNLLLQYGSFCTQLNRALTEIAVEQDKNTQEVFAQVKQIYKTPPIKDFGGFNFSQIL
PDPSKPSKRSFIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQSLQTYVTQQLIRAAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 25-(CoV2_S2_1_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGSENSVSYSNDSIAIPTNFTISVTTEIIPVSMPKVSVDCKMYICGDH
SECSNLLLQYGSFCTQLNRALHEIAEEQDKNMREVFAQVRQIYKTPPIKDFGGFNFSLIL
PDPSKPSKRSAIEDLLFNKVKLADAGFIEGYGDCLGDIAARDLICAQKFNGLTVLPPLLT
DEMIAAYTAALLAGTITAGWTFGAGSALQIPFAMQMAYRFNGIGVTQNVLYENQKLIANQ
FNKAIGAIQEGLDETAEALGKLQDVVNQNAQALNTLVKQLSSNFGAISSSLNDILSRLDP
PEAEVQIDRLINGRLQALNTFVTQLLIRAAEIRASAELAAEKMNECVLGQSKRVNFCGNG
YHLMSFPQAAPHGVVFLHVTYVPTEYRNFTTAPAICHNGKAHFPRDGVFVSNGTHWFVTQ
RNFYEPQIITTDNTFVSGDCDVVIGIVNNTVYDPLQPELDS
SEQ ID NO: 26-(CoV2_S2_2_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGSENSVSYSNDSIAIPTNFTISVTTEIIPVSMQKVSVDCKMYICGDH
SECSNLLLQYGSFCTQLNRALHEIAEEQDKNTLEVFAQVKQIYKTPPIKDFGGFNFSLIL
PDPSKPSKRSAIEDLLFNKVKLADAGFIEGYGDCLGDIAARDLICAQKFNGLTVLPPLLT
DEMIAAYTAALLAGTITAGWTFGAGSALQIPFAMQMAYRFNGIGVTQNVLYENQKLIANQ
FNKAIGAIQEGLDATAEALGKLQDVVNQNAQALNTLVKQLSSNFGAISSVLNDILSRLDP
PEAEVQIDRLINGRLQALNTFVTQLLIRAAEIRASAELAAEKMSECVLGQSKRVDFCGNG
YHLMSFPQAAPHGVVFLHVTYVPTDYRNFTTAPAICHNGKAHFPREGVFVSNGTHWFVTQ
RNFYEPQIITTDNTFVSGDCDVVIGIVNNTVYDPLQPELDS
SEQ ID NO: 27-(CoV2_S2_3_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATVVWIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGEENSVSYDNNSIAIPTNFTISVTTEIIPVSMQKVSVDCTMYICGDH
SECSNLLLQYGSFCTQLNRALHEIAVEQDKNTLEVFAQVKQIYKTPPIKDFGGFNFSQIL
PDPSKPSKRSAIEDLLFNKVTLADAGFIKGYGDCLGDIAARDLICAQKFNGLTVLPPLLT
DEMIAAYTSALLAGTITAGWTFGAGSALQIPFAMQMAYRFNGIGVTQNVLYENQKLIANQ
FNKAIGAIQDGLDSTASALGKLQDVVNQNAQALNTLVKQLSSNFGAISSVLNDILSRLDP
PEAEVQIDRLINGRLQALNTYVTQLLIRAAEIRASANLAAEKMSECVLGQSKRVDFCGKG
YHLMSFPQAAPHGVVFLHVTYVPTQYKNFTTAPAICHNGKAHFPREGVFVSNGTHWFVTQ
RNFYEPQPITTDNTFVSGDCDVVIGIVNNTVYDPLQPELDS
SEQ ID NO: 28-(CoV2_S2_4_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGEENSVAYSNNSIAIPTNFTISVTTEIIPVSMQKVSVDCTMYICGDS
EECSNLLLQYGSFCTQLNRALHEIAVEQDKNTQEVFAQVKQIYKTPPIKDFGGFNFSQIL
PDPSKPSKRSAIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQALNTYVTQQLIRAAEIRASANLAAEKMSECVLGQSKRVDFCGKG
YHLMSFPQSAPHGVVFLHVTYVPTQYKNFTTAPAICHNGKAHFPREGVFVSNGTHWFVTQ
RNFYEPQIITTDNTFVSGDCDVVIGIVNNTVYDPLQPELDS
SEQ ID NO: 29-(CoV2_S2_6_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEI1PVSMPKTSVDCTMYICGDS
TECSNLLLQYGSFCTQLNRALTEIAVEQDKNTQEVFAQVKQIYKTPPIKDFGGFNFSQIL
PDPSKPSKRSFIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQSLQTYVTQQLIRAAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 30-(CoV2_S2_1_hbnet_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIATNFTISVTTEILPVSMTKTSVDCTMYICGGS
TECSNLLLQYGSFCTQLNRALTEIAVEQDKNTQEVFAQVKQIYKTPPIKDFGGFNFSQIL
PDPSKPSKRSAIEDLLFNKVKLADAGFIKGYGDCLGDIAARDSICAQKFNGLTILSSLLT
DEMIAAFTSALLAGTITAGWTFGAGAALQIPFAMQMAYRFAGIGVTQNVLYENQKLIANQ
FNNAIGKIQDGLSSTASALGKLQDVVNQNAQALNTLVKQLSSNFGAISSVLNDILSNLDP
PEAEVQIDRLITGRLQSLQTYVTQQAIRAAEIRASANLAATKMSECVLGQSKLVDFCGKG
YHLMSFPQSAPHGVVFLHVTYVATQYKNFTTAPAICHDGKAHFPREGVFVSNGTHWFVTQ
RNFYEPQIITTDLTFVSGNCDVVIGIVNNTVYDPLQPELDS
SEQ ID NO: 31-(CoV2_S2_2_hbnet_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIATNFTISVTTEILPVSMTKTSVDCTMYICGGS
TECSNLLLQYGSFCTQLNRALTEIAVEQDKNTQEVFAQVKQIYKTPPIKDFGGFNFSLIL
PDPSKPSKRSAIEDLLFNKVKLADAGFIKGYGDCLGDIAARDSICAQKFNGLTILSHLLT
DEMIAAFTSALLAGTITAGWTFGAGAALQIPFAMQMAYRFAGIGVTQNVLYENQKLIANQ
FNNAIGKIQDGLSSTASALGKLQDVVNQNAQALNTLVKQLSSNFGAISSVLNDILSHLDP
PEAEVQIDRLITGRLQSLQTYVTQQLIRAAEIRASANLAATKMSECVLGQSKLVDFCGKG
YHLMSFPQSAPHGVVFLHVTYVATQYKNFTTAPAICHDGKAHFPREGVFVSNGTHWFVTQ
RNFYEPQIITTDLTFVSGNCDVVIGIVNNTVYDPLQPELDS
SEQ ID NO: 32-(CoV2_S2_3_hbnet_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIATNFTISVTTEILPVSMTKTSVDCTMYICGGS
TECSNLLLQYGSFCTQLNRALTEIAVEQDKNTQEVFAQVKQIYKTPPIKDFGGFNFSLIL
PDPSKPSKRSAIEDLLFNKVKLADAGFIKGYGDCLGDIAARDSICAQKFNGLTILSSLLT
DEMIAAFTSALLAGTITAGWTFGAGAALQIPFAMQMAYRFAGIGVTQNVLYENQKLIANQ
FNNAIGKIQDGLSSTASALGKLQDVVNQNAQALNTLVKQLSSNFGAISSVLNDILSNLDP
PEAEVQIDRLITGRLQSLQTYVTQQAIRAAEIRASANLAATKMSECVLGQSKLVDFCGKG
YHLMSFPQSAPHGVVFLHVTYVATQYKNFTTAPAICHDGKAHFPREGVFVSNGTHWFVTQ
RNFYEPQIITTDLTFVSGNCDVVIGIVNNTVYDPLQPELDS
SEQ ID NO: 33-(CoV2_S2_4_hbnet_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIATNFTISVTTEILPVSMTKTSVDCTMYICGGS
TECSNLLLQYGSFCTQLNRALTEIAVEQDKNTQEVFAQVKQIYKTPPIKDFGGFNFSLIL
PDPSKPSKRSAIEDLLFNKVKLADAGFIKGYGDCLGDIAARDSICAQKFNGLTILSHLLT
DEMIAAFTSALLAGTITAGWTFGAGAALQIPFAMQMAYRFAGIGVTQNVLYENQKLIANQ
FNNAIGKIQDGLSSTASALGKLQDVVNQNAQALNTLVKQLSSNFGAISSVLNDILSNLDP
PEAEVQIDRLITGRLQSLQTYVTQQAIRAAEIRASANLAATKMSECVLGQSKLVDFCGKG
YHLMSFPQSAPHGVVFLHVTYVATQYKNFTTAPAICHDGKAHFPREGVFVSNGTHWFVTQ
RNFYEPQIITTDLTFVSGNCDVVIGIVNNTVYDPLQPELDS
SEQ ID NO: 34-(Cov2_S2_5_hbnet_pross) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNEVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIATNFTISVTTEILPVSMSKTSVDCTMYICGGS
TECSNLLLQYGSFCTQLNRALTEIAVEQDKNTQEVFAQVKQIYKTPPIKDFGGFNFSQIL
PDPSKPSKRSAIEDLLFNKVKLADAGFIKGYGDCLGDIAARDSICAQKFNGLTILSSLLT
DEMIAAFTSALLAGTITAGWTFGAGAALQIPFAMQMAYRFAGIGVTQNVLYENQKLIANQ
FNKAIGKIQDGLSSTASALGKLQDVVNQNAQALNTLVKQLSSNFGAISSVLNDILSNLDP
PEAEVQIDRLITGRLQSLQTYVTQQAIRAAEIRASANLAATKMSECVLGQSKLVDFCGKG
YHLMSFPQSAPHGVVFLHVTYVATQYKNFTTAPAICHDGKAHFPREGVFVSNGTHWFVTQ
RNFYEPQIITTDLTFVSGNCDVVIGIVNNTVYDPLQPELDS
SEQ ID NO: 35-(CoV_2_S_openDS1, SEQ ID NO: 4 as parent) mutant Spike (S)
protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGCAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRCAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 36-(CoV_2_S_openDS2, SEQ ID NO: 4 as parent) mutant Spike (S)
protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGCCLGDIAARDLICAQKFNGLTVLCPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 37-(CoV_2_S_openDS3, SEQ ID NO: 4 as parent) mutant Spike (S)
protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDICDTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLCSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 38-(CoV_2_S_openDS4, SEQ ID NO: 4 as parent) mutant Spike (S)
protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILCITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLCCAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 39-(CoV_2_S_closedDS1, SEQ ID NO: 4 as parent) mutant Spike (S)
protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPCQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
CEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 40-(CoV_2_S_closedDS2, SEQ ID NO: 4 as parent) mutant Spike (S)
protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVCPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLCP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 41-(CoV_2_S_closedDS3, SEQ ID NO: 4 as parent) mutant Spike (S)
protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGCSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSCLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 42-(CoV_2_S_closedDS4, SEQ ID NO: 4 as parent) mutant Spike (S)
protein amino acid sequence:
AYTNSFTRGVYYPDCVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHCPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 43-(CoV_2_S_closedDS5, SEQ ID NO: 4 as parent) mutant Spike (S)
protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPCTVCGPKKSTNLVKNKCVNFNFCGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 44-(CoV_2_S_closedDS6, SEQ ID NO: 4 as parent) mutant Spike (S)
protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHACATVCGPKKSTNLVKNKCVNFNFNGLTGTGVLTESNKKFLPFQCFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNETISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 45-(CoV2_S_1_hbnet_openDS1, SEQ ID NO: 5 as parent) mutant Spike
(S) protein amino acid sequence:
AYTNSFTRGVYYPDKVSMSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALELLVDLPIGINITRFQTLLALHRSYLTPGDSSSGWTAGAAAYY
VGYLQPRTFLLKYNENGVITDAVDCALDPLSETKCTLKSFTVEKGIYITSLFEVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELNHAPATVCGPKKSTNLVKNKCVNFNFNGLTGTGVLTESNLKFVSTQLFR
VHSANTTLAVRDPQTLEILDIVSCSSGAVSVITPGTNTSNQVAVLYYNVWCTEVPVAIHA
DQLTPTWRVYSTGSNVFQTKAGCLIGAEHVNNSYECDIAIGGGICASYQTQTNSPGSASS
VASQSIIAYWISTGSWNSVDNSNDAIAIATNFTISVTTEILPVSMTKTWVICTLYICGGS
TECSNLLAQYGSFCTELNRALTGCAVEQDKNTWEVFAQVRTIFHTPSIKDFGGFNFSQIL
PDPSKPSKRSFIEDLLFNKVTLADAGFIKQYGDCLGDIAARDLSCHQDSRGLNILSSLLT
DELIAEFTSALLAGTITAGWSFTAGHALNIPWAVQMAWRFAGIGVTENVLAKNQKLIANQ
FNSAIGKIQDSLSSTASALGKLQDVVNQNAQALDELERELSSNFGAISSVLNDILSNLDP
PEAEVQIDRLILGRLMALAAFVTAQLIRCAEIRASANLAATKMAECVAGQSKLVGFCGEG
WHLMSFPQSAPHGVVFLHVTLVAGQTKNFTTALAICHDGKAHIPRNGVFVSNGTHWFVTQ
EQFYEPLIITTDLVLVSGNCDDVIGIVNNTVYDPKQPELDS
SEQ ID NO: 46-(CoV2_S2_1_hbnet_openDS1, SEQ ID NO: 10 as parent) mutant
Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYQISTGSWNSVENSNDAIAIATNFTISVTTEILPVSMTKTWVDCTLYICGGS
TECSNLLAQYGSFCTELNRMLTGCAVEQDKNTWEVFATVRTIFHTPSIKDFGGFNFSQIL
PDPSKPSKRSFIEDLLFNKVTLADAGFIKQYGDCLGDIAARDSSCAQKANGLNILSSLLT
DELIAEFTSALLAGTITAGWSFTAGAALNIPWWAQMAWRFAGIGVTENVLAKNQKLIANQ
FNSAIGKIQDSLSSTASALGKLQDVVNQNAQALAELEKELSSNFGAISSVLNDILSNLDP
PEAEVQIDRLILGRLMALAAFVTAQAIRCAEIRASANLAATKMRECVAGQSKLVGFCGEG
WHLMSFPQSAPHGVVFLHVTLVAGQYKNFTTAPAICHDGKAHIPRNGVFVSNGTHWFVTQ
EQFYEPLIITTDLVLVSGNCDDVIGIVNNTVYDPKQPELDS
SEQ ID NO: 47-(CoV2_S2_NTD_6_pross_openDSl, SEQ ID NO: 24 as parent) mutant
Spike (S) protein amino acid sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNATNVVIKVCEFQFCEDPFLGVYYH
KNNKSWMESEFHVYSSANNCTFEYVSQPFLMDLEGKQGNFKNLREFVFKNIDGYFKIYSK
HTPINLVRDLPQGFSALEPLVDLPIGINITRFQILLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEIIPVSMPKTSVDCTMYICGDS
TECSNLLLQYGSFCTQLNRALTECAVEQDKNTQEVFAQVKQIYKTPPIKDFGGFNFSQIL
PDPSKPSKRSFIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQSLQTYVTQQLIRCAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 48-(CoV2_S2_6_pross_openDSl, SEQ ID NO: 29 as parent) mutant
Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEIIPVSMPKTSVDCTMYICGDS
TECSNLLLQYGSFCTQLNRALTECAVEQDKNTQEVFAQVKQIYKTPPIKDFGGFNFSQIL
PDPSKPSKRSFIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQSLQTYVTQQLIRCAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 49-(CoV2_S2_1_hbnet_pross_openDS1, SEQ ID NO: 30 as parent)
mutant Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIATNFTISVTTEILPVSMTKTSVDCTMYICGGS
TECSNLLLQYGSFCTQLNRALTECAVEQDKNTQEVFAQVKQIYKTPPIKDFGGFNFSQIL
PDPSKPSKRSAIEDLLFNKVKLADAGFIKGYGDCLGDIAARDSICAQKFNGLTILSSLLT
DEMIAAFTSALLAGTITAGWTFGAGAALQIPFAMQMAYRFAGIGVTQNVLYENQKLIANQ
FNNAIGKIQDGLSSTASALGKLQDVVNQNAQALNTLVKQLSSNFGAISSVLNDILSNLDP
PEAEVQIDRLITGRLQSLQTYVTQQAIRCAEIRASANLAATKMSECVLGQSKLVDFCGKG
YHLMSFPQSAPHGVVFLHVTYVATQYKNFTTAPAICHDGKAHFPREGVFVSNGTHWFVTQ
RNFYEPQIITTDLTEVSGNCDVVIGIVNNTVYDPLQPELDS
SEQ ID NO: 50-(CoV2_S_1_hbnet_openDS2, SEQ ID NO: 5 as parent) mutant Spike
(S) protein amino acid sequence:
AYTNSFTRGVYYPDKVSMSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALELLVDLPIGINITRFQTLLALHRSYLTPGDSSSGWTAGAAAYY
VGYLQPRTFLLKYNENGVITDAVDCALDPLSETKCTLKSFTVEKGIYITSLFEVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELNHAPATVCGPKKSTNLVKNKCVNFNFNGLTGTGVLTESNLKFVSTQLFR
VHSANTTLAVRDPQTLEILDIVSCSSGAVSVITPGTNTSNQVAVLYYNVWCTEVPVAIHA
DQLTPTWRVYSTGSNVFQTKAGCLIGAEHVNNSYECDIAIGGGICASYQTQTNSPGSASS
VASQSIIAYWISTGSWNSVDNSNDAIAIATNFTISVTTEILPVSMTKTWVICTLYICGGS
TECSNLLAQYGSFCTELNRALTGIAVEQDKNTWEVFAQVRTIFHTPSIKDFGGFNFSQIL
PDPSKPSKRSFIEDLLFNKVTLADAGFIKQYGCCLGDIAARDLSCHQDSRGLNILCSLLT
DELIAEFTSALLAGTITAGWSFTAGHALNIPWAVQMAWRFAGIGVTENVLAKNQKLIANQ
FNSAIGKIQDSLSSTASALGKLQDVVNQNAQALDELERELSSNFGAISSVLNDILSNLDP
PEAEVQIDRLILGRLMALAAFVTAQLIRAAEIRASANLAATKMAECVAGQSKLVGFCGEG
WHLMSFPQSAPHGVVFLHVTLVAGQTKNFTTALAICHDGKAHIPRNGVFVSNGTHWFVTQ
EQFYEPLIITTDLVLVSGNCDDVIGIVNNTVYDPKQPELDS
SEQ ID NO: 51-(CoV2_S2_1_hbnet_openDS2, SEQ ID NO: 10 as parent) mutant
Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYQISTGSWNSVENSNDAIAIATNFTISVTTEILPVSMTKTWVDCTLYICGGS
TECSNLLAQYGSFCTELNRMLTGIAVEQDKNTWEVFATVRTIFHTPSIKDFGGFNFSQIL
PDPSKPSKRSFIEDLLFNKVTLADAGFIKQYGCCLGDIAARDSSCAQKANGLNILCSLLT
DELIAEFTSALLAGTITAGWSFTAGAALNIPWWAQMAWRFAGIGVTENVLAKNQKLIANQ
FNSAIGKIQDSLSSTASALGKLQDVVNQNAQALAELEKELSSNFGAISSVLNDILSNLDP
PEAEVQIDRLILGRLMALAAFVTAQAIRAAEIRASANLAATKMRECVAGQSKLVGFCGEG
WHLMSFPQSAPHGVVFLHVTLVAGQYKNFTTAPAICHDGKAHIPRNGVFVSNGTHWFVTQ
EQFYEPLIITTDLVLVSGNCDDVIGIVNNTVYDPKQPELDS
SEQ ID NO: 52-(CoV2_S2_NTD_6_pross_openDS2, SEQ ID NO: 24 as parent) mutant
Spike (S) protein amino acid sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNATNVVIKVCEFQFCEDPFLGVYYH
KNNKSWMESEFHVYSSANNCTFEYVSQPFLMDLEGKQGNFKNLREFVFKNIDGYFKIYSK
HTPINLVRDLPQGFSALEPLVDLPIGINITRFQILLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEIIPVSMPKTSVDCTMYICGDS
TECSNLLLQYGSFCTQLNRALTEIAVEQDKNTQEVFAQVKQIYKTPPIKDFGGFNFSQIL
PDPSKPSKRSFIEDLLFNKVTLADAGFIKGYGCCLGDIAARDLICAQKFNGLTVLCPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQSLQTYVTQQLIRAAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 53-(CoV2_S2_6_pross_openDS2, SEQ ID NO: 29 as parent) mutant
Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEIIPVSMPKTSVDCTMYICGDS
TECSNLLLQYGSFCTQLNRALTEIAVEQDKNTQEVFAQVKQIYKTPPIKDFGGFNFSQIL
PDPSKPSKRSFIEDLLFNKVTLADAGFIKGYGCCLGDIAARDLICAQKFNGLTVLCPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQSLQTYVTQQLIRAAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 54-(CoV2_S2_1_hbnet_pross_openDS2 , SEQ ID NO: 30 as parent)
mutant Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIATNFTISVTTEILPVSMTKTSVDCTMYICGGS
TECSNLLLQYGSFCTQLNRALTEIAVEQDKNTQEVFAQVKQIYKTPPIKDFGGFNFSQIL
PDPSKPSKRSAIEDLLFNKVKLADAGFIKGYGCCLGDIAARDSICAQKFNGLTILCSLLT
DEMIAAFTSALLAGTITAGWTFGAGAALQIPFAMQMAYRFAGIGVTQNVLYENQKLIANQ
FNNAIGKIQDGLSSTASALGKLQDVVNQNAQALNTLVKQLSSNFGAISSVLNDILSNLDP
PEAEVQIDRLITGRLQSLQTYVTQQAIRAAEIRASANLAATKMSECVLGQSKLVDFCGKG
YHLMSFPQSAPHGVVFLHVTYVATQYKNFTTAPAICHDGKAHFPREGVFVSNGTHWFVTQ
RNFYEPQIITTDLTFVSGNCDVVIGIVNNTVYDPLQPELDS
SEQ ID NO: 55-(CoV2_S_1_hbnet_openDS3, SEQ ID NO: 5 as parent) mutant Spike
(S) protein amino acid sequence:
AYTNSFTRGVYYPDKVSMSSVLHSTQDLFLPFFSNVTWFHAIHVSGTNGTKRFDNPVLPF
NDGVYFASTEKSNIIRGWIFGTTLDSKTOSLLIVNNATNVVIKVCEFQFCNDPFLGVYYH
KNNKSWMESEFRVYSSANNCTFEYVSQPFLMDLEGKQGNFKNLREEVFKNIDGYFKIYSK
HTPINLVRDLPQGFSALELLVDLPIGINITRFQTLLALHRSYLTPGDSSSGWTAGAAAYY
VGYLQPRTFLLKYNENGVITDAVDCALDPLSETKCTLKSFTVEKGIYITSLFEVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELNHAPATVCGPKKSTNLVKNKCVNFNFNGLTGTGVLTESNLKFVSTQLFR
VHSCNTTLAVRDPQTLEILDIVSCSSGAVSVITPGTNTSNQVAVLYYNVWCTEVPVAIHA
DQLTPTWRVYSTGSNVFQTKAGCLIGAEHVNNSYECDIAIGGGICASYQTQTNSPGSASS
VASQSIIAYWISTGSWNSVDNSNDAIAIATNFTISVTTEILPVSMTKTWVICTLYICGGS
TECSNLLAQYGSFCTELNRALTGIAVEQDKNTWEVFAQVRTIFHTPSIKDFGGFNFSQIL
PDPSKPSKRSFIEDLLFNKVTLADAGFIKQYGDCLGDIAARDLSCHQDSRGLNILSSLLT
DELIAEFTSALLAGTITAGWSFTAGHALNIPWAVQMAWRFAGIGVTENVLAKNQKLIANQ
FNSAIGKIQDSLSSTASALGKLQDVVNQNAQALDELERELCSNFGAISSVLNDILSNLDP
PEAEVQIDRLILGRLMALAAFVTAQLIRAAEIRASANLAATKMAECVAGQSKLVGFCGEG
WHLMSFPQSAPHGVVFLHVTLVAGQTKNFTTALAICHDGKAHIPRNGVFVSNGTHWFVTQ
EQFYEPLIITTDLVLVSGNCDDVIGIVNNTVYDPKQPELDS
SEQ ID NO: 56-(CoV2_S2_1_hbnet_openDS3, SEQ ID NO: 10 as parent) mutant
Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDICDTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYQISTGSWNSVENSNDAIAIATNFTISVTTEILPVSMTKTWVDCTLYICGGS
TECSNLLAQYGSFCTELNRMLTGIAVEQDKNTWEVFATVRTIFHTPSIKDFGGFNFSQIL
PDPSKPSKRSFIEDLLFNKVTLADAGFIKQYGDCLGDIAARDSSCAQKANGLNILSSLLT
DELIAEFTSALLAGTITAGWSFTAGAALNIPWWAQMAWRFAGIGVTENVLAKNQKLIANQ
FNSAIGKIQDSLSSTASALGKLQDVVNQNAQALAELEKELCSNFGAISSVLNDILSNLDP
PEAEVQIDRLILGRLMALAAFVTAQAIRAAEIRASANLAATKMRECVAGQSKLVGFCGEG
WHLMSFPQSAPHGVVFLHVTLVAGQYKNFTTAPAICHDGKAHIPRNGVFVSNGTHWFVTQ
EQFYEPLIITTDLVLVSGNCDDVIGIVNNTVYDPKQPELDS
SEQ ID NO: 57-(CoV2_S2_NTD_6_pross_openDS3, SEQ ID NO: 24 as parent) mutant
Spike (S) protein amino acid sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNATNVVIKVCEFQFCEDPFLGVYYH
KNNKSWMESEFHVYSSANNCTFEYVSQPFLMDLEGKQGNFKNLREFVFKNIDGYFKIYSK
HTPINLVRDLPQGFSALEPLVDLPIGINITRFQILLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDICDTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEIIPVSMPKTSVDCTMYICGDS
TECSNLLLQYGSFCTQLNRALTEIAVEQDKNTQEVFAQVKQIYKTPPIKDFGGFNFSQIL
PDPSKPSKRSFIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIODGLSSTASALGKLQDVVNONAOALNTLVKQLCSNFGAISSVLNDILSRLDP
PEAEVQIDRLINGRLQSLQTYVTQQLIRAAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 58-(CoV2_S2_6_pross_openDS3, SEQ ID NO: 29 as parent) mutant
Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDICDTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEI1PVSMPKTSVDCTMYICGDS
TECSNLLLQYGSFCTQLNRALTEIAVEQDKNTQEVFAQVKQIYKTPPIKDFGGFNFSQIL
PDPSKPSKRSFIEDLLFNKVTLADAGFIKGYGDCLGDIAARDLICAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLCSNFGAISSVLNDILSRLDP
PEAEVQIDRLINGRLQSLQTYVTQQLIRAAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 59-(CoV2_S2_1_hbnet_pross_openDS3, SEQ ID NO: 30 as parent)
mutant Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDICDTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIATNFTISVTTEILPVSMTKTSVDCTMYICGGS
TECSNLLLQYGSFCTQLNRALTEIAVEQDKNTQEVFAQVKQIYKTPPIKDFGGFNFSQIL
PDPSKPSKRSAIEDLLFNKVKLADAGFIKGYGDCLGDIAARDSICAQKFNGLTILSSLLT
DEMIAAFTSALLAGTITAGWTFGAGAALQIPFAMQMAYRFAGIGVTQNVLYENQKLIANQ
FNNAIGKIQDGLSSTASALGKLQDVVNQNAQALNTLVKQLCSNFGAISSVLNDILSNLDP
PEAEVQIDRLITGRLQSLQTYVTQQAIRAAEIRASANLAATKMSECVLGQSKLVDFCGKG
YHLMSFPQSAPHGVVFLHVTYVATQYKNFTTAPAICHDGKAHFPREGVFVSNGTHWFVTQ
RNFYEPQIITTDLTFVSGNCDVVIGIVNNTVYDPLQPELDS
SEQ ID NO: 60-(CoV2_S_1_hbnet_openDS4, SEQ ID NO: 5 as parent) mutant Spike
(S) protein amino acid sequence:
AYTNSFTRGVYYPDKVSMSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALELLVDLPIGINITRFQTLLALHRSYLTPGDSSSGWTAGAAAYY
VGYLQPRTFLLKYNENGVITDAVDCALDPLSETKCTLKSFTVEKGIYITSLFEVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGENCYFPLQSYGFQPTNGVGYQ
PYRVVVLSFELNHAPATVCGPKKSTNLVKNKCVNFNFNGLTGTGVLTESNLKFVSTQLFR
VHSANTTLAVRDPQTLEILCIVSCSSGAVSVITPGTNTSNQVAVLYYNVWCTEVPVAIHA
DQLTPTWRVYSTGSNVFQTKAGCLIGAEHVNNSYECDIAIGGGICASYQTQTNSPGSASS
VASQSIIAYWISTGSWNSVDNSNDAIAIATNFTISVTTEILPVSMTKTWVICTLYICGGS
TECSNLLAQYGSFCTELNRALTGIAVEQDKNTWEVFAQVRTIFHTPSIKDFGGFNFSQIL
PDPSKPSKRSFIEDLLFNKVTLADAGFIKQYGDCLGDIAARDLCCHQDSRGLNILSSLLT
DELIAEFTSALLAGTITAGWSFTAGHALNIPWAVQMAWRFAGIGVTENVLAKNQKLIANQ
FNSAIGKIQDSLSSTASALGKLQDVVNQNAQALDELERELSSNFGAISSVLNDILSNLDP
PEAEVQIDRLILGRLMALAAFVTAQLIRAAEIRASANLAATKMAECVAGQSKLVGFCGEG
WHLMSFPQSAPHGVVFLHVTLVAGQTKNFTTALAICHDGKAHIPRNGVFVSNGTHWFVTQ
EQFYEPLIITTDLVLVSGNCDDVIGIVNNTVYDPKQPELDS
SEQ ID NO: 61-(CoV2_S2_1_hbnet_openDS4, SEQ ID NO: 10 as parent) mutant
Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILCITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYQISTGSWNSVENSNDAIAIATNFTISVTTEILPVSMTKTWVDCTLYICGGS
TECSNLLAQYGSFCTELNRMLTGIAVEQDKNTWEVFATVRTIFHTPSIKDFGGFNFSQIL
PDPSKPSKRSFIEDLLFNKVTLADAGFIKQYGDCLGDIAARDSCCAQKANGLNILSSLLT
DELIAEFTSALLAGTITAGWSFTAGAALNIPWWAQMAWRFAGIGVTENVLAKNQKLIANQ
FNSAIGKIQDSLSSTASALGKLQDVVNQNAQALAELEKELSSNFGAISSVLNDILSNLDP
PEAEVQIDRLILGRLMALAAFVTAQAIRAAEIRASANLAATKMRECVAGQSKLVGFCGEG
WHLMSFPQSAPHGVVFLHVTLVAGQYKNFTTAPAICHDGKAHIPRNGVFVSNGTHWFVTQ
EQFYEPLIITTDLVLVSGNCDDVIGIVNNTVYDPKQPELDS
SEQ ID NO: 62-(CoV2_S2_NTD_6_pross_openDS4, SEQ ID NO: 24 as parent) mutant
Spike (S) protein amino acid sequence:
AYTNSFTRGVYYPDKVFRSNVLHLTQDLFLPFFSNVTWFHAIHVSGTNGTKRFDNPVLPF
NDGVYFASTEKSNIIRGWIFGTTLDSKTQSLLIVNNATNVVIKVCEFQFCEDPFLGVYYH
KNNKSWMESEFHVYSSANNCTFEYVSQPFLMDLEGKQGNFKNLREFVFKNIDGYFKIYSK
HTPINLVRDLPQGFSALEPLVDLPIGINITRFQILLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILCITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEIIPVSMPKTSVDCTMYICGDS
TECSNLLLQYGSFCTQLNRALTEIAVEQDKNTQEVFAQVKQIYKTPPIKDFGGFNFSQIL
PDPSKPSKRSFIEDLLFNKVTLADAGFIKGYGDCLGDIAARDLCCAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQSLQTYVTQQLIRAAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 63-(CoV2_S2_6_pross_openDS4, SEQ ID NO: 29 as parent) mutant
Spike (S) protein amino acid sequence:
AYTNSETRGVYYPDKVFRSSVLHSTQDLFLPFFSNVTWFHAIHVSGTNGTKRFDNPVLPE
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILCITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEIIPVSMPKTSVDCTMYICGDS
TECSNLLLQYGSFCTQLNRALTEIAVEQDKNTQEVFAQVKQIYKTPPIKDFGGFNFSQIL
PDPSKPSKRSFIEDLLFNKVTLADAGFIKGYGDCLGDIAARDLCCAQKFNGLTVLPPLLT
DEMIAAYTSALLAGTITAGWTFGAGAALQIPFAMQMAYRFNGIGVTQNVLYENQKLIANQ
FNKAIGKIQDGLSSTASALGKLQDVVNQNAQALNTLVKQLSSNFGAISSVLNDILSRLDP
PEAEVQIDRLINGRLQSLQTYVTQQLIRAAEIRASANLAAEKMSECVLGQSKRVDFCGKG
YHLMSFPQSAPHGVVFLHVTYVPTQHKNFTTAPAICHDGKAHFPREGVFVSNGTHWFVTQ
RNFYEPQIITTDNTFVSGNCDVVIGIVNNTVYDPLQPELDS
SEQ ID NO: 64-(CoV2_S2_1_hbnet_pross_openDS4, SEQ ID NO: 30 as parent)
mutant Spike (S) protein amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILCITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIATNFTISVTTEILPVSMTKTSVDCTMYICGGS
TECSNLLLQYGSFCTQLNRALTEIAVEQDKNTQEVFAQVKQIYKTPPIKDFGGFNFSQIL
PDPSKPSKRSAIEDLLFNKVKLADAGFIKGYGDCLGDIAARDSCCAQKFNGLTILSSLLT
DEMIAAFTSALLAGTITAGWTFGAGAALQIPFAMQMAYRFAGIGVTQNVLYENQKLIANQ
FNNAIGKIQDGLSSTASALGKLQDVVNQNAQALNTLVKQLSSNFGAISSVLNDILSNLDP
PEAEVQIDRLITGRLQSLQTYVTQQAIRAAEIRASANLAATKMSECVLGQSKLVDFCGKG
YHLMSFPQSAPHGVVFLHVTYVATQYKNFTTAPAICHDGKAHFPREGVFVSNGTHWFVTQ
RNFYEPQIITTDLTFVSGNCDVVIGIVNNTVYDPLQPELDS
SEQ ID NO: 65-(CoV2_RBD_K417F_K391F) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGFIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 66-(CoV2_RBD_K417L_K391L) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGLIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 67-(CoV2_RBD_K417M_K391M) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGMIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 68-(CoV2_RBD_K417W_K391W) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNWIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGWIADYNYKLPDDFTGCVIAWNSNNLDSKVG
GNYNYLYRLFRKSNLKPFERDISTEIYQAGSTPCNGVEGENCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 69-(CoV2_RBD_K417Y_K391Y) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGYIADYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 70-(CoV2_RBD_Y449A_Y423A) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNANYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 71-(Cov2_RBD_Y453A_Y427A) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLARLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 72-(CoV2_RBD_L455A_L429A) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRAFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 73- (CoV2_RBD_L455H_L429H) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRHFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 74-(CoV2_RBD_L455M_L429M) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRMFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 75-(CoV2_RBD_L455N_L429N) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRNFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 76-(CoV2_RBD_L455W_L429W) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRWFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 77-(CoV2_RBD_F456H_F430H) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLHRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 78-(CoV2_RBD_F4561_F4301 ) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLIRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 79-(Cov2_RBD_F456W_F430W) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLWRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 80-(CoV2_RBD_F456Y_F430Y) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLYRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 81-(CoV2_RBD_Y473W_Y447W) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIWQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNETISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 82-(CoV2_RBD_A475M_A449M) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQMGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 83-(CoV2_RBD_G476T_G450T) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQATSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 84-(CoV2_RBD_F486H_F460H) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGHNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 85-(CoV2_RBD_F4861_F4601) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGINCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 86-(CoV2_RBD_F486L_F460L) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGLNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTEVSGNCDVVIGIVNNTVYDPLQPELDS
SEQ ID NO: 87-(CoV2_RBD_F486M_F460M) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGMNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 88-(CoV2_RBD_F486N_F460N) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGNNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 89-(CoV2_RBD_F486P_F460P) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGPNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 90-(CoV2_RBD_F486T_F460T) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGTNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 91-(CoV2_RBD_F486W_F460W) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGWNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 92-(CoV2_RBD_F486Y_F460Y) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTOSLLIVNNATNVVIKVCEFOFCNDPFLGVYYH
KNNKSWMESEFRVYSSANNCTFEYVSQPFLMDLEGKQGNFKNLREEVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGYNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 93-(CoV2_RBD_N487F_N461F) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFFCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 94-(CoV2_RBD_N487L_N461L) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFLCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVELHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 95-(CoV2_RBD_N487M_N461M) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFMCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 96-(CoV2_RBD_N487Q_N461Q) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFQCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 97-(CoV2_RBD_Q493A_Q467A) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLASYGFQPTNGVGYQ
PYRVVVLSFELLHAPATVCGPKKSTNLVKNKCVNFNFNGLTGTGVLTESNKKFLPFOOFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 98-(CoV2_RBD_Q493Y_Q467Y) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLYSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 99-(CoV2_RBD_Q493F_Q467F) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLFSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 100-(CoV2_RBD_Q493R_Q467R) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLRSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 101-(CoV2_RBD_Q493M_Q467M) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLMSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 102-(CoV2_RBD_Q493C_Q467C) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLCSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 103-(CoV2_RBD_Q493G_Q467G) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLGSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 104-(CoV2_RBD_Q493V_Q467V) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLVSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 105-(CoV2_RBD_K417N_A419T_K391N_A393T) mutant Spike (S) protein
amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGNITDYNYKLPDDFTGCVIAWNSNNLDSKVG
GNYNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNENGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 106-(CoV2_RBD_Y449N_Y451T_Y423N_Y425T) mutant Spike (S) protein
amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNNNTLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 107-(CoV2_RBD_Y453N_L455T_Y427N_L429T) mutant Spike (S) protein
amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLNRTFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 108-(CoV2_RBD_L455N_R457T_L429N_R431T) mutant Spike (S) protein
amino acid sequence:
AYTNSETRGVYYPDKVFRSSVLHSTQDLFLPFFSNVTWFHAIHVSGTNGTKRFDNPVLPE
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRNFTKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 109-(CoV2_RBD_F456N_K458T_F430N_K432T) mutant Spike (S) protein
amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLNRTSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 110-(CoV2_RBD_Y473N_A475T_Y447N_A449T) mutant Spike (S) protein
amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEINQTGSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 111-(CoV2_RBD_A475N_S477T_A449N_S451T) mutant Spike (S) protein
amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQNGTTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 112-(CoV2_RBD_G476N_G450N) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQANSTPCNGVEGFNCYFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 113-(CoV2_RBD_Y489T_Y463T) mutant Spike (S) protein amino acid
sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCTFPLQSYGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 114-(CoV2_RBD_Q493N_Y495T_Q467N_Y469T) mutant Spike (S) protein
amino acid sequence:
AYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYH
KNNKSWMESEFRVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLNSTGFQPTNGVGYQ
PYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQTNSPGSASS
VASQSIIAYTMSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDS
SEQ ID NO: 115-a wild type amino acid sequence of Human Severe Acute
Respiratory Syndrome (SARS) coronavirus (SARS-CoV-1) Spike (S) glycoprotein
having the following features N'-C' (Li F. et al. 2005 Science
309(5742):1864-1868; submitted as UniProtKB Accession No. P59594 entitled
SPIKE CVHSA entry 135 dated 22April2020; see also ″SARS-CoV″ in Wrapp et al.
2020 Science 367(6483):1260-1263 and Supplementary Materials):
Signal peptide residues 1-13 (underlined)
                  10 20 30 40 50 60
MFIFLLFLTL TSGSDLDRCT TFDDVQAPNY TQHTSSMRGV YYPDEIFRSD TLYLTQDLFL
                  70 80 90 100 110 120
PFYSNVTGFH TINHTFGNPV IPFKDGIYFA ATEKSNVVRG WVFGSTMNNK SQSVIIINNS
                  130 140 150 160 170 180
TNVVIRACNF ELCDNPFFAV SKPMGTQTHT MIFDNAFNCT FEYISDAFSL DVSEKSGNFK
                  190 200 210 220 230 240
HLREFVFKNK DGFLYVYKGY QPIDVVRDLP SGFNTLKPIF KLPLGINITN FRAILTAFSP
                  250 260 270 280 290 300
AQDIWGTSAA AYFVGYLKPT TFMLKYDENG TITDAVDCSQ NPLAELKCSV KSFEIDKGIY
                  310 320 330 340 350 360
QTSNFRVVPS GDVVRFPNIT NLCPFGEVFN ATKFPSVYAW ERKKISNCVA DYSVLYNSTF
                  370 380 390 400 410 420
FSTFKCYGVS ATKLNDLCFS NVYADSFVVK GDDVRQIAPG QTGVIADYNY KLPDDFMGCV
                  430 440 450 460 470 480
LAWNTRNIDA TSTGNYNYKY RYLRHGKLRP FERDISNVPF SPDGKPCTPP ALNCYWPLND
                  490 500 510 520 530 540
YGFYTTTGIG YQPYRVVVLS FELLNAPATV CGPKLSTDLI KNQCVNFNFN GLTGTGVLTP
                  550 560 570 580 590 600
SSKRFQPFQQ FGRDVSDFTD SVRDPKTSEI LDISPCSFGG VSVITPGTNA SSEVAVLYQD
                  610 620 630 640 650 660
VNCTDVSTAI HADQLTPAWR IYSTGNNVFQ TQAGCLIGAE HVDTSYECDI PIGAGICASY
                  670 680 690 700 710 720
HTVSLLRSTS QKSIVAYTMS LGADSSIAYS NNTIAIPTNF SISITTEVMP VSMAKTSVDC
                  730 740 750 760 770 780
NMYICGDSTE CANLLLQYGS FCTQLNRALS GIAAEQDRNT REVFAQVKQM YKTPTLKYFG
                  790 800 810 820 830 840
GFNFSQILPD PLKPTKRSFI EDLLFNKVTL ADAGFMKQYG ECLGDINARD LICAQKFNGL
                  850 860 870 880 890 900
TVLPPLLTDD MIAAYTAALV SGTATAGWTF GAGAALQIPF AMQMAYRFNG IGVTQNVLYE
                  910 920 930 940 950 960
NQKQIANQFN KAISQIQESL TTTSTALGKL QDVVNQNAQA LNTLVKQLSS NFGAISSVLN
                  970 980 990 1000 1010 1020
DILSRLDKVE AEVQIDRLIT GRLQSLQTYV TQQLIRAAEI RASANLAATK MSECVLGQSK
                  1030 1040 1050 1060 1070 1080
RVDFCGKGYH LMSFPQAAPH GVVFLHVTYV PSQERNFTTA PAICHEGKAY FPREGVFVFN
                  1090 1100 1110 1120 1130 1140
GTSWFITQRN FFSPQIITTD NTFVSGNCDV VIGIINNTVY DPLQPELDSF KEELDKYFKN
                  1150 1160 1170 1180 1190 1200
HTSPDVDLGD ISGINASVVN IQKEIDRLNE VAKNLNESLI DLQELGKYEQ YIKWPWYVWL
                  1210 1220 1230 1240 1250 1255
GFIAGLIAIV MVTILLCCMT SCCSCLKGAC SCGSCCKFDE DDSEPVLKGV KLHYT
SEQ ID NO: 116-residues 14-1255 of the SARS-CoV-1 Spike (S) protein amino
acid sequence SEQ ID NO: 115
                  10 20 30 40 50 60
SDLDRCTTFD DVQAPNYTQH TSSMRGVYYP DEIFRSDTLY LTQDLFLPFY SNVTGFHTIN
                  70 80 90 100 110 120
HTFGNPVIPF KDGIYFAATE KSNVVRGWVF GSTMNNKSQS VIIINNSTNV VIRACNFELC
                  130 140 150 160 170 180
DNPFFAVSKP MGTQTHTMIF DNAFNCTFEY ISDAFSLDVS EKSGNFKHLR EFVFKNKDGF
                  190 200 210 220 230 240
LYVYKGYQPI DVVRDLPSGF NTLKPIFKLP LGINITNFRA ILTAFSPAQD IWGTSAAAYF
                  250 260 270 280 290 300
VGYLKPTTFM LKYDENGTIT DAVDCSQNPL AELKCSVKSF EIDKGIYQTS NFRVVPSGDV
                  310 320 330 340 350 360
VRFPNITNLC PFGEVFNATK FPSVYAWERK KISNCVADYS VLYNSTFFST FKCYGVSATK
                  370 380 390 400 410 420
LNDLCFSNVY ADSFVVKGDD VRQIAPGQTG VIADYNYKLP DDFMGCVLAW NTRNIDATST
                  430 440 450 460 470 480
GNYNYKYRYL RHGKLRPFER DISNVPFSPD GKPCTPPALN CYWPLNDYGF YTTTGIGYQP
                  490 500 510 520 530 540
YRVVVLSFEL LNAPATVCGP KLSTDLIKNQ CVNFNFNGLT GTGVLTPSSK RFQPFQQFGR
                  550 560 570 580 590 600
DVSDFTDSVR DPKTSEILDI SPCSFGGVSV ITPGTNASSE VAVLYQDVNC TDVSTAIHAD
                  610 620 630 640 650 660
QLTPAWRIYS TGNNVFQTQA GCLIGAEHVD TSYECDIPIG AGICASYHTV SLLRSTSQKS
                  670 680 690 700 710 720
IVAYTMSLGA DSSIAYSNNT IAIPTNFSIS ITTEVMPVSM AKTSVDCNMY ICGDSTECAN
                  730 740 750 760 770 780
LLLQYGSFCT QLNRALSGIA AEQDRNTREV FAQVKQMYKT PTLKYFGGFN FSQILPDPLK
                  790 800 810 820 830 840
PTKRSFIEDL LFNKVTLADA GFMKQYGECL GDINARDLIC AQKFNGLTVL PPLLTDDMIA
                  850 860 870 880 890 900
AYTAALVSGT ATAGWTFGAG AALQIPFAMQ MAYRFNGIGV TQNVLYENQK QIANQFNKAI
                  910 920 930 940 950 960
SQIQESLTTT STALGKLQDV VNQNAQALNT LVKQLSSNFG AISSVLNDIL SRLDKVEAEV
                  970 980 990 1000 1010 1020
QIDRLITGRL QSLQTYVTQQ LIRAAEIRAS ANLAATKMSE CVLGQSKRVD FCGKGYHLMS
                  1030 1040 1050 1060 1070 1080
FPQAAPHGVV FLHVTYVPSQ ERNFTTAPAI CHEGKAYFPR EGVFVFNGTS WFITQRNFFS
                  1090 1100 1110 1120 1130 1140
PQIITTDNTF VSGNCDVVIG IINNTVYDPL QPELDSFKEE LDKYFKNHTS PDVDLGDISG
                  1150 1160 1170 1180 1190 1200
INASVVNIQK EIDRLNEVAK NLNESLIDLQ ELGKYEQYIK WPWYVWLGFI AGLIAIVMVT
                  1210 1220 1230 1240 1242
ILLCCMTSCC SCLKGACSCG SCCKFDEDDS EPVLKGVKLH YT
SEQ ID NO: 117-a wild type amino acid sequence of Middle East Respiratory
Syndrome (MERS) coronavirus (MERS-CoV) Spike (S) glycoprotein having the
following features N'-C' (Millet and Whittaker; submitted as GenBank
Accession No. AFS88936.1 Version 1 dated December 4, 2012 entitled ″S protein
[Human betacoronavirus 2c EMC/2012]″ encoded by GenBank Accession No.
JX869059.2 see also Yang et al. 2014 Virol Immunol 27(10): 543-550 and Yuan
et al. 2017 Nat. Comm. 8(15092), 9 pgs & Suppl. Materials):
Signal peptide residues 1-18 (underlined)
                  10 20 30 40 50 60
MIHSVFLLMF LLTPTESYVD VGPDSVKSAC IEVDIQQTFF DKTWPRPIDV SKADGIIYPQ
                  70 80 90 100 110 120
GRTYSNITIT YQGLFPYQGD HGDMYVYSAG HATGTTPQKL FVANYSQDVK QFANGFVVRI
                  130 140 150 160 170 180
GAAANSTGTV IISPSTSATI RKIYPAFMLG SSVGNFSDGK MGRFFNHTLV LLPDGCGTLL
                  190 200 210 220 230 240
RAFYCILEPR SGNHCPAGNS YTSFATYHTP ATDCSDGNYN RNASLNSFKE YFNLRNCTFM
                  250 260 270 280 290 300
YTYNITEDEI LEWFGITQTA QGVHLFSSRY VDLYGGNMFQ FATLPVYDTI KYYSIIPHSI
                  310 320 330 340 350 360
RSIQSDRKAW AAFYVYKLQP LTFLLDFSVD GYIRRAIDCG FNDLSQLHCS YESFDVESGV
                  370 380 390 400 410 420
YSVSSFEAKP SGSWEQAEG VECDFSPLLS GTPPQVYNFK RLVFTNCNYN LTKLLSLFSV
                  430 440 450 460 470 480
NDFTCSQISP AAIASNCYSS LILDYFSYPL SMKSDLSVSS AGPISQFNYK QSFSNPTCLI
                  490 500 510 520 530 540
LATVPHNLTT ITKPLKYSYI NKCSRLLSDD RTEVPQLVNA NQYSPCVSIV PSTVWEDGDY
                  550 560 570 580 590 600
YRKQLSPLEG GGWLVASGST VAMTEQLQMG FGITVQYGTD TNSVCPKLEF ANDTKIASQL
                  610 620 630 640 650 660
GNCVEYSLYG VSGRGVFQNC TAVGVRQQRF VYDAYQNLVG YYSDDGNYYC LRACVSVPVS
                  670 680 690 700 710 720
VIYDKETKTH ATLFGSVACE HISSTMSQYS RSTRSMLKRR DSTYGPLQTP VGCVLGLVNS
                  730 740 750 760 770 780
SLFVEDCKLP LGQSLCALPD TPSTLTPRSV RSVPGEMRLA SIAFNHPIQV DQLNSSYFKL
                  790 800 810 820 830 840
SIPTNFSFGV TQEYIQTTIQ KVTVDCKQYV CNGFQKCEQL LREYGQFCSK INQALHGANL
                  850 860 870 880 890 900
RQDDSVRNLF ASVKSSQSSP IIPGFGGDFN LTLLEPVSIS TGSRSARSAI EDLLFDKVTI
                  910 920 930 940 950 960
ADPGYMQGYD DCMQQGPASA RDLICAQYVA GYKVLPPLMD VNMEAAYTSS LLGSIAGVGW
                  970 980 990 1000 1010 1020
TAGLSSFAAI PFAQSIFYRL NGVGITOOVL SENQKLIANK FNQALGAMQT GFTTTNEAFQ
                  1030 1040 1050 1060 1070 1080
KVQDAVNNNA QALSKLASEL SNTFGAISAS IGDIIQRLDV LEQDAQIDRL INGRLTTLNA
                  1090 1100 1110 1120 1130 1140
FVAQQLVRSE SAALSAQLAK DKVNECVKAQ SKRSGFCGQG THIVSFVVNA PNGLYFMHVG
                  1150 1160 1170 1180 1190 1200
YYPSNHIEVV SAYGLCDAAN PTNCIAPVNG YFIKTNNTRI VDEWSYTGSS FYAPEPITSL
                  1210 1220 1230 1240 1250 1260
NTKYVAPQVT YQNISTNLPP PLLGNSTGID FQDELDEFFK NVSTSIPNFG SLTQINTTLL
                  1270 1280 1290 1300 1310 1320
DLTYEMLSLQ QVVKALNESY IDLKELGNYT YYNKWPWYIW LGFIAGLVAL ALCVFFILCC
                  1330 1340 1350 1353
TGCGTNCMGK LKCNRCCDRY EEYDLEPHKV HVH
SEQ ID NO: 118-residues 19-1353 of the MERS-CoV-1 Spike (S) protein amino
acid sequence SEQ ID NO: 117
                  10 20 30 40 50 60
VDVGPDSVKS ACIEVDIQQT FFDKTWPRPI DVSKADGIIY PQGRTYSNIT ITYQGLFPYQ
                  70 80 90 100 110 120
GDHGDMYVYS AGHATGTTPQ KLFVANYSQD VKQFANGFVV RIGAAANSTG TVIISPSTSA
                  130 140 150 160 170 180
TIRKIYPAFM LGSSVGNFSD GKMGRFFNHT LVLLPDGCGT LLRAFYCILE PRSGNHCPAG
                  190 200 210 220 230 240
NSYTSFATYH TPATDCSDGN YNRNASLNSF KEYFNLRNCT FMYTYNITED EILEWFGITQ
                  250 260 270 280 290 300
TAQGVHLFSS RYVDLYGGNM FQFATLPVYD TIKYYSIIPH SIRSIQSDRK AWAAFYVYKL
                  310 320 330 340 350 360
QPLTFLLDFS VDGYIRRAID CGFNDLSQLH CSYESFDVES GVYSVSSFEA KPSGSVVEQA
                  370 380 390 400 410 420
EGVECDFSPL LSGTPPQVYN FKRLVFTNCN YNLTKLLSLF SVNDFTCSQI SPAAIASNCY
                  430 440 450 460 470 480
SSLILDYFSY PLSMKSDLSV SSAGPISQFN YKQSFSNPTC LILATVPHNL TTITKPLKYS
                  490 500 510 520 530 540
YINKCSRLLS DDRTEVPQLV NANQYSPCVS IVPSTVWEDG DYYRKQLSPL EGGGWLVASG
                  550 560 570 580 590 600
STVAMTEQLQ MGFGITVQYG TDTNSVCPKL EFANDTKIAS QLGNCVEYSL YGVSGRGVFQ
                  610 620 630 640 650 660
NCTAVGVRQQ RFVYDAYQNL VGYYSDDGNY YCLRACVSVP VSVIYDKETK THATLFGSVA
                  670 680 690 700 710 720
CEHISSTMSQ YSRSTRSMLK RRDSTYGPLQ TPVGCVLGLV NSSLFVEDCK LPLGQSLCAL
                  730 740 750 760 770 780
PDTPSTLTPR SVRSVPGEMR LASIAFNHPI QVDQLNSSYF KLSIPTNFSF GVTQEYIQTT
                  790 800 810 820 830 840
IQKVTVDCKQ YVCNGFQKCE QLLREYGQFC SKINQALHGA NLRQDDSVRN LFASVKSSQS
                  850 860 870 880 890 900
SPIIPGFGGD FNLTLLEPVS ISTGSRSARS AIEDLLFDKV TIADPGYMQG YDDCMQQGPA
                  910 920 930 940 950 960
SARDLICAQY VAGYKVLPPL MDVNMEAAYT SSLLGSIAGV GWTAGLSSFA AIPFAQSIFY
                  970 980 990 1000 1010 1020
RLNGVGITQQ VLSENQKLIA NKFNQALGAM QTGFTTTNEA FQKVQDAVNN NAQALSKLAS
                  1030 1040 1050 1060 1070 1080
ELSNTFGAIS ASIGDIIQRL DVLEQDAQID RLINGRLTTL NAFVAQQLVR SESAALSAQL
                  1090 1100 1110 1120 1130 1140
AKDKVNECVK AQSKRSGFCG QGTHIVSFVV NAPNGLYFMH VGYYPSNHIE VVSAYGLCDA
                  1150 1160 1170 1180 1190 1200
ANPTNCIAPV NGYFIKTNNT RIVDEWSYTG SSFYAPEPIT SLNTKYVAPQ VTYQNISTNL
                  1210 1220 1230 1240 1250 1260
PPPLLGNSTG IDFQDELDEF FKNVSTSIPN FGSLTQINTT LLDLTYEMLS LQQVVKALNE
                  1270 1280 1290 1300 1310 1320
SYIDLKELGN YTYYNKWPWY IWLGFIAGLV ALALCVFFIL CCTGCGTNCM GKLKCNRCCD
              1330 1335
RYEEYDLEPH KVHVH
SEQ ID NO: 119-SAM VEE TC-83 replicon 1-7561 60
```

| | |
|---|---|
| auaggcggcg caugagagaa gcccagacca auuaccuacc caaaauggag aaaguucacg | 60 |
| uugacaucga ggaagacagc ccauuccuca gagcuuugca gcggagcuuc ccgcaguuug | 120 |
| agguagaagc caagcagguc acugauaaug accaugcuaa ugccagagcg uuuucgcauc | 180 |
| uggcuucaaa acugaucgaa acggaggugg acccauccga cacgauccuu gacauuggaa | 240 |
| gugcgcccgc ccgcagaaug uauucuaagc acaaguauca uuguaucugu ccgaugagau | 300 |
| gugeggaaga uccggacaga uuguauaagu augcaacuaa gcugaagaaa aacuguaagg | 360 |
| aaauaacuga uaaggaauug gacaagaaaa ugaaggagcu cgccgccguc augagcgacc | 420 |
| cugaccugga aacugagacu augugccucc acgacgacga gucgugucgc uacgaagggc | 480 |
| aagucgcugu uuaccaggau guauacgcgg uugacggacc gacaagucuc uaucaccaag | 540 |
| ccaauaaggg aguuagaguc gccuacugga uaggcuuuga caccaccccu uuuauguuua | 600 |
| agaacuuggc uggagcauau ccaucauacu cuaccaacug ggccgacgaa accguguuaa | 660 |
| cggcucguaa cauaggccua ugcagcucug acguuaugga gcggucacgu agagggaugu | 720 |
| ccauucuuag aaagaaguau uugaaaccau ccaacaaugu ucuauucucu guuggcucga | 780 |
| ccaucuacca cgagaagagg gacuuacuga ggagcuggca ccugccgucu guauuucacu | 840 |
| uacguggcaa gcaaaauuac acaugucggu gugagacuau aguuaguugc gacggguacg | 900 |
| ucguuaaaag aauagcuauc aguccaggcc uguaugggaa gccuucaggc uaugcugcua | 960 |
| cgaugcaccg cgagggauuc uugugcugca aagugacaga cacauugaac ggggagaggg | 1020 |
| ucucuuuucc cgugugcacg uaugugccag cuacauugug ugaccaaaug acuggcauac | 1080 |
| uggcaacaga ugucagugcg gacgacgcgc aaaaacugcu gguugggcuc aaccagcgua | 1140 |
| uagucgucaa cggucgcacc cagagaaaca ccaauaccau gaaaaauuac cuuuugcccg | 1200 |
| uaguggccca ggcauuugcu aggugggcaa aggaauauaa ggaagaucaa gaagaugaaa | 1260 |
| ggccacuagg acuacgagau agacaguuag ucauggggug uuguugggcu uuuagaaggc | 1320 |
| acaagauaac aucuauuuau aagcgcccgg auacccaaac caucaucaaa gugaacagcg | 1380 |
| auuuccacuc auucgugcug cccaggauag gcaguaacac auuggagauc gggcugagaa | 1440 |
| caagaaucag gaaaauguua gaggagcaca aggagccguc accucucauu accgccgagg | 1500 |
| acguacaaga agcuaagugc gcagccgaug aggcuaagga ggugcgugaa gccgaggagu | 1560 |
| ugcgcgcagc ucuaccaccu uuggcagcug auguugagga gcccacucug gaagccgaug | 1620 |
| ucgacuugau guuacaagag gcuggggccg gcucagugga gacaccucgu ggcuugauaa | 1680 |
| agguuaccag cuacgauggc gaggacaaga ucggcucuua cgcugugcuu ucuccgcagg | 1740 |
| cuguacucaa gagugaaaaa uuaucuugca uccacccucu cgcugaacaa gucauaguga | 1800 |
| uaacacacuc uggccgaaaa gggcguuaug ccguggaacc auaccauggu aaaguagugg | 1860 |
| ugccagaggg acaugcaaua cccguccagg acuuucaagc ucugagugaa agugccacca | 1920 |
| uuguguacaa cgaacgugag uucguaaaca gguaccugca ccauauugcc acacauggag | 1980 |
| gagcgcugaa cacugaugaa gaauauuaca aaacugucaa gcccagcgag cacgacggcg | 2040 |
| aauaccugua cgacaucgac aggaaacagu gcgucaagaa agaacuaguc acugggcuag | 2100 |
| ggcucacagg cgagcuggug gauccucccu uccaugaauu cgccuacgag agucugagaa | 2160 |
| cacgaccagc cgcuccuuac caaguaccaa ccauaggggu guauggcgug ccaggaucag | 2220 |
| gcaagucugg caucauuaaa agcgcaguca ccaaaaaaga ucuaguggug agcgccaaga | 2280 |
| aagaaaacug ugcagaaauu auaagggacg ucaagaaaau gaaagggcug gacgucaaug | 2340 |
| ccagaacugu ggacucagug cucuugaaug gaugcaaaca ccccguagag acccuguaua | 2400 |
| uugacgaagc uuuugcuugu caugcaggua cucucagagc gcucauagcc auuauaagac | 2460 |
| cuaaaaaggc agugcucugc ggggauccca aacagugcgg uuuuuuuaac augaugugcc | 2520 |
| ugaaagugca uuuuaaccac gagauuugca cacaagucuu ccacaaaagc aucucucgcc | 2580 |
| guugcacuaa aucugugacu ucggucgucu caaccuuguu uuacgacaaa aaaaugagaa | 2640 |
| cgacgaaucc gaaagagacu aagauuguga uugacacuac cggcaguacc aaaccuaagc | 2700 |
| aggacgaucu cauucucacu uguuucagag ggugggugaa gcaguugcaa auagauuaca | 2760 |
| aaggcaacga aauaaugacg gcagcugccu cucaagggcu gacccguaaa gguguguaug | 2820 |
| ccguucggua caaggugaau gaaaauccuc uguacgcacc caccucagaa caugugaacg | 2880 |
| uccuacugac ccgcacggag gaccgcaucg uguggaaaac acuagccggc gacccaugga | 2940 |
| uaaaaacacu gacugccaag uacccuggga auuucacugc cacgauagag gaguggcaag | 3000 |
| cagagcauga ugccaucaug aggcacaucu uggagagacc ggacccuacc gacgucuucc | 3060 |
| agaauaaggc aaacgugugu ugggccaagg cuuuagugcc ggugcugaag accgcuggca | 3120 |
| uagacaugac cacugaacaa uggaacacug uggauuauuu ugaaacggac aaagcucacu | 3180 |
| cagcagagau aguauugaac caacuaugcg ugagguucuu uggacucgau cuggacuccg | 3240 |
| gucuauuuuc ugcacccacu guuccguuau ccauuaggaa uaaucacugg gauaacuccc | 3300 |
| cgucgccuaa cauguacggg cugaauaaag aagugguccg ucagcucucu cgcagguacc | 3360 |
| cacaacugcc ucgggcaguu gccacuggaa gagucuauga caugaacacu gguacacugc | 3420 |
| gcaauuauga uccgcgcaua aaccuaguac cuguaaacag aagacugccu caugcuuuag | 3480 |
| uccuccacca uaaugaacac ccacagagug acuuuucuuc auucgucagc aaauugaagg | 3540 |
| gcagaacugu ccuggugguc ggggaaaagu uguccguccc aggcaaaaug guugacuggu | 3600 |
| ugucagaccg gccugaggcu accuucagag cucggcugga uuuaggcauc ccaggugaug | 3660 |
| ugcccaaaua ugacauaaua uuuguuaaug ugaggacccc auauaaauac caucacuauc | 3720 |
| agcaguguga agaccaugcc auuaagcuua gcauguugac caagaaagcu ugucugcauc | 3780 |
| ugaaucccgg cggaaccugu gucagcauag guuaugguua cgcugacagg gccagcgaaa | 3840 |
| gcaucauugg ugcuauagcg cggcaguuca aguuuucccg gguaugcaaa ccgaaauccu | 3900 |
| cacuugaaga gaeggaaguu cuguuuguau ucauugggua cgaucgcaag gcccguacgc | 3960 |
| acaauccuua caagcuuuca ucaaccuuga ccaacauuua uacagguucc agacuccacg | 4020 |
| aagccggaug ugcacccuca uaucaugugg ugcgagggga uauugccacg gccaccgaag | 4080 |
| gagugauuau aaaugcugcu aacagcaaag gacaaccugg cggaggggug ugcggagcgc | 4140 |
| uguauaagaa auucccggaa agcuucgauu uacagccgau cgaaguagga aaagcgcgac | 4200 |
| uggucaaagg ugcagcuaaa cauaucauuc augccguagg accaaacuuc aacaaaguuu | 4260 |
| cggagguuga aggugacaaa caguuggcag aggcuuauga guccaucgcu aagauuguca | 4320 |
| acgauaacaa uuacaaguca guagcgauuc cacuguuguc caccggcauc uuuuccggga | 4380 |
| acaaagaucg acuaacccaa ucauugaacc auuugcugac agcuuuagac accacugaug | 4440 |
| cagauguagc cauauacugc agggacaaga aaugggaaau gacucucaag gaagcagugg | 4500 |
| cuaggagaga agcaguggag gagauaugca uauccgacga cucuucagug acagaaccug | 4560 |
| augcagagcu ggugagggug cauccgaaga guucuuuggc uggaaggaag ggcuacagca | 4620 |
| caagcgaugg caaaacuuuc ucauauuugg aagggaccaa guuucaccag gcggccagg | 4680 |
| auauagcaga aauuaaugcc auguggcccg uugcaacgga ggccaaugag cagguaugca | 4740 |
| uguauauccu cggagaaagc augagcagua uuaggucgaa augccccguc gaagagucgg | 4800 |
| aagccuccac accaccuagc acgcugccuu gcuugugcau ccaugccaug acuccagaaa | 4860 |
| gaguacagcg ccuaaaagcc ucacguccag aacaaauuac ugugugcuca uccuuuccau | 4920 |
| ugccgaagua uagaaucacu ggugugcaga agauccaaug cucccagccu auauuguucu | 4980 |
| caccgaaagu gccugcguau auucauccaa ggaaguaucu cguggaaaca ccaccgguag | 5040 |
| acgagacucc ggagccaucg gcagagaacc aauccacaga ggggacaccu gaacaaccac | 5100 |
| cacuuauaac cgaggaugag accaggacua gaacgccuga gccgaucauc aucgaagagg | 5160 |
| aagaagagga uagcauaagu uugcugucag auggcccgac ccaccaggug cugcaagucg | 5220 |
| aggcagacau ucacgggccg cccucuguau cuagcucauc cugguccauu ccucaugcau | 5280 |
| ccgacuuuga uguggacagu uuauccauac uugacacccu ggagggagcu agcgugacca | 5340 |
| gcggggcaac gucagccgag acuaacucuu acuucgcaaa gaguauggag uuucuggcgc | 5400 |
| gaccggugcc ugcgccucga acaguauuca ggaacccucc acaucccgcu ccgcgcacaa | 5460 |
| gaacaccguc acuugcaccc agcagggccu gcucgagaac cagccuaguu uccaccccgc | 5520 |
| caggcgugaa uagggugauc acuagagagg agcucgaggc gcuuaccccg ucacgcacuc | 5580 |
| cuagcagguc ggucucgaga accagccugg ucuccaaccc gccaggcgua aauaggguga | 5640 |
| uuacaagaga ggaguuugag gcguucguag cacaacaaca augacgguuu gaugcgggug | 5700 |
| cauacaucuu uuccuccgac accggucaag ggcauuuaca acaaaaauca guaaggcaaa | 5760 |
| cggugcuauc cgaaguggug uuggagagga ccgaauugga gauuucguau gccccgcgcc | 5820 |
| ucgaccaaga aaaagaagaa uuacuacgca agaaauuaca guuaaauccc acaccugcua | 5880 |
| acagaagcag auaccagucc aggaaggugg agaacaugaa agccauaaca gcuagacgua | 5940 |
| uucugcaagg ccuagggcau uauuugaagg cagaaggaaa aguggagugc uaccgaaccc | 6000 |
| ugcauccugu uccuuuguau ucaucuagug ugaaccgugc cuuuucaagc cccaaggucg | 6060 |
| caguggaagc cuguaacgcc auguugaaag agaacuuucc gacuguggcu ucuuacugua | 6120 |
| uuauuccaga guacgaugcc uauuuggaca ugguugaegg agcuucaugc ugcuuagaca | 6180 |
| cugccaguuu uugcccugca aagcugcgca gcuuuccaaa gaaacacucc uauuuggaac | 6240 |
| ccacaauacg aucggcagug ccuucagcga uccagaacac gcuccagaac guccuggcag | 6300 |
| cugccacaaa aagaaauugc aaugucacgc aaaugagaga auugcccgua uuggauucgg | 6360 |
| cggccuuuaa uguggaaugc uucaagaaau augcguguaa uaaugaauau ugggaaacgu | 6420 |
| uuaaagaaaa ccccaucagg cuuacugaag aaaacguggu aaauuacauu accaaauuaa | 6480 |
| aaggaccaaa agcugcugcu cuuuuugcga agacacauaa uuugaauaug uugcaggaca | 6540 |
| uaccaaugga cagguuugua auggacuuaa agagagacgu gaaagugacu ccaggaacaa | 6600 |
| aacauacuga agaacggccc aagguacagg ugauccaggc ugccgauccg cuagcaacag | 6660 |
| cguaucugug cggaauccac cgagagcugg uuaggagauu aaaugcgguc cugcuuccga | 6720 |
| acauucauac acuguuugau augucggcug aagacuuuga cgcuauuaua gccgagcacu | 6780 |
| uccagccugg ggauuguguu cuggaaacug acaucgcguc guuugauaaa agugaggacg | 6840 |
| acgccauggc ucugaccgcg uuaaugauuc uggaagacuu agguguggac gcagagcugu | 6900 |
| ugacgcugau ugaggcggcu uucggcgaaa uuucaucaau acauuugccc acuaaaacua | 6960 |
| aauuuaaauu cggagccaug augaaaucug gaauguuccu cacacuguuu gugaacacag | 7020 |
| ucauuaacau uguaaucgca agcagagugu ugagagaacg gcuaaccgga ucaccaugug | 7080 |
| cagcauucau uggagaugac aauaucguga aaggagucaa aucggacaaa uuaauggcag | 7140 |
| acaggugcgc caccugguug aauauggaag ucaagauuau agaugcugug gugggcgaga | 7200 |
| aagcgccuua uuucugugga ggguuuauuu ugugugacuc cgugaccggc acagcgugcc | 7260 |
| guguggcaga cccccuaaaa aggcuguuua agcuuggcaa accucuggca gcagacgaug | 7320 |
| aacaugauga ugacaggaga agggcauugc augaagaguc aacacgcugg aaccgagugg | 7380 |
| guauucuuuc agagcugugc aaggcaguag aaucaaggua ugaaaccgua ggaacuucca | 7440 |
| ucauaguuau ggccaugacu acucuagcua gcaguguuaa aucauucagc uaccugagag | 7500 |
| gggccccuau aacucucuac ggcuaaccug aauggacuac gacauagucu aguccgccaa | 7560 |
| g | 7561 |

SEQ ID NO: 120-SAM VEE TC-83 replicon 7562-7747

| | |
|---|---|
| ucuagacggc gcgcccaccc agcggccgca uacagcagca auuggcaagc ugcuuacaua | 60 |
| gaacucgcgg cgauuggcau gccgccuuaa aauuuuuauu uuauuuuucu UUUCUUUUCC | 120 |
| gaaucggauu uuguuuuuaa uauuucaaaa aaaaaaaaaa aaaaaaaaaa aaaaaaaaaa | 180 |
| aaaaaa | 186 |

SEQ ID NO: 121-a Glycine/Serine/Alanine linker
10
GGGGSGGGGS
SEQ ID NO: 122-a PADRE linker
10 13
AKFVAAWTLK AAA
SEQ ID NO: 123-a D linker
10 15
QSIALSSLMV AQAIP
SEQ ID NO: 124-a TpD linker
10 20 30 32
ILMQYIKANS KFIGIPMGLP QSIALSSLMV AQ
SEQ ID NO: 125-B.1.351_PROSS_0_5
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEIYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQ
GVNCTEVPVAIHADQLTPTWRVYSTGSNVFQTRAGCLIGAEHVNNSYECDIPIGAGICAS
YQTQTNSPGSASSVANQSIIAYTMSLGVENPIPYSNNVIAIPTNFTISVTTEVIPVSMTK
TSVDCAQYICGDNEECEQLLLQYGSFCDQLNRALHEIAVKQDEALLEVFAQVKQIYKTPE
IKDFGGFNFSQILPDPSKSSYRSAIEDLLFNKVKLSDPGFIKQYQDCLGDNSARDLICAQ
FFNGLTVLPPLLTDEMIAAYTSALLAGTITAGWTFGAGSALAIPFALQMAYRFNGIGVTQ
NVLYENQKLIANQFNKAITKIQESLTTTSQALAKLQDVVNQNAQALNTLVKQLSNKFGAI
SSVLNDILSRLDPPEAKVQIDRLITGRLQALQTYVTQQLIRAAEIKASAQLAATKMSECV
LGQSTRVNFCGKGYHLMSFPQSAPHGVVFLHVTYVPSQFKNFTTAPAICHDGRAYFPREG
VFVSNGTEWFVTQRNFYEPQPITTDNTFVSGNCDVVIGIVNNTVYDPLQPDLDS
SEQ ID NO: 126-B.1.351_PROSS_1_5
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEIYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQ
GVNCTEVPVAIHADQLTPTWRVYSTGSNVFQTRAGCLIGAEHVNNSYECDIPIGAGICAS
YQTQTNSPGSASSVANQSIIAYTMSLGVENPIPYSNNVIAIPTNFTISVTTEIIPVSMTK
TSVDCAQYICGDNSECENLLLQYGSFCDQLNRALHEIAVKQDEALLEVFAQVKQIYKTPP
IKDFGGFNFSQILPDPSKPSYRSAIEDLLFNKVKLSDPGFIKQYEDCLGDNSARDLICAQ
FFNGLTVLPPLLTDEMIAAYTSALLAGTITAGWTFGAGSALAIPFALQMAYRFNGIGVTQ
NVLYENQKLIANQFNKAITKIQESLTSTNQALAKLQDVVNQNAQALNTLVKQLSNNFGAI
SSVLNDILSRLDPPEAKVQIDRLITGRLQALQTYVTQQLIRAAEIKASAELAATKMSECV
LGQSKRVNFCGKGYHLMSFPQSAPHGVVFLHVTYVPSQYKNFTTAPAICHDGRAHFPREG
VFVSNGTDWYVTQRNFYEPQPITTDNTFVSGNCDVVIGIVNNTVYDPLQPDLDS
SEQ ID NO: 127-B.1.351_PROSS_3_5
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEIYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQ
GVNCTEVPVAIHADQLTPTWRVYSTGSNVFQTRAGCLIGAEHVNNSYECDIPIGAGICAS
YQTQTNSPGSASSVASQSIIAYTMSLGVENPIPYSNNVIAIPTNFTISVTTEIIPVSMTK
TSVDCAQYICGDSTECENLLLQYGSFCDQLNRALHEIAVKQDENTQEVFAQVKQIYKTPP
IKDFGGFNFSQILPDPSKPSYRSVIEDLLFNKVTLSDPGFIKQYQDCLGDPSARDLICAQ
KFNGLTVLPPLLTDEMIAAYTSALLAGTITAGWTFGAGSALAIPFAMQMAYRFNGIGVTQ
NVLYENQKLIANQFNKAIGKIQDSLSSTSSALAKLQDVVNQNAQALNTLVKQLSNNFGAI
SSVLNDILSRLDPPEAKVQIDRLITGRLQALQTYVTQQLIRAAEIKASAELAATKMSECV
LGQSKRVNFCGKGYHLMSFPQSAPHGVVFLHVTYVPAQYKNFTTAPAICHDGRAHFPREG
VFVSNGTHWFVTQRNFYEPQPITTDNTFVSGNCDVVIGIVNNTVYDPLQPELDS
SEQ ID NO: 128-B.1.351_PROSS_4_0
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEIYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQ
GVNCTEVPVAIHADQLTPTWRVYSTGSNVFQTRAGCLIGAEHVNNSYECDIPIGAGICAS
YQTQTNSPGSASSVAQQSIIAYTMSLGVENPIPYSNNVIAIPTNFTISVTTEIIPVSMTK
TSVDCAQYICGDSTECENLLLQYGSFCTQLNRALHEIAVEQDKNTQEVFAQVKQIYKTPP
IKDFGGFNFSQILPDPSKPSYRSVIEDLLFNKVTLSDPGFIKQYQDCLGDPAARDLICAQ
KFNGLTVLPPLLTDEMIAAYTSALLAGTITAGWTFGAGSALAIPFAMQMAYRFNGIGVTQ
NVLYENQKLIANQFNKAIGKIQDSLSSTSSALAKLQDVVNQNAQALNTLVKQLSNNFGAI
SSVLNDILSRLDPPEAKVQIDRLITGRLQALQTYVTQQLIRAAEIKASAELAATKMSECV
LGQSKRVNFCGKGYHLMSFPQSAPHGVVFLHVTYVPAQYKNFTTAPAICHDGRAHFPREG
VFVSNGTHWFVTQRNFYEPQPITTDNTFVSGNCDVVIGIVNNTVYDPLQPELDS
SEQ ID NO: 129-B.1.351_PROSS_5_5
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEPYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQ
GVNCTEVPVAIHADQLTPTWRVYSTGSNVFQTRAGCLIGAEHVNNSYECDIPIGAGICAS
YQTQTNSPGSASSVASQSIIAYTMSLGVENPIPYSNNVIAIPTNFTISVTTEIIPVSMTK
TSVDCAQYICGDSTECENLLLQYGSFCTQLNRALTEIAVEQDKNTQEVFAQVKQIYKTPP
IKDFGGFNFSQILPDPSKPSYRSFIEDLLFNKVTLADPGFIKQYQDCLGDPAARDLICAQ
KFNGLTVLPPLLTDEMIAAYTSALLAGTITSGWTFGAGSALAIPFAMQMAYRFNGIGVTQ
NVLYENQKLIANQFNKAIGKIQDSLSSTSSALGKLQDVVNQNAQALNTLVKQLSSNFGAI
SSVLNDILSRLDPPEAEVQIDRLITGRLQALQTYVTQQLIRAAEIKASANLAATKMSECV
LGQSKRVDFCGKGYHLMSFPQSAPHGVVFLHVTYVPAQYKNFTTAPAICHDGKAHFPREG
VFVSNGTHWFVTORNFYEPQPITTDNTFVSGNCDVVIGIVNNTVYDPLQPELDS
SEQ ID NO: 130-B.1.351_Buried_PROSS_1_0
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEIYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQ
GVNCTEVPVAIHADQLTPTWRVYSTGSNVFQTRAGCLIGAEHVNNSYECDIPIGAGICAS
YQTQTNSPGSASSVASQSIIAYTMSLGVENSIAYSNNVISIPTNFTISVTTEIIPVSMTK
TSVDCAQYICGDNTECENLLLQYGSFCDQLNRALHGIAVEQDKNLQEVFAQVKQIYKTPP
IKDFGGFNFSQILPDPSKPSKRSFIEDLLFNKVTLADAGFIKQYGDCLGDNAARDLICAQ
SFNGLTVLPPLLTDEMIAAYTSALLAGTITAGWTFGAGAALAIPFALQMAYRFNGIGVTQ
NVLYENQKLIANQFNSAITKIQDSLSSTASALAKLQDVVNQNAQALNTLVKQLSNKFGAI
SSVLNDILSRLDPPEAEVQIDRLITGRLQALQTYVTQQLIRAAEIKASANLAATKMSECV
LGQSKRVNFCGKGYHLMSFPQSAPHGVVFLHVTYVPAQEKNFTTAPAICHDGKAYFPREG
VFVSNGTHWFVTQRNFYEPQIITTDNTFVSGNCDVVIGIVNNTVYDPLQPELDS
SEQ ID NO: 131-B.1.351_Buried_PROSS_1_5
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEPYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQ
GVNCTEVPVAIHADQLTPTWRVYSTGSNVFQTRAGCLIGAEHVNNSYECDIPIGAGICAS
YQTQTNSPGSASSVASQSIIAYTMSLGVENSIAYSNNVIAIPTNFTISVTTEIIPVSMTK
TSVDCAQYICGDNTECENLLLQYGSFCDQLNRALHGIAVEQDKALQEVFAQVKQIYKTPP
IKDFGGFNFSQILPDPSKPSKRSFIEDLLFNKVTLADAGFIKQYGDCLGDNAARDLICAQ
KFNGLTVLPPLLTDEMIAAYTSALLAGTITAGWTFGAGAALAIPFALQMAYRFNGIGVTQ
NVLYENQKLIANQFNSAITKIQDSLSSTASALAKLQDVVNQNAQALNTLVKQLSNNFGAI
SSVLNDILSRLDPPEAEVQIDRLITGRLQALQTYVTQQLIRAAEIKASANLAATKMSECV
LGQSKRVNFCGKGYHLMSFPQSAPHGVVFLHVTYVPAQEKNFTTAPAICHDGKAHFPREG
VFVSNGTHWYVTQRNFYEPQIITTDNTFVSGNCDVVIGIVNNTVYDPLQPELDS
SEQ ID NO: 132-B.1.351_Buried_PROSS_3_0
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEPYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDTADTTDAVRDPQTLETLDTTPCSFGGVSVTTPGTNTSNQVAVLYQ
GVNCTEVPVATHADQLTPTWRVYSTGSNVFQTRAGCLTGAEHVNNSYECDTPTGAGTCAS
YQTQTNSPGSASSVASQSTTAYTMSLGVENSTAYSNNVTATPTNFTTSVTTETTPVSMTK
TSVDCTQYTCGDSTECENLLLQYGSFCDQLNRALHGTAVEQDKNTQEVFAQVKQTYKTPP
TKDFGGFNFSQTLPDPSKPSKRSFTEDLLFNKVTLADAGFTKQYGDCLGDPAARDLTCAQ
KFNGLTVLPPLLTDEMTAAYTSALLAGTTTAGWTFGAGAALATPFAMQMAYRFNGTGVTQ
NVLYENQKLIANQFNSAIGKIQDSLSSTASALAKLQDVVNQNAQALNTLVKQLSNNFGAI
SSVLNDILSRLDPPEAEVQIDRLITGRLQALQTYVTQQLIRAAEIKASANLAATKMSECV
LGQSKRVNFCGKGYHLMSFPQSAPHGVVFLHVTYVPAQEKNFTTAPAICHDGKAHFPREG
VFVSNGTHWFVTQRNFYEPQIITTDNTFVSGNCDVVIGIVNNTVYDPLQPELDS
SEQ ID NO: 133-B.1.351_Buried_PROSS_5_0
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEIYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQ
GVNCTEVPVAIHADQLTPTWRVYSTGSNVFQTRAGCLIGAEHVNNSYECDIPIGAGICAS
YQTQTNSPGSASSVASQSIIAYTMSLGVENSIAYSNNVIAIPTNFTISVTTEIIPVSMTK
TSVDCAQYICGDSTECENLLLQYGSFCTQLNRALHGIAVEQDKNIQEVFAQVKQIYKTPP
IKDFGGFNFSQILPDPSKPSKRSFIEDLLFNKVTLADAGFIKQYGDCLGDPAARDLICAQ
KFNGLTVLPPLLTDEMIAAYTSALLAGTITAGWTFGAGAALAIPFAMQMAYRFNGIGVTQ
NVLYENQKLIANQFNSAIGKIQDSLSSTASALAKLQDVVNQNAQALNTLVKQLSSNFGAI
SSVLNDILSRLDPPEAEVQIDRLITGRLQALQTYVTQQLIRAAEIKASANLAATKMSECV
LGQSKRVDFCGKGYHLMSFPQSAPHGVVFLHVTYVPAQEKNFTTAPAICHDGKAHFPREG
VFVSNGTHWFVTQRNFYEPQIITTDNTFVSGNCDVVIGIVNNTVYDPLQPELDS
SEQ ID NO: 134-B.1.351_Buried_PROSS_6_0
QCVNFTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRGLPQGFSALEPLVDLPIGINITRFQTLLALHISYLTPGD
SSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEPYQAGSTPCNGVKGFNCYFPLQ
SYGFQPTYGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNQVAVLYQ
GVNCTEVPVAIHADQLTPTWRVYSTGSNVFQTRAGCLIGAEHVNNSYECDIPIGAGICAS
YQTQTNSPGSASSVASQSIIAYTMSLGVENSIAYSNNVIAIPTNFTISVTTEIIPVSMTK
TSVDCAQYICGDSTECENLLLQYGSFCTQLNRALTGIAVEQDKNTQEVFAQVKQIYKTPP
IKDFGGFNFSQILPDPSKPSKRSFIEDLLFNKVTLADAGFIKQYGDCLGDPAARDLICAQ
KFNGLTVLPPLLTDEMIAAYTSALLAGTITSGWTFGAGAALAIPFAMQMAYRFNGIGVTQ
NVLYENQKLIANQFNSAIGKIQDSLSSTASALGKLQDVVNQNAQALNTLVKQLSSNFGAI
SSVLNDILSRLDPPEAEVQIDRLITGRLQALQTYVTQQLIRAAEIKASANLAATKMSECV
LGQSKRVDFCGKGYHLMSFPQSAPHGVVFLHVTYVPAQEKNFTTAPAICHDGKAHFPREG
VFVSNGTHWFVTORNFYEPQIITTDNTFVSGNCDVVIGIVNNTVYDPLQPELDS

1-29. (canceled)

30. A betacoronavirus Spike (S) protein, or fragment thereof, comprising an amino acid sequence that has amino acid substitutions, wherein said amino acid substitutions are selected from (A), (B), (C), (D-A), (D-B), (D-C), (D-D), (D-E), (D-F), (E), and (F), wherein:
(A) is:
(a) the substitute amino acids listed throughout rows 3-134 of column #4 in Table 1, wherein each substitute amino acid is located at the position that corresponds to the residue number of SEQ ID NO: 3 that is listed in the same row of column #1 in Table 1;
(b) the substitute amino acids listed throughout rows 3-134 of column #5 in Table 1, wherein each substitute amino acid is located at the position that corresponds to the residue number of SEQ ID NO: 3 that is listed in the same row of column #1 in Table 1;
(c) the substitute amino acids listed throughout rows 3-134 of column #6 in Table 1, wherein each substitute amino acid is located at the position that corresponds to the residue number of SEQ ID NO: 3 that is listed in the same row of column #1 in Table 1;
(d) the substitute amino acids listed throughout rows 3-134 of column #7 in Table 1, wherein each substitute amino acid is located at the position that corresponds to the residue number of SEQ ID NO: 3 that is listed in the same row of column #1 in Table 1;
(e) the substitute amino acids listed throughout rows 3-134 of column #8 in Table 1, wherein each substitute amino acid is located at the position that corresponds to the residue number of SEQ ID NO: 3 that is listed in the same row of column #1 in Table 1;
(f) the substitute amino acids listed throughout rows 3-134 of column #9 in Table 1, wherein each substitute amino acid is located at the position that corresponds to the residue number of SEQ ID NO: 3 that is listed in the same row of column #1 in Table 1;
(g) the substitute amino acids listed throughout rows 3-134 of column #10 in Table 1, wherein each substitute amino acid is located at the position that corresponds to the residue number of SEQ ID NO: 3 that is listed in the same row of column #1 in Table 1;
(h) the substitute amino acids listed throughout rows 3-134 of column #11 in Table 1, wherein each substitute amino acid is located at the position that corresponds to the residue number of SEQ ID NO: 3 that is listed in the same row of column #1 in Table 1;
(i) the substitute amino acids listed throughout rows 3-134 of column #12 in Table 1, wherein each substitute amino acid is located at the position that corresponds to the residue number of SEQ ID NO: 3 that is listed in the same row of column #1 in Table 1; or
(j) the substitute amino acids listed throughout rows 3-134 of column #13 in Table 1, wherein each substitute amino acid is located at the position that corresponds to the residue number of SEQ ID NO: 3 that is listed in the same row of column #1 in Table 1;
(B) is:
(k) the substitute amino acids listed throughout rows 3-145 of column #4 in Table 2, wherein each substitute amino acid is located at the position that corresponds to the residue number of SEQ ID NO: 3 that is listed in the same row of column #1 in Table 2;
(l) the substitute amino acids listed throughout rows 3-145 of column #5 in Table 2, wherein each substitute amino acid is located at the position that corresponds to the residue number of SEQ ID NO: 3 that is listed in the same row of column #1 in Table 2;
(m) the substitute amino acids listed throughout rows 3-145 of column #6 in Table 2, wherein each substitute amino acid is located at the position that corresponds to the residue number of SEQ ID NO: 3 that is listed in the same row of column #1 in Table 2;
(n) the substitute amino acids listed throughout rows 3-145 of column #7 in Table 2, wherein each substitute amino acid is located at the position that corresponds to the residue number of SEQ ID NO: 3 that is listed in the same row of column #1 in Table 2;
(o) the substitute amino acids listed throughout rows 3-145 of column #8 in Table 2, wherein each substitute amino acid is located at the position that corresponds to the residue number of SEQ ID NO: 3 that is listed in the same row of column #1 in Table 2;
(p) the substitute amino acids listed throughout rows 3-145 of column #9 in Table 2, wherein each substitute amino acid is located at the position that corresponds to the residue number of SEQ ID NO: 3 that is listed in the same row of column #1 in Table 2;
(q) the substitute amino acids listed throughout rows 3-145 of column #10 in Table 2, wherein each substitute amino acid is located at the position that corresponds to the residue number of SEQ ID NO: 3 that is listed in the same row of column #1 in Table 2;
(r) the substitute amino acids listed throughout rows 3-145 of column #11 in Table 2, wherein each substitute amino acid is located at the position that corresponds to the residue number of SEQ ID NO: 3 that is listed in the same row of column #1 in Table 2;
(s) the substitute amino acids listed throughout rows 3-145 of column #12 in Table 2, wherein each substitute amino acid is located at the position that corresponds to the residue number of SEQ ID NO: 3 that is listed in the same row of column #1 in Table 2;
(t) the substitute amino acids listed throughout rows 3-145 of column #13 in Table 2, wherein each substitute amino acid is located at the position that corresponds to the residue number of SEQ ID NO: 3 that is listed in the same row of column #1 in Table 2;
(u) the substitute amino acids listed throughout rows 3-145 of column #14 in Table 2, wherein each substitute amino acid is located at the position that corresponds to the residue number of SEQ ID NO: 3 that is listed in the same row of column #1 in Table 2;
(v) the substitute amino acids listed throughout rows 3-145 of column #15 in Table 2, wherein each substitute amino acid is located at the position that corresponds to the residue number of SEQ ID NO: 3 that is listed in the same row of column #1 in Table 2;
(w) the substitute amino acids listed throughout rows 3-145 of column #16 in Table 2, wherein each substitute amino acid is located at the position that corresponds to the residue number of SEQ ID NO: 3 that is listed in the same row of column #1 in Table 2;
(x) the substitute amino acids listed throughout rows 3-145 of column #17 in Table 2, wherein each substitute amino acid is located at the position that corresponds to the residue number of SEQ ID NO: 3 that is listed in the same row of column #1 in Table 2; or
(y) the substitute amino acids listed throughout rows 3-145 of column #18 in Table 2, wherein each substitute amino acid is located at the position that corresponds to the residue number of SEQ ID NO: 3 that is listed in the same row of column #1 in Table 2;
(C) is:
(I) the substitute amino acids listed throughout rows 3-34 of column #4 in Table 3, wherein each substitute amino acid is located at the position that corresponds to the residue number of SEQ ID NO: 3 that is listed in the same row of column #1 in Table 3;
(II) the substitute amino acids listed throughout rows 3-34 of column #5 in Table 3, wherein each substitute amino acid is located at the position that corresponds to the residue number of SEQ ID NO: 3 that is listed in the same row of column #1 in Table 3;
(III) the substitute amino acids listed throughout rows 3-34 of column #6 in Table 3, wherein each substitute amino acid is located at the position that corresponds to the residue number of SEQ ID NO: 3 that is listed in the same row of column #1 in Table 3;
(IV) the substitute amino acids listed throughout rows 3-34 of column #7 in Table 3, wherein each substitute amino acid is located at the position that corresponds to the residue number of SEQ ID NO: 3 that is listed in the same row of column #1 in Table 3; or
(V) the substitute amino acids listed throughout rows 3-34 of column #8 in Table 3, wherein each substitute amino acid is located at the position that corresponds to the residue number of SEQ ID NO: 3 that is listed in the same row of column #1 in Table 3;
(D-A) is:
Glycine (G) at the position that corresponds to residue 588 of the sequence SEQ ID NO: 3,
G at the position that corresponds to residue 656 of the sequence SEQ ID NO: 3,
Serine (S) at the position that corresponds to residue 657 of the sequence SEQ ID NO: 3,
S at the position that corresponds to residue 659 of the sequence SEQ ID NO: 3,
Proline (P) at the position that corresponds to residue 960 of the sequence SEQ ID NO: 3,
P at the position that corresponds to residue 961 of the sequence SEQ ID NO: 3, and
one of (i)-(x):
(i) Cysteines at the positions that correspond to residues 744 and 989 of the sequence SEQ ID NO: 3,
(ii) Cysteines at the positions that correspond to residues 813 and 836 of the sequence SEQ ID NO: 3,
(iii) Cysteines at the positions that correspond to residues 544 and 941 of the sequence SEQ ID NO: 3,
(iv) Cysteines at the positions that correspond to residues 824 and 560 of the sequence SEQ ID NO: 3,
(v) Cysteines at the positions that correspond to residues 387 and 961 of the sequence SEQ ID NO: 3,
(vi) Cysteines at the positions that correspond to residues 357 and 959 of the sequence SEQ ID NO: 3,
(vii) Cysteines at the positions that correspond to residues 356 and 957 of the sequence SEQ ID NO: 3,
(viii) Cysteines at the positions that correspond to residues 15 and 494 of the sequence SEQ ID NO: 3,
(ix) Cysteines at the positions that correspond to residues 496 and 518 of the sequence SEQ ID NO: 3,
(x) Cysteines at the positions that correspond to residues 495 and 538 of the sequence SEQ ID NO: 3;
(D-B) is the substitute amino acids listed throughout rows 3-134 of column #4 in Table 1, wherein each substitute amino acid is located at the position that corresponds to the residue number of SEQ ID NO: 3 that is listed in the same row of column #1 in Table 1, and one of (i)-(iv):
(i) Cysteines at the positions that correspond to residues 744 and 989 of the sequence SEQ ID NO: 3,
(ii) Cysteines at the positions that correspond to residues 813 and 836 of the sequence SEQ ID NO: 3,
(iii) Cysteines at the positions that correspond to residues 544 and 941 of the sequence SEQ ID NO: 3,
(iv) Cysteines at the positions that correspond to residues 824 and 560 of the sequence SEQ ID NO: 3;
(D-C) is the substitute amino acids listed throughout rows 3-134 of column #9 in Table 1, wherein each substitute amino acid is located at the position that corresponds to the residue number of SEQ ID NO: 3 that is listed in the same row of column #1 in Table 1, and one of (i)-(iv):
(i) Cysteines at the positions that correspond to residues 744 and 989 of the sequence SEQ ID NO: 3,
(ii) Cysteines at the positions that correspond to residues 813 and 836 of the sequence SEQ ID NO: 3,
(iii) Cysteines at the positions that correspond to residues 544 and 941 of the sequence SEQ ID NO: 3,
(iv) Cysteines at the positions that correspond to residues 824 and 560 of the sequence SEQ ID NO: 3;
(D-D) is the substitute amino acids listed throughout rows 3-145 of column #13 in Table 2, wherein each substitute amino acid is located at the position that corresponds to the residue number of SEQ ID NO: 3 that is listed in the same row of column #1 in Table 2, and one of (i)-(iv):
(i) Cysteines at the positions that correspond to residues 744 and 989 of the sequence SEQ ID NO: 3,
(ii) Cysteines at the positions that correspond to residues 813 and 836 of the sequence SEQ ID NO: 3,
(iii) Cysteines at the positions that correspond to residues 544 and 941 of the sequence SEQ ID NO: 3,
(iv) Cysteines at the positions that correspond to residues 824 and 560 of the sequence SEQ ID NO: 3;
(D-E) is the substitute amino acids listed throughout rows 3-145 of column #18 in Table 2, wherein each substitute amino acid is located at the position that corresponds to the residue number of SEQ ID NO: 3 that is listed in the same row of column #1 in Table 2, and one of (i)-(iv):
(i) Cysteines at the positions that correspond to residues 744 and 989 of the sequence SEQ ID NO: 3,
(ii) Cysteines at the positions that correspond to residues 813 and 836 of the sequence SEQ ID NO: 3,
(iii) Cysteines at the positions that correspond to residues 544 and 941 of the sequence SEQ ID NO: 3,
(iv) Cysteines at the positions that correspond to residues 824 and 560 of the sequence SEQ ID NO: 3;
(D-F) is the substitute amino acids listed throughout rows 3-34 of column #4 in Table 3, wherein each substitute amino acid is located at the position that corresponds to the residue number of SEQ ID NO: 3 that is listed in the same row of column #1 in Table 3, and one of (i)-(iv):
(i) Cysteines at the positions that correspond to residues 744 and 989 of the sequence SEQ ID NO: 3,
(ii) Cysteines at the positions that correspond to residues 813 and 836 of the sequence SEQ ID NO: 3,
(iii) Cysteines at the positions that correspond to residues 544 and 941 of the sequence SEQ ID NO: 3,
(iv) Cysteines at the positions that correspond to residues 824 and 560 of the sequence SEQ ID NO: 3;
(E) is:
Glycine (G) at the position that corresponds to residue 588 of the sequence SEQ ID NO: 3,
G at the position that corresponds to residue 656 of the sequence SEQ ID NO: 3,
Serine (S) at the position that corresponds to residue 657 of the sequence SEQ ID NO: 3,
S at the position that corresponds to residue 659 of the sequence SEQ ID NO: 3,
Proline (P) at the position that corresponds to residue 960 of the sequence SEQ ID NO: 3,
P at the position that corresponds to residue 961 of the sequence SEQ ID NO: 3, and
one of (i)-(xi):
(i) F, L, M, W, or Y at the position that corresponds to residue 391 of the sequence SEQ ID NO: 3;
(ii) A at the position that corresponds to residue 423 of the sequence SEQ ID NO: 3;
(iii) A at the position that corresponds to residue 427 of the sequence SEQ ID NO: 3;
(iv) A, H, M, N, or W at the position that corresponds to residue 429 of the sequence SEQ ID NO: 3;
(v) H, I, W, or Y at the position that corresponds to residue 430 of the sequence SEQ ID NO: 3;
(vi) W at the position that corresponds to residue 447 of the sequence SEQ ID NO: 3;
(vii) M at the position that corresponds to residue 449 of the sequence SEQ ID NO: 3;
(viii) T at the position that corresponds to residue 450 of the sequence SEQ ID NO: 3;
(ix) H, I, L, M, N, P, T, W, or Y at the position that corresponds to residue 460 of the sequence SEQ ID NO: 3;
(x) F, L, M, or Q at the position that corresponds to residue 461 of the sequence SEQ ID NO: 3; or
(xi) A, Y, F, R, M, C, G, or V at the position that corresponds to residue 467 of the sequence SEQ ID NO: 3; and
(F) is:
Glycine (G) at the position that corresponds to residue 588 of the sequence SEQ ID NO: 3,
G at the position that corresponds to residue 656 of the sequence SEQ ID NO: 3,
Serine (S) at the position that corresponds to residue 657 of the sequence SEQ ID NO: 3,
S at the position that corresponds to residue 659 of the sequence SEQ ID NO: 3,
Proline (P) at the position that corresponds to residue 960 of the sequence SEQ ID NO: 3,
P at the position that corresponds to residue 961 of the sequence SEQ ID NO: 3, and
one of (i)-(x):
(i) N at the position that corresponds to residue 391 of the sequence SEQ ID NO: 3 and T at the position that corresponds to residue 393 of the sequence SEQ ID NO: 3;
(ii) N at the position that corresponds to residue 423 of the sequence SEQ ID NO: 3 and T at the position that corresponds to residue 425 of the sequence SEQ ID NO: 3;
(iii) N at the position that corresponds to residue 427 of the sequence SEQ ID NO: 3 and T at the position that corresponds to residue 429 of the sequence SEQ ID NO: 3;
(iv) N at the position that corresponds to residue 429 of the sequence SEQ ID NO: 3 and T at the position that corresponds to residue 431 of the sequence SEQ ID NO: 3;
(v) N at the position that corresponds to residue 430 of the sequence SEQ ID NO: 3 and T at the position that corresponds to residue 432 of the sequence SEQ ID NO: 3;
(vi) N at the position that corresponds to residue 447 of the sequence SEQ ID NO: 3 and T at the position that corresponds to residue 449 of the sequence SEQ ID NO: 3;
(vii) N at the position that corresponds to residue 449 of the sequence SEQ ID NO: 3 and T at the position that corresponds to residue 451 of the sequence SEQ ID NO: 3;
(viii) N at the position that corresponds to residue 450 of the sequence SEQ ID NO: 3;
(ix) T at the position that corresponds to residue 463 of the sequence SEQ ID NO: 3; or
(x) N at the position that corresponds to residue 467 of the sequence SEQ ID NO: 3 and T at the position that corresponds to residue 469 of the sequence SEQ ID NO: 3.

31. The betacoronavirus Spike (S) protein, or fragment thereof, of claim 30, wherein (A) is selected, and comprising:
an amino acid sequence that has the substitutions of (a) and has at least 80% sequence identity to the entire sequence SEQ ID NO: 5,
an amino acid sequence that has the substitutions of (b) and has at least 80% sequence identity to the entire sequence SEQ ID NO: 6,
an amino acid sequence that has the substitutions of (c) and has at least 80% sequence identity to the entire sequence SEQ ID NO: 7,
an amino acid sequence that has the substitutions of (d) and has at least 80% sequence identity to the entire sequence SEQ ID NO: 8,
an amino acid sequence that has the substitutions of (e) and has at least 80% sequence identity to the entire sequence SEQ ID NO: 9,
an amino acid sequence that has the substitutions of (f) and has at least 80% sequence identity to the entire sequence SEQ ID NO: 10,
an amino acid sequence that has the substitutions of (g) and has at least 80% sequence identity to the entire sequence SEQ ID NO: 11,
an amino acid sequence that has the substitutions of (h) and has at least 80% sequence identity to the entire sequence SEQ ID NO: 12,
an amino acid sequence that has the substitutions of (i) and has at least 80% sequence identity to the entire sequence SEQ ID NO: 13, or
an amino acid sequence that has the substitutions of (j) and has at least 80% sequence identity to the entire sequence SEQ ID NO: 14.

32. The betacoronavirus Spike (S) protein, or fragment thereof, of claim 30, wherein (B) is selected, and comprising:
an amino acid sequence that has the substitutions of (k) and has at least 80% sequence identity to the entire sequence SEQ ID NO: 15,
an amino acid sequence that has the substitutions of (l) and has at least 80% sequence identity to the entire sequence SEQ ID NO: 16,
an amino acid sequence that has the substitutions of (m) and has at least 80% sequence identity to the entire sequence SEQ ID NO: 17,
an amino acid sequence that has the substitutions of (n) and has at least 80% sequence identity to the entire sequence SEQ ID NO: 18,
an amino acid sequence that has the substitutions of (o) and has at least 80% sequence identity to the entire sequence SEQ ID NO: 19,
an amino acid sequence that has the substitutions of (p) and has at least 80% sequence identity to the entire sequence SEQ ID NO: 20,
an amino acid sequence that has the substitutions of (q) and has at least 80% sequence identity to the entire sequence SEQ ID NO: 21,
an amino acid sequence that has the substitutions of (r) and has at least 80% sequence identity to the entire sequence SEQ ID NO: 22,
an amino acid sequence that has the substitutions of (s) and has at least 80% sequence identity to the entire sequence SEQ ID NO: 23,
an amino acid sequence that has the substitutions of (t) and has at least 80% sequence identity to the entire sequence SEQ ID NO: 24,
an amino acid sequence that has the substitutions of (u) and has at least 80% sequence identity to the entire sequence SEQ ID NO: 25,
an amino acid sequence that has the substitutions of (v) and has at least 80% sequence identity to the entire sequence SEQ ID NO: 26,
an amino acid sequence that has the substitutions of (w) and has at least 80% sequence identity to the entire sequence SEQ ID NO: 27,
an amino acid sequence that has the substitutions of (x) and has at least 80% sequence identity to the entire sequence SEQ ID NO: 28, or
an amino acid sequence that has the substitutions of (y) and has at least 80% sequence identity to the entire sequence SEQ ID NO: 29.

33. The betacoronavirus Spike (S) protein, or fragment thereof, of claim 30, wherein (C) is selected, and comprising:
an amino acid sequence that has the substitutions of (I) and has at least 80% sequence identity to the entire sequence SEQ ID NO: 30,
an amino acid sequence that has the substitutions of (II) and has at least 80% sequence identity to the entire sequence SEQ ID NO: 31,
an amino acid sequence that has the substitutions of (III) and has at least 80% sequence identity to the entire sequence SEQ ID NO: 32,
an amino acid sequence that has the substitutions of (IV) and has at least 80% sequence identity to the entire sequence SEQ ID NO: 33, or
an amino acid sequence that has the substitutions of (V) and has at least 80% sequence identity to the entire sequence SEQ ID NO: 34.

34. The betacoronavirus Spike (S) protein, or fragment thereof, of claim 30, wherein one of (D-A), (D-B), (D-C), (D-D), (D-E), and (D-F) is selected, and comprising:
an amino acid sequence that has the substitutions of (D-A), (i) is selected, and has at least 80% sequence identity to the entire sequence SEQ ID NO: 35,
an amino acid sequence that has the substitutions of (D-A), (ii) is selected, and has at least 80% sequence identity to the entire sequence SEQ ID NO: 36,
an amino acid sequence that has the substitutions of (D-A), (iii) is selected, and has at least 80% sequence identity to the entire sequence SEQ ID NO: 37,
an amino acid sequence that has the substitutions of (D-A), (iv) is selected, and has at least 80% sequence identity to the entire sequence SEQ ID NO: 38,
an amino acid sequence that has the substitutions of (D-A), (v) is selected, and has at least 80% sequence identity to the entire sequence SEQ ID NO: 39,
an amino acid sequence that has the substitutions of (D-A), (vi) is selected, and has at least 80% sequence identity to the entire sequence SEQ ID NO: 40,
an amino acid sequence that has the substitutions of (D-A), (vii) is selected, and has at least 80% sequence identity to the entire sequence SEQ ID NO: 41,
an amino acid sequence that has the substitutions of (D-A), (viii) is selected, and has at least 80% sequence identity to the entire sequence SEQ ID NO: 42,
an amino acid sequence that has the substitutions of (D-A), (ix) is selected, and has at least 80% sequence identity to the entire sequence SEQ ID NO: 43,
an amino acid sequence that has the substitutions of (D-A), (x) is selected, and has at least 80% sequence identity to the entire sequence SEQ ID NO: 44,
an amino acid sequence that has the substitutions of (D-B), (i) is selected, and has at least 80% sequence identity to the entire sequence SEQ ID NO: 45,
an amino acid sequence that has the substitutions of (D-B), (ii) is selected, and has at least 80% sequence identity to the entire sequence SEQ ID NO: 50,
an amino acid sequence that has the substitutions of (D-B), (iii) is selected, and has at least 80% sequence identity to the entire sequence SEQ ID NO: 55,
an amino acid sequence that has the substitutions of (D-B), (iv) is selected, and has at least 80% sequence identity to the entire sequence SEQ ID NO: 60,
an amino acid sequence that has the substitutions of (D-C), (i) is selected, and has at least 80% sequence identity to the entire sequence SEQ ID NO: 46,
an amino acid sequence that has the substitutions of (D-C), (ii) is selected, and has at least 80% sequence identity to the entire sequence SEQ ID NO: 51,
an amino acid sequence that has the substitutions of (D-C), (iii) is selected, and has at least 80% sequence identity to the entire sequence SEQ ID NO: 56,
an amino acid sequence that has the substitutions of (D-C), (iv) is selected, and has at least 80% sequence identity to the entire sequence SEQ ID NO: 61,
an amino acid sequence that has the substitutions of (D-D), (i) is selected, and has at least 80% sequence identity to the entire sequence SEQ ID NO: 47,
an amino acid sequence that has the substitutions of (D-D), (ii) is selected, and has at least 80% sequence identity to the entire sequence SEQ ID NO: 52,
an amino acid sequence that has the substitutions of (D-D), (iii) is selected, and has at least 80% sequence identity to the entire sequence SEQ ID NO: 57,
an amino acid sequence that has the substitutions of (D-D), (iv) is selected, and has at least 80% sequence identity to the entire sequence SEQ ID NO: 62,
an amino acid sequence that has the substitutions of (D-E), (i) is selected, and has at least 80% sequence identity to the entire sequence SEQ ID NO: 48,
an amino acid sequence that has the substitutions of (D-E), (ii) is selected, and has at least 80% sequence identity to the entire sequence SEQ ID NO: 53,
an amino acid sequence that has the substitutions of (D-E), (iii) is selected, and has at least 80% sequence identity to the entire sequence SEQ ID NO: 58,
an amino acid sequence that has the substitutions of (D-E), (iv) is selected, and has at least 80% sequence identity to the entire sequence SEQ ID NO: 63,
an amino acid sequence that has the substitutions of (D-F), (i) is selected, and has at least 80% sequence identity to the entire sequence SEQ ID NO: 49,
an amino acid sequence that has the substitutions of (D-F), (ii) is selected, and has at least 80% sequence identity to the entire sequence SEQ ID NO: 54,
an amino acid sequence that has the substitutions of (D-F), (iii) is selected, and has at least 80% sequence identity to the entire sequence SEQ ID NO: 59, or
an amino acid sequence that has the substitutions of (D-F), (iv) is selected, and has at least 80% sequence identity to the entire sequence SEQ ID NO: 64.

35. The betacoronavirus Spike (S) protein, or fragment thereof, of claim 30, wherein (E) is selected, and comprising an amino acid sequence that has at least 80% sequence identity to the entire sequence of one or more of SEQ ID NOs: 65-104.

36. The betacoronavirus Spike (S) protein, or fragment thereof, of claim 30, wherein (F) is selected, and comprising:
an amino acid sequence that has the substitutions of (i) and has at least 80% sequence identity to the entire sequence SEQ ID NO: 105,
an amino acid sequence that has the substitutions of (ii) and has at least 80% sequence identity to the entire sequence SEQ ID NO: 106,
an amino acid sequence that has the substitutions of (iii) and has at least 80% sequence identity to the entire sequence SEQ ID NO: 107,
an amino acid sequence that has the substitutions of (iv) and has at least 80% sequence identity to the entire sequence SEQ ID NO: 108,
an amino acid sequence that has the substitutions of (v) and has at least 80% sequence identity to the entire sequence SEQ ID NO: 109,
an amino acid sequence that has the substitutions of (vi) and has at least 80% sequence identity to the entire sequence SEQ ID NO: 110,
an amino acid sequence that has the substitutions of (vii) and has at least 80% sequence identity to the entire sequence SEQ ID NO: 111,
an amino acid sequence that has the substitutions of (viii) and has at least 80% sequence identity to the entire sequence SEQ ID NO: 112,
an amino acid sequence that has the substitutions of (ix) and has at least 80% sequence identity to the entire sequence SEQ ID NO: 113, or
an amino acid sequence that has the substitutions of (x) and has at least 80% sequence identity to the entire sequence SEQ ID NO: 114.

37. The betacoronavirus S protein, or S protein fragment, of claim 30, comprising an amino acid sequence with at least 80% sequence identity to the entire sequence of one or more of SEQ ID NOs: 5-114.

38. A betacoronavirus Spike (S) protein, or fragment thereof, claim 30, wherein (A) is selected, which comprises one of the following SEQ ID NOs: 22-29.

39. A nucleic acid molecule comprising a polynucleotide sequence that encodes the betacoronavirus S protein, or S protein fragment, of claim 30.

40. The nucleic acid molecule of claim 39 that is a Self-Amplifying RNA Molecule comprising, from 5′-3′, a polynucleotide comprising the sequence SEQ ID NO: 119; a polynucleotide sequence that encodes the betacoronavirus S protein, or S protein fragment;
and a polynucleotide comprising the sequence SEQ ID NO: 120.

41. A betacoronavirus Spike (S) protein, or fragment thereof, comprising an amino acid sequence that has amino acid substitutions, wherein said amino acid substitutions are (A) or (B), wherein:
(A) is:
G at the position that corresponds to residue 202 of any of SEQ ID NOS:125-134;
Asparagine (N) at the position that corresponds to residue 404 of any of SEQ ID NOS:125-134;
Lysine (K) at the position that corresponds to residue 471 of any of SEQ ID NOS:125-134;
Tyrosine (Y) at the position that corresponds to residue 488 of any of SEQ ID NOS:125-134;
G at the position that corresponds to residue 601 of any of SEQ ID NOS:125-134;
Isoleucine (I) at the position that corresponds to residue 692 and Glutamine (Q) that corresponds to residue 727 of any of SEQ ID NOS:125-134; and
one of (i)-(v)
(i) P at the positions that correspond to residues 691, 693, 818, and 1101 of any of SEQ ID NOS:125-134;
(ii) Glutamate (E) at the position that corresponds to residue 756 of any of SEQ ID NOS:125-134;
(iii) Y at the position that corresponds to residue 801 of any of SEQ ID NOS:125-134;
(iv) Serine (S) at the position that corresponds to residue 879 of any of SEQ ID NOS:125-134; and
(v) K at the position that corresponds to residue 916 of any of SEQ ID NOS:125-134; and
(B) is:
G at the position that corresponds to residue 202 of any of SEQ ID NOS:125-134;
Asparagine (N) at the position that corresponds to residue 404 of any of SEQ ID NOS:125-134;
Lysine (K) at the position that corresponds to residue 471 of any of SEQ ID NOS:125-134;
Tyrosine (Y) at the position that corresponds to residue 488 of any of SEQ ID NOS:125-134;
G at the position that corresponds to residue 601 of any of SEQ ID NOS:125-134;
Isoleucine (I) at the position that corresponds to residue 692 and Glutamine (Q) that corresponds to residue 727 of any of SEQ ID NOS:125-134; and
one of (i)-(v):
(i) S at the position that corresponds to residue 691 of any of SEQ ID NOS:125-134;
(ii) A at the positions that correspond to residues 693 and 818 of any of SEQ ID NOS:125-134;
(iii) I at the position that corresponds to residue 1101 of any of SEQ ID NOS:125-134;
(iv) G at the position that corresponds to residue 756 of any of SEQ ID NOS:125-134;
(v) K at the position that corresponds to residue 801 of any of SEQ ID NOS:125-134;
(iv) A at the position that corresponds to residue 879 of any of SEQ ID NOS:125-134; and
(v) S at the position that corresponds to residue 916 of any of SEQ ID NOS:125-134.

42. The betacoronavirus Spike (S) protein, or fragment thereof, of claim 41 comprising:
an amino acid sequence that has the substitutions of (A), (i) is selected, and has at least 80% sequence identity to the entire sequence SEQ ID NO: 125;
an amino acid sequence that has the substitutions of (A), (i) is selected, and has at least 80% sequence identity to the entire sequence SEQ ID NO: 126;
an amino acid sequence that has the substitutions of (A), (i) is selected, and has at least 80% sequence identity to the entire sequence SEQ ID NO: 127;
an amino acid sequence that has the substitutions of (A), (i) is selected, and has at least 80% sequence identity to the entire sequence SEQ ID NO: 128; or
an amino acid sequence that has the substitutions of (A), (i) is selected, and has at least 80% sequence identity to the entire sequence SEQ ID NO: 129.

43. The betacoronavirus Spike (S) protein, or fragment thereof, of claim 42, comprising an amino acid sequence of any one of SEQ ID NOs: 125-129.

44. The betacoronavirus Spike (S) protein, or fragment thereof, of claim 41 comprising:
an amino acid sequence that has the substitutions of (A), (i) is selected, and has at least 80% sequence identity to the entire sequence SEQ ID NO: 130;
an amino acid sequence that has the substitutions of (A), (i) is selected, and has at least 80% sequence identity to the entire sequence SEQ ID NO: 131;
an amino acid sequence that has the substitutions of (A), (i) is selected, and has at least 80% sequence identity to the entire sequence SEQ ID NO: 132;
an amino acid sequence that has the substitutions of (A), (i) is selected, and has at least 80% sequence identity to the entire sequence SEQ ID NO: 133; or
an amino acid sequence that has the substitutions of (A), (i) is selected, and has at least 80% sequence identity to the entire sequence SEQ ID NO: 134.

45. The betacoronavirus Spike (S) protein, or fragment thereof, of claim 44, comprising an amino acid sequence of any one of SEQ ID NOs: 130-134.

46. A nucleic acid molecule comprising a polynucleotide sequence that encodes the betacoronavirus S protein, or S protein fragment, of claim 41.

47. The nucleic acid molecule of claim 46 that is a Self-Amplifying RNA Molecule comprising, from 5′-3′, a polynucleotide comprising the sequence SEQ ID NO: 119; a polynucleotide sequence that encodes the betacoronavirus S protein, or S protein fragment;
and a polynucleotide comprising the sequence SEQ ID NO: 120.

48. An immunogenic composition comprising (i) the betacoronavirus S protein, or S protein fragment of claim 30, optionally further comprising an adjuvant; or (ii) a nucleic acid molecule that encodes the betacoronavirus S protein, or S protein fragment.

49. A method of inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases; comprising
delivering to a subject an immunologically effective amount of the immunogenic composition of claim 48.

50. An immunogenic composition comprising (i) the betacoronavirus S protein, or S protein fragment of claim 41, optionally further comprising an adjuvant; or (ii) a nucleic acid molecule that encodes the betacoronavirus S protein, or S protein fragment.

51. A method of inducing an immune response against betacoronavirus; inducing neutralizing antibodies against betacoronavirus; reducing cell entry by betacoronavirus; reducing cell-to-cell spread of betacoronavirus; reducing betacoronavirus entry into cells; or preventing, or reducing the severity of, betacoronavirus-associated diseases;
comprising delivering to a subject an immunologically effective amount of the immunogenic composition of claim 50.
